SEQUENCE LISTING (229 sequences)

SEQ ID NO: 1 - DNA, 30 bp, synthetic construct
  misc_feature 1..30: note = A 30 nucleotide sequence representing the 5' junction region ofcorn genomic DNA and the integrated transgenic expressioncassette.
  source 1..30: mol_type = other DNA
acgctggatccgaaggacgtgtctacattc

SEQ ID NO: 2 - DNA, 30 bp, synthetic construct
  misc_feature 1..30: note = A 30 nucleotide sequence representing the 3' junction regions ofthe integrated transgenic expression cassette and the corngenomic DNA.
  source 1..30: mol_type = other DNA
ccgcacaacaaacgcgacgactaacccgca

SEQ ID NO: 3 - DNA, 60 bp, synthetic construct
  misc_feature 1..60: note = A 60 nucleotide sequence representing the 5' junction region ofcorn genomic DNA and the integrated transgenic expressioncassette.
  source 1..60: mol_type = other DNA
taatccgggccatcgacgctggatccgaaggacgtgtctacattcacgtccaaatggggg

SEQ ID NO: 4 - DNA, 60 bp, synthetic construct
  misc_feature 1..60: note = A 60 nucleotide sequence representing the 3' junction region ofthe integrated transgenic expression cassette and the corngenomic DNA.
  source 1..60: mol_type = other DNA
gaggttgatttgcggccgcacaacaaacgcgacgactaacccgcaaatggcgcggctcgc

SEQ ID NO: 5 - DNA, 100 bp, synthetic construct
  misc_feature 1..100: note = A 100 nucleotide sequence representing the 3' junction region ofthe integrated transgenic expression cassette and the corngenomic DNA.
  source 1..100: mol_type = other DNA
ccgaaccgataaccctcttctaatccgggccatcgacgctggatccgaaggacgtgtctacattcacgtccaaatgggggcttagatgagaaacttcacg

SEQ ID NO: 6 - DNA, 100 bp, synthetic construct
  misc_feature 1..100: note = A 100 nucleotide sequence representing the 3' junction region ofthe integrated transgenic expression cassette and the corngenomic DNA.
  source 1..100: mol_type = other DNA
ccacctcatttaaatagagtgaggttgatttgcggccgcacaacaaacgcgacgactaacccgcaaatggcgcggctcgccagagtaatgctagggtggt

SEQ ID NO: 7 - DNA, 1180 bp, synthetic construct
  misc_feature 1..1180: note = A 1,180 nucleotide sequence representing 1,000 nucleotides of 5'flanking corn genomic DNA and 180 nucleotides of inserted T-DNA.
  source 1..1180: mol_type = other DNA
gattaacaagaacattattggttaaaaaagagtgatcaagggcacaactttccttcaacgagctcctgctcagtattttccatctgctgggtaccaggttccttggtcacttgctcgtctactcgtaacaatacaaacaaacatggtataggagaaattaacatcacaacaaacatgagcacaaactgcataataatgatctatgcgttgctacgagatcgtaggttcgggaactactaaattcggagttaaagtaaacaagatatggttttccaaagtgtaagtgactataattcatgtgaaaaatatattaaacagtagtaactgatatattccaggtcataacttgatcctagctcttgttttggtcaaacagtaattatgagcaatttaattcttgctaaaagttatttacataaacacaagctacatagatatctaattatagaagtatgggagatgtcacaattagtactattaatgcgtagataattttattacgaagataacgtaattggaacgggttaaaaaggagttaaaataagtaaaatatgaattttacaagtttttagattcacttttatattaaacattcattttccaatcttatttacccaggtcaaataatccatggacagcggacgcaaaaaccagggcgtttagggttcagtttgcaaattccgggacctgggggtaatgatctttgtgtacaggtgtactacgggttgattagtaaaaacaccagggctcttatgaaaaatcgcttggccgaaagggtatcatgcaatcagggccatggatcttggatctacggtcaggattaaattatcgttactatgaatcgatattcaataggaaccctaggataggatatgcacgactcagattttatgttgaccaaatggacgtggttcacccgatctatcatcaacgctcctgattccttactcccgaaccgataaccctcttctaatccgggccatcgacgctggatccgaaggacgtgtctacattcacgtccaaatgggggcttagatgagaaacttcacgatttggcgcgactaactaagcactagcgtacgggacccagatatcgaattcaagcttataacttcgtataatgtatgctatacgaagttatgtcgactaactataacggtcctaaggtagcgacttaggc

SEQ ID NO: 8 - DNA, 1107 bp, synthetic construct
  misc_feature 1..1107: note = A 1,107 nucleotide sequence representing 107 nucleotides ofinserted T-DNA and 1,000 nucleotides of 3' flanking corn genomicDNA after the inserted T-DNA.
  source 1..1107: mol_type = other DNA
cctgcaggtgtttaaactagggataacagggtaataggtctcacgcggcaaatcctaccacctcatttaaatagagtgaggttgatttgcggccgcacaacaaacgcgacgactaacccgcaaatggcgcggctcgccagagtaatgctagggtggtgcatcaatggataaaaacctgccgcacgcaggcacaaacgctaaagggaccaatggcgaggaagaacataggggtagtttacggttgcccggtgcagggacgagcagttcacgctccgcggtagcctggtccggtcttctacgtacgacgatgaagtcctgttctccagtcaccaaacacgcaagggaagcccccaagcgtccagcgcgcgaccctgccgaacccccagcttgacgccaagattcaagggcagcggcagcggccatttccaatgcgttctctctccgtgggtctattcccagcggctatggctctcagtactggttatgggcgcatgatgcagagagatcaaccgagagtgtgcgatctggtatttattcagggagtccaggttctgctggggagggagatattagctggcagactccaggcttgttgcaacggccaagtggagttgtgagtttgttgtgtcggggcctcagcgaaggcgaaggtgctgacaagcggtccccacggtgcagtgtgtgtggttgagcgcggcgtggtgcgggagccgtgcaatgcggatccggattaggccggtgttgggctgaacgagcagcaaggagattcaggttgggttctgtttcatttttatttttttttctcttttctctctattttttcatttcacaagcccaattttttttcaaatccaaacaaggttctaattcaaaaactccataggtgcaaattattatttttgggatgtagtttaaccatatagtttattatattatcgttcactacttatttgatttggaaacaaagtaacatatagtctttcatcacaaattacttgagttcaattatattttaaaaattatttctaatacaaattaaatgaacagtcaaaatctaaggcatgatgcataatttcaacttctatttcatttttatagggaacatgt

SEQ ID NO: 9 - DNA, 2733 bp, synthetic construct
  misc_feature 1..2733: note = A 2,733 nucleotide sequence corresponding to the transgenicinserted T-DNA of corn event ZM_BCS216090.
  source 1..2733: mol_type = other DNA
gacgtgtctacattcacgtccaaatgggggcttagatgagaaacttcacgatttggcgcgactaactaagcactagcgtacgggacccagatatcgaattcaagcttataacttcgtataatgtatgctatacgaagttatgtcgactaactataacggtcctaaggtagcgacttaggctgagcccgggcaggcctacccataatacccataatagctgtttgccaatcgttcttcttggcgcgccaagacgcaaactcggaccgacggtaccctcagcgctgtgcctgttgcgatcctacaaaagggagtagtaatatttaatgagcttgaaggaggatatcaactctctccaaggtttattggagacctttatgctcatggttttattaaacaaataaacttcacaaccaaggttcctgaagggctaccgccaatcatagcggaaaaacttcaagactataagttccctggatcaaataccgtcttaatagaacgagagattcctcgctggaacttcaatgaaatgaaaagagaaacacagatgaggaccaacttatatatcttcaagaattatcgctgtttctatggctattcaccattaaggccatacgaacctataactcctgaagaatttgggtttgattactacagttgggaaaatatggttgatgaagacgaaggagaagttgtatacatctccaagtatactaagattatcaaagtcactaaagagcatgcatgggcttggccagaacatgatggagacacaatgtcctgcaccacatcaatagaagatgaatggatccatcgtatggacaatgcttaaagaagctttatcaaaagcaactttaagtacgaatcaataaagaaggaccagaagatataaagcgggaacatcttcacatgctaccacatggctagcatctttactttagcatctctattattgtaagagtgtataatgaccagtgtgcccctggactccagtatataaggagcaccagagtagtgtaatagatcatcgatcaagcaagcgagacgtcaaacttctaagagagcctgcaggaccaggtgggcccaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggggcagagccgtgcccgtctcatcccctgcccgtgcaagcagctaggtaggacgatttgagcgtggtgttaggccgaaccgctgaaggaagattgctccactgttgactgcattagttgcaccgcatgatggagaaatgtattgcttatattcagcaatataatgttcctccatcatccagtgcaattaatatagtcgatagtggaagaacggtaacatatgtggtttgcagcaggtgagcaggatgggtgtggatgattgaatatctctgttcagtgttttcatcatctgactgaacactgaatcagcttgctgacgttagaggtttcagtttacctaatttatggtctgtacccatgaaaagtgggaaaaggctgaagaattcgatttctttctttctttcaatgtttcgccagcagaacacgcgctgaggttaattaatccagggcgcctgcttgtttgctgccaagagagtgttttgtgtactgctgctgccgagaaatatatatttttttctttcaccacctcgtgtgcagcagttgttttttgtttggatggataaatgtttctagtactgtggaggctgcatctgcatctgtttgtaaatggatgaaatatgaataaaaagttttgtttctcataccccatgtgtcttgtgtttgcatgcacgccgtgctagtttggtttttggtttctagagaaaacattttgtttgcttgtttctctataggatgtgaagaacctgcaggtgtttaaactagggataacagggtaataggtctcacgcggcaaatcctaccacctcatttaaatagagtgaggttgatttgcggccgcacaacaaacgc

SEQ ID NO: 10 - DNA, 4733 bp, synthetic construct
  misc_feature 1..4733: note = A 5,731 nucleotide sequence corresponding to the contignucleotide sequence of the 5' genomic flanking DNA, the insertedT-DNA, and the 3' flanking corn genomic DNA corresponding toevent ZM_BCS216090.
  source 1..4733: mol_type = other DNA
gattaacaagaacattattggttaaaaaagagtgatcaagggcacaactttccttcaacgagctcctgctcagtattttccatctgctgggtaccaggttccttggtcacttgctcgtctactcgtaacaatacaaacaaacatggtataggagaaattaacatcacaacaaacatgagcacaaactgcataataatgatctatgcgttgctacgagatcgtaggttcgggaactactaaattcggagttaaagtaaacaagatatggttttccaaagtgtaagtgactataattcatgtgaaaaatatattaaacagtagtaactgatatattccaggtcataacttgatcctagctcttgttttggtcaaacagtaattatgagcaatttaattcttgctaaaagttatttacataaacacaagctacatagatatctaattatagaagtatgggagatgtcacaattagtactattaatgcgtagataattttattacgaagataacgtaattggaacgggttaaaaaggagttaaaataagtaaaatatgaattttacaagtttttagattcacttttatattaaacattcattttccaatcttatttacccaggtcaaataatccatggacagcggacgcaaaaaccagggcgtttagggttcagtttgcaaattccgggacctgggggtaatgatctttgtgtacaggtgtactacgggttgattagtaaaaacaccagggctcttatgaaaaatcgcttggccgaaagggtatcatgcaatcagggccatggatcttggatctacggtcaggattaaattatcgttactatgaatcgatattcaataggaaccctaggataggatatgcacgactcagattttatgttgaccaaatggacgtggttcacccgatctatcatcaacgctcctgattccttactcccgaaccgataaccctcttctaatccgggccatcgacgctggatccgaaggacgtgtctacattcacgtccaaatgggggcttagatgagaaacttcacgatttggcgcgactaactaagcactagcgtacgggacccagatatcgaattcaagcttataacttcgtataatgtatgctatacgaagttatgtcgactaactataacggtcctaaggtagcgacttaggctgagcccgggcaggcctacccataatacccataatagctgtttgccaatcgttcttcttggcgcgccaagacgcaaactcggaccgacggtaccctcagcgctgtgcctgttgcgatcctacaaaagggagtagtaatatttaatgagcttgaaggaggatatcaactctctccaaggtttattggagacctttatgctcatggttttattaaacaaataaacttcacaaccaaggttcctgaagggctaccgccaatcatagcggaaaaacttcaagactataagttccctggatcaaataccgtcttaatagaacgagagattcctcgctggaacttcaatgaaatgaaaagagaaacacagatgaggaccaacttatatatcttcaagaattatcgctgtttctatggctattcaccattaaggccatacgaacctataactcctgaagaatttgggtttgattactacagttgggaaaatatggttgatgaagacgaaggagaagttgtatacatctccaagtatactaagattatcaaagtcactaaagagcatgcatgggcttggccagaacatgatggagacacaatgtcctgcaccacatcaatagaagatgaatggatccatcgtatggacaatgcttaaagaagctttatcaaaagcaactttaagtacgaatcaataaagaaggaccagaagatataaagcgggaacatcttcacatgctaccacatggctagcatctttactttagcatctctattattgtaagagtgtataatgaccagtgtgcccctggactccagtatataaggagcaccagagtagtgtaatagatcatcgatcaagcaagcgagacgtcaaacttctaagagagcctgcaggaccaggtgggcccaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggggcagagccgtgcccgtctcatcccctgcccgtgcaagcagctaggtaggacgatttgagcgtggtgttaggccgaaccgctgaaggaagattgctccactgttgactgcattagttgcaccgcatgatggagaaatgtattgcttatattcagcaatataatgttcctccatcatccagtgcaattaatatagtcgatagtggaagaacggtaacatatgtggtttgcagcaggtgagcaggatgggtgtggatgattgaatatctctgttcagtgttttcatcatctgactgaacactgaatcagcttgctgacgttagaggtttcagtttacctaatttatggtctgtacccatgaaaagtgggaaaaggctgaagaattcgatttctttctttctttcaatgtttcgccagcagaacacgcgctgaggttaattaatccagggcgcctgcttgtttgctgccaagagagtgttttgtgtactgctgctgccgagaaatatatatttttttctttcaccacctcgtgtgcagcagttgttttttgtttggatggataaatgtttctagtactgtggaggctgcatctgcatctgtttgtaaatggatgaaatatgaataaaaagttttgtttctcataccccatgtgtcttgtgtttgcatgcacgccgtgctagtttggtttttggtttctagagaaaacattttgtttgcttgtttctctataggatgtgaagaacctgcaggtgtttaaactagggataacagggtaataggtctcacgcggcaaatcctaccacctcatttaaatagagtgaggttgatttgcggccgcacaacaaacgcgacgactaacccgcaaatggcgcggctcgccagagtaatgctagggtggtgcatcaatggataaaaacctgccgcacgcaggcacaaacgctaaagggaccaatggcgaggaagaacataggggtagtttacggttgcccggtgcagggacgagcagttcacgctccgcggtagcctggtccggtcttctacgtacgacgatgaagtcctgttctccagtcaccaaacacgcaagggaagcccccaagcgtccagcgcgcgaccctgccgaacccccagcttgacgccaagattcaagggcagcggcagcggccatttccaatgcgttctctctccgtgggtctattcccagcggctatggctctcagtactggttatgggcgcatgatgcagagagatcaaccgagagtgtgcgatctggtatttattcagggagtccaggttctgctggggagggagatattagctggcagactccaggcttgttgcaacggccaagtggagttgtgagtttgttgtgtcggggcctcagcgaaggcgaaggtgctgacaagcggtccccacggtgcagtgtgtgtggttgagcgcggcgtggtgcgggagccgtgcaatgcggatccggattaggccggtgttgggctgaacgagcagcaaggagattcaggttgggttctgtttcatttttatttttttttctcttttctctctattttttcatttcacaagcccaattttttttcaaatccaaacaaggttctaattcaaaaactccataggtgcaaattattatttttgggatgtagtttaaccatatagtttattatattatcgttcactacttatttgatttggaaacaaagtaacatatagtctttcatcacaaattacttgagttcaattatattttaaaaattatttctaatacaaattaaatgaacagtcaaaatctaaggcatgatgcataatttcaacttctatttcatttttatagggaacatgt

SEQ ID NO: 11 - DNA, 19 bp, synthetic construct
  misc_feature 1..19: note = A 19 nucleotide sequence corresponding to the thermalamplification primer, SQ51606 used to identify corn eventZM_BCS216090 DNA in a sample.
  source 1..19: mol_type = other DNA
atcgacgctggatccgaag

SEQ ID NO: 12 - DNA, 21 bp, synthetic construct
  misc_feature 1..21: note = A 21 nucleotide sequence corresponding to a thermal amplificationprimer, SQ51629 used to identify corn event ZM_BCS216090 DNA in asample.
  source 1..21: mol_type = other DNA
ttagttagtcgcgccaaatcg

SEQ ID NO: 13 - DNA, 19 bp, synthetic construct
  misc_feature 1..19: note = A 19 mic;eptode seqiemce cprres[pmdomg tp a [rpbe referred tp asPB50583 used to identify corn event ZM_BCS216090 DNA in a sample.
  source 1..19: mol_type = other DNA
aagtttctcatctaagccc

SEQ ID NO: 14 - DNA, 24 bp, synthetic construct
  misc_feature 1..24: note = A 24 nucleotide sequence corresponding to a thermal amplificationprimer referred to as SQ20222 used as an internal control for theevent and zygosity assay for corn event ZM_BCS216090.
  source 1..24: mol_type = other DNA
gccctatgacttaccgagagttca

SEQ ID NO: 15 - DNA, 28 bp, synthetic construct
  misc_feature 1..28: note = A 28 nucleotide sequence corresponding to a thermal amplificationprimer referred to as SQ20221 used as an internal control for theevent and zygosity assay for corn event ZM_BCS216090.
  source 1..28: mol_type = other DNA
gttgctatgtactaacagaactgcatgt

SEQ ID NO: 16 - DNA, 17 bp, synthetic construct
  misc_feature 1..17: note = A 17 nucleotide sequence corresponding to a probe referred to asPB50298 used as an internal control for the event and zygosityassay for corn event ZM_BCS216090.
  source 1..17: mol_type = other DNA
ttgttgtgtggctccat

SEQ ID NO: 17 - DNA, 1000 bp, Zea mays
  misc_feature 1..1000: note = A 1,000 nucletodie sequence of the 5' flanking corn genomic DNAup to the inserted T-DNA in event ZM-BCS216090.
  source 1..1000: mol_type = genomic DNA
gattaacaagaacattattggttaaaaaagagtgatcaagggcacaactttccttcaacgagctcctgctcagtattttccatctgctgggtaccaggttccttggtcacttgctcgtctactcgtaacaatacaaacaaacatggtataggagaaattaacatcacaacaaacatgagcacaaactgcataataatgatctatgcgttgctacgagatcgtaggttcgggaactactaaattcggagttaaagtaaacaagatatggttttccaaagtgtaagtgactataattcatgtgaaaaatatattaaacagtagtaactgatatattccaggtcataacttgatcctagctcttgttttggtcaaacagtaattatgagcaatttaattcttgctaaaagttatttacataaacacaagctacatagatatctaattatagaagtatgggagatgtcacaattagtactattaatgcgtagataattttattacgaagataacgtaattggaacgggttaaaaaggagttaaaataagtaaaatatgaattttacaagtttttagattcacttttatattaaacattcattttccaatcttatttacccaggtcaaataatccatggacagcggacgcaaaaaccagggcgtttagggttcagtttgcaaattccgggacctgggggtaatgatctttgtgtacaggtgtactacgggttgattagtaaaaacaccagggctcttatgaaaaatcgcttggccgaaagggtatcatgcaatcagggccatggatcttggatctacggtcaggattaaattatcgttactatgaatcgatattcaataggaaccctaggataggatatgcacgactcagattttatgttgaccaaatggacgtggttcacccgatctatcatcaacgctcctgattccttactcccgaaccgataaccctcttctaatccgggccatcgacgctggatccgaag

SEQ ID NO: 18 - DNA, 1000 bp, Zea mays
  misc_feature 1..1000: note = A 1,000 nucleotide sequence of the 3' flanking corn genomic DNAimmediately after the inserted T-DNA in event ZM-BCS216090.
  source 1..1000: mol_type = genomic DNA
gacgactaacccgcaaatggcgcggctcgccagagtaatgctagggtggtgcatcaatggataaaaacctgccgcacgcaggcacaaacgctaaagggaccaatggcgaggaagaacataggggtagtttacggttgcccggtgcagggacgagcagttcacgctccgcggtagcctggtccggtcttctacgtacgacgatgaagtcctgttctccagtcaccaaacacgcaagggaagcccccaagcgtccagcgcgcgaccctgccgaacccccagcttgacgccaagattcaagggcagcggcagcggccatttccaatgcgttctctctccgtgggtctattcccagcggctatggctctcagtactggttatgggcgcatgatgcagagagatcaaccgagagtgtgcgatctggtatttattcagggagtccaggttctgctggggagggagatattagctggcagactccaggcttgttgcaacggccaagtggagttgtgagtttgttgtgtcggggcctcagcgaaggcgaaggtgctgacaagcggtccccacggtgcagtgtgtgtggttgagcgcggcgtggtgcgggagccgtgcaatgcggatccggattaggccggtgttgggctgaacgagcagcaaggagattcaggttgggttctgtttcatttttatttttttttctcttttctctctattttttcatttcacaagcccaattttttttcaaatccaaacaaggttctaattcaaaaactccataggtgcaaattattatttttgggatgtagtttaaccatatagtttattatattatcgttcactacttatttgatttggaaacaaagtaacatatagtctttcatcacaaattacttgagttcaattatattttaaaaattatttctaatacaaattaaatgaacagtcaaaatctaaggcatgatgcataatttcaacttctatttcatttttatagggaacatgt

SEQ ID NO: 19 - DNA, 5000 bp, Zea mays
  source 1..5000: mol_type = genomic DNA
ctactgtgaggaggcggaggagcggctgctggtgtgcgagtacatgaagaacggcgcactctacgaccacctgcaccccaaggcctcggcgacggcggcgccgtcgcccgtggcgtcgtcatggaagctgcgcatcaagatcctgatgtgcatattaccatactagtgaacaatcaggggctaactctttttaggactcgataaacaacatcaacagaagtatgtaacatgggaaactgctgtgcatatttaggttcatgatgtttcccatcctctgctcaaaagtcttggcgtgaagaatctcccagctgtaattggccgaactgtgaatggtgaggagctccttctgaaggatggcatttcgatgaaagatctcagatctggtgtcaaggaattgaagacactgctagatagttttgagaagaagaataagaagttattatcgaatctagctaataagaaaccttccggtcaatgggaagaaatcaagaagattcctctgctcacagcttccaactttgaggaaatctgtggagagaagactctagtatgcatcattggtgttttcgggtcaaacaaagccaaggggtagcttgaagcagtcctgtctgaggtatatctcctattttcaaaagccaaattatagagcagaaacacatgttttcttgtttgcctgaatgcatagtaatatatattaccttgtggttctttccttaatcttcagtatagccatttgttaacacatatcatcgctttcctgttgattgcatccagatatctaagaagactctgataagggggcagaactataattccagaaatgttgtctcctatgctttgctggataaggacaaacaatctgcattcctgtcatcgtttgacaagtctggatacaggtcatctgacaagctgctcatcgcctacaaggaacccattattattatgttattattatttagaactgaaggaagatttggatgcaatgtttcccatgtgaatacatacagtggaattgcaggaaaatcaatggataagtttggtgcattgatatcatctttcagataccccggcccccgtgccagtggccttcgtgctcctttaaaggatgtgaaaaacacactttctgtgaggtatgcatctctagaaccttttttcagacttgaaacttctaaggaaattaagtatgattggtttacttactggcgacagtacagtcttgttatgattatgtatctattttttatgccattcttatttctgctgcaaatctattcttaaagctcaagaaaaaggcacattcagacgcacagcaaagaaaacaggtctaggcccataacttgaattttgattgatgtcttttttgaaatctaatgattccattgagagttcattttcctctccaagattcaaccttatagcattgctgttgtctggtgatagatatgcattaccgagcttcaacgaactgaactcgagctttgctgtttcatctatgatcgttatttccataaccgagtccgccattcagttatgcagagagcgacaactttaaatgcattttacaatgggtacatatgcattgcaaattctgacctcgcatgatgttggtgaacattctcgcactcccagagcagattgaatttttcttatcaaatgaggccatgccatagaggagattgctaatttttcgtcctcatggtggcaggtgcatgttcaatcagatcctgctggacgtctggttatcacaggggatccagagcagcctgataatccttggaggatcactccgttcaagaatgtggtaggctgacagcttcactatcagaaacacatttgtgaatttagtttaacatgtagtgattttttttatcttaggtggtcaacttgccatcaagaatcaatcctcaccaaacatccgctgttatcacgcatggtcagttgtttgtgcgtgcaccatttgggtcatccgtgatttgtggtctttcaggaattccttgccccaaaatttatcttgttttctttgatttcatgtattttgtttattgtgcagggtttatgtcagttctgttgaggaaataggatttcatttgagcagatacatgatgtcaagctgacttagcagaacccttctgtggtttgatgctggttgctggtgttgtgatctgttgccaccttgtttattgttacagtagtgccataaaattagctggttcatgtgtgtattccagtttcgtagagaaaggagatgttggtcgtagtagggctaatttgacagtatgattacttattattctttaaatacctctctggatcactccaggcctccagggcgccctaaagctgcattttttttctttactaggtgagtgcccatgcgttgcaagtgttgtttcaagatggtcatttggttgacgaggcaatgtcgagcgcttacaccacaactacatacattctagtggccaacatcatgaacccgagtcgggtcgtggcgaaggtttgctctcgaggatagcgagtgtctagagtgcgaggtgatgggtgtgctgattgacaagaccaagcacgtgtcgctcaacaggtgggccaaacttttatcggataaagtaggcccagtacaccgctatcgacgtctacatgtcctacgagactggtcggctgctgctcatcagggagcgcgtggaagatgcggtcaccgtcggtgcaacaatctcgttgtgtcatgttctatgttatcttagtgggactatttcttgttgcctattgtttactctctctagctccttacaacttcatgtctaatgtgatgcgatgtgaattggactagtgatttctgtcacacccggattaaaggggcaaacccaggtgcgtctcatatgtgcgccaaacgagatcaacacatataatgacttagtgaatagagacgaatgtcacaatctttattacataacgaaatgttttacaaaataaattataatatatagaacgaactaatgtcattattgtcccttggcgccaacaagttgactgggagacgacacctagacctcgtcgaactcctcgtagtagtcttcctctagggccacctgctcttcacctatggggggagttttattgcaggagtgagctcacagatgttcatcgctcagcaagtgtggagaataatgtgcatgggctcacttacggtggggctcatatatagtgtcaagctgatcaacaaataatggttaaggcaaagcattgcttttaataagttggtcaaattttattagtagttactaaatataagtaaataccatcccaacaaaataatagataaaattaataataaaaccacaatacgatgcatatgacaaattaagtttaattccataagttaatcatgcaagagtcctcaatcgctcttgaccacaagcacgactgatataccagttttacaatctgcagaggttgcacatctttacccataatacatgttccctttgtcgcctcggattgatcaaacccttaaacattgccaaggtgagtaggcagggtacactacaatgcctttacaaagttccactagaggagagaacctcctatggtttctgggccgaaagttgtacaggaatccctcggtccaaaagctaaaaagatagtccgacccgaggacctccctacacccgcatcctccactccgcttgcccctttcgggaaaggattaactcacactagctttcctaattaatcagccaagggtgtcccattctaccattgtgatagcactattatctcaagttaagctccatgttccagttaacaccataatcttatcatgaacatttaaataatccaataataatagttaagcatgatcatgaatcatttgtatcttaacatccaaaaccacatagagcaatagcagatactactcaaaagttcagtggcaagcaaggtataaagtaactcaaactagggtgacctattgggtaccatcaaattaacctgagcatagtatagtgattaacaagaacattattggttaaaaaagagtgatcaagggcacaactttccttcaacgagctcctgctcagtattttccatctgctgggtaccaggttccttggtcacttgctcgtctactcgtaacaatacaaacaaacatggtataggagaaattaacatcacaacaaacatgagcacaaactgcataataatgatctatgcgttgctacgagatcgtaggttcgggaactactaaattcggagttaaagtaaacaagatatggttttccaaagtgtaagtgactataattcatgtgaaaaatatattaaacagtagtaactgatatattccaggtcataacttgatcctagctcttgttttggtcaaacagtaattatgagcaatttaattcttgctaaaagttatttacataaacacaagctacatagatatctaattatagaagtatgggagatgtcacaattagtactattaatgcgtagataattttattacgaagataacgtaattggaacgggttaaaaaggagttaaaataagtaaaatatgaattttacaagtttttagattcacttttatattaaacattcattttccaatcttatttacccaggtcaaataatccatggacagcggacgcaaaaaccagggcgtttagggttcagtttgcaaattccgggacctgggggtaatgatctttgtgtacaggtgtactacgggttgattagtaaaaacaccagggctcttatgaaaaatcgcttggccgaaagggtatcatgcaatcagggccatggatcttggatctacggtcaggattaaattatcgttactatgaatcgatattcaataggaaccctaggataggatatgcacgactcagattttatgttgaccaaatggacgtggttcacccgatctatcatcaacgctcctgattccttactcccgaaccgataaccctcttctaatccgggccatcgacgctggatccgaag

SEQ ID NO: 20 - DNA, 5000 bp, Zea mays
  source 1..5000: mol_type = genomic DNA
cttgccaataaaacatagacttacaataactctaattttattacaccaagaactagctaacaatttaaaccatattttaagggtttacaaatcctaccccctaaaaaataatctcggccttgagattacgatgaactaggaaaggggtggggaaattctactcgtacctcaacttcttgctcgtaggtggcttcatcttctgtgtgatgactccattgtactttgcacatcttaatcactttattccttgtaacacgagtcaaggtatcaatgatcttgataggatattctgtataagtcagatcaccttgaacactaagttcatccatagttagttgctcttctggcaccctaagacacttcttcaactgagatacatgtaagacatcatgtatatccgctagatggttgggtagctccagttgatatgcaactgcccccactcgctttaggattttgaagggtccaataaagcgaggggacaacttacctttaactttaaatttcctcatacaccgaattggtgacaccttgagatacacgtgatctctacagcttttctgtctggactgcgcagttctcaagttctcccttattattcgaatttgttcctctgcttcttggcttattattggcccaaagaactgcctttcaccagtttgatcctaatacagcaggattctacacttccttccatataaggcctccaacggggacatcttcaaactggcctgataactattattataggagaactccgcatagggtaaactcttgtctcaactgccatcgtgcttaagatcacaagctcttaacatatcttctagcacttgatttgttctcttggtctgcccatcagtttagggatggtaggctgaactaaagtttagcttcgtgtccatactctcatgaaggttcttccaaaatcgggaagtaaactgagatccacgatctaaaactatcttcttaggtaccccatgtaggcacatacactacacgacggttgatctttagcgacccttattagggactgacggttggtcgctaaaagttatggactgacggttggtcgctaaatgcgcttataacgacggtctatgggtcgctaaaagtctagaaatttaacgacttatggtcggtcgctatagaggtaagtgtgtgactgatggtgggtcgctataggggtacgtgatttttgtgtttcttatttactcaccaaagcccatctaaaggcctactaaacaaacaaaaccctattccacgcaccaaaccacaagcgcagccgccgtcacttctgccccatcgtggccccatcccgccgctcgtcgacgatgccaccccgcccctcggccgtggtcgccgtcactataccacaacaccaaaatagcgtcagatctcagtcactaaaagtagtaaattgacagtgaatcagtcgctaaaacatagctacacaccatcagtcgatatttctaaatatactgttaaactgtccatcggtacttttatttatgtttcacgttgagctgtccgtcggcattaccatcttaggagtcagaccatccgtcgttgtagaatgtcccaaaactaaagctctttgcgcgtattggactagcttgcttgtgtctaaaattttccatgtggtgcacagtgcactggcccggcccacaaaacatgtcaagttgacctagccaaatccaaatctcttcaatccctcgaccagtcgaccgcgccgctcacccgtgctgcccagatctcctctcactccaccgtgtgcccagccctccgccgctagtcccctcccggtcctccccgcgcgttgtcatcgccggtcgcccagtcctccctgcaggacgccatgctgcccagccttcctcgtagtcgtcctcagctcgtgtcttagcgctctcccatggtggtaggccgacggctcctctcctacctcagagatagatctacacccacggcagtggctgctctgatgacatcaatcaatacaatgggggttcctgctacggatccatctcctcaagtgttgatttaaggtcaagtgtgttgtgtacttgtgtgtaggcctattgttgctgcttcccaaaatgcctcgaatctgtgttccaacgacagcatgtctcctcctgtgcactcctgtgaagacttaaggtgagttttatatgtcacttttctttaccgtgaatatttgaggactaaacacacaagaactaatgtgctaggacataactcttatctcatttactgaatattttatttagcctagtggctactttgctatattttttgtccaatcattcaactgaaagtattatttttttccaaactgagatcagaccacagaaaaaatagatataaagcataatagatgtgcctagtagatattcttgcattggttatggctgctactagtcatgtggctgatttttagatgtgctcaaaataaaatattatttttattagccaaggagtagtatttggagtagtattcttggattcattctctagtgtctcggttggtctttgtctgtcaattttaaatgccaaatcttgtacccaactattctgccagcgcatggtgacataatactgtcacacccggttttagaaggcaaaccgaatgcgaaccatgtacgtgccaggatcagcaattcacgtacacagcagttacatacatggacatcatcacacagtgctcaaatagtattaaaaagggaaataatagtcggttacatcatatgtctgagacatccacatagtctttacaataaatcaaagtgcggaaaagaaacatagataaacgcggccttcacaggcagccaactaggggttgccgctaacccacgcctagatctcgtcgtagtcttggaacttctggaagtctccttccatggcttcatcctctcctgagcagtggttgcaatgctgacaacctggggatgggggggggtttggtgtgtagagcaagggtgagtacacatcaacatactcagcaagtgtcctgtttggctgtagtggactagctttatgtgggcataagttaagcagttgcttttagttggtcagattattatttactagtagaaagccaggttttaacattgacccaagttattagcccgatgtactctttccaaacggaaagaataccacttaccagcaccatagtcataaccagaaccatcaacctcattgccacctgtaaaccaaaatgtctctgatcaagtaccactaatcactggagctcccttggccgctcataaccgcgagcacggctgatatatcagttttcaaacactctgcagaggttgtgcactttacccacaagccgtgattccctttttctgcccggagatcatgactctccattgatcactaccaaggtgacccagcagggcatcactatgtagcctttacaaagattccccggggctgtagccgcctgttaggtttcctaaatgcaccgcactcctccccaaggggcgaacccaaacttggcagagcgagccgcatacaccgagccccattgacggcacgacggttaagcgaactacaccccggatcctctaattattcagctaagggcaccccattccaccctcatggttgcactgttttcccggacggtcatccaaagaacaggttcttacggagaggcactcgagaaaccgctcgagccccgttgaagaccacaagtataacatcataataagaagagggaaaacaacgtatcattgatcattctcatcatgttcattgattagagttgagcaatagcataagactaagcaataataatccaacccaaataggtaaccaaggacatggataacaaagctagtaaatccttaggcataaatgtgtaatgcgggagatgaattaaagaatgaataggacagagataggtcaagtgacacttgcctccaccaacagactgctgctcaggggcttctcctgcgagttcttcgggctcttcaaccggatcgttctctatgacgactaacccgcaaatggcgcggctcgccagagtaatgctagggtggtgcatcaatggataaaaacctgccgcacgcaggcacaaacgctaaagggaccaatggcgaggaagaacataggggtagtttacggttgcccggtgcagggacgagcagttcacgctccgcggtagcctggtccggtcttctacgtacgacgatgaagtcctgttctccagtcaccaaacacgcaagggaagcccccaagcgtccagcgcgcgaccctgccgaacccccagcttgacgccaagattcaagggcagcggcagcggccatttccaatgcgttctctctccgtgggtctattcccagcggctatggctctcagtactggttatgggcgcatgatgcagagagatcaaccgagagtgtgcgatctggtatttattcagggagtccaggttctgctggggagggagatattagctggcagactccaggcttgttgcaacggccaagtggagttgtgagtttgttgtgtcggggcctcagcgaaggcgaaggtgctgacaagcggtccccacggtgcagtgtgtgtggttgagcgcggcgtggtgcgggagccgtgcaatgcggatccggattaggccggtgttgggctgaacgagcagcaaggagattcaggttgggttctgtttcatttttatttttttttctcttttctctctattttttcatttcacaagcccaattttttttcaaatccaaacaaggttctaattcaaaaactccataggtgcaaattattatttttgggatgtagtttaaccatatagtttattatattatcgttcactacttatttgatttggaaacaaagtaacatatagtctttcatcacaaattacttgagttcaattatattttaaaaattatttctaatacaaattaaatgaacagtcaaaatctaaggcatgatgcataatttcaacttctatttcatttttatagggaacatgt

SEQ ID NO: 21 - DNA, 3684 bp, synthetic construct
  source 1..3684: mol_type = other DNA
atgtcgaagctcgagaagttcaccaactgctactcgctgagcaagacgctgcggttcaaggcgatccccgtcgggaagacccaggagaacatcgacaacaagcggctcctggtcgaggacgagaagcgcgccgaggactacaagggcgtcaagaagctgctggaccggtactacctctccttcatcaacgacgtcctgcactcgatcaagctcaagaacctgaacaactacatctcgctgttccgcaagaagacacggaccgagaaggagaacaaggagctcgagaacctcgagatcaacctgcgcaaggagatcgcgaaggcgttcaagggcaacgaggggtacaagagcctgttcaagaaagacatcatcgagaccatcctgccggagttcctggacgacaaggacgagatcgcgctggtgaactcgttcaacgggttcaccacggccttcaccgggtttttcgacaaccgggagaacatgttcagcgaggaggccaagtcgaccagcatcgccttccggtgcatcaacgagaacctcacccgctacatcagcaacatggacatcttcgagaaggtggacgccatcttcgacaagcacgaggtccaggagatcaaggaaaagatcctgaactcggactacgacgtggaagacttctttgagggcgagttcttcaacttcgtcctcacccaggagggcatcgacgtctacaacgccatcatcggcggcttcgtgacggagagcggcgagaagatcaagggcctcaacgagtacatcaacctctacaaccagaagactaagcagaagctcccgaagttcaagccgctgtacaagcaagtcctgagcgaccgggagtccctctcgttctacggcgagggctacacgagcgacgaggaggtgctggaggtgttccgcaacacgctgaacaagaacagcgagatcttcagctcgatcaagaaactcgagaagctgttcaagaacttcgacgagtacagcagcgccggcatcttcgtcaagaacgggcccgcgatcagcaccatcagcaaggacatcttcggggagtggaacgtgatccgcgacaagtggaacgccgagtacgacgacatccacctcaagaaaaaggcggtggtcacggagaagtacgaggacgaccgccggaagtccttcaagaaaatcgggagcttcagcctcgagcagctccaggagtacgcggacgccgacctgagcgtggtggagaagctcaaggagatcatcatccagaaggtcgacgagatctacaaggtctacggctcgagcgagaagctgttcgacgcggacttcgtgctggagaagtccctcaagaagaacgacgccgtggtggccatcatgaaggatctgctcgacagcgtgaagtcgttcgagaactacatcaaggcattctttggggagggcaaggagacgaaccgggacgagtccttctacggggacttcgtgctcgcgtacgacatcctcctgaaggtcgaccacatctacgacgcgatccggaactacgtcacgcagaagccctacagcaaggacaagttcaagctctacttccagaacccgcagttcatgggcgggtgggacaaggacaaggagaccgactaccgggccacgatcctgcggtacgggtccaagtactacctcgccatcatggacaagaagtacgccaagtgcctccagaagattgacaaggacgacgtgaacgggaactacgagaagatcaactacaagctcctcccggggcccaacaagatgctgccgaaggtgttcttcagcaagaagtggatggcctactacaacccctcggaggacatccagaagatatacaagaacggcacgttcaaaaagggggacatgttcaacctgaacgactgccacaagctgatcgactttttcaaggacagcatcagccgctacccgaagtggtcgaacgcctacgacttcaacttctcggagacggagaagtacaaggacattgcgggcttctaccgggaggtggaggagcagggctacaaggtctccttcgagagcgcctccaagaaagaggtggacaagctcgtggaggagggcaagctgtacatgttccagatctacaacaaggacttctcggacaagtcgcacggcaccccgaacctccacacgatgtacttcaagctgctgttcgacgagaacaaccacgggcagatccgcctcagcggcggggcggagctgttcatgcgccgcgcgtccctcaagaaggaggagctggtcgtgcaccccgccaactccccgatcgcgaacaagaaccccgacaaccccaagaagacaaccaccctctcgtacgacgtctacaaggacaagcggttctcggaggaccagtacgagctgcacatcccgatcgccatcaacaagtgccccaagaacatcttcaagatcaacaccgaggtgcgggtgctgctcaagcacgacgacaacccctacgtcatcgggatcgaccgcggcgagcggaacctgctctacatcgtggtcgtggacgggaaggggaacatcgtggagcagtacagcctgaacgagatcatcaacaacttcaacggcatccgcatcaagacggactaccacagcctcctggacaagaaggagaaggagcggttcgaggcgcggcagaactggacctccatcgagaacatcaaggagctgaaggccggctacatcagccaggtcgtgcacaagatctgcgagctcgtggagaagtacgacgcggtgatcgcgctggaggacttgaacagcgggttcaagaactcccgggtcaaggtcgagaagcaggtctaccagaagttcgagaagatgctgatcgacaagctcaactacatggtggacaagaagtccaacccctgcgccaccggcggcgccctcaagggctaccagatcaccaacaagttcgagtccttcaagtcgatgtctacgcagaacgggttcattttctacatcccggcgtggctcaccagcaagatcgacccgagcacgggcttcgtcaacctcctgaagaccaagtacaccagcatcgcggacagcaagaagttcatctcctcgttcgaccgcatcatgtacgtccccgaggaagacctgttcgagttcgccctcgactacaagaacttctcccggacggacgccgactacatcaaaaagtggaagctctacagctacggcaaccggatccgcatcttccgcaaccccaagaagaacaatgtgttcgactgggaggaggtgtgcctgacgagcgcctacaaggagctcttcaacaagtacggcatcaactaccagcaaggggacatccgcgcgctgctctgcgagcagtccgacaaggcgttctactcgtcgttcatggccctgatgagcctcatgctccagatgcgcaacagcatcaccggccggacggacgtggacttcctgatcagcccggtcaagaacagcgacggcattttctacgacagccggaactacgaggcccaggagaacgccatcctccccaagaacgccgacgcgaacggcgcctacaacatcgcgcggaaggtgctgtgggccatcggccagtttaaaaaggcggaggacgagaagctggacaaggtcaagatcgccatcagcaacaaggagtggctcgagtacgcgcagacgagcgtgaagcac

SEQ ID NO: 22 - protein, 1228 aa, unidentified
  source 1..1228: note = Lachnospiraceae bacterium ND2006
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 23 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDKETDYRATILRYGSKYYLAIMDKKYAKCLQKIDKDDVNGNYEKINYKLLPGPNKMLPRVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 24 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDVETDRRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 25 - protein, 1300 aa, Francisella sp.
  source 1..1300: note = Francisella novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 26 - DNA, 21 bp, synthetic construct
  source 1..21: mol_type = other DNA
gaatttctactaagtgtagat

SEQ ID NO: 27 - DNA, 21 bp, synthetic construct
  source 1..21: mol_type = other DNA
gtaatttctactgttgtagat

SEQ ID NO: 28 - DNA, 51 bp, synthetic construct
  source 1..51: mol_type = other DNA
gtaatttctactgttgtagatagtttgcaaattccgggacctggttttttt

SEQ ID NO: 29 - DNA, 51 bp, synthetic construct
  source 1..51: mol_type = other DNA
gtaatttctactgttgtagatatgcaccaccctagcattactctttttttt

SEQ ID NO: 30 - DNA, 50 bp, Zea mays
  source 1..50: mol_type = genomic DNA
gattaacaagaacattattggttaaaaaagagtgatcaagggcacaactt

SEQ ID NO: 31 - DNA, 50 bp, Zea mays
  source 1..50: mol_type = genomic DNA
tccttcaacgagctcctgctcagtattttccatctgctgggtaccaggtt

SEQ ID NO: 32 - DNA, 50 bp, Zea mays
  source 1..50: mol_type = genomic DNA
ccttggtcacttgctcgtctactcgtaacaatacaaacaaacatggtata

SEQ ID NO: 33 - DNA, 50 bp, Zea mays
  source 1..50: mol_type = genomic DNA
ggagaaattaacatcacaacaaacatgagcacaaactgcataataatgat

SEQ ID NO: 34 - DNA, 50 bp, Zea mays
  source 1..50: mol_type = genomic DNA
ctatgcgttgctacgagatcgtaggttcgggaactactaaattcggagtt

SEQ ID NO: 35 - DNA, 50 bp, Zea mays
  source 1..50: mol_type = genomic DNA
aaagtaaacaagatatggttttccaaagtgtaagtgactataattcatgt

SEQ ID NO: 36 - DNA, 50 bp, Zea mays
  source 1..50: mol_type = genomic DNA
gaaaaatatattaaacagtagtaactgatatattccaggtcataacttga

SEQ ID NO: 37 - DNA, 50 bp, Zea mays
  source 1..50: mol_type = genomic DNA
tcctagctcttgttttggtcaaacagtaattatgagcaatttaattcttg

SEQ ID NO: 38 - DNA, 50 bp, Zea mays
  source 1..50: mol_type = genomic DNA
ctaaaagttatttacataaacacaagctacatagatatctaattatagaa

SEQ ID NO: 39 - DNA, 50 bp, Zea mays
  source 1..50: mol_type = genomic DNA
gtatgggagatgtcacaattagtactattaatgcgtagataattttatta

SEQ ID NO: 40 - DNA, 50 bp, Zea mays
  source 1..50: mol_type = genomic DNA
cgaagataacgtaattggaacgggttaaaaaggagttaaaataagtaaaa

SEQ ID NO: 41 - DNA, 50 bp, Zea mays
  source 1..50: mol_type = genomic DNA
tatgaattttacaagtttttagattcacttttatattaaacattcatttt

SEQ ID NO: 42 - DNA, 50 bp, Zea mays
  source 1..50: mol_type = genomic DNA
ccaatcttatttacccaggtcaaataatccatggacagcggacgcaaaaa

SEQ ID NO: 43 - DNA, 50 bp, Zea mays
  source 1..50: mol_type = genomic DNA
ccagggcgtttagggttcagtttgcaaattccgggacctgggggtaatga

SEQ ID NO: 44 - DNA, 50 bp, Zea mays
  source 1..50: mol_type = genomic DNA
tctttgtgtacaggtgtactacgggttgattagtaaaaacaccagggctc

SEQ ID NO: 45 - DNA, 50 bp, Zea mays
  source 1..50: mol_type = genomic DNA
ttatgaaaaatcgcttggccgaaagggtatcatgcaatcagggccatgga

SEQ ID NO: 46 - DNA, 50 bp, Zea mays
  source 1..50: mol_type = genomic DNA
tcttggatctacggtcaggattaaattatcgttactatgaatcgatattc

SEQ ID NO: 47 - DNA, 50 bp, Zea mays
  source 1..50: mol_type = genomic DNA
aataggaaccctaggataggatatgcacgactcagattttatgttgacca

SEQ ID NO: 48 - DNA, 50 bp, Zea mays
  source 1..50: mol_type = genomic DNA
aatggacgtggttcacccgatctatcatcaacgctcctgattccttactc

SEQ ID NO: 49 - DNA, 50 bp, Zea mays
  source 1..50: mol_type = genomic DNA
ccgaaccgataaccctcttctaatccgggccatcgacgctggatccgaag

SEQ ID NO: 50 - DNA, 50 bp, Zea mays
  source 1..50: mol_type = genomic DNA
ctactgtgaggaggcggaggagcggctgctggtgtgcgagtacatgaaga

SEQ ID NO: 51 - DNA, 50 bp, Zea mays
  source 1..50: mol_type = genomic DNA
acggcgcactctacgaccacctgcaccccaaggcctcggcgacggcggcg

SEQ ID NO: 52 - DNA, 50 bp, Zea mays
  source 1..50: mol_type = genomic DNA
ccgtcgcccgtggcgtcgtcatggaagctgcgcatcaagatcctgatgtg

SEQ ID NO: 53 - DNA, 50 bp, Zea mays
  source 1..50: mol_type = genomic DNA
catattaccatactagtgaacaatcaggggctaactctttttaggactcg

SEQ ID NO: 54 - DNA, 50 bp, Zea mays
  source 1..50: mol_type = genomic DNA
ataaacaacatcaacagaagtatgtaacatgggaaactgctgtgcatatt

SEQ ID NO: 55 - DNA, 50 bp, Zea mays
  source 1..50: mol_type = genomic DNA
taggttcatgatgtttcccatcctctgctcaaaagtcttggcgtgaagaa

SEQ ID NO: 56 - DNA, 50 bp, Zea mays
  source 1..50: mol_type = genomic DNA
tctcccagctgtaattggccgaactgtgaatggtgaggagctccttctga

SEQ ID NO: 57 - DNA, 50 bp, Zea mays
  source 1..50: mol_type = genomic DNA
aggatggcatttcgatgaaagatctcagatctggtgtcaaggaattgaag

SEQ ID NO: 58 - DNA, 50 bp, Zea mays
  source 1..50: mol_type = genomic DNA
acactgctagatagttttgagaagaagaataagaagttattatcgaatct

SEQ ID NO: 59 - DNA, 50 bp, Zea mays
  source 1..50: mol_type = genomic DNA
agctaataagaaaccttccggtcaatgggaagaaatcaagaagattcctc

SEQ ID NO: 60 - DNA, 50 bp, Zea mays
  source 1..50: mol_type = genomic DNA
tgctcacagcttccaactttgaggaaatctgtggagagaagactctagta

SEQ ID NO: 61 - DNA, 50 bp, Zea mays
  source 1..50: mol_type = genomic DNA
tgcatcattggtgttttcgggtcaaacaaagccaaggggtagcttgaagc

SEQ ID NO: 62 - DNA, 50 bp, Zea mays
  source 1..50: mol_type = genomic DNA
agtcctgtctgaggtatatctcctattttcaaaagccaaattatagagca

SEQ ID NO: 63 - DNA, 50 bp, Zea mays
  source 1..50: mol_type = genomic DNA
gaaacacatgttttcttgtttgcctgaatgcatagtaatatatattacct

SEQ ID NO: 64 - DNA, 50 bp, Zea mays
  source 1..50: mol_type = genomic DNA
tgtggttctttccttaatcttcagtatagccatttgttaacacatatcat

SEQ ID NO: 65 - DNA, 50 bp, Zea mays
  source 1..50: mol_type = genomic DNA
cgctttcctgttgattgcatccagatatctaagaagactctgataagggg

SEQ ID NO: 66 - DNA, 50 bp, Zea mays
  source 1..50: mol_type = genomic DNA
gcagaactataattccagaaatgttgtctcctatgctttgctggataagg

SEQ ID NO: 67 - DNA, 50 bp, Zea mays
  source 1..50: mol_type = genomic DNA
acaaacaatctgcattcctgtcatcgtttgacaagtctggatacaggtca

SEQ ID NO: 68 - DNA, 50 bp, Zea mays
  source 1..50: mol_type = genomic DNA
tctgacaagctgctcatcgcctacaaggaacccattattattatgttatt

SEQ ID NO: 69 - DNA, 50 bp, Zea mays
  source 1..50: mol_type = genomic DNA
attatttagaactgaaggaagatttggatgcaatgtttcccatgtgaata

SEQ ID NO: 70 - DNA, 50 bp, Zea mays
  source 1..50: mol_type = genomic DNA
catacagtggaattgcaggaaaatcaatggataagtttggtgcattgata

SEQ ID NO: 71 - DNA, 50 bp, Zea mays
  source 1..50: mol_type = genomic DNA
tcatctttcagataccccggcccccgtgccagtggccttcgtgctccttt

SEQ ID NO: 72 - DNA, 50 bp, Zea mays
  source 1..50: mol_type = genomic DNA
aaaggatgtgaaaaacacactttctgtgaggtatgcatctctagaacctt

SEQ ID NO: 73 - DNA, 50 bp, Zea mays
  source 1..50: mol_type = genomic DNA
ttttcagacttgaaacttctaaggaaattaagtatgattggtttacttac

SEQ ID NO: 74 - DNA, 50 bp, Zea mays
  source 1..50: mol_type = genomic DNA
tggcgacagtacagtcttgttatgattatgtatctattttttatgccatt

SEQ ID NO: 75 - DNA, 50 bp, Zea mays
  source 1..50: mol_type = genomic DNA
cttatttctgctgcaaatctattcttaaagctcaagaaaaaggcacattc

SEQ ID NO: 76 - DNA, 50 bp, Zea mays
  source 1..50: mol_type = genomic DNA
agacgcacagcaaagaaaacaggtctaggcccataacttgaattttgatt

SEQ ID NO: 77 - DNA, 50 bp, Zea mays
  source 1..50: mol_type = genomic DNA
gatgtcttttttgaaatctaatgattccattgagagttcattttcctctc

SEQ ID NO: 78 - DNA, 50 bp, Zea mays
  source 1..50: mol_type = genomic DNA
caagattcaaccttatagcattgctgttgtctggtgatagatatgcatta

SEQ ID NO: 79 - DNA, 50 bp, Zea mays
  source 1..50: mol_type = genomic DNA
ccgagcttcaacgaactgaactcgagctttgctgtttcatctatgatcgt

SEQ ID NO: 80 - DNA, 50 bp, Zea mays
  source 1..50: mol_type = genomic DNA
tatttccataaccgagtccgccattcagttatgcagagagcgacaacttt

SEQ ID NO: 81 - DNA, 50 bp, Zea mays
  source 1..50: mol_type = genomic DNA
aaatgcattttacaatgggtacatatgcattgcaaattctgacctcgcat

SEQ ID NO: 82 - DNA, 50 bp, Zea mays
  source 1..50: mol_type = genomic DNA
gatgttggtgaacattctcgcactcccagagcagattgaatttttcttat

SEQ ID NO: 83 - DNA, 50 bp, Zea mays
  source 1..50: mol_type = genomic DNA
caaatgaggccatgccatagaggagattgctaatttttcgtcctcatggt

SEQ ID NO: 84 - DNA, 50 bp, Zea mays
  source 1..50: mol_type = genomic DNA
ggcaggtgcatgttcaatcagatcctgctggacgtctggttatcacaggg

SEQ ID NO: 85 - DNA, 50 bp, Zea mays
  source 1..50: mol_type = genomic DNA
gatccagagcagcctgataatccttggaggatcactccgttcaagaatgt

SEQ ID NO: 86 - DNA, 50 bp, Zea mays
  source 1..50: mol_type = genomic DNA
ggtaggctgacagcttcactatcagaaacacatttgtgaatttagtttaa

SEQ ID NO: 87 - DNA, 50 bp, Zea mays
  source 1..50: mol_type = genomic DNA
catgtagtgattttttttatcttaggtggtcaacttgccatcaagaatca

SEQ ID NO: 88 - DNA, 50 bp, Zea mays
  source 1..50: mol_type = genomic DNA
atcctcaccaaacatccgctgttatcacgcatggtcagttgtttgtgcgt

SEQ ID NO: 89 - DNA, 50 bp, Zea mays
  source 1..50: mol_type = genomic DNA
gcaccatttgggtcatccgtgatttgtggtctttcaggaattccttgccc

SEQ ID NO: 90 - DNA, 50 bp, Zea mays
  source 1..50: mol_type = genomic DNA
caaaatttatcttgttttctttgatttcatgtattttgtttattgtgcag

SEQ ID NO: 91 - DNA, 50 bp, Zea mays
  source 1..50: mol_type = genomic DNA
ggtttatgtcagttctgttgaggaaataggatttcatttgagcagataca

SEQ ID NO: 92 - DNA, 50 bp, Zea mays
  source 1..50: mol_type = genomic DNA
tgatgtcaagctgacttagcagaacccttctgtggtttgatgctggttgc

SEQ ID NO: 93 - DNA, 50 bp, Zea mays
  source 1..50: mol_type = genomic DNA
tggtgttgtgatctgttgccaccttgtttattgttacagtagtgccataa

SEQ ID NO: 94 - DNA, 50 bp, Zea mays
  source 1..50: mol_type = genomic DNA
aattagctggttcatgtgtgtattccagtttcgtagagaaaggagatgtt

SEQ ID NO: 95 - DNA, 50 bp, Zea mays
  source 1..50: mol_type = genomic DNA
ggtcgtagtagggctaatttgacagtatgattacttattattctttaaat

SEQ ID NO: 96 - DNA, 50 bp, Zea mays
  source 1..50: mol_type = genomic DNA
acctctctggatcactccaggcctccagggcgccctaaagctgcattttt

SEQ ID NO: 97 - DNA, 50 bp, Zea mays
  source 1..50: mol_type = genomic DNA
tttctttactaggtgagtgcccatgcgttgcaagtgttgtttcaagatgg

SEQ ID NO: 98 - DNA, 50 bp, Zea mays
  source 1..50: mol_type = genomic DNA
tcatttggttgacgaggcaatgtcgagcgcttacaccacaactacataca

SEQ ID NO: 99 - DNA, 50 bp, Zea mays
  source 1..50: mol_type = genomic DNA
ttctagtggccaacatcatgaacccgagtcgggtcgtggcgaaggtttgc

SEQ ID NO: 100 - DNA, 50 bp, Zea mays
  source 1..50: mol_type = genomic DNA
tctcgaggatagcgagtgtctagagtgcgaggtgatgggtgtgctgattg

SEQ ID NO: 101 - DNA, 50 bp, Zea mays
  source 1..50: mol_type = genomic DNA
acaagaccaagcacgtgtcgctcaacaggtgggccaaacttttatcggat

SEQ ID NO: 102 - DNA, 50 bp, Zea mays
  source 1..50: mol_type = genomic DNA
aaagtaggcccagtacaccgctatcgacgtctacatgtcctacgagactg

SEQ ID NO: 103 - DNA, 50 bp, Zea mays
  source 1..50: mol_type = genomic DNA
gtcggctgctgctcatcagggagcgcgtggaagatgcggtcaccgtcggt

SEQ ID NO: 104 - DNA, 50 bp, Zea mays
  source 1..50: mol_type = genomic DNA
gcaacaatctcgttgtgtcatgttctatgttatcttagtgggactatttc

SEQ ID NO: 105 - DNA, 50 bp, Zea mays
  source 1..50: mol_type = genomic DNA
ttgttgcctattgtttactctctctagctccttacaacttcatgtctaat

SEQ ID NO: 106 - DNA, 50 bp, Zea mays
  source 1..50: mol_type = genomic DNA
gtgatgcgatgtgaattggactagtgatttctgtcacacccggattaaag

SEQ ID NO: 107 - DNA, 50 bp, Zea mays
  source 1..50: mol_type = genomic DNA
gggcaaacccaggtgcgtctcatatgtgcgccaaacgagatcaacacata

SEQ ID NO: 108 - DNA, 50 bp, Zea mays
  source 1..50: mol_type = genomic DNA
taatgacttagtgaatagagacgaatgtcacaatctttattacataacga

SEQ ID NO: 109 - DNA, 50 bp, Zea mays
  source 1..50: mol_type = genomic DNA
aatgttttacaaaataaattataatatatagaacgaactaatgtcattat

SEQ ID NO: 110 - DNA, 50 bp, Zea mays
  source 1..50: mol_type = genomic DNA
tgtcccttggcgccaacaagttgactgggagacgacacctagacctcgtc

SEQ ID NO: 111 - DNA, 50 bp, Zea mays
  source 1..50: mol_type = genomic DNA
gaactcctcgtagtagtcttcctctagggccacctgctcttcacctatgg

SEQ ID NO: 112 - DNA, 50 bp, Zea mays
  source 1..50: mol_type = genomic DNA
ggggagttttattgcaggagtgagctcacagatgttcatcgctcagcaag

SEQ ID NO: 113 - DNA, 50 bp, Zea mays
  source 1..50: mol_type = genomic DNA
tgtggagaataatgtgcatgggctcacttacggtggggctcatatatagt

SEQ ID NO: 114 - DNA, 50 bp, Zea mays
  source 1..50: mol_type = genomic DNA
gtcaagctgatcaacaaataatggttaaggcaaagcattgcttttaataa

SEQ ID NO: 115 - DNA, 50 bp, Zea mays
  source 1..50: mol_type = genomic DNA
gttggtcaaattttattagtagttactaaatataagtaaataccatccca

SEQ ID NO: 116 - DNA, 50 bp, Zea mays
  source 1..50: mol_type = genomic DNA
acaaaataatagataaaattaataataaaaccacaatacgatgcatatga

SEQ ID NO: 117 - DNA, 50 bp, Zea mays
  source 1..50: mol_type = genomic DNA
caaattaagtttaattccataagttaatcatgcaagagtcctcaatcgct

SEQ ID NO: 118 - DNA, 50 bp, Zea mays
  source 1..50: mol_type = genomic DNA
cttgaccacaagcacgactgatataccagttttacaatctgcagaggttg

SEQ ID NO: 119 - DNA, 50 bp, Zea mays
  source 1..50: mol_type = genomic DNA
cacatctttacccataatacatgttccctttgtcgcctcggattgatcaa

SEQ ID NO: 120 - DNA, 50 bp, Zea mays
  source 1..50: mol_type = genomic DNA
acccttaaacattgccaaggtgagtaggcagggtacactacaatgccttt

SEQ ID NO: 121 - DNA, 50 bp, Zea mays
  source 1..50: mol_type = genomic DNA
acaaagttccactagaggagagaacctcctatggtttctgggccgaaagt

SEQ ID NO: 122 - DNA, 50 bp, Zea mays
  source 1..50: mol_type = genomic DNA
tgtacaggaatccctcggtccaaaagctaaaaagatagtccgacccgagg

SEQ ID NO: 123 - DNA, 50 bp, Zea mays
  source 1..50: mol_type = genomic DNA
acctccctacacccgcatcctccactccgcttgcccctttcgggaaagga

SEQ ID NO: 124 - DNA, 50 bp, Zea mays
  source 1..50: mol_type = genomic DNA
ttaactcacactagctttcctaattaatcagccaagggtgtcccattcta

SEQ ID NO: 125 - DNA, 50 bp, Zea mays
  source 1..50: mol_type = genomic DNA
ccattgtgatagcactattatctcaagttaagctccatgttccagttaac

SEQ ID NO: 126 - DNA, 50 bp, Zea mays
  source 1..50: mol_type = genomic DNA
accataatcttatcatgaacatttaaataatccaataataatagttaagc

SEQ ID NO: 127 - DNA, 50 bp, Zea mays
  source 1..50: mol_type = genomic DNA
atgatcatgaatcatttgtatcttaacatccaaaaccacatagagcaata

SEQ ID NO: 128 - DNA, 50 bp, Zea mays
  source 1..50: mol_type = genomic DNA
gcagatactactcaaaagttcagtggcaagcaaggtataaagtaactcaa

SEQ ID NO: 129 - DNA, 50 bp, Zea mays
  source 1..50: mol_type = genomic DNA
actagggtgacctattgggtaccatcaaattaacctgagcatagtatagt

SEQ ID NO: 130 - DNA, 50 bp, Zea mays
  source 1..50: mol_type = genomic DNA
gacgactaacccgcaaatggcgcggctcgccagagtaatgctagggtggt

SEQ ID NO: 131 - DNA, 50 bp, Zea mays
  source 1..50: mol_type = genomic DNA
gcatcaatggataaaaacctgccgcacgcaggcacaaacgctaaagggac

SEQ ID NO: 132 - DNA, 50 bp, Zea mays
  source 1..50: mol_type = genomic DNA
caatggcgaggaagaacataggggtagtttacggttgcccggtgcaggga

SEQ ID NO: 133 - DNA, 50 bp, Zea mays
  source 1..50: mol_type = genomic DNA
cgagcagttcacgctccgcggtagcctggtccggtcttctacgtacgacg

SEQ ID NO: 134 - DNA, 50 bp, Zea mays
  source 1..50: mol_type = genomic DNA
atgaagtcctgttctccagtcaccaaacacgcaagggaagcccccaagcg

SEQ ID NO: 135 - DNA, 50 bp, Zea mays
  source 1..50: mol_type = genomic DNA
tccagcgcgcgaccctgccgaacccccagcttgacgccaagattcaaggg

SEQ ID NO: 136 - DNA, 50 bp, Zea mays
  source 1..50: mol_type = genomic DNA
cagcggcagcggccatttccaatgcgttctctctccgtgggtctattccc

SEQ ID NO: 137 - DNA, 50 bp, Zea mays
  source 1..50: mol_type = genomic DNA
agcggctatggctctcagtactggttatgggcgcatgatgcagagagatc

SEQ ID NO: 138 - DNA, 50 bp, Zea mays
  source 1..50: mol_type = genomic DNA
aaccgagagtgtgcgatctggtatttattcagggagtccaggttctgctg

SEQ ID NO: 139 - DNA, 50 bp, Zea mays
  source 1..50: mol_type = genomic DNA
gggagggagatattagctggcagactccaggcttgttgcaacggccaagt

SEQ ID NO: 140 - DNA, 50 bp, Zea mays
  source 1..50: mol_type = genomic DNA
ggagttgtgagtttgttgtgtcggggcctcagcgaaggcgaaggtgctga

SEQ ID NO: 141 - DNA, 50 bp, Zea mays
  source 1..50: mol_type = genomic DNA
caagcggtccccacggtgcagtgtgtgtggttgagcgcggcgtggtgcgg

SEQ ID NO: 142 - DNA, 50 bp, Zea mays
  source 1..50: mol_type = genomic DNA
gagccgtgcaatgcggatccggattaggccggtgttgggctgaacgagca

SEQ ID NO: 143 - DNA, 50 bp, Zea mays
  source 1..50: mol_type = genomic DNA
gcaaggagattcaggttgggttctgtttcatttttatttttttttctctt

SEQ ID NO: 144 - DNA, 50 bp, Zea mays
  source 1..50: mol_type = genomic DNA
ttctctctattttttcatttcacaagcccaattttttttcaaatccaaac

SEQ ID NO: 145 - DNA, 50 bp, Zea mays
  source 1..50: mol_type = genomic DNA
aaggttctaattcaaaaactccataggtgcaaattattatttttgggatg

SEQ ID NO: 146 - DNA, 50 bp, Zea mays
  source 1..50: mol_type = genomic DNA
tagtttaaccatatagtttattatattatcgttcactacttatttgattt

SEQ ID NO: 147 - DNA, 50 bp, Zea mays
  source 1..50: mol_type = genomic DNA
ggaaacaaagtaacatatagtctttcatcacaaattacttgagttcaatt

SEQ ID NO: 148 - DNA, 50 bp, Zea mays
  source 1..50: mol_type = genomic DNA
atattttaaaaattatttctaatacaaattaaatgaacagtcaaaatcta

SEQ ID NO: 149 - DNA, 50 bp, Zea mays
  source 1..50: mol_type = genomic DNA
aggcatgatgcataatttcaacttctatttcatttttatagggaacatgt

SEQ ID NO: 150 - DNA, 50 bp, Zea mays
  source 1..50: mol_type = genomic DNA
cttgccaataaaacatagacttacaataactctaattttattacaccaag

SEQ ID NO: 151 - DNA, 50 bp, Zea mays
  source 1..50: mol_type = genomic DNA
aactagctaacaatttaaaccatattttaagggtttacaaatcctacccc

SEQ ID NO: 152 - DNA, 50 bp, Zea mays
  source 1..50: mol_type = genomic DNA
ctaaaaaataatctcggccttgagattacgatgaactaggaaaggggtgg

SEQ ID NO: 153 - DNA, 50 bp, Zea mays
  source 1..50: mol_type = genomic DNA
ggaaattctactcgtacctcaacttcttgctcgtaggtggcttcatcttc

SEQ ID NO: 154 - DNA, 50 bp, Zea mays
  source 1..50: mol_type = genomic DNA
tgtgtgatgactccattgtactttgcacatcttaatcactttattccttg

SEQ ID NO: 155 - DNA, 50 bp, Zea mays
  source 1..50: mol_type = genomic DNA
taacacgagtcaaggtatcaatgatcttgataggatattctgtataagtc

SEQ ID NO: 156 - DNA, 50 bp, Zea mays
  source 1..50: mol_type = genomic DNA
agatcaccttgaacactaagttcatccatagttagttgctcttctggcac

SEQ ID NO: 157 - DNA, 50 bp, Zea mays
  source 1..50: mol_type = genomic DNA
cctaagacacttcttcaactgagatacatgtaagacatcatgtatatccg

SEQ ID NO: 158 - DNA, 50 bp, Zea mays
  source 1..50: mol_type = genomic DNA
ctagatggttgggtagctccagttgatatgcaactgcccccactcgcttt

SEQ ID NO: 159 - DNA, 50 bp, Zea mays
  source 1..50: mol_type = genomic DNA
aggattttgaagggtccaataaagcgaggggacaacttacctttaacttt

SEQ ID NO: 160 - DNA, 50 bp, Zea mays
  source 1..50: mol_type = genomic DNA
aaatttcctcatacaccgaattggtgacaccttgagatacacgtgatctc

SEQ ID NO: 161 - DNA, 50 bp, Zea mays
  source 1..50: mol_type = genomic DNA
tacagcttttctgtctggactgcgcagttctcaagttctcccttattatt

SEQ ID NO: 162 - DNA, 50 bp, Zea mays
  source 1..50: mol_type = genomic DNA
cgaatttgttcctctgcttcttggcttattattggcccaaagaactgcct

SEQ ID NO: 163 - DNA, 50 bp, Zea mays
  source 1..50: mol_type = genomic DNA
ttcaccagtttgatcctaatacagcaggattctacacttccttccatata

SEQ ID NO: 164 - DNA, 50 bp, Zea mays
  source 1..50: mol_type = genomic DNA
aggcctccaacggggacatcttcaaactggcctgataactattattatag

SEQ ID NO: 165 - DNA, 50 bp, Zea mays
  source 1..50: mol_type = genomic DNA
gagaactccgcatagggtaaactcttgtctcaactgccatcgtgcttaag

SEQ ID NO: 166 - DNA, 50 bp, Zea mays
  source 1..50: mol_type = genomic DNA
atcacaagctcttaacatatcttctagcacttgatttgttctcttggtct

SEQ ID NO: 167 - DNA, 50 bp, Zea mays
  source 1..50: mol_type = genomic DNA
gcccatcagtttagggatggtaggctgaactaaagtttagcttcgtgtcc

SEQ ID NO: 168 - DNA, 50 bp, Zea mays
  source 1..50: mol_type = genomic DNA
atactctcatgaaggttcttccaaaatcgggaagtaaactgagatccacg

SEQ ID NO: 169 - DNA, 50 bp, Zea mays
  source 1..50: mol_type = genomic DNA
atctaaaactatcttcttaggtaccccatgtaggcacatacactacacga

SEQ ID NO: 170 - DNA, 50 bp, Zea mays
  source 1..50: mol_type = genomic DNA
cggttgatctttagcgacccttattagggactgacggttggtcgctaaaa

SEQ ID NO: 171 - DNA, 50 bp, Zea mays
  source 1..50: mol_type = genomic DNA
gttatggactgacggttggtcgctaaatgcgcttataacgacggtctatg

SEQ ID NO: 172 - DNA, 50 bp, Zea mays
  source 1..50: mol_type = genomic DNA
ggtcgctaaaagtctagaaatttaacgacttatggtcggtcgctatagag

SEQ ID NO: 173 - DNA, 50 bp, Zea mays
  source 1..50: mol_type = genomic DNA
gtaagtgtgtgactgatggtgggtcgctataggggtacgtgatttttgtg

SEQ ID NO: 174 - DNA, 50 bp, Zea mays
  source 1..50: mol_type = genomic DNA
tttcttatttactcaccaaagcccatctaaaggcctactaaacaaacaaa

SEQ ID NO: 175 - DNA, 50 bp, Zea mays
  source 1..50: mol_type = genomic DNA
accctattccacgcaccaaaccacaagcgcagccgccgtcacttctgccc

SEQ ID NO: 176 - DNA, 50 bp, Zea mays
  source 1..50: mol_type = genomic DNA
catcgtggccccatcccgccgctcgtcgacgatgccaccccgcccctcgg

SEQ ID NO: 177 - DNA, 50 bp, Zea mays
  source 1..50: mol_type = genomic DNA
ccgtggtcgccgtcactataccacaacaccaaaatagcgtcagatctcag

SEQ ID NO: 178 - DNA, 50 bp, Zea mays
  source 1..50: mol_type = genomic DNA
tcactaaaagtagtaaattgacagtgaatcagtcgctaaaacatagctac

SEQ ID NO: 179 - DNA, 50 bp, Zea mays
  source 1..50: mol_type = genomic DNA
acaccatcagtcgatatttctaaatatactgttaaactgtccatcggtac

SEQ ID NO: 180 - DNA, 50 bp, Zea mays
  source 1..50: mol_type = genomic DNA
ttttatttatgtttcacgttgagctgtccgtcggcattaccatcttagga

SEQ ID NO: 181 - DNA, 50 bp, Zea mays
  source 1..50: mol_type = genomic DNA
gtcagaccatccgtcgttgtagaatgtcccaaaactaaagctctttgcgc

SEQ ID NO: 182 - DNA, 50 bp, Zea mays
  source 1..50: mol_type = genomic DNA
gtattggactagcttgcttgtgtctaaaattttccatgtggtgcacagtg

SEQ ID NO: 183 - DNA, 50 bp, Zea mays
  source 1..50: mol_type = genomic DNA
cactggcccggcccacaaaacatgtcaagttgacctagccaaatccaaat

SEQ ID NO: 184 - DNA, 50 bp, Zea mays
  source 1..50: mol_type = genomic DNA
ctcttcaatccctcgaccagtcgaccgcgccgctcacccgtgctgcccag

SEQ ID NO: 185 - DNA, 50 bp, Zea mays
  source 1..50: mol_type = genomic DNA
atctcctctcactccaccgtgtgcccagccctccgccgctagtcccctcc

SEQ ID NO: 186 - DNA, 50 bp, Zea mays
  source 1..50: mol_type = genomic DNA
cggtcctccccgcgcgttgtcatcgccggtcgcccagtcctccctgcagg

SEQ ID NO: 187 - DNA, 50 bp, Zea mays
  source 1..50: mol_type = genomic DNA
acgccatgctgcccagccttcctcgtagtcgtcctcagctcgtgtcttag

SEQ ID NO: 188 - DNA, 50 bp, Zea mays
  source 1..50: mol_type = genomic DNA
cgctctcccatggtggtaggccgacggctcctctcctacctcagagatag

SEQ ID NO: 189 - DNA, 50 bp, Zea mays
  source 1..50: mol_type = genomic DNA
atctacacccacggcagtggctgctctgatgacatcaatcaatacaatgg

SEQ ID NO: 190 - DNA, 50 bp, Zea mays
  source 1..50: mol_type = genomic DNA
gggttcctgctacggatccatctcctcaagtgttgatttaaggtcaagtg

SEQ ID NO: 191 - DNA, 50 bp, Zea mays
  source 1..50: mol_type = genomic DNA
tgttgtgtacttgtgtgtaggcctattgttgctgcttcccaaaatgcctc

SEQ ID NO: 192 - DNA, 50 bp, Zea mays
  source 1..50: mol_type = genomic DNA
gaatctgtgttccaacgacagcatgtctcctcctgtgcactcctgtgaag

SEQ ID NO: 193 - DNA, 50 bp, Zea mays
  source 1..50: mol_type = genomic DNA
acttaaggtgagttttatatgtcacttttctttaccgtgaatatttgagg

SEQ ID NO: 194 - DNA, 50 bp, Zea mays
  source 1..50: mol_type = genomic DNA
actaaacacacaagaactaatgtgctaggacataactcttatctcattta

SEQ ID NO: 195 - DNA, 50 bp, Zea mays
  source 1..50: mol_type = genomic DNA
ctgaatattttatttagcctagtggctactttgctatattttttgtccaa

SEQ ID NO: 196 - DNA, 50 bp, Zea mays
  source 1..50: mol_type = genomic DNA
tcattcaactgaaagtattatttttttccaaactgagatcagaccacaga

SEQ ID NO: 197 - DNA, 50 bp, Zea mays
  source 1..50: mol_type = genomic DNA
aaaaatagatataaagcataatagatgtgcctagtagatattcttgcatt

SEQ ID NO: 198 - DNA, 50 bp, Zea mays
  source 1..50: mol_type = genomic DNA
ggttatggctgctactagtcatgtggctgatttttagatgtgctcaaaat

SEQ ID NO: 199 - DNA, 50 bp, Zea mays
  source 1..50: mol_type = genomic DNA
aaaatattatttttattagccaaggagtagtatttggagtagtattcttg

SEQ ID NO: 200 - DNA, 50 bp, Zea mays
  source 1..50: mol_type = genomic DNA
gattcattctctagtgtctcggttggtctttgtctgtcaattttaaatgc

SEQ ID NO: 201 - DNA, 50 bp, Zea mays
  source 1..50: mol_type = genomic DNA
caaatcttgtacccaactattctgccagcgcatggtgacataatactgtc

SEQ ID NO: 202 - DNA, 50 bp, Zea mays
  source 1..50: mol_type = genomic DNA
acacccggttttagaaggcaaaccgaatgcgaaccatgtacgtgccagga

SEQ ID NO: 203 - DNA, 50 bp, Zea mays
  source 1..50: mol_type = genomic DNA
tcagcaattcacgtacacagcagttacatacatggacatcatcacacagt

SEQ ID NO: 204 - DNA, 50 bp, Zea mays
  source 1..50: mol_type = genomic DNA
gctcaaatagtattaaaaagggaaataatagtcggttacatcatatgtct

SEQ ID NO: 205 - DNA, 50 bp, Zea mays
  source 1..50: mol_type = genomic DNA
gagacatccacatagtctttacaataaatcaaagtgcggaaaagaaacat

SEQ ID NO: 206 - DNA, 50 bp, Zea mays
  source 1..50: mol_type = genomic DNA
agataaacgcggccttcacaggcagccaactaggggttgccgctaaccca

SEQ ID NO: 207 - DNA, 50 bp, Zea mays
  source 1..50: mol_type = genomic DNA
cgcctagatctcgtcgtagtcttggaacttctggaagtctccttccatgg

SEQ ID NO: 208 - DNA, 50 bp, Zea mays
  source 1..50: mol_type = genomic DNA
cttcatcctctcctgagcagtggttgcaatgctgacaacctggggatggg

SEQ ID NO: 209 - DNA, 50 bp, Zea mays
  source 1..50: mol_type = genomic DNA
ggggggtttggtgtgtagagcaagggtgagtacacatcaacatactcagc

SEQ ID NO: 210 - DNA, 50 bp, Zea mays
  source 1..50: mol_type = genomic DNA
aagtgtcctgtttggctgtagtggactagctttatgtgggcataagttaa

SEQ ID NO: 211 - DNA, 50 bp, Zea mays
  source 1..50: mol_type = genomic DNA
gcagttgcttttagttggtcagattattatttactagtagaaagccaggt

SEQ ID NO: 212 - DNA, 50 bp, Zea mays
  source 1..50: mol_type = genomic DNA
tttaacattgacccaagttattagcccgatgtactctttccaaacggaaa

SEQ ID NO: 213 - DNA, 50 bp, Zea mays
  source 1..50: mol_type = genomic DNA
gaataccacttaccagcaccatagtcataaccagaaccatcaacctcatt

SEQ ID NO: 214 - DNA, 50 bp, Zea mays
  source 1..50: mol_type = genomic DNA
gccacctgtaaaccaaaatgtctctgatcaagtaccactaatcactggag

SEQ ID NO: 215 - DNA, 50 bp, Zea mays
  source 1..50: mol_type = genomic DNA
ctcccttggccgctcataaccgcgagcacggctgatatatcagttttcaa

SEQ ID NO: 216 - DNA, 50 bp, Zea mays
  source 1..50: mol_type = genomic DNA
acactctgcagaggttgtgcactttacccacaagccgtgattcccttttt

SEQ ID NO: 217 - DNA, 50 bp, Zea mays
  source 1..50: mol_type = genomic DNA
ctgcccggagatcatgactctccattgatcactaccaaggtgacccagca

SEQ ID NO: 218 - DNA, 50 bp, Zea mays
  source 1..50: mol_type = genomic DNA
gggcatcactatgtagcctttacaaagattccccggggctgtagccgcct

SEQ ID NO: 219 - DNA, 50 bp, Zea mays
  source 1..50: mol_type = genomic DNA
gttaggtttcctaaatgcaccgcactcctccccaaggggcgaacccaaac

SEQ ID NO: 220 - DNA, 50 bp, Zea mays
  source 1..50: mol_type = genomic DNA
ttggcagagcgagccgcatacaccgagccccattgacggcacgacggtta

SEQ ID NO: 221 - DNA, 50 bp, Zea mays
  source 1..50: mol_type = genomic DNA
agcgaactacaccccggatcctctaattattcagctaagggcaccccatt

SEQ ID NO: 222 - DNA, 50 bp, Zea mays
  source 1..50: mol_type = genomic DNA
ccaccctcatggttgcactgttttcccggacggtcatccaaagaacaggt

SEQ ID NO: 223 - DNA, 50 bp, Zea mays
  source 1..50: mol_type = genomic DNA
tcttacggagaggcactcgagaaaccgctcgagccccgttgaagaccaca

SEQ ID NO: 224 - DNA, 50 bp, Zea mays
  source 1..50: mol_type = genomic DNA
agtataacatcataataagaagagggaaaacaacgtatcattgatcattc

SEQ ID NO: 225 - DNA, 50 bp, Zea mays
  source 1..50: mol_type = genomic DNA
tcatcatgttcattgattagagttgagcaatagcataagactaagcaata

SEQ ID NO: 226 - DNA, 50 bp, Zea mays
  source 1..50: mol_type = genomic DNA
ataatccaacccaaataggtaaccaaggacatggataacaaagctagtaa

SEQ ID NO: 227 - DNA, 50 bp, Zea mays
  source 1..50: mol_type = genomic DNA
atccttaggcataaatgtgtaatgcgggagatgaattaaagaatgaatag

SEQ ID NO: 228 - DNA, 50 bp, Zea mays
  source 1..50: mol_type = genomic DNA
gacagagataggtcaagtgacacttgcctccaccaacagactgctgctca

SEQ ID NO: 229 - DNA, 50 bp, Zea mays
  source 1..50: mol_type = genomic DNA
ggggcttctcctgcgagttcttcgggctcttcaaccggatcgttctctat

(19) United States
(12) Patent Application Publication
(10) Pub. No.: US 2026/0286373 A1
(43) Pub. Date: Sep. 24, 2026

(54) MODIFICATIONS OF TRANSGENIC CORN EVENT ZM_BCS216090 AND METHODS THEREOF

(71) Applicant: Monsanto Technology LLC, St. Louis, MO (US)

(72) Inventor: Linda Rymarquis, High Ridge, MO (US)

(21) Appl. No.: 19/474,152

(22) PCT Filed: Apr. 29, 2024
(86) PCT No.: PCT/US2024/026785

§ 371 Date: Oct. 9, 2025

Related U.S. Application Data
(60) Provisional application No. 63/499,279, filed on May 1, 2023.

(51) Int. Cl. C12N 15/82; C12N 9/22; C12N 15/11; C12Q 1/6895

(52) U.S. Cl. CPC C12N 15/8213; C12N 9/226; C12N 15/11; C12Q 1/6895; C12N 2310/20; C12Q 2600/13; C12Q 2600/156

(57) ABSTRACT

A modified corn event ZM_BCS216090 and plants, plant cells, seeds, plant parts, progeny plants and commodity products comprising a modified corn event ZM_BCS216090 are provided. Polynucleotides and sequences specific for a modified corn event ZM_BCS216090, methods for making a modified corn event ZM_BCS216090, methods for making and using plants, plant cells, seeds, plant parts, progeny plants, and commodity products comprising a modified corn event ZM_BCS216090, and methods for detecting a modified corn event ZM_BCS216090, or a polynucleotide or DNA sequence specific for a modified corn event ZM_BCS216090, in a DNA molecule or sample, are provided.

### CROSS-REFERENCE TO RELATED APPLICATIONS

This application claims the priority of U.S. Provisional Appl. Ser. 63/499,279, filed May 1, 2023, the entire disclosure of which is incorporated herein by reference.

### INCORPORATION OF SEQUENCE LISTING

The sequence listing contained in the file named “MONS573WO_ST26.xml” is 240 kilobytes (measured in Microsoft Windows®), was created on Apr. 18, 2024, is filed herewith by electronic submission, and is incorporated herein by reference.

### FIELD OF THE INVENTION

The present invention relates to recombinant DNA molecules present in and/or isolated from a modified corn event ZM_BCS216090. The invention also relates to transgenic corn plants, plant parts, and seeds, cells, and agricultural products containing a modified corn event ZM_BCS216090, as well as methods of using the same and making and detecting the presence of a modified corn event ZM_BCS216090.

### BACKGROUND

Corn (Zea mays) is an important crop and is a primary food source in many areas of the world. The methods of biotechnology have been applied to corn for improvement of the agronomic traits and quality of the product. Improved agronomic traits can include increased yield potential and stress tolerance such as increased lodging resistance, which may be accomplished through the expression of a transgene inserted into the genome of the corn plant.

The expression of transgenes in a transgenic plant, plant part, seed or cell, and thus their effectiveness, may be influenced by many different factors, such as the regulatory elements used in the transgene cassette, the chromosomal location of the transgene insertion site, the chromatin structure of the genome at or near the transgene insertion site, and the presence or proximity of any endogenous cis and/or trans regulatory elements or genes close to the transgene insertion site. These differences may result in variation in the level of transgene expression or in the spatial or temporal pattern of transgene expression between different transgenic insertion events of the same expression cassette. Different transgenic events may also have different levels or patterns of transgene expression in different plant germplasms and growth conditions and across different plant tissues and developmental stages. In addition, transformation events can also vary in terms of the molecular quality of the event. For example, a transgenic insertion event may be truncated relative to the intended insertion or contain additional vector backbone sequences. There may also be undesirable phenotypic or agronomic differences between some events. Since Agrobacterium-mediated transformation with a T-DNA construct containing a transgene expression cassette is largely variable and random in terms of where the transgene can be inserted into the plant genome, a variety of different transgenic events can be generated with unique chromosomal insertion sites.

For these reasons, the performance of different transformation events from the same transformation vector construct can vary, and the identification of transformation events conferring the most beneficial traits or characteristics without other potential off-types or concerns is needed to select a superior event for commercial use. Therefore, a number of individual plant transformation events must be produced and analyzed to select an event having superior commercial properties, which can be a significant undertaking that involves analysis and selection among many different transformation events.

### SUMMARY OF THE INVENTION

In one aspect, the present disclosure provides a recombinant DNA molecule comprising: (a) a first nucleotide sequence (i) comprising an expression cassette that encodes a microRNA (miRNA) that suppresses the expression of the endogenous GA20ox3 and GA20ox5 genes in corn, or (ii) selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9; and (b) a second nucleotide sequence that (i) comprises at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 17 or 19, or (ii) is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 17 or 19, or (iii) is selected from the group consisting of SEQ ID NOs: 30-49 and SEQ ID NOs: 50-129. In some embodiments, the recombinant DNA molecule may further comprise (c) a third nucleotide sequence that (i) comprises at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 18 or 20, or (ii) is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 18 or 20, or (iii) is selected from the group consisting of SEQ ID NOs: 130-149 and SEQ ID NOs: 150-229. The third nucleotide sequence, in certain embodiments, is selected from the group consisting of SEQ ID NOs: 130-149 and SEQ ID NOs: 150-229. The recombinant DNA molecule, particular embodiments, has a deletion of one or more consecutive nucleotides between the second nucleotide sequence and the third nucleotide sequence relative to SEQ ID NO: 10, 18 or 20. In various embodiments, the second nucleotide sequence is selected from the group consisting of SEQ ID NOs: 30-49 and SEQ ID NOs: 50-129. In some embodiments, the recombinant DNA molecule has a deletion of one or more consecutive nucleotides between the first nucleotide sequence and the second nucleotide sequence relative to SEQ ID NO: 10, 17 or 19.

In another aspect, the present disclosure provides a recombinant DNA molecule comprising: (a) a first nucleotide sequence (i) comprising an expression cassette that encodes a microRNA (miRNA) that suppresses the expression of the endogenous GA20ox3 and GA20ox5 genes in corn, or (ii) selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9; and (b) a second nucleotide sequence that (i) comprises at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 18 or 20, or (ii) is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 18 or 20, or (iii) is selected from the group consisting of SEQ ID NOS: 130-149 and SEQ ID NOs: 150-229. In certain embodiments, the second nucleotide sequence is selected from the group consisting of SEQ ID NOs: 130-149 and SEQ ID NOs: 150-229. The recombinant DNA molecule, in some embodiments, has a deletion of one or more consecutive nucleotides between the first nucleotide sequence and the second nucleotide sequence relative to SEQ ID NO: 10, 18 or 20. In particular embodiments, the recombinant DNA molecule may further comprise nucleotides 1,000-1,001 or 3,733-3,734 of SEQ ID NO: 10.

In many embodiments, a recombinant DNA molecule of the present disclosure may be comprised in a corn plant, corn plant part, corn plant cell, corn plant seed, corn progeny plant, or commodity or fuel product made from corn and corn plant parts. In some embodiments, a recombinant DNA molecule of the present disclosure may comprise an amplicon diagnostic for the presence of DNA comprising a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9. A recombinant DNA molecule of the present disclosure, in particular embodiments, is derived from a corn plant, corn plant part, corn seed, processed corn seed, corn plant cell or tissue, animal feed comprising corn, corn oil, corn meal, corn flour, corn flakes, corn bran, food made comprising corn, corn biomass, or fuel products made from corn and corn plant parts.

In yet another aspect, the present disclosure provides a recombinant DNA molecule comprising a polynucleotide segment of sufficient length to function as a DNA probe that hybridizes specifically under stringent hybridization conditions with a polynucleotide having a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 17, SEQ ID NO: 18, SEQ ID NO: 19, and SEQ ID NO: 20. In still yet another aspect, the present disclosure provides a pair of DNA molecules that can function as DNA primers when used together in an amplification reaction comprising a first DNA molecule and a second DNA molecule, wherein the first DNA molecule and the second DNA molecule are different, (a) wherein the first DNA molecule is: (i) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9, or a complement thereof, or (ii) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 17 or 19, or a complement thereof; and wherein the second DNA molecule is: (i) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9, or a complement thereof, or (ii) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 17 or 19, or a complement thereof, or (iii) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 18 or 20, or a complement thereof. In certain embodiments, the first DNA molecule and the second DNA molecule can be used together in an amplification reaction with a sample from a plant, plant part, plant seed, plant cell, food or animal feed, or commodity or fuel product made from a plant or plant part to produce an amplicon diagnostic for the presence of a modified corn event ZM_BCS216090 DNA in the sample. The amplicon, in some embodiments, comprises a nucleotide sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10. The modified corn event ZM_BCS216090 DNA, in many embodiments, is a further modified corn event ZM_BCS216090 DNA. In yet another embodiment, the present disclosure provides a DNA detection kit comprising: a) a DNA probe of the present disclosure; and/or b) a pair of DNA molecules that function as DNA primers as provided by the present disclosure.

In one aspect, the present disclosure provides a method of detecting the presence of a DNA segment diagnostic for a modified corn event ZM_BCS216090 DNA in a sample, the method comprising: contacting the sample with the DNA molecule that functions as a DNA probe of the present disclosure; subjecting the sample and the DNA molecule to stringent hybridization conditions; and detecting hybridization of the DNA molecule to the DNA segment in the sample, wherein the detection is diagnostic for the presence of the modified corn event ZM_BCS216090 DNA in the sample. In another aspect, the present disclosure provides a method of detecting the presence of a DNA segment diagnostic for a modified corn event ZM_BCS216090 DNA in a sample, the method comprising: contacting the sample with a pair of DNA molecules capable of functioning as DNA primers when used together in an amplification reaction provided by the present disclosure; performing an amplification reaction sufficient to produce a DNA amplicon; and detecting the presence of the DNA amplicon in the reaction, wherein the presence of the DNA amplicon is diagnostic for the presence of the modified corn event ZM_BCS216090 DNA in the sample. In yet another aspect, the present disclosure provides a method of detecting the presence of a DNA segment diagnostic for a modified corn event ZM_BCS216090 DNA in a sample, the method comprising performing a sequencing reaction with the sample, wherein the production in the sequencing reaction of a target nucleotide sequence comprising at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9, or a complement thereof, is diagnostic for the modified corn event ZM_BCS216090 DNA in the sample. In particular embodiments, the modified corn event ZM_BCS216090 is a further modified corn event ZM_BCS216090.

In another aspect, the present disclosure provides a modified corn plant, corn plant part, corn seed, or corn cell comprising a modified corn event ZM_BCS216090 or comprising a recombinant DNA molecule as provided herein. In some embodiments, the modified corn plant, corn plant part, corn seed, or corn cell may comprise a recombinant DNA molecule or DNA segment comprising a nucleotide sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10, or a complement thereof; or a recombinant DNA molecule or DNA segment comprising a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10, SEQ ID NO: 17, SEQ ID NO: 18, SEQ ID NO: 19, or SEQ ID NO: 20, or a complement thereof. The modified corn plant, corn plant part, corn seed, or corn cell, in particular embodiments may exhibit reduced expression of at least a first endogenous gibberellin 20-oxidase (GA20ox) gene. In some embodiments, the gibberellin 20-oxidase (GA20ox) gene is selected from the group consisting of gibberellin 20-oxidase 3 (GA20ox3) and gibberellin 20-oxidase 5 (GA20ox5). The modified corn plant, corn plant part, corn seed, or corn cell, in certain embodiments, has reduced expression of an endogenous gibberellin 20-oxidase 3 (GA20ox3) gene and an endogenous gibberellin 20-oxidase 5 (GA20ox5) gene. In particular embodiments, the corn plant, corn plant part, corn seed, or corn cell is further defined as a progeny plant of any generation of a corn plant comprising a modified corn event ZM_BCS216090, or a corn plant part, corn seed, or corn cell derived therefrom. The modified corn plant, in some embodiments, has a reduced plant height relative to a control corn plant. The modified corn plant, in many embodiments, has an increased lodging resistance relative to a control corn plant. In a number of embodiments, the recombinant DNA molecule is chromosome 1, or the DNA segment is present in chromosome 1 of the modified corn plant, corn plant part, corn seed, or corn cell. The modified corn event ZM_BCS216090, in some embodiments, is a further modified corn event ZM_BCS216090. In certain embodiments, the modified corn event ZM_BCS216090 of the modified corn plant, corn plant part, corn seed, or corn cell comprises a genetic modification, mutation or edit, relative to the corn event ZM_BCS216090, introduced via a targeted genome editing technique.

In yet another aspect, the present disclosure provides a method of producing a progeny corn plant comprising a modified corn event ZM_BCS216090 comprising: sexually crossing a first modified corn plant that comprises a modified corn event ZM_BCS216090 with itself or a second corn plant; collecting one or more seeds produced from the cross; growing the seed to produce one or more progeny plants; and selecting at least a first progeny plant or seed comprising a modified corn event ZM_BCS216090. In certain embodiments, the at least a first progeny plant has a reduced plant height and/or increased lodging resistance relative to a control corn plant. The modified corn event ZM_BCS216090, in some embodiments, is a further modified corn event ZM_BCS216090. In many embodiments, the methods of the present disclosure may further comprise collecting seed from the at least first progeny plant comprising a modified corn event ZM_BCS216090. The present disclosure, in additional embodiments, provides a hybrid modified corn plant or seed comprising a modified corn event ZM_BCS216090 produced by the methods described herein. In some embodiments, the hybrid modified corn plan or seed of claim 39, wherein the modified corn event ZM_BCS216090 is a further modified corn event ZM_BCS216090.

Aspects of the present disclosure provide a nonliving corn plant material comprising a detectable amount of a recombinant DNA molecule as described herein. In some embodiments, the present disclosure provides microorganisms and commodity products comprising a recombinant DNA molecule of the present disclosure. The microorganism, in a number of embodiments, is a plant cell. In some embodiments, a commodity product is produced from a modified corn plant, corn plant part, corn seed, or corn tissue or cell comprising a modified corn event ZM_BCS216090. The modified corn event ZM_BCS216090, in certain embodiments, is a further modified corn event ZM_BCS216090. Non-limiting examples of commodity products include whole or processed corn seed, animal feed comprising corn, corn oil, corn meal, corn flour, corn flakes, corn bran, corn biomass, and fuel products produced using corn and corn plant parts. In certain aspects, the present disclosure provides a method of producing a commodity product, the method comprising: a) obtaining a modified corn plant, corn plant part, or corn seed comprising a modified corn event ZM_BCS216090; and b) producing a commodity product from the transgenic corn plant, corn plant part, or corn seed. The modified corn event ZM_BCS216090, in further embodiments, is a further modified corn event ZM_BCS216090.

In one aspect, the present disclosure provides a corn plant, corn plant part, or corn seed comprising a DNA molecule or segment functional as a template when tested in a DNA amplification method to produce an amplicon diagnostic for the presence of a modified corn event ZM_BCS216090 DNA.

In another aspect, the present disclosure provides a method of determining the zygosity of a corn plant, corn plant part, or corn seed comprising a modified corn event ZM_BCS216090 comprising: contacting a sample comprising DNA from the corn plant, corn plant part, or corn seed with a pair of DNA molecules that function as DNA primers, as provided by the present disclosure; performing a nucleic acid amplification reaction with the sample and the pair of DNA molecules; and detecting in the nucleic acid amplification reaction a first amplicon diagnostic for a modified corn event ZM_BCS216090 and a second amplicon diagnostic for native corn genomic DNA not comprising the modified corn event ZM_BCS216090, wherein the presence of only the first amplicon is diagnostic of a corn plant, corn plant part, or corn seed homozygous for the modified corn event ZM_BCS216090, and the presence of both the first amplicon and the second amplicon is diagnostic of a corn plant, corn plant part, or corn seed heterozygous for the modified corn event ZM_BCS216090. In yet another aspect, the present disclosure provides a method of determining the zygosity of a corn plant, corn plant part, or corn seed comprising a modified corn event ZM_BCS216090 comprising: contacting a sample comprising DNA from the corn plant, corn plant part, or corn seed with a first primer pair that can produce a first amplicon of all or part of the modified corn event ZM_BCS216090 and a second primer pair that can produce a second amplicon of a standard genomic sequence known to be single copy and homozygous in the corn plant, corn plant part, or corn seed; contacting the sample with a first probe that specifically hybridizes to the first amplicon and/or all or part of the modified corn event ZM_BCS216090, and a second probe that specifically hybridizes to the standard genomic sequence; performing a DNA amplification reaction using real-time PCR with the sample and determining the cycle thresholds (Ct values) of the first amplicon and the second amplicon; calculating the difference (ΔCt) between the Ct values of the second amplicon and the first amplicon; and determining the zygosity of the modified corn event ZM_BCS216090, wherein a ΔCt of about zero (0) indicates homozygosity of the modified corn event ZM_BCS216090 and a ΔCt of about one (1) indicates heterozygosity of the modified corn event ZM_BCS216090. In some embodiments, the first and second primer pairs comprise SEQ ID NO: 11 combined with SEQ ID NO: 12, and SEQ ID NO: 14 combined with SEQ ID NO: 15.

In still yet another aspect, the present disclosure provides a method of determining the zygosity of a corn plant, corn plant part, or corn seed comprising a modified corn event ZM_BCS216090 comprising: contacting a sample comprising DNA from the corn plant, corn plant part, or corn seed with a primer pair capable of producing a first amplicon diagnostic for the modified corn event ZM_BCS216090 and a second amplicon diagnostic for native corn genomic DNA not comprising the modified corn event ZM_BCS216090; performing a nucleic acid amplification reaction with the sample and the set of primer pairs; and detecting the first amplicon and the second amplicon, wherein the presence of only the first amplicon is diagnostic of a corn plant, corn plant part, or corn seed homozygous for the modified corn event ZM_BCS216090, the presence of only the second amplicon is diagnostic of a corn plant, corn plant part, or corn seed homozygous for native corn genomic DNA not comprising the modified corn event ZM_BCS216090, and the presence of both the first amplicon and the second amplicon is diagnostic of a corn plant, corn plant part, or corn seed heterozygous for the modified corn event ZM_BCS216090. In one aspect, the present disclosure provides a method of determining the zygosity of a corn plant, corn plant part, or corn seed comprising a modified corn event ZM_BCS216090 comprising: contacting a sample comprising DNA from the corn plant, corn plant part, or corn seed with a probe set which contains at least a first probe that specifically hybridizes to the modified corn event ZM_BCS216090 and at least a second probe that specifically hybridizes to corn genomic DNA that was disrupted by insertion of the heterologous DNA of corn event ZM_BCS216090 and is disrupted by the modified corn event ZM_BCS216090 DNA, wherein the second probe does not hybridize to the modified corn event ZM_BCS216090 DNA; and hybridizing the probe set with the sample under stringent hybridization conditions, wherein detecting hybridization of only the first probe under the hybridization conditions is diagnostic for a corn plant, corn plant part, or corn seed homozygous for the modified corn event ZM_BCS216090, and wherein detecting hybridization of both the first probe and the second probe under the hybridization conditions is diagnostic for a corn plant, corn plant part, or corn seed heterozygous for the modified corn event ZM_BCS216090. The probe set, in particular embodiments, comprises SEQ ID NO: 13 and SEQ ID NO: 16. In some embodiments, the modified corn event ZM_BCS216090 is a further modified corn event ZM_BCS216090.

Aspects of the present disclosure provide a population of transgenic corn plants, wherein each transgenic corn plant comprises a modified corn event ZM_BCS216090. In some embodiments, the population of corn plants has a reduced plant height on average relative to a population of control corn plants lacking the modified corn event ZM_BCS216090. The modified corn event ZM_BCS216090, in additional embodiments, is a further modified corn event ZM_BCS216090. In particular embodiments, the population of corn plants has an increased lodging resistance on average relative to a population of control corn plants lacking the modified corn event ZM_BCS216090.

In one aspect, the present disclosure provides a method of modifying a corn plant, the method comprising: (a) introducing a site-specific nuclease or a recombinant DNA construct comprising an expression cassette encoding a site-specific nuclease into at least one cell of an explant of a corn plant comprising corn event ZM_BCS216090, or a plant part thereof, to produce a modified corn event ZM_BCS216090 via a targeted genome editing technique; and (b) developing or regenerating a modified corn plant from the explant, wherein the modified corn plant comprises the modified corn event ZM_BCS216090. The modified corn event ZM_BCS216090, in some embodiments, is a further modified corn event ZM_BCS216090. Non-limiting examples site-specific nucleases include a zinc-finger nuclease (ZFN), a meganuclease, an RNA-guided endonuclease, a TALE-endonuclease (TALEN), a recombinase, and a transposase. In some embodiments, the site-specific nuclease is an RNA-guided endonuclease or a CRISPR/Cas nuclease. The introducing step (a), in particular embodiments, comprises introducing the recombinant DNA construct into the at least one cell of the explant, wherein the recombinant DNA construct further comprises an expression cassette encoding a first guide RNA (gRNA). The recombinant DNA construct, in additional embodiments, further comprises an expression cassette encoding a second guide RNA (gRNA). The introducing step (a), in certain embodiments, further comprises introducing a first guide RNA (gRNA) or a second recombinant DNA construct comprising an expression cassette encoding a first guide RNA (gRNA) into the at least one cell of the explant. The introducing step (a), in some embodiments, comprises introducing at least two guide RNAs (gRNAs) comprising the first gRNA and a second gRNA into the at least one cell of the explant. In a number of embodiments, the second recombinant DNA construct further comprises an expression cassette encoding a second guide RNA (gRNA). The introducing step (a), in many embodiments, comprises introducing a first gRNA and second recombinant DNA construct comprising an expression cassette encoding a second guide RNA (gRNA) into the at least one cell of the explant. In some embodiments, the site-specific nuclease has a first target site in the genome of the corn plant at or near corn event ZM_BCS216090. In particular embodiments, the site-specific nuclease has a second target site in the genome of the corn plant at or near corn event ZM_BCS216090. The introducing step (a), in certain embodiments, comprises introducing a second site-specific nuclease or a recombinant DNA construct comprising an expression cassette encoding a second site-specific nuclease into at least one cell of the explant, and wherein the second site-specific nuclease has a second target site in the genome of the corn plant at or near corn event ZM_BCS216090. In some embodiments, the first gRNA has a first target site in a flanking DNA sequence, 5′ flank, 3′ flank, junction sequence, or insertion sequence of corn event ZM_BCS216090, or a complement thereof. In particular embodiments, the first gRNA has a first target site comprising a target sequence that is: (i) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 17 or 19, or a complement thereof; or (ii) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 18 or 20, or a complement thereof; or (iii) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9, or a complement thereof. In some embodiments, the first gRNA has a second target site comprising a target sequence that is: (i) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 17 or 19, or a complement thereof; or (ii) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 18 or 20, or a complement thereof; or (iii) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9, or a complement thereof. In certain embodiments, (i) the first gRNA has a first target site comprising a target sequence that is: (1) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 17 or 19, or a complement thereof; or (2) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 18 or 20, or a complement thereof; or (3) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9, or a complement thereof; and (ii) the second gRNA has a second target site comprising a target sequence that is: (1) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 17 or 19, or a complement thereof; or (2) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 18 or 20, or a complement thereof; or (3) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9, or a complement thereof. In some embodiments, the modified corn event ZM_BCS216090 comprises a deletion or excision of intervening genomic DNA between the first target site and the second target site, relative to the corn event ZM_BCS216090. In certain embodiments, the methods of the present disclosure may further comprise selecting the modified corn plant comprising the modified corn event ZM_BCS216090, and sexually crossing the modified corn plant with itself or a second corn plant to produce one or more modified progeny corn plants.

Aspects of the present disclosure provide a method of introducing a target site into a corn plant, the method comprising: (a) introducing a cognate target site into the corn event ZM_BCS216090 locus of at least one cell of a corn plant or corn plant part comprising the corn event ZM_BCS216090 or an explant thereof via a targeted genome editing technique, wherein the cognate target site is identical or similar to an originator target site for a site-specific nuclease present in the corn event ZM_BCS216090 locus, and (b) developing or regenerating a modified corn plant comprising a modified corn event ZM_BCS216090 comprising the cognate target site. In some embodiments, the methods of the present disclosure may further comprise: (c) introducing a site-specific nuclease or a recombinant DNA construct comprising an expression cassette encoding a site-specific nuclease into at least one cell of an explant of a corn plant comprising the modified corn event ZM_BCS216090 or a plant part thereof, to produce a further modified corn event ZM_BCS216090 via a targeted genome editing technique, wherein the target site of the site-specific nuclease includes the cognate target site and the originator target site; and (d) developing or regenerating a second modified corn plant comprising the further modified corn event ZM_BCS216090.

In certain aspects, the present disclosure provides a method of introducing a target site into a corn plant, the method comprising: (a) introducing a cognate target site into the corn event ZM_BCS216090 locus of at least one cell of a corn plant or corn plant part comprising the corn event ZM_BCS216090 or an explant thereof via a targeted genome editing technique to produce a modified corn event ZM_BCS216090 comprising the cognate target site, wherein the cognate target site is identical or similar to an originator target site for a site-specific nuclease present in the corn event ZM_BCS216090 locus, and (b) introducing a site-specific nuclease or a recombinant DNA construct comprising an expression cassette encoding a site-specific nuclease into at least one cell of an explant of a modified corn plant comprising the modified corn event ZM_BCS216090, or a plant part thereof, to produce a further modified corn event ZM_BCS216090 via a targeted genome editing technique, wherein the target site of the site-specific nuclease includes the cognate target site and the originator target site; and (c) developing or regenerating a second modified corn plant comprising the further modified corn event ZM_BCS216090. In some embodiments, the further modified corn event ZM_BCS216090 of the second modified corn plant comprises a deletion or excision of intervening genomic DNA between the originator target site and the cognate target site, relative to the corn event ZM_BCS216090 or modified corn event ZM_BCS216090. In certain embodiments, the methods of the present disclosure may further comprise selecting the second modified corn plant or a progeny plant of the second modified corn plant comprising the further modified corn event ZM_BCS216090, and sexually crossing the second modified corn plant or the progeny plant with itself or another corn plant to produce one or more modified progeny corn plants comprising the further modified corn event ZM_BCS216090. In additional embodiments, the modified corn event ZM_BCS216090 is a further modified corn event ZM_BCS216090.

In some aspects, the present disclosure provides a method of modifying an explant of a corn plant or plant part, the method comprising: introducing a site-specific nuclease or a recombinant DNA construct comprising an expression cassette encoding a site-specific nuclease into at least one cell of an explant of a corn plant or plant part comprising corn event ZM_BCS216090 to produce a modified corn event ZM_BCS216090 into the at least one cell of the explant. In certain embodiments, the modified corn event ZM_BCS216090 is a further modified corn event ZM_BCS216090.

### BRIEF DESCRIPTION OF THE DRAWINGS

FIG. 1 represents the sequence of corn event ZM_BCS216090. Horizontal lines and boxes correspond to the positions of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9, relative to SEQ ID NO: 10. The horizontal arrows labeled SQ51606 (SEQ ID NO: 11) and SQ51629 (SEQ ID NO: 12) represent the approximate position of a pair of primers that can be used to detect corn event ZM_BCS216090 and the horizontal line labeled PB50583 (SEQ ID NO: 13) represents the approximate position of a DNA probe that can be used to detect corn event ZM_BCS216090.

FIG. 2 shows the pM578 construct vector map used for Agrobacterium-mediated transformation of corn to produce transgenic events provided herein including ZM_BCS216090.

FIG. 3 represents the miRNA expression cassette of corn event ZM_BCS216090 relative to SEQ ID NO: 9 with their respective genetic elements labeled as described in Table 1.

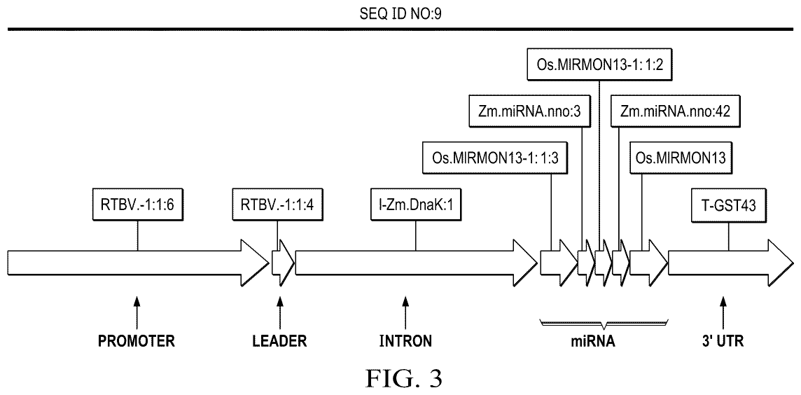

FIG. 4 represents the approximate timing of creation, testing, characterization, and selection of the ZM_BCS216090 corn event as described herein.

FIG. 5 is a diagrammatic representation of the breeding process to produce the marker-free corn event ZM_BCS216090. R₀ generation events (“transformants”) are those that are derived from the initial transformation with the transformation vector used to generate corn event ZM_BCS216090. Subsequent “R” generations (R₁, and R₂) represent successive generations produced through self-pollination of plants derived from the initial Ro transformant that resulted in corn event ZM_BCS216090. The R₂ transformants, which are homozygous for the T-DNA insertion, are cross-pollinated with an elite transgenic corn line comprising a transgene cassette for the expression of Cre-recombinase, resulting in an F₁ generation, wherein many of the progeny have lost the CP4 EPSPS selectable marker cassette due to Cre-recombinase excision. Hemizygous T-DNA positive, CP4 EPSPS negative plants are selected and self-pollinated, resulting in an F₂ generation. F₂ plants homozygous for the inserted T-DNA without the CP4 EPSPS marker gene and lacking the Cre-recombinase transgene cassette are selected and self-pollinated giving rise to an F₃ generation. The F₃ generation plants are self-pollinated giving rise to a pure line of F₄ Gold Standard Seed.

FIG. 6 shows a representation of the insertion site of corn event ZM_BCS216090 relative to the endogenous corn genome on chromosome 1.

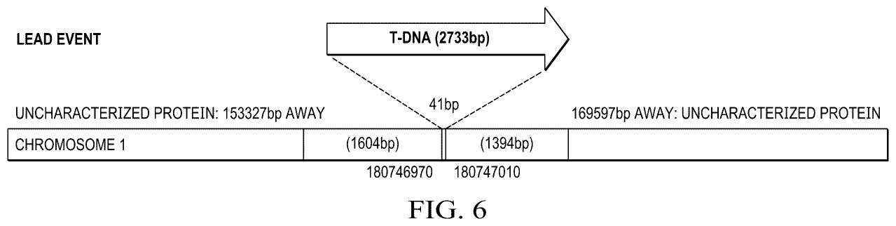

### BRIEF DESCRIPTION OF THE SEQUENCES

SEQ ID NO: 1 is a 30 nucleotide sequence representing the 5′ junction region of corn genomic DNA and the integrated transgenic expression cassette. SEQ ID NO: 1 is found within SEQ ID NO: 10 at nucleotide positions 986 to 1,015.
SEQ ID NO: 2 is a 30 nucleotide sequence representing the 3′ junction region of the integrated transgenic expression cassette and the corn genomic DNA. SEQ ID NO: 2 is found within SEQ ID NO: 10 at nucleotide positions 3,719 to 3,748.
SEQ ID NO: 3 is a 60 nucleotide sequence representing the 5′ junction region of corn genomic DNA and the integrated transgenic expression cassette. SEQ ID NO: 3 is found within SEQ ID NO: 10 at nucleotide positions 971 to 1,030.
SEQ ID NO: 4 is a 60 nucleotide sequence representing the 3′ junction region of the integrated transgenic expression cassette and the corn genomic DNA. SEQ ID NO: 4 is found within SEQ ID NO: 10 at nucleotide positions 3,704 to 3,763.
SEQ ID NO: 5 is a 100 nucleotide sequence representing the 5′ junction region of corn genomic DNA and the integrated transgenic expression cassette. SEQ ID NO: 5 is found within SEQ ID NO: 10 at nucleotide positions 951 to 1,050.
SEQ ID NO: 6 is a 100 nucleotide sequence representing the 3′ junction region of the integrated transgenic expression cassette and the corn genomic DNA. SEQ ID NO: 6 is found within SEQ ID NO: 10 at nucleotide positions 3,684 to 3,783.
SEQ ID NO: 7 is a 1,180 nucleotide sequence representing 1000 nucleotides of 5′ flanking corn genomic DNA and 180 nucleotides of the inserted T-DNA. SEQ ID NO: 7 is found within SEQ ID NO: 10 at nucleotide positions 1 to 1,180.
SEQ ID NO: 8 is a 1,107 nucleotide sequence representing 107 nucleotides of the inserted T-DNA and 1,000 nucleotides of 3′ flanking corn genomic DNA after the inserted T-DNA. SEQ ID NO: 8 is found within SEQ ID NO: 10 at nucleotide positions 3,627 to 4,733.
SEQ ID NO: 9 is a 2,733 nucleotide sequence corresponding to the transgenic inserted T-DNA of corn event ZM_BCS216090. SEQ ID NO: 9 is found within SEQ ID NO: 10 at nucleotide positions 1,001 to 3,733.
SEQ ID NO: 10 is a 5,731 nucleotide sequence corresponding to the contig nucleotide sequence of the 5′ genomic flanking DNA nucleotide sequence, the inserted T-DNA nucleotide sequence in event ZM_BCS216090, and the 3′ genomic flanking DNA nucleotide sequence; and includes SEQ ID NO: 17 (nucleotides 1-1,000), SEQ ID NO: 9 (nucleotides 1,001 to 3,733), and SEQ ID NO: 18 (nucleotides 3,734 to 4,733).
SEQ ID NO: 11 is a 19 nucleotide sequence corresponding to a thermal amplification primer referred to as SQ51606 used to identify corn event ZM_BCS216090 DNA in a sample, and is identical to the nucleotide sequence corresponding to positions 982 to 1,000 of SEQ ID NO: 10.
SEQ ID NO: 12 is a 21 nucleotide sequence corresponding to a thermal amplification primer referred to as SQ51629 used to identify corn event ZM_BCS216090 DNA in a sample, and is identical to the reverse complement of the nucleotide sequence corresponding to positions 1,049 to 1,069 of SEQ ID NO: 10.
SEQ ID NO: 13 is a 19 nucleotide sequence corresponding to a probe referred to as PB50583 used to identify corn event ZM_BCS216090 DNA in a sample, and is identical to the reverse complement of the nucleotide sequence corresponding to positions 1,028 to 1,046 of SEQ ID NO: 10.
SEQ ID NO: 14 is a 24 nucleotide sequence corresponding to a thermal amplification primer referred to as SQ20222 used as an internal control for the event and zygosity assay for corn event ZM_BCS216090 and hybridizes to a region of the corn genome.
SEQ ID NO: 15 is a 28 nucleotide sequence corresponding to a thermal amplification primer referred to as SQ20221 used as an internal control for the event and zygosity assay for corn event ZM_BCS216090 and hybridizes to a region of the corn genome.
SEQ ID NO: 16 is a 17 nucleotide sequence corresponding to a probe referred to as PB50298 used as an internal control for the event and zygosity assay for corn event ZM_BCS216090 and hybridizes to a region of the corn genome.
SEQ ID NO: 17 is a 1,000 nucleotide sequence of the 5′ flanking corn genomic DNA up to, but not including, the 5′ junction. SEQ ID NO: 17 is found within SEQ ID NO: 10 at nucleotide positions 1 to 1,000.
SEQ ID NO: 18 is a 1,000 nucleotide sequence of the 3′ flanking corn genomic DNA starting from, but not including, the 3′ junction. SEQ ID NO: 18 is found within SEQ ID NO: 10 at nucleotide positions 3,734 to 4,733.
SEQ ID NO: 19 is a 5,000-nucleotide sequence representing corn genomic DNA that flanks the transgenic insert at the 5′ end of the insert. Nucleotides 4,001-5,000 of SEQ ID NO:19 are identical to nucleotides SEQ ID NO: 17. The remaining nucleotides of SEQ ID NO:19 (nucleotides 1-4,000) are based on the genomic sequence of corn B73 germplasm.
SEQ ID NO: 20 is a 5,000-nucleotide sequence representing corn genomic DNA that flanks the transgenic insert at the 3′ end of the insert. Nucleotides 1-1,000 of SEQ ID NO:20 are identical to nucleotides SEQ NO: 18. The remaining nucleotides of SEQ ID NO:20 (nucleotides 1,001-5,000) are based on the genomic sequence of corn B73 germplasm.
SEQ ID NOs: 21 and 22 are the nucleotide and amino acid sequences, respectively, of LbCpf1 (also known as LbCas12a) of Lachnospiraceae bacterium ND2006.
SEQ ID NO: 23 is the amino acid sequence for LbCas12a_V1 (G532R/K595R, TYC variant).
SEQ ID NO: 24 is the amino acid sequence for LbCas12a_V2 (G532R/K538V/Y542R, TAT variant).
SEQ ID NO: 25 is the amino acid sequence for Cas12a of Francisella_novicida (FnCas12a).
SEQ ID NO: 26 is the nucleotide sequence for the gRNA repeat for LbCas12a.
SEQ ID NO: 27 is the nucleotide sequence for the gRNA repeat for FnCas12a.
SEQ ID NO: 28 is the nucleotide sequence for the gRNA gRNA_5F-63.
SEQ ID NO: 29 is the nucleotide sequence for the gRNA gRNA_3F-4.
SEQ ID NOs: 30-129 are 50-nucleotide sequences in the 5′ flank genomic sequence of event Zm_BCS216090. SEQ ID NOs: 30-49 are based on the 1 kb genomic sequence of SEQ ID NO: 17, and SEQ ID NOs: 50-129 are based on the additional 4 kb genomic sequence of corn B73 germplasm and SEQ ID NO: 19.
SEQ ID NOs: 130-229 are 50-nucleotide sequences in the 3′ flank genomic sequence of event Zm_BCS216090. SEQ ID NOs: 130-149 are based on the 1 kb genomic sequence of SEQ ID NO: 19, and SEQ ID NOs: 150-229 are based on the additional 4 kb genomic sequence of corn B73 germplasm and SEQ ID NO: 20.

### DETAILED DESCRIPTION

Plant height is an important agronomic trait in crops, as it can directly affect yield potential and lodging resistance. Manipulation of GA levels in semi-dwarf wheat, rice and sorghum plant varieties led to increased yield and reduced lodging in cereal crops during the 20th century, which was largely responsible for the Green Revolution. Gibberellins (gibberellic acids or GAs) are plant hormones that regulate various plant growth and developmental processes, including stem elongation, germination, dormancy, flowering, flower development, and leaf and fruit senescence. Bioactive GAs in corn include GA₁, GA₃, GA₄, and GA₇. GA biosynthesis is regulated by genes encoding GA20-oxidases (GA20ox) and GA3-oxidases (GA3ox) which catalyze steps in the synthesis of bioactive GAs, whereas GA catabolismis regulated by genes encoding GA2-oxidases (GA2ox) which reduce the level of active GAs. By manipulating genes in the GA biosynthesis or catabolismpathways, the level of active GAs can be lowered to reduce plant height.

The present disclosure provides a transgenic corn event, designated ZM_BCS216090, that comprises a transgene expression cassette that encodes a microRNA (miRNA) that suppresses the expression of the endogenous GA20ox3 and GA20ox5 genes in corn. The reduction in gibberellin levels caused by event ZM_BCS216090 results in a decreased internode length and overall decreased plant height, without any observable off-types. These short-stature corn or maize plants comprising the ZM_BCS216090 event are less susceptible to crop loss due to lodging and green snap. Corn or maize plants comprising the ZM_BCS216090 event therefore provide corn growers with a new option for increasing yield potential and reducing crop losses due to lodging or green snap especially when confronted with adverse or extreme weather or wind events.

Plant transformation techniques, such as Agrobacterium mediated or particle bombardment transformation, can be used to insert foreign DNA (also known as transgenic DNA) randomly into a chromosome of the genome of a plant cell to produce a genetically engineered plant cell, also referred to as a “transgenic” or “recombinant” cell. Using these non-targeted transformation techniques, many individual cells can be transformed, each resulting in a unique “transgenic event” or “event” due to the random (or largely random) insertion of the foreign DNA into the genome. A transgenic plant can then be regenerated from each individual transgenic cell. This results in every cell of the transgenic plant containing the uniquely inserted transgenic event as a stable part of its genome. The transgenic plant can then be used to produce progeny plants, each containing the unique transgenic event. The term “transgenic” refers to a plant, plant part, plant cell, plant tissue, or DNA molecule, construct, or sequence, as the case may be, comprising a transgene—e.g., a “transgenic cell” refers to a cell comprising a transgene.

Corn event ZM_BCS216090 was produced by an Agrobacterium-mediated transformation process of corn immature embryos with a single T-DNA binary system. In this system, an Agrobacterium strain employing one binary plasmid vector with a single T-DNA was utilized. The T-DNA construct comprised a transgene cassette for the expression of a microRNA (miRNA) that suppresses the expression of the endogenous gibberellin oxidase genes GA20ox3 and GA20ox5, and a transgene cassette used for the selection of transformed corn cells using glyphosate selection (CP4). The glyphosate selection cassette was flanked on both sides with LoxP recognition sites which are recognized by Cre-recombinase, derived from Enterobacteria phage P1 (Gilbertson, TRENDS in Biotechnology, 21 (12): 550-555, 2003).

As specifically described herein, corn event ZM_BCS216090 was produced by a complex research and development process in which: (1) a DNA construct and vector comprising two expression cassettes as identified above within a T-DNA region bounded by left and right border sequences was designed and selected based on individual testing of each expression cassette as well as in combination; (2) thousands of corn cells were transformed with the construct used to generate event ZM_BCS216090 and other events, creating a population of transgenic plants in which each plant contained a unique transgenic event that was regenerated; (3) more than a hundred transgenic plants were advanced through a series of self and out-crosses for further testing and event selection after excluding many events based on molecular quality screening and gold standard seed and product development; (4) the glyphosate selection cassette in corn event ZM_BCS216090 was removed through in vivo Cre-excision to create a “marker-free” events; and (5) the final lead event ZM_BCS216090 was selected from many different events after a rigorous multi-year event selection process involving testing and analysis of their molecular and genomic characteristics, efficacy and performance data, breeding and trait considerations, and agronomic properties in a variety of genetic backgrounds for each event that was advanced for testing. Corn event ZM_BCS216090 was produced and selected as a uniquely superior event, useful for broad-scale agronomic purposes.

The T-DNA from the transformation plasmid vector and inserted into the genome of corn event ZM_BCS216090 was characterized by detailed molecular analysis. This analysis included: the insert number (number of integration sites within the corn genome), the genomic insert location (the specific site in the corn genome where the insertion occurred), the copy number (the number of copies of the T-DNA within one locus), and the integrity of the inserted transgenic DNA. The detailed molecular analysis demonstrated that the integrated T-DNA containing the transgenic miRNA cassette remained intact after integration and Cre-excision of the glyphosate (CP4) EPSPS selection cassette. As used herein, an “expression cassette” or “cassette” or “transgene” is a recombinant DNA molecule or sequence comprising a combination of distinct elements that can express an RNA and/or protein encoded by a coding sequence of the transgene in a transformed plant cell comprising the transgene. As provided herein, an “expression cassette” or “cassette” or “transgene” includes one or more regulatory element(s) operably linked to the coding or transcribable DNA sequence for the miRNA including the promoter, leader, intron, and terminator sequences. The “expression cassette” or “cassette” or “transgene” is recombinant and heterologous with respect to the transformed plant cell genome. For purposes of the present disclosure, such an “expression cassette” or “cassette” or “transgene” is a recombinant DNA molecule or sequence that encodes a miRNA as described herein. Table 1 provides a list of the elements contained in SEQ ID NO: 10 after Cre excision of the CP4 cassette, the DNA sequence that corresponds to corn event ZM_BCS216090.

TABLE 1
Description of corn event ZM_BCS216090.

| | Position in | |
|---|---|---|
| | SEQ ID | |
| Element | NO: 10 | Description |
| 5′ Flanking DNA | 1-1,000 | DNA sequence flanking the 5′ end |
| | | of the transgenic insert |
| Left Border Region | 1,001-1,040 | DNA region from Agrobacterium |
| | | tumefaciens containing the left border |
| | | sequence |
| Intervening Sequence | 1,041-1,296 | Sequence used in DNA cloning |
| RTBV.−1:1:6 | 1,297-2,023 | Promoter sequence from Rice tungro |
| | | bacilliform virus |
| RTBV.−1:1:4 | 2,024-2,062 | Leader sequence from Rice tungro |
| | | bacilliform virus |
| Intervening Sequence | 2,063-2,082 | Sequence used in DNA cloning |
| I-Zm.DnaK:1 | 2,083-2,886 | Intron sequence of a heat shock |
| | | gene from maize (Zea mays) |
| Os.MIRMON13-1:1:3 | 2,887-2,998 | Backbone sequence of a miRNA |
| | | gene from rice (Oryza sativa) |
| Zm.miRNA.nno:3 | 2,999-3,019 | Transgenic miRNA sequence |
| Os.MIRMON13-1:1:2 | 3,020-3,053 | Loop sequence of the miRNA gene |
| | | from rice (Oryza sativa) |
| Zm.miRNA.nno:42 | 3,054-3,074 | Transgenic miRNA* sequence |
| Os.MIRMON13 | 3,075-3,294 | Backbone sequence of the miRNA |
| | | from rice (Oryza sativa) |
| Intervening Sequence | 3,295-3,326 | Sequence used in DNA cloning |
| T-GST43 | 3,327-3,626 | 3′ UTR sequence |
| Intervening Sequence | 3,627-3,733 | Sequence used in DNA cloning |
| 3′ Flanking Sequence | 3,734-4,733 | DNA sequence flanking the 3′ |
| | | end of the transgenic insert |

Corn event ZM_BCS216090 is characterized as an insertion into a single locus in the corn genome, resulting in two new loci or junction sequences (e.g., sequences set forth in SEQ ID NO: 1, SEQ ID NO: 2, SEQ ID NO: 3, SEQ ID NO: 4, SEQ ID NO: 5, and SEQ ID NO: 6) spanning a portion of the inserted DNA and the corn genomic DNA that are not known to appear or exist naturally in the corn genome or other transgenic corn events-they are unique to event ZM_BCS216090. SEQ ID NO: 1, SEQ ID NO: 2, and SEQ ID NO: 3 span one of the junctions, and SEQ ID NO: 4, SEQ ID NO: 5, and SEQ ID NO: 6 span the other junction. These junction sequences are useful in detecting the presence of the event ZM_BCS216090 in corn cells, corn tissue, corn seed, and corn plants or corn plant products, such as corn commodity products. Polynucleotide or DNA molecular probes and/or primer pairs are described herein that have been, or could be, developed for use in identifying the presence of these various junction segments in biological samples containing or derived from, or suspected of containing or being derived from, corn cells, corn seed, corn plant parts, corn plants, or corn plant tissue that contain the event ZM_BCS216090. As used herein, a “corn event ZM_BCS216090 locus” refers to the genomic locus of the corn event ZM_BCS216090 or a modified corn event ZM_BCS216090 or a further modified corn event ZM_BCS216090, wherein the corn event ZM_BCS216090 locus includes the flanking, junction and insertion sequences of the corn event ZM_BCS216090 or the modified corn event ZM_BCS216090 or further modified corn event ZM_BCS216090. A modified corn event ZM_BCS216090 or a further modified corn event ZM_BCS216090 comprises one or more mutations, edits and/or genetic modifications in the corn event ZM_BCS216090 locus, such as one or more mutations, edits and/or genetic modifications in a flanking, junction and/or insertion sequence(s) of the corn event ZM_BCS216090 locus, relative to the corn event ZM_BCS216090.

According to present embodiments, a modified corn event ZM_BCS216090 and methods of making a modified corn event ZM_BCS216090 are provided. As is described further herein, various mutagenesis or targeted genome editing techniques and related tools are known and could be made or engineered to permit genetic modification or mutation of the transgenic insert, junction and/or the flanking genomic DNA of corn event ZM_BCS216090, such as by deletion, insertion, transposition, inversion, and/or substitution of nucleic acid sequence(s), and/or by insertion or introduction of a guide RNA target site or a cognate target site or CgRRS, and the transgenic event as modified may still be uniquely characterized by the presence of heterologous DNA and/or one or more sequences of the insertion, junction(s) and/or flanking sequence(s) of corn event ZM_BCS216090 described herein at the same position or location in the genome previously occupied by the unmodified corn event ZM_BCS216090 relative to flanking portions or sequences of the native corn genome. According to present embodiments, a modified transgenic event derived from corn event ZM_BCS216090 may comprise all or part of the insertion sequence and/or transgene cassette of corn event ZM_BCS216090, one or more of the junction sequence(s) of corn event ZM_BCS216090, and/or one or more flanking sequence(s) of corn event ZM_BCS216090 as described herein. As used herein, a “modified corn event ZM_BCS216090” refers to any genomic DNA or sequence of the corn event ZM_BCS216090 locus comprising one or more mutations, edits or genetic modifications relative to the genomic DNA or sequence of the corn event ZM_BCS216090, wherein such mutations or edits are introduced or made by a mutagenesis or targeted genome editing technique of a corn plant, plant part, tissue or cell comprising the corn event ZM_BCS216090. A “modified corn event ZM_BCS216090” includes, as a type of modified corn event ZM_BCS216090, a “further modified corn event ZM_BCS216090” made by first inserting a target site or cognate target site or CgRRS into the corn event ZM_BCS216090 locus and then further modifying the corn event ZM_BCS216090 locus as described herein. For clarity, a “modified corn event ZM_BCS216090” includes genomic DNA or sequences of the corn event ZM_BCS216090 locus comprising one or more mutations, edits or genetic modifications relative to the genomic DNA or sequence of the corn event ZM_BCS216090, wherein such mutations or edits are introduced or made by a mutagenesis or targeted genome editing technique of a corn plant, plant part, tissue or cell comprising the corn event ZM_BCS216090, wherein such modified corn event ZM_BCS216090 is not a further modified corn event ZM_BCS216090. Methods and techniques of mutagenesis are known in the art and include, for example, chemical mutagenesis (i.e., treatment with a chemical mutagen, such as an azide, hydroxylamine, nitrous acid, acridine, nucleotide base analog, or alkylating agent—e.g., EMS (ethylmethane sulfonate), MNU (N-methyl-N-nitrosourea), etc.), physical mutagenesis (e.g., gamma rays, X-rays, UV, ion beam, other forms of radiation, etc.), and insertional mutagenesis (e.g., transposon or T-DNA insertion). As used herein, a “modified corn plant” refers to a corn or maize plant comprising a modified corn event ZM_BCS216090 or a further modified corn event ZM_BCS216090. Thus, a modified corn plant part, plant seed, plant tissue, or plant cell comprising a modified corn event ZM_BCS216090 or a further modified corn event ZM_BCS216090 that is derived, taken or descended from a modified corn plant and/or created by genetic modification, mutation or editing of the transgenic insert, junction and/or the flanking genomic DNA of corn event ZM_BCS216090 in a corn plant part, plant seed, plant tissue, or plant cell using a mutagenesis or targeted genome editing technique.

As used herein, a “target site” for genome editing refers to the location of a polynucleotide sequence within a plant genome, which may be within or near corn event ZM_BCS216090, that is bound and cleaved by a site-specific nuclease introducing a double stranded break (or single-stranded nick) into the nucleic acid backbone of the polynucleotide sequence and/or its complementary DNA strand. A target site may comprise at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 29, or at least 30 consecutive nucleotides. A “target site” for an RNA-guided nuclease may comprise the sequence of either complementary strand of a double-stranded nucleic acid (DNA) molecule or chromosome at the target site that is bound or hybridized to a guide RNA of a ribonucleoprotein complex comprising the RNA-guided nuclease. A site-specific nuclease may bind to a target site, such as via a non-coding guide RNA (e.g., without being limiting, a CRISPR RNA (crRNA) or a single-guide RNA (sgRNA) as described further below). A non-coding guide RNA (gRNA) provided herein may be complementary to a target site (e.g., complementary to either strand of a double-stranded nucleic acid molecule or chromosome at the target site). It will be appreciated that perfect identity or complementarity may not be required for a non-coding guide RNA to bind or hybridize to a target site. For example, at least 1, at least 2, at least 3, at least 4, at least 5, at least 6, at least 7, or at least 8 mismatches (or more) between a target site and a non-coding RNA may be tolerated. A “target site” also refers to the location of a polynucleotide sequence within a plant genome, which may be within or near corn event ZM_BCS216090 or a modified corn event ZM_BCS216090, that is bound and cleaved by another site-specific nuclease that may not be guided by a non-coding RNA molecule, such as a meganuclease, zinc finger nuclease (ZFN), or a transcription activator-like effector nuclease (TALEN), to introduce a double stranded break (or single-stranded nick) into the polynucleotide sequence and/or its complementary DNA strand. As used herein, a “target region” or a “targeted region” refers to a polynucleotide sequence or region that is flanked by two or more target sites. Without being limiting, in some embodiments a target region may be subjected to a mutation, deletion, insertion or inversion. As used herein, “flanked” when used to describe a target region of a polynucleotide sequence or molecule, refers to two or more target sites of the polynucleotide sequence or molecule surrounding the target region, with one target site on each side of the target region.

As used herein, a “donor molecule”, “donor template”, or “donor template molecule” (collectively a “donor template”), which may be a recombinant DNA donor template, is defined as a nucleic acid molecule having a nucleic acid template or insertion sequence for site-directed, targeted insertion or recombination into the genome of a plant cell via repair of a nick or double-stranded DNA break in the genome of a plant cell. For example, a “donor template” may be used for site-directed integration of a guide RNA target site or a cognate target site or CgRRS into a target site within the genome of a plant. A targeted genome editing technique provided herein may comprise the use of one or more, two or more, three or more, four or more, or five or more donor molecules or templates. A “donor template” may be a single-stranded or double-stranded DNA or RNA molecule or plasmid. An “insertion sequence” of a donor template is a sequence designed for targeted insertion into the genome of a plant cell, which may be of any suitable length, such as to include a guide RNA target site or a cognate target site or CgRRS. Such an insertion sequence of a donor template is distinct and different from the transgenic insertion or insert of corn event ZM_BCS216090, although they may potentially have sequence(s) in common. A donor template may also have at least one homology sequence or homology arm, such as two homology arms, to direct the integration of a mutation or insertion sequence into a target site within the genome of a plant via homologous recombination, wherein the homology sequence or homology arm(s) are identical or complementary, or have a percent identity or percent complementarity, to a sequence at or near the target site within the genome of the plant. When a donor template comprises homology arm(s) and an insertion sequence, the homology arm(s) will flank or surround the insertion sequence of the donor template.

As used herein, a “targeted genome editing technique” refers to any method, protocol, or technique that can be used to make a targeted mutation or edit, such as one or more insertions, deletions, substitutions, inversions, transpositions, mutations and/or other genetic modifications at or near a target site in the genome of a plant, and/or a deletion or excision of a target region between two target sites in the genome of a plant, using a site-specific nuclease, such as a meganuclease, a zinc-finger nuclease (ZFN), an RNA-guided endonuclease (e.g., the CRISPR/Cas9 system), a TALE-endonuclease (TALEN), a recombinase, or a transposase. A site-specific nuclease may introduce a double stranded break (or single-stranded nick) into the nucleic acid backbone of the polynucleotide sequence and/or its complementary DNA strand. Following the introduction of the single or double-stranded break or nick or cleavage of DNA at or near the target site(s) by the site-specific nuclease, the genomic sequence can be repaired via a double strand break repair pathway, which may include, for example, non-homologous end-joining (NHEJ), microhomology-mediated end joining (MMEJ), homologous recombination, synthesis-dependent strand annealing (SDSA), single-strand annealing (SSA), or a combination of any thereof, at or near the target site(s). However, if the repair is imperfect, a mutation or edit may be introduced at or near the target site(s), and a target region between two or more target sites may be deleted or excised.

A “site-specific nuclease” provided herein may be selected from the group consisting of a zinc-finger nuclease (ZFN), a meganuclease, an RNA-guided endonuclease, a TALE-endonuclease (TALEN), a recombinase, a transposase, or any combination thereof. See, e.g., Khandagale, K. et al., “Genome editing for targeted improvement in plants,” Plant Biotechnol Rep 10:327-343 (2016); and Gaj, T. et al., “ZFN, TALEN and CRISPR/Cas-based methods for genome engineering,” Trends Biotechnol. 31 (7): 397-405 (2013), the contents and disclosures of which are incorporated herein by reference. A recombinase may be a serine recombinase attached to a DNA recognition motif, a tyrosine recombinase attached to a DNA recognition motif or other recombinase enzyme known in the art. A recombinase or transposase may be a DNA transposase or recombinase attached to a DNA binding domain. A tyrosine recombinase attached to a DNA recognition motif may be selected from the group consisting of a Cre recombinase, a Flp recombinase, and a Tnp1 recombinase. According to some embodiments, a Cre recombinase or a Gin recombinase provided herein is tethered to a zinc-finger DNA binding domain. In another embodiment, a serine recombinase attached to a DNA recognition motif provided herein is selected from the group consisting of a PhiC31 integrase, an R4 integrase, and a TP-901 integrase. In another embodiment, a DNA transposase attached to a DNA binding domain provided herein is selected from the group consisting of a TALE-piggyBac and TALE-Mutator.

According to embodiments of the present disclosure, an RNA-guided endonuclease may be selected from the group consisting of Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, and homologs or modified versions thereof, Argonaute (non-limiting examples of Argonaute proteins include Thermus thermophilus Argonaute (TtAgo), Pyrococcus furiosus Argonaute (PfAgo), Natronobacterium gregoryi Argonaute (NgAgo) and homologs or modified versions thereof. According to some embodiments, an RNA-guided endonuclease may be a Cas9 or Cas12a or Cpf1 enzyme.

In an aspect, a site-specific nuclease provided herein is selected from the group consisting of a zinc-finger nuclease, a meganuclease, an RNA-guided nuclease, a TALE-nuclease, a recombinase, a transposase, or any combination thereof. In another aspect, a site-specific nuclease provided herein is selected from the group consisting of a Cas9 or a Cas12a or Cpf1. In another aspect, a site-specific nuclease provided herein is selected from the group consisting of a Cas1, a Cas1B, a Cas2, a Cas3, a Cas4, a Cas5, a Cas6, a Cas7, a Cas8, a Cas9, a Cas10, a Cas12a, a Csy1, a Csy2, a Csy3, a Csel, a Cse2, a Csc1, a Csc2, a Csa5, a Csn2, a Csm2, a Csm3, a Csm4, a Csm5, a Csm6, a Cmr1, a Cmr3, a Cmr4, a Cmr5, a Cmr6, a Csb1, a Csb2, a Csb3, a Csx17, a Csx14, a Csx10, a Csx16, a CsaX, a Csx3, a Csx1, a Csx15, a Csf1, a Csf2, a Csf3, a Csf4, a Cpf1, CasX, CasY, a homolog thereof, or a modified version thereof. In another aspect, an RNA-guided nuclease provided herein is selected from the group consisting of a Cas9 or a Cas12a or Cpf1. In another aspect, an RNA guided nuclease provided herein is selected from the group consisting of a Cas1, a Cas1B, a Cas2, a Cas3, a Cas4, a Cas5, a Cas6, a Cas7, a Cas8, a Cas9, a Cas10, a Cas12a, a Csy1, a Csy2, a Csy3, a Csel, a Cse2, a Csc1, a Csc2, a Csa5, a Csn2, a Csm2, a Csm3, a Csm4, a Csm5, a Csm6, a Cmr1, a Cmr3, a Cmr4, a Cmr5, a Cmr6, a Csb1, a Csb2, a Csb3, a Csx17, a Csx14, a Csx10, a Csx16, a CsaX, a Csx3, a Csx1, a Csx15, a Csf1, a Csf2, a Csf3, a Csf4, a Cpf1, CasX, CasY, a homolog thereof, or a modified version thereof. In another aspect, a method and/or a composition provided herein comprises at least one, at least two, at least three, at least four, at least five, at least six, at least seven, at least eight, at least nine, or at least ten site-specific nucleases. In yet another aspect, a method and/or a composition provided herein comprises at least one, at least two, at least three, at least four, at least five, at least six, at least seven, at least eight, at least nine, or at least ten polynucleotides encoding at least one, at least two, at least three, at least four, at least five, at least six, at least seven, at least eight, at least nine, or at least ten site-specific nucleases.

For RNA-guided endonucleases, a guide RNA (gRNA) molecule is further provided to direct the endonuclease to a target site in the genome of the plant via base-pairing or hybridization to cause a DSB or nick at or near the target site. The gRNA may be transformed or introduced into a plant cell or tissue (perhaps along with a nuclease, or nuclease-encoding DNA molecule, construct, or vector) as a gRNA molecule, or as a recombinant DNA molecule, construct or vector comprising a polynucleotide or transcribable DNA sequence encoding the guide RNA operably linked to a plant-expressible promoter. As understood in the art, a “guide RNA” may comprise, for example, a CRISPR RNA (crRNA), a single-chain guide RNA (sgRNA), or any other RNA molecule that may guide or direct an endonuclease to a specific target site in the genome. A “single-chain guide RNA” (or “sgRNA”) is an RNA molecule comprising a crRNA covalently linked a tracrRNA by a linker sequence, which may be expressed as a single RNA transcript or molecule. The guide RNA comprises a guide or targeting sequence that is identical or complementary to a target site within the plant genome, such as within or near corn event ZM_BCS216090 or a modified corn event ZM_BCS216090. A protospacer-adjacent motif (PAM) may be present in the genome immediately adjacent and upstream or downstream of the genomic target site sequence complementary to the targeting sequence of the guide RNA as known in the art. See, e.g., Wu, X. et al., “Target specificity of the CRISPR-Cas9 system,” Quant Biol. 2 (2): 59-70 (2014), the content and disclosure of which is incorporated herein by reference. The guide RNA may typically be a non-coding RNA molecule that does not encode a protein. The guide sequence of the guide RNA may be at least 10 nucleotides in length, such as 12-40 nucleotides, 12-30 nucleotides, 12-20 nucleotides, 12-35 nucleotides, 12-30 nucleotides, 15-30 nucleotides, 17-30 nucleotides, or 17-25 nucleotides in length, or about 12, 13, 14, 15, 16, 17, 18, 19, 20, 21, 22, 23, 24, 25 or more nucleotides in length. The guide sequence may be at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a DNA sequence at the genomic target site. As used herein, the term “consecutive” in reference to a polynucleotide or protein sequence means without deletions or gaps in the sequence.

In addition to the guide sequence, a guide RNA may further comprise one or more other structural or scaffold sequence(s), which may bind or interact with an RNA-guided endonuclease. Such scaffold or structural sequences may further interact with other RNA molecules (e.g., tracrRNA). Methods and techniques for designing targeting constructs and guide RNAs for genome editing and site-directed integration at a target site within the genome of a plant using an RNA-guided endonuclease are known in the art.

According to some embodiments, recombinant DNA molecules, constructs and vectors are provided comprising a polynucleotide or transcribable DNA sequence encoding a site-specific nuclease, such as a zinc-finger nuclease (ZFN), a meganuclease, an RNA-guided endonuclease, a TALE-endonuclease (TALEN), a recombinase, or a transposase, wherein the coding sequence is operably linked to a plant expressible promoter. For RNA-guided endonucleases, recombinant DNA molecules, constructs and vectors are further provided comprising a polynucleotide or transcribable DNA sequence encoding a guide RNA, wherein the guide RNA comprises a guide sequence of sufficient length having a percent identity or complementarity to a target site within the genome of a plant. According to some embodiments, recombinant DNA molecules, constructs and vectors are provided comprising a first polynucleotide or transcribable DNA sequence encoding a site-specific nuclease and a second polynucleotide or transcribable DNA sequence encoding one or more gRNAs. According to some embodiments, each polynucleotide or transcribable DNA sequence of a recombinant DNA molecule, construct and vector that encodes a site-specific nuclease and/or a guide RNA may be operably linked to a plant expressible promoter, such as an inducible promoter, a constitutive promoter, a tissue-specific promoter, etc.

According to some embodiments, recombinant DNA molecules, constructs and vectors are provided comprising a polynucleotide or transcribable DNA sequence encoding a site-specific nuclease, such as a zinc-finger nuclease (ZFN), a meganuclease, an RNA-guided endonuclease, a TALE-endonuclease (TALEN), a recombinase, or a transposase, wherein the coding sequence is operably linked to a plant expressible promoter. For RNA-guided endonucleases, recombinant DNA molecules, constructs and vectors are further provided comprising a polynucleotide or transcribable DNA sequence encoding a guide RNA, wherein the guide RNA comprises a guide sequence of sufficient length having a percent identity or complementarity to a target site within the genome of a plant. According to some embodiments, recombinant DNA molecules, constructs and vectors are provided comprising a first polynucleotide or transcribable DNA sequence encoding a site-specific nuclease and a second polynucleotide or transcribable DNA sequence encoding one or more gRNAs. According to some embodiments, each polynucleotide or transcribable DNA sequence of a recombinant DNA molecule, construct and vector that encodes a site-specific nuclease and/or a guide RNA may be operably linked to a plant expressible promoter, such as an inducible promoter, a constitutive promoter, a tissue-specific promoter, etc.

According to some embodiments, a recombinant DNA molecule, construct or vector may comprise a first polynucleotide sequence encoding a site-specific nuclease and a second polynucleotide sequence encoding a guide RNA(s) that may be introduced into a plant cell together via plant transformation techniques. Alternatively, two recombinant DNA molecules, constructs or vectors may be provided including a first recombinant DNA molecule, construct or vector and a second DNA molecule, construct or vector that may be introduced into a plant cell together or sequentially via plant transformation techniques, wherein the first recombinant DNA molecule, construct or vector comprises a polynucleotide sequence encoding a site-specific nuclease and the second recombinant DNA molecule, construct or vector comprises a polynucleotide sequence encoding a guide RNA(s). According to some embodiments, a recombinant DNA molecule, construct or vector comprising a polynucleotide sequence encoding a site-specific nuclease may be introduced via plant transformation techniques into a plant cell that already comprises (or is transformed with) a recombinant DNA construct or vector comprising a polynucleotide sequence encoding a guide RNA(s). Alternatively, a recombinant DNA molecule, construct or vector comprising a polynucleotide sequence encoding a guide RNA may be introduced via plant transformation techniques into a plant cell that already comprises (or is transformed with) a recombinant DNA construct or vector comprising a polynucleotide sequence encoding a site-specific nuclease. According to yet further embodiments, a first plant comprising (or transformed with) a recombinant DNA construct or vector comprising a polynucleotide sequence encoding a site-specific nuclease may be crossed with a second plant comprising (or transformed with) a recombinant DNA construct or vector comprising a polynucleotide sequence encoding a guide RNA(s). Such recombinant DNA molecules, constructs or vectors may be transiently transformed into a plant cell or stably transformed or more preferably integrated into the genome of a plant cell.

In an aspect, molecules or vectors comprising polynucleotides encoding a site-specific nuclease, and optionally one or more, two or more, three or more, or four or more gRNAs are provided to a plant cell by transformation methods known in the art (e.g., without being limiting, particle bombardment, PEG-mediated protoplast transfection or Agrobacterium-mediated transformation). In an aspect, molecules or vectors comprising polynucleotides encoding a Cas9 nuclease, and optionally one or more, two or more, three or more, or four or more gRNAs are provided to a plant cell by transformation methods known in the art (e.g., without being limiting, particle bombardment, PEG-mediated protoplast transfection or Agrobacterium-mediated transformation). In another aspect, vectors comprising polynucleotides encoding a Cpf1 and, optionally one or more, two or more, three or more, or four or more crRNAs are provided to a cell by transformation methods known in the art (e.g., without being limiting, viral transfection, particle bombardment, PEG-mediated protoplast transfection or Agrobacterium-mediated transformation).

Several site-specific nucleases, such as recombinases, zinc finger nucleases (ZFNs), meganucleases, and TALENs, are not RNA-guided and instead rely on their protein structure to determine their target site for causing the DSB or nick, or they are fused, tethered, or attached to a DNA-binding protein domain or motif. The protein structure of the site-specific nuclease (or the fused/attached/tethered DNA binding domain) may target the site-specific nuclease to the target site. According to many of these embodiments, non-RNA-guided site-specific nucleases, such as recombinases, zinc finger nucleases (ZFNs), meganucleases, and TALENs, may be designed, engineered and constructed according to known methods to target and bind to a target site in the genome of a plant, to create a DSB or nick at or near such genomic target site or locus. For example, an engineered site-specific nuclease, such as a recombinase, zinc finger nuclease (ZFN), meganuclease, or TALEN, may be designed to target and bind to a genomic target site within the genome of a plant to create a DSB or nick at the genomic target site.

In an aspect, a targeted genome editing technique described herein may comprise the use of a zinc finger nuclease (ZFN). ZFNs are synthetic proteins consisting of an engineered zinc finger DNA-binding domain fused to a cleavage domain (or a cleavage half-domain), which may be derived from a restriction endonuclease (e.g., FokI). The DNA binding domain may be canonical (C2H2) or non-canonical (e.g., C3H or C4). The DNA-binding domain can comprise one or more zinc fingers (e.g., 2, 3, 4, 5, 6, 7, 8, 9 or more zinc fingers) depending on the target site. Multiple zinc fingers in a DNA-binding domain may be separated by linker sequence(s). ZFNs can be designed to cleave almost any stretch of double-stranded DNA by modification of the zinc finger DNA-binding domain. ZFNs form dimers from monomers composed of a non-specific DNA cleavage domain (e.g., derived from the FokI nuclease) fused to a DNA-binding domain comprising a zinc finger array engineered to bind a target site DNA sequence. The DNA-binding domain of a ZFN may typically be composed of 3-4 (or more) zinc-fingers. The amino acids at positions −1, +2, +3, and +6 relative to the start of the zinc finger α-helix, which contribute to site-specific binding to the target site, can be changed and customized to fit specific target sequences. The other amino acids may form a consensus backbone to generate ZFNs with different sequence specificities. Methods and rules for designing ZFNs for targeting and binding to specific target sequences are known in the art. See, e.g., US Patent App. Nos. 2005/0064474, 2009/0117617, and 2012/0142062, the contents and disclosures of which are incorporated herein by reference. The FokI nuclease domain may require dimerization to cleave DNA and therefore two ZFNs with their C-terminal regions are needed to bind opposite DNA strands of the cleavage site (separated by 5-7 bp). The ZFN monomer can cut the target site if the two-ZF-binding sites are palindromic. A ZFN, as used herein, is broad and includes a monomeric ZFN that can cleave double stranded DNA without assistance from another ZFN. The term ZFN may also be used to refer to one or both members of a pair of ZFNs that are engineered to work together to cleave DNA at the same site.

Without being limited by any scientific theory because the DNA-binding specificities of zinc finger domains can be re-engineered using one of various methods, customized ZFNs can theoretically be constructed to target nearly any target sequence (e.g., at or near corn event ZM_BCS216090 in a plant genome). Publicly available methods for engineering zinc finger domains include Context-dependent Assembly (CoDA), Oligomerized Pool Engineering (OPEN), and Modular Assembly. In an aspect, a method and/or composition provided herein comprises one or more, two or more, three or more, four or more, or five or more ZFNs. In another aspect, a ZEN provided herein is capable of generating a targeted DSB or nick. In an aspect, vectors comprising polynucleotides encoding one or more, two or more, three or more, four or more, or five or more ZFNs are provided to a cell by transformation methods known in the art (e.g., without being limiting, viral transfection, particle bombardment, PEG-mediated protoplast transfection, or Agrobacterium-mediated transformation). The ZFNs may be introduced as ZEN proteins, as polynucleotides encoding ZFN proteins, and/or as combinations of proteins and protein-encoding polynucleotides.

In an aspect, a targeted genome editing technique described herein may comprise the use of a meganuclease. Meganucleases, which are commonly identified in microbes, such as the LAGLIDADG family of homing endonucleases, are unique enzymes with high activity and long recognition sequences (>14 bp) resulting in site-specific digestion of target DNA. Engineered versions of naturally occurring meganucleases typically have extended DNA recognition sequences (for example, 14 to 40 bp). According to some embodiments, a meganuclease may comprise a scaffold or base enzyme selected from the group consisting of I-CreI, I-Ceul, I-Msol, I-Scel, I-Anil, and I-Dmol. The engineering of meganucleases can be more challenging than ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity. Thus, a meganuclease may be selected or engineered to bind to a genomic target site or sequence in a plant. In an aspect, a method and/or composition provided herein comprises one or more, two or more, three or more, four or more, or five or more meganucleases. In another aspect, a meganuclease provided herein is capable of generating a targeted DSB. In an aspect, vectors comprising polynucleotides encoding one or more, two or more, three or more, four or more, or five or more meganucleases are provided to a cell by transformation methods known in the art (e.g., without being limiting, viral transfection, particle bombardment, PEG-mediated protoplast transfection or Agrobacterium-mediated transformation).

In an aspect, a targeted genome editing technique described herein may comprise the use of a transcription activator-like effector nuclease (TALEN). TALENs are artificial restriction enzymes generated by fusing the transcription activator-like effector (TALE) DNA binding domain to a nuclease domain (e.g., FokI). In some aspects, the nuclease is selected from a group consisting of PvuII, MutH, TevI, FokI, AlwI, MlyI, SbfI, SdaI, StsI, CleDORF, Clo051, and Pept071. For FokI nuclease, when each member of a TALEN pair binds to the DNA sites flanking a target site, the FokI monomers dimerize and cause a double-stranded DNA break at the target site. Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity. The term TALEN, as used herein, is broad and includes a monomeric TALEN that can cleave double stranded DNA without assistance from another TALEN. The term TALEN also refers to one or both members of a pair of TALENs that work together to cleave DNA at the same site.

Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity. PvuII, MutH, and TevI cleavage domains are useful alternatives to FokI and FokI variants for use with TALEs. PvuII functions as a highly specific cleavage domain when coupled to a TALE (see Yank et al. 2013. PLOS One. 8: e82539). MutH is capable of introducing strand-specific nicks in DNA (see Gabsalilow et al. 2013. Nucleic Acids Research. 41: e83). TevI introduces double-stranded breaks in DNA at targeted sites (see Beurdeley et al., 2013. Nature Communications. 4:1762).

Transcription activator-like effectors (TALEs) can be engineered to bind practically any DNA sequence, such as at or near the genomic locus of corn event ZM_BCS216090 in a plant. TALE has a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs. The relationship between amino acid sequence and DNA recognition of the TALE binding domain allows for designable proteins. Software programs such as DNA Works can be used to design TALE constructs. Other methods of designing TALE constructs are known to those of skill in the art. See Doyle et al., Nucleic Acids Research (2012) 40: W117-122; Cermak et al., Nucleic Acids Research (2011). 39: e82; and tale-nt.cac.cornell.edu/about. In an aspect, a method and/or composition provided herein comprises one or more, two or more, three or more, four or more, or five or more TALENs. In another aspect, a TALEN provided herein is capable of generating a targeted DSB. In an aspect, vectors comprising polynucleotides encoding one or more, two or more, three or more, four or more, or five or more TALENs are provided to a cell by transformation methods known in the art (e.g., without being limiting, viral transfection, particle bombardment, PEG-mediated protoplast transfection or Agrobacterium-mediated transformation). See, e.g., US Patent App. Nos. 2011/0145940, 2011/0301073, and 2013/0117869, the contents and disclosures of which are incorporated herein by reference.

In an aspect, a targeted genome editing technique described herein may comprise the use of a recombinase. In some embodiments, a tyrosine recombinase attached, etc., to a DNA recognition domain or motif may be selected from the group consisting of a Cre recombinase, a Flp recombinase, and a Tnp1 recombinase. In an aspect, a Cre recombinase or a Gin recombinase provided herein may be tethered to a zinc-finger DNA binding domain. The Flp-FRT site-directed recombination system may come from the 2u plasmid from the baker's yeast Saccharomyces cerevisiae. In this system, Flp recombinase (flippase) may recombine sequences between flippase recognition target (FRT) sites. FRT sites comprise 34 nucleotides. Flp may bind to the “arms” of the FRT sites (one arm is in reverse orientation) and cleaves the FRT site at either end of an intervening nucleic acid sequence. After cleavage, Flp may recombine nucleic acid sequences between two FRT sites. Cre-lox is a site-directed recombination system derived from the bacteriophage P1 that is similar to the Flp-FRT recombination system. Cre-lox can be used to invert a nucleic acid sequence, delete a nucleic acid sequence, or translocate a nucleic acid sequence. In this system, Cre recombinase may recombine a pair of lox nucleic acid sequences. Lox sites comprise 34 nucleotides, with the first and last 13 nucleotides (arms) being palindromic. During recombination, Cre recombinase protein binds to two lox sites on different nucleic acids and cleaves at the lox sites. The cleaved nucleic acids are spliced together (reciprocally translocated) and recombination is complete. In another aspect, a lox site provided herein is a loxP, lox 2272, loxN, lox 511, lox 5171, lox71, lox66, M2, M3, M7, or M11 site.

As used herein, the term “derived” or “derived from” in reference to a particular DNA molecule, amplicon or sequence in relation to a corn cell, corn tissue, corn seed, corn plant, corn plant part and/or corn plant product, such as a corn commodity product, means that the DNA molecule, amplicon or sequence is taken, purified, isolated, or made, directly or indirectly, from such corn cell, corn tissue, corn seed, corn plant, corn plant part and/or corn plant product, such as a corn commodity product, as the case may be. Alternatively, the term “derived” or “derived from” in reference to a corn plant product, such as a corn commodity product, in relation to a corn cell, corn tissue, corn seed, corn plant, and/or corn plant part, means that the corn plant product is taken, purified, isolated, or made, directly or indirectly, from such corn cell, corn tissue, corn seed, corn plant, and/or corn plant part, as the case may be. “Capable of being detected” refers to the ability of a particular DNA molecule, segment, or sequence to be detected in a sample, such as by amplification and determining its presence, size, or sequence such as by DNA sequence analysis, and/or binding of a probe to the target DNA molecule, segment, or sequence.

A “sample” is intended to refer to any composition comprising or derived from, either directly or indirectly, a biological sample, source, or material. The sample may generally comprise corn DNA and/or substantially or completely pure, purified, or isolated corn DNA. A “biological sample” contains biological materials, including but not limited to DNA obtained or derived from, either directly or indirectly, the genome of a corn cell(s), corn tissue(s), corn seed(s), corn plant(s), corn plant part(s) and/or corn plant product(s), such as a corn commodity product(s). Such corn cell(s), corn tissue(s), corn seed(s), corn plant(s), corn plant part(s) and/or corn plant product(s), such as a corn commodity product(s), may comprise corn event ZM_BCS216090 or a modified corn event ZM_BCS216090 or DNA molecule(s) and/or DNA segment(s) comprising corn event ZM_BCS216090 or a modified corn event ZM_BCS216090. In some embodiments, a sample or biological sample may comprise corn cell(s), corn tissue(s), corn seed(s), corn plant(s), corn plant part(s), and/or corn plant product(s), whose cells or cellular membranes have been fractured (e.g., disrupted or opened) to release the contents of the corn cell(s) including genomic DNA and/or make the contents of the corn cell(s) including genomic DNA accessible or usable for assays or testing. “Directly” refers to directly obtaining DNA by a skilled artisan from the corn genome by fracturing corn cells (or by obtaining samples of corn that contain fractured corn cells) and exposing or using the genomic DNA from corn cells for the purposes of detection. “Indirectly” refers to obtaining by a skilled artisan a target or specific reference DNA (i.e., a novel and unique junction segment(s) described herein as being diagnostic for the presence of the event ZM_BCS216090 or a modified corn event ZM_BCS216090) in a particular sample, by means other than by obtaining directly via fracturing of corn cells or obtaining a sample of corn that contains fractured corn cells. Such indirect means include, but are not limited to, amplification of a DNA segment that contains a DNA sequence targeted by a particular probe(s) and/or primer set(s) designed to bind with specificity to or near the target sequence, or amplification of a DNA segment comprising all or part of a target sequence that can be measured and characterized (e.g., measured by migration or separation from other segments of DNA and/or identification in an effective matrix, such as an agarose or acrylamide gel or the like, or characterized by direct sequence analysis of the amplicon(s), or cloning of the amplicon(s) into a vector(s) and direct sequencing of the inserted amplicon(s) present within such vector(s)).

Detailed molecular analysis demonstrated that event ZM_BCS216090 contains a single T-DNA insertion with one copy of the SD419 transgenic miRNA expression cassette. No additional elements from the transformation construct other than portions of the Agrobacterium tumefaciens left border region used for transgenic DNA transfer from the plant transformation plasmid to the corn genome were identified in event ZM_BCS216090. Finally, thermal amplification producing specific amplicons diagnostic for the presence of event ZM_BCS216090 in a sample and DNA sequence analyses were performed to determine the assigned 5′ and 3′ insert-to-plant genome junctions, confirm the organization of the elements within the insert, and determine the complete DNA sequence of the inserted transgenic DNA (SEQ ID NO: 9). SEQ ID NO: 7 is a sequence corresponding to the 5′ corn genomic DNA sequence of SEQ ID NO: 10 and a portion of the inserted T-DNA sequence presented as SEQ ID NO: 9. SEQ ID NO: 8 is a sequence corresponding to the 3′ corn genomic DNA sequence of SEQ ID NO: 10 and a portion of the inserted T-DNA sequence presented as SEQ ID NO: 9. SEQ ID NO: 10 corresponds to corn event ZM_BCS216090 and contains a contiguous sequence (contig) comprising the 5′ corn genomic flanking sequence, the transgene insert of event ZM_BCS216090, and the 3′ corn genomic flanking sequence, and thus contains the 5′ and 3′ insert-to-plant genome junction sequences. As used herein, the 5′ and 3′ designations in reference to the junction, direction and side of the transgenic event insertion is relative to the 5′ to 3′ direction of the transgene, with the 5′ junction and genomic sequence being upstream of the transgene, and the 3′ junction and genomic sequence being downstream of the transgene.

Unless otherwise noted herein, terms are to be understood according to conventional usage by those of ordinary skill in the relevant art. Definitions of common terms in molecular biology may be found in Rieger et al., Glossary of Genetics: Classical and Molecular, 5^{th} edition, Springer-Verlag: New York, 1991; and Lewin, Genes V, Oxford University Press: New York, 1994, along with other sources known to those skilled in the relevant art. As used herein, the term “corn” or “maize” means plant species within Zea mays and all plant varieties belonging to the genus Zea that can be bred with Zea mays plants, including wild maize species.

The present disclosure provides for transgenic corn or maize plants which have been transformed with a DNA construct that contains an expression cassette encoding a transgenic miRNA to suppress expression of endogenous GA20 oxidase target genes. The terms “suppress” and “suppression” as used herein, refer to a lowering, reduction, or elimination of the expression level of a mRNA and/or protein encoded by a target gene in a plant, plant cell, or plant tissue at one or more stage(s) of plant development, as compared to the expression level of such target gene mRNA and/or protein in a wild-type or control plant, cell, or tissue at the same stage(s) of plant development. Corn plants transformed according to the methods and with the DNA construct disclosed herein have a short stature (shorter plant height) phenotype.

A transgenic plant is produced by transformation of a plant cell with a recombinant DNA construct that includes the expression cassette and transgene encoding the miRNA as described herein. Such recombinant DNA construct is also heterologous with respect to the plant cell, regeneration of at least one plant from the plant cell comprising an insertion of the transgene into the genome of the plant cell, and selection of a particular plant characterized by insertion of the transgene into a particular genomic location of the plant based on a number of efficacious and quality characteristics and features of the event and regenerated transgenic plant and progeny as described further herein. The term “transgenic event” or “event” refers to the inserted transgenic DNA in the plant genome and flanking genomic sequences immediately adjacent to the inserted transgenic DNA in the genome of the transformed plant, but also refers to a DNA molecule comprising the inserted transgenic DNA in the plant genome and flanking genomic sequences. Each event is unique and would be expected to be transferred to progeny plants that receive the transgenic DNA and event through genetic inheritance and/or segregation from a parent as the result of a sexual or self-cross of a first parental line that includes the inserted transgenic DNA and event either with itself or a second parental line that may or may not contain the same transgenic DNA and event. The parental line that includes the inserted transgenic DNA and event may itself be the original transformant or a progeny plant of said original transformant that may have been generated by “selfing” the transformant with itself or crossing the transformant or a progeny plant of the transformant that includes the inserted transgenic DNA and event with another plant. For purposes of the present disclosure, the “event” refers to corn event ZM_BCS216090 or a modified corn event ZM_BCS216090.

As used herein, the term “flanking” in reference to a transgenic event refers to the plant genomic sequence(s) immediately adjacent to the transgenic DNA insertion in the genome of a transformed plant, plant part, plant tissue, or plant cell comprising the transgenic event on the 5′ and/or 3′ side(s) or end(s) of the transgenic event insertion (i.e., the transgenic insertion of corn event ZM_BCS216090 or a modified corn event ZM_BCS216090). Likewise, “flanking DNA,” “flanking sequence” or “flanking DNA sequence” each refers to a length of genomic DNA sequence immediately adjacent to the transgenic DNA insertion in the genome of the transformed plant on the 5′ and/or 3′ side(s) or end(s) of the insertion. A “5′ flank” means the corn genomic DNA sequence adjacent to and upstream (or on the 5′ end) of the transgenic DNA insertion. For example, a “5′ flank” can include the corn genomic DNA sequence immediately adjacent to and upstream (on the 5′ end) of the transgenic insertion, or any corn genomic DNA sequence upstream (on the 5′ end) of the transgenic insertion that is not immediately adjacent to the transgenic insertion but is within about 5000 nucleotides, within about 4000 nucleotides, within about 3000 nucleotides, within about 2000 nucleotides, or within about 1000 nucleotides upstream of the transgenic insertion. Likewise, a “3′ flank” means the corn genomic DNA sequence adjacent to and downstream (or on the 3′ end) of the transgenic insert. For example, a “3′ flank” can include the corn genomic DNA sequence immediately adjacent to and downstream (on the 3′ end) of the transgenic insertion, or any corn genomic DNA sequence downstream (on the 3′ end) of the transgenic insertion that is not immediately adjacent to the transgenic insertion but is within about 5000 nucleotides, within about 4000 nucleotides, within about 3000 nucleotides, within about 2000 nucleotides, or within about 1000 nucleotides downstream of the transgenic insertion.

SEQ ID NOs: 17 and 18 are 1,000 nucleotide sequences representing corn (Zea mays) genomic DNA that flanks the transgenic insert at the 5′ and 3′ ends of the insert in corn event ZM_BCS216090, respectively. SEQ ID NOs: 19 and 20 are 5,000 nucleotide sequences representing corn (Zea mays) genomic DNA that flanks the transgenic insert at the 5′ and 3′ ends of the insert, respectively. Nucleotides 4,001-5,000 of SEQ ID NO: 19 are identical to nucleotides 1-1,000 of SEQ ID NO: 17. The remaining nucleotides of SEQ ID NO: 19 (nucleotides 1-4,000) are based on the genomic sequence of the B73 corn cultivar (Zm-B73-REFERENCE-GRAMENE-4.0, NCBI). Similarly, nucleotides 1-1000 of SEQ ID NO: 20 are identical to nucleotides 1-1,000 of SEQ NO: 18. The remaining nucleotides of SEQ ID NO: 20 (nucleotides 1,001-5,000) are based on the genomic sequence of the B73 corn cultivar.

The present disclosure provides the original transformant plant and progeny of the transformant that include the transgenic DNA and event. Such progeny may be produced by a sexual cross or outcross between plants comprising the same transgenic DNA and event, or between a plant comprising the transgenic DNA and event with another plant, or by any other method known in the art including any cell or tissue culture method, wherein the progeny includes the transgenic DNA and event. Such other plant may be a transgenic plant comprising the same and/or a different transgene or may be a non-transgenic plant, and each parental plant in a cross or outcross may be the same or different germplasm or breeding line. Even after repeated back-crossing to a recurrent parent, the transgenic DNA and event is present in progeny of the cross at the same chromosomal location. Thus, a “transgenic plant” can be the original transformant plant regenerated from the transformed plant cell and comprising the transgenic DNA and event, or a progeny plant of the original transformant plant, which may be separated from the transformant by one or more generations, that retains the transgenic DNA and event at the same specific location and sequence context in the plant's genome. The transformant or progeny plant may be homozygous or heterozygous for event ZM_BCS216090 or a modified corn event ZM_BCS216090. In addition, a “transgenic plant” can include a plant produced from a transformed plant cell or tissue, or from another transgenic plant or plant part, by or using cell or tissue culture methods known in the art. A “transgenic plant” may comprise a plant having a transgene or transgenic event stably inserted into the genome of at least one cell of the plant or a modified transgenic event (i.e., corn event ZM_BCS216090 or a modified corn event ZM_BCS216090 in at least one cell of the plant), and the plant may be chimeric or non-chimeric with respect to the transgene and/or event or modified event. A transgenic plant is chimeric with respect to a transgene, event, or modified event if not all cells of the plant comprise the transgene, event, or modified event.

As used herein, the term “recombinant” refers to a non-natural DNA, protein, or combination that would not normally be found in nature, such as a combination of DNA sequences, proteins that would not naturally occur together, and is the result of human intervention. A “recombinant DNA molecule” is a DNA molecule comprising a combination of DNA sequences that would not naturally occur together and is the result of human intervention. Two or more elements of such combination of DNA sequences may be operably linked to one another. For example, a recombinant DNA molecule may comprise a combination of at least two DNA sequences that are heterologous with respect to each other, such as a DNA molecule that comprises a coding or transcribable DNA sequence operably linked to a heterologous promoter and/or other regulatory expression element(s), and/or a plant genomic DNA sequence comprising all or part of a transgene and a heterologous and flanking genomic sequence(s) adjacent to the transgene, and/or a DNA molecule that is artificially synthesized and comprises a polynucleotide sequence that deviates from any polynucleotide sequence that would normally exist in nature. A recombinant DNA molecule may comprise all or part of a junction sequence of the genome of a plant and all or part of the transgene insertion into the genome of the plant, and/or may comprise a recombinant or heterologous DNA fragment of event ZM_BCS216090 or a modified corn event ZM_BCS216090. An example of a recombinant DNA molecule is a DNA molecule comprising at least one of SEQ ID NOs: 1-10. As used herein, a recombinant plant, plant part, plant cell or plant tissue is a plant, plant part, plant cell or plant tissue that would not normally exist in nature, is the result of human intervention, and contains a transgene incorporated into the genome of the plant, plant part, plant cell or plant tissue. As a result of such genomic insertion, the recombinant plant is something new and distinctly different from any related wild-type or naturally occurring plant, plant part, plant cell or plant tissue. An example of a recombinant plant, plant part, plant cell or plant tissue is a corn or maize plant, plant part, plant cell or plant tissue containing the event ZM_BCS216090 or a modified corn event ZM_BCS216090.

As used herein, the term “heterologous” in reference to a combination of two or more DNA sequences or elements means that the two or more DNA sequences or elements do not normally exist together as such combination in nature without human intervention. As used herein, the term “heterologous” in reference to a DNA molecule, construct or sequence in relation to a plant, microorganism, plant cell or plant genome means that the DNA molecule, construct or sequence does not exist in nature as part of such plant, microorganism, plant cell or plant genome, and/or does not exist in the same physical or genomic location, context or orientation as part of such plant, microorganism, plant cell or plant genome in nature, without human intervention.

The present disclosure provides DNA molecules and fragments and their corresponding DNA sequences. The terms “DNA” and “DNA molecule” as used herein refer to a deoxyribonucleic acid (DNA) molecule. A DNA molecule may be of genomic or synthetic origin and/or comprise a recombinant or heterologous DNA molecule or sequence. A DNA molecule may be described in reference to its 5′ (upstream) end and 3′ (downstream) end. As used herein, the term “DNA sequence” refers to the polynucleotide sequence of the DNA molecule—i.e., the sequence of consecutive nucleotides in the DNA molecule. As used herein in reference to nucleotides of a polynucleotide or DNA sequence or molecule, the terms “consecutive” and “contiguous” are interchangeable and synonymous and refer to the 5′ to 3′ order of nucleotides in a polynucleotide or DNA sequence, strand or molecule without any gap or interruption between them. By convention, DNA sequences of the disclosure and fragments thereof are disclosed with reference to the 5′ to 3′ direction of only one strand of the two, anti-parallel and complementary DNA strands of a DNA molecule. By implication and intent, the complementary sequences of the sequences provided here (i.e., the sequences of the complementary, opposing, or antiparallel strand), also referred to in the art as the reverse complementary or reverse complement sequences, are within the scope of the present disclosure and are expressly intended to be within the potential scope of the subject matter as claimed. A DNA molecule, or a fragment derived therefrom, can also be extracted from plant part(s), plant cell(s) and/or tissue(s) or a homogenate, extract or lysate from plant part(s), plant cell(s) and/or tissue(s), or can be produced as an amplicon from extracted, purified or isolated DNA from plant part(s), plant cell(s) and/or tissue(s), or a homogenate, extract or lysate from plant part(s), plant cell(s) and/or tissue(s), which may further comprise event ZM_BCS216090.

As used herein, the term “fragment” refers to a smaller piece or sequence of a larger or whole DNA molecule or sequence. For example, a fragment of SEQ ID NO: 9 or 10 may include a sequence that is at least about 12 consecutive nucleotides, at least about 13 consecutive nucleotides, at least about 14 consecutive nucleotides, at least about 15 consecutive nucleotides, at least about 16 consecutive nucleotides, at least about 17 consecutive nucleotides, at least about 18 consecutive nucleotides, at least about 19 consecutive nucleotides, at least about 20 consecutive nucleotides, at least about 21 consecutive nucleotides, at least about 22 consecutive nucleotides, at least about 23 consecutive nucleotides, at least about 24 consecutive nucleotides, at least about 25 consecutive nucleotides, at least about 30 consecutive nucleotides, at least about 35 consecutive nucleotides, at least about 40 consecutive nucleotides, at least about 45 consecutive nucleotides, at least about 50 consecutive nucleotides, at least about 60 consecutive nucleotides, at least about 70 consecutive nucleotides, at least about 80 consecutive nucleotides, at least about 90 consecutive nucleotides, at least about 100 consecutive nucleotides, at least about 200 consecutive nucleotides, at least about 300 consecutive nucleotides, at least about 400 consecutive nucleotides, or at least about 500 consecutive nucleotides of the larger, whole or complete DNA molecule or sequence of SEQ ID NO: 9 or 10.

According to present embodiments, a fragment of the DNA sequence of the 5′ flank (SEQ ID NO: 17 or SEQ ID NO: 19) or the 3′ flank (SEQ ID NO: 18 or SEQ ID NO: 20) of corn event ZM_BCS216090 can comprise at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, or at least 500 consecutive nucleotides of SEQ ID NO: 17 or SEQ ID NO: 19 or SEQ ID NO: 18 or SEQ ID NO: 20. It is possible that different maize germplasms may have differences in their genomic sequences, which may include differences in the flanking sequence(s), 5′ flank and/or 3′ flank of corn event ZM_BCS216090. These differences may result from introgression of the corn event ZM_BCS216090 or a modified corn event ZM_BCS216090 into a different germplasm and/or spontaneous, mutagenic or genome editing changes that occur in a given germplasm or line. According to some embodiments, DNA molecules, constructs or polynucleotides are provided comprising a sequence or flanking sequence, or a 5′ flank or 3′ flank, that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to SEQ ID NO: 17 or 19 or SEQ ID NO: 18 or 20, or a fragment of either thereof. According to some embodiments, DNA molecules, constructs or polynucleotides are provided comprising a sequence or flanking sequence, or a 5′ flank or 3′ flank, that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, or at least 500 consecutive nucleotides of SEQ ID NO: 17 or 19 or SEQ ID NO: 18 or 20.

As used herein, the term “isolated” in reference to a molecule means that the molecule is at least partially separated from other molecules or sequences that are normally associated with the molecule in its native or natural state. In some embodiments, the term “isolated” refers to a DNA molecule that is at least partially separated from the nucleic acids or polynucleotide or DNA sequence(s) that normally flank and are covalently linked to the sequence of the DNA molecule in its native or natural state. An “isolated” DNA molecule may have a DNA sequence corresponding to a portion of the genome of a plant cell without other genomic DNA sequence(s) that normally flank and are covalently linked to the DNA sequence in nature. Such an “isolated” DNA molecule may comprise all or part of a transgene and/or transgenic event, which may comprise all or part of corn event ZM_BCS216090 or a modified corn event ZM_BCS216090 or the transgene or expression cassette described herein. Nucleic acid sequences or elements, such as a coding sequence, intron sequence, untranslated leader sequence, promoter sequence, transcriptional termination sequence, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” within the scope of this disclosure so long as the element or subpart is not within the genome of the organism, and at the location within the genome of the organism, in which it is naturally found. An “isolated” DNA molecule may be any recombinant DNA molecule or amplification product or amplicon, and/or may comprise any DNA sequence removed from its natural or biological state and covalently fused to another DNA molecule or sequence with which it is not associated in nature. Such an isolated DNA molecule could be created by the use of biotechnology techniques, such as by making a recombinant DNA or integrating a foreign or heterologous DNA molecule into the chromosome of a cell, plant, or seed. Thus, any DNA molecule comprising a transgenic, recombinant, chimeric or artificial nucleotide sequence, transgene or expression cassette would be considered to be an “isolated” DNA molecule since these sequences are not naturally occurring, regardless of whether the sequence, transgene or expression cassette is present within a plasmid, vector or construct used to transform plant cells, within the genome of a plant, plant part, plant tissue, or plant cell, or is present in detectable amounts in tissues, progeny, biological samples or commodity products derived from a plant, plant part, plant tissue, or plant cell. A recombinant DNA molecule or sequence, or any fragment derived therefrom, comprising all or part of a transgene or junction sequence of corn event would therefore also be considered to be “isolated.” An “isolated” DNA molecule may be extracted or purified from a transgenic plant(s), plant part(s), plant cell(s) and/or tissue(s), or may be present in a homogenate, extract or lysate from any such transgenic plant(s), plant part(s), plant cell(s) and/or tissue(s), or may be produced as an amplicon or amplification product from plant genomic DNA and/or extracted or purified DNA from transgenic plant(s), plant part(s), plant cell(s) and/or tissue(s), or a homogenate, extract or lysate from plant(s), plant part(s), plant cell(s) and/or tissue(s). For the purposes of this disclosure, any transgenic polynucleotide or DNA sequence, i.e., the nucleotide sequence of the DNA inserted into the genome of a plant or bacterium, or present in an extrachromosomal vector, would be considered to be an “isolated” nucleotide or DNA sequence whether it is present within the plasmid or similar structure used to transform the cells, within the genome of the plant or bacterium, or present in detectable amounts in tissues, progeny, biological samples or commodity products derived from the plant or bacterium. An “isolated” DNA molecule is a chemical or biochemical molecule, regardless of whether the molecule is referred to as a nucleic acid, a nucleic acid sequence, a polynucleotide sequence, a DNA sequence, a nucleic acid molecule, a polynucleotide molecule, a DNA molecule, or the like. An “isolated” molecule can provide industrial applicability when present in a plant cell or in a plant genome or when present outside of a plant cell, and therefore, provides and exhibits (and is intended to provide and exhibit) utility regardless of where the molecule is located.

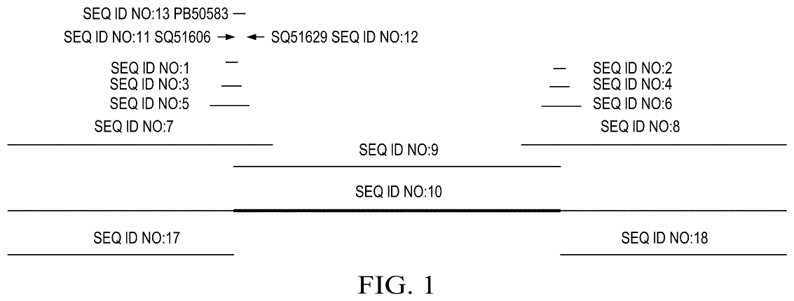

The phosphodiester bond linkage between one end of a transgenic insert (or insertion) into the genome of a plant and the flanking corn genomic DNA is referred to as a “junction.” In other words, a “junction” is the connection point or covalent linkage of one end of a transgenic insert and the flanking genomic DNA. One junction is found at the 5′ end of the transgenic insertion and the other is found at the 3′ end of the transgenic insert, referred to herein as the 5′ and 3′ junction, respectively. A “junction sequence” refers to a DNA sequence of any length of consecutive nucleotides that spans the 5′ or 3′ junction of a transgenic event in the plant genome. For a “junction sequence” to be specific to a junction between a transgenic event and a flanking genomic sequence, the junction sequence will generally comprise a sufficient number of consecutive nucleotides at one end of the insertion and a sufficient number of consecutive nucleotides of the flanking genomic sequence. According to some embodiments, a “junction sequence” may comprise (i) at least five (5) consecutive nucleotides, at least ten (10) consecutive nucleotides, at least fifteen (15) consecutive nucleotides, at least twenty (20) consecutive nucleotides, or at least thirty (30) consecutive nucleotides at one end of the insertion and (ii) at least five (5) consecutive nucleotides, at least ten (10) consecutive nucleotides, at least fifteen (15) consecutive nucleotides, at least twenty (20) consecutive nucleotides, or at least thirty (30) consecutive nucleotides of flanking genomic DNA sequence, although it is understood that any length of consecutive nucleotides spanning a junction of a transgenic event in a plant genome may be a junction sequence. A variety of junction sequences of corn event ZM_BCS216090 can be determined by one of skill in the art using SEQ ID NO: 10. Examples of junction sequences of event ZM_BCS216090 are provided as SEQ ID NOs: 1-8. FIG. 1 illustrates the physical arrangement and locations of the junction sequences, arranged from 5′ to 3′ (left to right), relative to SEQ ID NO: 10. The junction sequence(s) of a modified corn event ZM_BCS216090 may be modified, mutated or edited relative to such junction sequence(s) of corn event ZM_BCS216090. The junction sequences of event ZM_BCS216090 may be present as part of the genome of a corn plant, plant part, plant seed, or plant tissue or cell containing event ZM_BCS216090 or a modified corn event ZM_BCS216090, a DNA molecule containing all or part of event ZM_BCS216090 or a modified corn event ZM_BCS216090, or a microorganism containing event ZM_BCS216090 or a modified corn event ZM_BCS216090. The identification of any one or more of the junction sequences in a DNA molecule or sample from a plant, plant part, plant seed, or plant tissue or cell indicates that the plant, plant part, plant seed, or plant tissue or cell contains or comprises event ZM_BCS216090 or a modified corn event ZM_BCS216090, or the DNA molecule contains or comprises event ZM_BCS216090 or a modified corn event ZM_BCS216090 or was obtained from a corn plant, plant part, plant seed, or plant tissue or cell containing or comprising event ZM_BCS216090 or a modified corn event ZM_BCS216090, and is diagnostic in each case for the presence of event ZM_BCS216090 or a modified corn event ZM_BCS216090.

A junction sequence for event ZM_BCS216090 or a modified corn event ZM_BCS216090 may be represented by a sequence from the group consisting of SEQ ID NO: 1, SEQ ID NO: 2, SEQ ID NO: 3, SEQ ID NO: 4, SEQ ID NO: 5, SEQ ID NO: 6, and SEQ ID NO: 10. For example, the junction sequences may be or comprise SEQ ID NO: 1 and SEQ ID NO: 2, SEQ ID NO: 1 and SEQ ID NO: 4, SEQ ID NO: 1 and SEQ ID NO: 6, SEQ ID NO: 3 and SEQ ID NO: 2, SEQ ID NO: 3 and SEQ ID NO: 4, SEQ ID NO: 3 and SEQ ID NO: 6, SEQ ID NO: 5 and SEQ ID NO: 2, SEQ ID NO: 5 and SEQ ID NO: 4, or SEQ ID NO: 5 and SEQ ID NO: 6. The junction sequences may comprise a first or 5′ junction sequence and a second or 3′ junction sequence, wherein the first or 5′ junction sequence is or comprises one or more of SEQ ID NO: 1, SEQ ID NO: 3, and/or SEQ ID NO: 5, and wherein the second or 3′ junction sequence is or comprises one or more of SEQ ID NO: 2, SEQ ID NO: 4, and/or SEQ ID NO: 6. Alternatively or additionally, a first or 5′ junction sequence may be or comprise SEQ ID NO: 7, and/or a second or 5′ junction sequence may be or comprise SEQ ID NO: 8.

The junction sequences described herein are diagnostic for the presence of all or part of event ZM_BCS216090, and if unmodified may be diagnostic for the presence of all or part of or a modified corn event ZM_BCS216090, and/or a DNA molecule comprising all or part of the miRNA-encoding transgene, construct or expression cassette described herein. Thus, the identification or detection, directly or indirectly, of one or more of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 10 in a sample or DNA molecule derived from a corn plant, corn plant part, corn seed, or corn tissue or cell, or a commodity product from a corn plant, corn plant part, corn seed, or corn tissue or cell, is diagnostic that the corn plant, corn plant part, corn seed, or corn tissue or cell, or a commodity product from a corn plant, corn plant part, corn seed, or corn tissue or cell has or comprises all or part of corn event ZM_BCS216090 or a modified corn event ZM_BCS216090. The identification or detection, directly or indirectly, of a 5′ junction sequence and a 3′ junction sequence (each as provided or described herein) in a sample or DNA molecule derived from a corn plant, corn plant part, corn seed, or corn tissue or cell, or a commodity product from a corn plant, corn plant part, corn seed, or corn tissue or cell, is diagnostic that the corn plant, corn plant part, corn seed, or corn tissue or cell, or a commodity product from a corn plant, corn plant part, corn seed, or corn tissue or cell has or comprises corn event ZM_BCS216090 or a modified corn event ZM_BCS216090. The present disclosure thus provides a DNA molecule that contains at least one of the nucleotide sequences provided as SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10. Any segment of DNA derived from transgenic corn event ZM_BCS216090 that is sufficient to include at least one of the sequences provided as SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10 is within the scope of the present disclosure. In addition, any DNA or polynucleotide molecule or sequence comprising a sequence complementary to any of the sequences described herein is also within the scope of the present disclosure.

The disclosure provides DNA, polynucleotide or nucleic acid molecules, which may be single or double stranded, that can be used either as primers or probes for detecting the presence of DNA comprising all or part of event ZM_BCS216090 or a modified corn event ZM_BCS216090 in a sample derived from a corn plant, corn plant part, corn seed, or corn tissue or cell, or a commodity product from a corn plant, corn plant part, corn seed, or corn tissue or cell. Such primers or probes are specific for a target nucleic acid, polynucleotide, or DNA sequence and, as such, are useful for the identification of corn event ZM_BCS216090 nucleic acid, polynucleotide or DNA sequence, or a nucleic acid, polynucleotide, or DNA sequence of a modified corn event ZM_BCS216090, by the methods described herein. A primer or probe can hybridize to a target nucleic acid, polynucleotide, or DNA sequence to allow for specific detection or amplification of a nucleic acid, polynucleotide or DNA molecule or sequence that comprises, or is covalently linked and associated with, the target nucleic acid, polynucleotide, or DNA sequence. According to present embodiments, the primers and/or probe may be chosen to identify and distinguish detection of a particular transgenic event and not only the presence of a transgene in a plant genome. The target nucleic acid, polynucleotide or DNA molecule or sequence may comprise all or part of corn event ZM_BCS216090 or a modified corn event ZM_BCS216090, a junction sequence and/or flanking genomic DNA. Probes and primers according to the present disclosure may have (i) complete or 100% sequence complementarity (i.e., 100% complementary) to a target DNA sequence or (ii) incomplete sequence complementarity to a target DNA sequence, such as at least 60% complementary, at least 65% complementary, at least 70% complementary, at least 75% complementary, at least 80% complementary, at least 85% complementary, at least 90% complementary, at least 95% complementary, or at least 99% complementary to the target DNA sequence as long as the probe or primer has sufficient complementarity to the target DNA sequence to hybridize to the target DNA sequence under stringent hybridization conditions that are suitable and necessary for use of the probe or primer in the relevant amplification or detection assay, reaction or method. As understood in the art, the percentage complementarity of a primer or probe may be lower if the length of the primer or probe is longer and depends on the stringency and use.

A “probe” is a nucleic acid molecule that is complementary to a strand of target nucleic acid and is useful in hybridization methods. A probe may be attached a conventional detectable label or reporter molecule, e.g., a radioactive isotope, fluorophore, ligand, chemiluminescent agent, or enzyme. Such a probe is complementary to a strand of a target nucleic acid and, in the case of the present disclosure, to a strand of DNA from event ZM_BCS216090 or a modified corn event ZM_BCS216090 whether from an event ZM_BCS216090 containing plant, or a plant containing or a modified corn event ZM_BCS216090, or from a sample that includes event ZM_BCS216090 DNA or DNA from a modified corn event ZM_BCS216090. Probes according to the present invention include not only deoxyribonucleic or ribonucleic acids, but also polyamides and other probe materials that bind specifically to a target DNA sequence and can be used to detect the presence of that target DNA sequence. An exemplary DNA sequence useful as a probe for detecting corn event ZM_BCS216090 or a modified corn event ZM_BCS216090 is provided as SEQ ID NO: 13 (PB50583). A “probe” may also be used to bind a template DNA in a sample comprising all or part of a DNA or nucleotide sequence of corn event ZM_BCS216090 to purify the template DNA from the remainder of the sample using purification methods or techniques known in the art, for example, if the probe is bound or can be bound to a substrate or a particle or bead that can be purified or separated. Such a template DNA may comprise all or part of a DNA or nucleotide sequence of corn event ZM_BCS216090 or a modified corn event ZM_BCS216090, or a portion or fragment thereof, such as a nucleotide sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10, or a complement thereof.

A “primer” is a DNA molecule or oligonucleotide that is designed for use in specific annealing or hybridization methods that involve an in vitro amplification reaction. A pair of primers may be used with template DNA (such as a sample of corn genomic DNA) in a thermal amplification reaction (such as polymerase chain reaction (PCR)) or any other suitable amplification method known in the art to produce an amplification product or amplicon, where the amplicon produced from such reaction would have a DNA sequence corresponding to sequence of the template DNA located between the two sites where the primers hybridized to the template DNA. As understood in the art, an “amplification product” or “amplicon” is a DNA molecule or segment produced by an amplification reaction. Amplification or amplifying refers to making multiple copies of a target DNA molecule or segment from a template DNA. A single “primer” may also be used to initiate a sequencing reaction to determine a DNA sequence of a template DNA according to sequencing methods known in the art. Such a sequencing reaction may be used to determine the presence or absence of a DNA molecule or nucleotide sequence, or a portion or fragment thereof, from corn event ZM_BCS216090 or a modified corn event ZM_BCS216090. Such a template DNA may comprise all or part of a DNA or nucleotide sequence of corn event ZM_BCS216090 or a modified corn event ZM_BCS216090, or a portion or fragment thereof, such as a nucleotide sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10, or a complement thereof.

DNA amplification reactions, methods and techniques are known to those skilled in art. DNA amplification can be accomplished by any of the various nucleic acid amplification methods known in the art, including thermal and isothermal amplification methods including the polymerase chain reaction or PCR. Amplification methods are known in the art and are described, inter alia, in U.S. Pat. Nos. 4,683,195 and 4,683,202 and in PCR Protocols: A Guide to Methods and Applications, ed. Innis et al., Academic Press, San Diego, 1990. PCR amplification methods have been developed to amplify up to 22 kb (kilobase) of genomic DNA and up to 42 kb of bacteriophage DNA (Cheng et al., Proc. Natl. Acad. Sci. USA 91:5695-5699, 1994). These methods as well as other methods known in the art of DNA amplification may be used in the practice of the present invention. Examples of DNA amplification methods include PCR, Recombinase Polymerase Amplification (RPA) (see for example U.S. Pat. No. 7,485,428), Strand Displacement Amplification (SDA) (see for example, U.S. Pat. Nos. 5,455,166 and 5,470,723), Transcription-Mediated Amplification (TMA) (see for example, Guatelli et al., Proc. Natl. Acad. Sci. USA 87:1874-1878, 1990), Rolling Circle Amplification (RCA) (see for example, Fire and Xu, Proc. Natl. Acad Sci. USA 92:4641-4645, 1995; Lui, et al., J. Am. Chem. Soc. 118:1587-1594, 1996; Lizardi, et al., Nature Genetics 19:225-232, 1998; U.S. Pat. Nos. 5,714,320 and 6,235,502), Helicase Dependent Amplification (HDA) (see for example Vincent et al., EMBO Reports 5 (8): 795-800, 2004; U.S. Pat. No. 7,282,328), and Multiple Displacement Amplification (MDA) (see for example Dean et al., Proc. Natl. Acad Sci. USA 99:5261-5266, 2002). A sequence of the heterologous DNA insert and/or flanking genomic DNA sequence from corn event ZM_BCS216090 can be verified or tested by amplifying such DNA molecules from corn seed containing event ZM_BCS216090 DNA or corn plants grown from the corn seed containing event ZM_BCS216090 DNA, using primers derived from the sequences provided herein, followed by standard DNA sequencing of the PCR amplicon or a cloned DNA fragment thereof.

According to present embodiments, the sequence of an amplicon of an amplification reaction may comprise one or more of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10, or a fragment thereof. According to present embodiments, the sequence of an amplicon comprises at least one junction sequence or two junction sequences, such as a 5′ junction sequence and/or a 3′ junction sequence for corn event ZM_BCS216090 or a modified corn event ZM_BCS216090.

A primer is typically designed to hybridize in a sequence-specific manner to a complementary target DNA strand to form a hybrid between the primer and target DNA strand, and the primer hybridized or bound to the complementary target DNA strand is a point of recognition for a polymerase to begin extension of the primer (i.e., polymerization of additional nucleotides into a lengthening nucleotide molecule) using as a template the target DNA strand. Primer pairs refer to use of two primers binding opposite strands of a double stranded DNA or polynucleotide segment for the purpose of amplifying the polynucleotide or DNA segment between the positions targeted for binding by the individual primers of the primer pair to the original template DNA or an amplicon of the amplification reaction, typically in a thermal cycling amplification reaction or other conventional DNA amplification method. Primer pairs are typically designed to hybridize to different nearby target positions of a template DNA molecule on opposing strands of the template DNA molecule such that the intervening region or sequence between the two primers can be specifically amplified for use or detection through multiple rounds of amplification.

According to present embodiments, a DNA molecule, construct, segment, amplicon, fragment or polynucleotide is provided that contains or comprises at least one of the nucleotide sequences provided as SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10. According to present embodiments, a DNA molecule, construct, segment, amplicon, fragment or polynucleotide is provided that contains or comprises at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, or at least 90 consecutive nucleotides of SEQ ID NO: 1, SEQ ID NO: 2, SEQ ID NO: 3, SEQ ID NO: 4, SEQ ID NO: 5, or SEQ ID NO: 6. According to present embodiments, a DNA molecule, construct, segment, amplicon, fragment or polynucleotide is provided that (i) contains or comprises at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of any one of SEQ ID NOs: 1-10, (ii) is at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 nucleotides in length, and (iii) comprises nucleotides 1,000-1,001 and/or 3,733-3,734 of SEQ ID NO: 10.

According to present embodiments, a DNA molecule, construct, segment, amplicon, fragment, or polynucleotide is provided that contains or comprises (i) an expression cassette that encodes a microRNA (miRNA) that suppresses the expression of the endogenous GA20ox3 and GA20ox5 genes in corn or maize, and (ii) a junction sequence. According to present embodiments, a DNA molecule, construct, segment, amplicon, fragment, or polynucleotide is provided that contains or comprises (i) an expression cassette that encodes a microRNA (miRNA) that suppresses the expression of the endogenous GA20ox3 and GA20ox5 genes in corn or maize, and (ii) a 5′ flank and/or a 3′ flank sequence. The expression cassette may comprise in operable linkage: (a) a promoter sequence from Rice tungro bacilliform virus, (b) a transcribable DNA sequence encoding a miRNA sequence that suppresses the expression of the endogenous GA20ox3 and GA20ox5 genes in corn or maize, and (c) a transcription termination or 3′ UTR sequence. The expression cassette may further comprise any of the elements described in Table 1, which may be operably linked, including, for example, a leader, intron, intervening sequence(s), miRNA and/or the MIRMON13 backbone and/or loop sequence(s).

According to present embodiments, a DNA molecule, construct, segment, amplicon, fragment or polynucleotide is provided that contains or comprises (i) an expression cassette that encodes a microRNA (miRNA) that suppresses the expression of the endogenous GA20ox3 and GA20ox5 genes in corn or maize, and (ii) a polynucleotide sequence comprising one or more of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10. According to present embodiments, a DNA molecule, construct, segment, amplicon, fragment or polynucleotide is provided that contains or comprises (i) an expression cassette that encodes a microRNA (miRNA) that suppresses the expression of the endogenous GA20ox3 and GA20ox5 genes in corn or maize, and (ii) a polynucleotide sequence comprising at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of one or more of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.

According to present embodiments, a DNA molecule, construct, segment, amplicon, fragment or polynucleotide is provided that contains or comprises (i) an expression cassette that encodes a microRNA (miRNA) that suppresses the expression of the endogenous GA20ox3 and GA20ox5 genes in corn or maize, and (ii) a polynucleotide sequence comprising at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 17 or SEQ ID NO: 19 or SEQ ID NO: 18 or SEQ ID NO: 20. According to some embodiments, a DNA molecule, construct, segment, amplicon, fragment or polynucleotide is provided that contains or comprises (i) an expression cassette that encodes a microRNA (miRNA) that suppresses the expression of the endogenous GA20ox3 and GA20ox5 genes in corn or maize, and (ii) a sequence or flanking sequence, or a 5′ flank or 3′ flank, that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to SEQ ID NO: 17 or 19 or SEQ ID NO: 18 or 20, or a fragment of either thereof. According to some embodiments, a DNA molecule, construct, segment, amplicon, fragment or polynucleotide is provided that contains or comprises (i) an expression cassette that encodes a microRNA (miRNA) that suppresses the expression of the endogenous GA20ox3 and GA20ox5 genes in corn or maize, and (ii) a sequence or flanking sequence, or a 5′ flank or 3′ flank, that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 17 or 19 or SEQ ID NO: 18 or 20. According to present embodiments, a DNA molecule, construct, segment, amplicon, fragment or polynucleotide is provided that contains or comprises (i) an expression cassette that encodes a microRNA (miRNA) that suppresses the expression of the endogenous GA20ox3 and GA20ox5 genes in corn or maize, and (ii) a polynucleotide sequence comprising one or more polynucleotide sequences selected from SEQ ID NOs: 30-49 and SEQ ID NOs: 50-129. According to present embodiments, a DNA molecule, construct, segment, amplicon, fragment or polynucleotide is provided that contains or comprises (i) an expression cassette that encodes a microRNA (miRNA) that suppresses the expression of the endogenous GA20ox3 and GA20ox5 genes in corn or maize, and (ii) a polynucleotide sequence comprising one or more polynucleotide sequences selected from SEQ ID NOs: 130-149 and SEQ ID NOs: 150-229.

According to present embodiments, a DNA molecule, construct, segment, amplicon, fragment or polynucleotide is provided that contains or comprises (i) an expression cassette that encodes a microRNA (miRNA) that suppresses the expression of the endogenous GA20ox3 and GA20ox5 genes in corn or maize, (ii) a polynucleotide sequence comprising at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 17 or SEQ ID NO: 19, and (iii) a polynucleotide sequence comprising at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 18 or SEQ ID NO: 20. According to some embodiments, a DNA molecule, construct, segment, amplicon, fragment or polynucleotide is provided that contains or comprises (i) an expression cassette that encodes a microRNA (miRNA) that suppresses the expression of the endogenous GA20ox3 and GA20ox5 genes in corn or maize, (ii) a sequence or flanking sequence, or a 5′ flank, that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to SEQ ID NO: 17 or 19, or a fragment of either thereof, and (iii) a sequence or flanking sequence, or a 3′ flank, that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to SEQ ID NO: 18 or 20, or a fragment of either thereof. According to some embodiments, a DNA molecule, construct, segment, amplicon, fragment or polynucleotide is provided that contains or comprises (i) an expression cassette that encodes a microRNA (miRNA) that suppresses the expression of the endogenous GA20ox3 and GA20ox5 genes in corn or maize, and (ii) a sequence or flanking sequence, or a 5′ flank, that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 17 or 19, and (iii) a sequence or flanking sequence, or a 3′ flank, that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 18 or 20. According to present embodiments, a DNA molecule, construct, segment, amplicon, fragment or polynucleotide is provided that contains or comprises (i) an expression cassette that encodes a microRNA (miRNA) that suppresses the expression of the endogenous GA20ox3 and GA20ox5 genes in corn or maize, (ii) a polynucleotide sequence comprising one or more polynucleotide sequences selected from SEQ ID NOs: 30-49 and SEQ ID NOs: 50-129, and (iii) a polynucleotide sequence comprising one or more polynucleotide sequences selected from SEQ ID NOs: 130-149 and SEQ ID NOs: 150-229.

According to present embodiments, a DNA molecule, construct, segment, amplicon, fragment or polynucleotide is provided that contains or comprises an expression cassette that encodes a microRNA (miRNA) that suppresses the expression of the endogenous GA20ox3 and GA20ox5 genes in corn or maize, wherein the DNA molecule, construct, segment, amplicon, fragment or polynucleotide comprises at its 5′ end at least 50 consecutive nucleotides of SEQ ID NO: 17 or 19. According to present embodiments, a DNA molecule, construct, segment, amplicon, fragment or polynucleotide is provided that contains or comprises an expression cassette that encodes a microRNA (miRNA) that suppresses the expression of the endogenous GA20ox3 and GA20ox5 genes in corn or maize, wherein the DNA molecule, construct, segment, amplicon, fragment or polynucleotide comprises at its 3′ end at least 50 consecutive nucleotides of SEQ ID NO: 18 or 20. According to present embodiments, a DNA molecule, construct, segment, amplicon, fragment or polynucleotide is provided that contains or comprises an expression cassette that encodes a microRNA (miRNA) that suppresses the expression of the endogenous GA20ox3 and GA20ox5 genes in corn or maize, wherein the DNA molecule, construct, segment, amplicon, fragment or polynucleotide comprises at its 5′ end a polynucleotide sequence selected from SEQ ID NOs: 30-49 and SEQ ID NOs: 50-129. According to present embodiments, a DNA molecule, construct, segment, amplicon, fragment or polynucleotide is provided that contains or comprises an expression cassette that encodes a microRNA (miRNA) that suppresses the expression of the endogenous GA20ox3 and GA20ox5 genes in corn or maize, wherein the DNA molecule, construct, segment, amplicon, fragment or polynucleotide comprises at its 3′ end a polynucleotide sequence selected from SEQ ID NOs: 130-149 and SEQ ID NOs: 150-229.

According to present embodiments, a DNA molecule, construct, segment, amplicon, fragment or polynucleotide is provided that contains or comprises an expression cassette that encodes a microRNA (miRNA) that suppresses the expression of the endogenous GA20ox3 and GA20ox5 genes in corn or maize, wherein the DNA molecule, construct, segment, amplicon, fragment or polynucleotide comprises at its 5′ end at least 50 consecutive nucleotides of SEQ ID NO: 17 or 19, wherein the DNA molecule, construct, segment, amplicon, fragment or polynucleotide comprises at its 3′ end at least 50 consecutive nucleotides of SEQ ID NO: 18 or 20, wherein the DNA molecule, construct, segment, amplicon, fragment or polynucleotide comprises at its 5′ end a polynucleotide sequence selected from SEQ ID NOs: 30-49 and SEQ ID NOs: 50-129, and/or wherein the DNA molecule, construct, segment, amplicon, fragment or polynucleotide comprises at its 3′ end a polynucleotide sequence selected from SEQ ID NOs: 130-149 and SEQ ID NOs: 150-229.

According to present embodiments, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 17 or SEQ ID NO: 19 at the 5′ end of a DNA molecule, construct, segment, amplicon, fragment or polynucleotide may be immediately adjacent to and upstream (on the 5′ end) of the transgenic insertion, or may not be immediately adjacent to, but further upstream (on the 5′ end) and within about 5000 nucleotides, within about 4000 nucleotides, within about 3000 nucleotides, within about 2000 nucleotides, or within about 1000 nucleotides of the transgenic insertion. According to present embodiments, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 18 or SEQ ID NO: 20 at the 3′ end of a DNA molecule, construct, segment, amplicon, fragment or polynucleotide may be immediately adjacent to and downstream (on the 3′ end) of the transgenic insertion, or may not be immediately adjacent to but further downstream (on the 3′ end) and within about 5000 nucleotides, within about 4000 nucleotides, within about 3000 nucleotides, within about 2000 nucleotides, or within about 1000 nucleotides of the transgenic insertion. Illustrative examples of sequences comprising 50 consecutive nucleotides of SEQ ID NO: 17 or 19 are provided in SEQ ID NOs: 30-49. Illustrative examples of 50 consecutive nucleotides of SEQ ID NO: 18 or 20 are provided in SEQ ID NOs: 130-149. Illustrative examples of 50 consecutive nucleotides of SEQ ID NO: 19 are provided in SEQ ID NOs: 50-129. Illustrative examples of 50 consecutive nucleotides of SEQ ID NO: 20 are provided in SEQ ID NOs: 150-229. However, any sequence comprising at least 50 consecutive nucleotides of SEQ ID NO: 17 or SEQ ID NO:19, or at least 50 consecutive nucleotides of SEQ ID NO: 18 or SEQ ID NO: 20 is within the scope of the present disclosure. A DNA molecule, construct, segment, amplicon, fragment, or polynucleotide can comprise at the 5′ and/or 3′ end of the construct (i) at least 50 consecutive nucleotides of SEQ ID NO: 17 or SEQ ID NO: 19; and/or (ii) at least 50 consecutive nucleotides of SEQ ID NO: 19 or SEQ ID NO: 20, respectively.

According to present embodiments, a DNA molecule, construct, segment, amplicon, fragment or polynucleotide is provided that contains or comprises (i) SEQ ID NO: 9 or a polynucleotide sequence comprising at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 9, and (ii) a junction sequence. According to present embodiments, a DNA molecule, construct, segment, amplicon, fragment or polynucleotide is provided that contains or comprises (i) SEQ ID NO: 9 or a polynucleotide sequence comprising at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 9, and (ii) a 5′ flank and/or a 3′ flank sequence.

According to present embodiments, a DNA molecule, construct, segment, amplicon, fragment or polynucleotide is provided that contains or comprises (i) SEQ ID NO: 9 or a polynucleotide sequence comprising at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 9, and (ii) a polynucleotide sequence comprising at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 17 or SEQ ID NO: 19 or SEQ ID NO: 18 or SEQ ID NO: 20. According to some embodiments, a DNA molecule, construct, segment, amplicon, fragment or polynucleotide is provided that contains or comprises (i) SEQ ID NO: 9 or a polynucleotide sequence comprising at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 9, and (ii) a sequence or flanking sequence, or a 5′ flank or 3′ flank, that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to SEQ ID NO: 17 or 19 or SEQ ID NO: 18 or 20, or a fragment of either thereof.

According to some embodiments, a DNA molecule, construct, segment, amplicon, fragment or polynucleotide is provided that contains or comprises (i) SEQ ID NO: 9 or a polynucleotide sequence comprising at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 9, and (ii) a sequence or flanking sequence, or a 5′ flank or 3′ flank, that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 17 or 19 or SEQ ID NO: 18 or 20. According to present embodiments, a DNA molecule, construct, segment, amplicon, fragment or polynucleotide is provided that contains or comprises (i) SEQ ID NO: 9 or a polynucleotide sequence comprising at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 9, and (ii) a polynucleotide sequence comprising one or more polynucleotide sequences selected from SEQ ID NOs: 30-49 and SEQ ID NOs: 50-129.

According to present embodiments, a DNA molecule, construct, segment, amplicon, fragment or polynucleotide is provided that contains or comprises (i) SEQ ID NO: 9 or a polynucleotide sequence comprising at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 9, and (ii) a polynucleotide sequence comprising one or more polynucleotide sequences selected from SEQ ID NOs: 130-149 and SEQ ID NOs: 150-229.

According to present embodiments, a DNA molecule, construct, segment, amplicon, fragment or polynucleotide is provided that contains or comprises (i) SEQ ID NO: 9 or a polynucleotide sequence comprising at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 9, (ii) a polynucleotide sequence comprising at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 17 or SEQ ID NO: 19, and (iii) a polynucleotide sequence comprising at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 18 or SEQ ID NO: 20. According to some embodiments, a DNA molecule, construct, segment, amplicon, fragment or polynucleotide is provided that contains or comprises (i) SEQ ID NO: 9 or a polynucleotide sequence comprising at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 9, (ii) a sequence or flanking sequence, or a 5′ flank, that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to SEQ ID NO: 17 or 19, or a fragment of either thereof, and (iii) a sequence or flanking sequence, or a 3′ flank, that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to SEQ ID NO: 18 or 20, or a fragment of either thereof. According to some embodiments, a DNA molecule, construct, segment, amplicon, fragment or polynucleotide is provided that contains or comprises (i) SEQ ID NO: 9 or a polynucleotide sequence comprising at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 9, and (ii) a sequence or flanking sequence, or a 5′ flank, that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 17 or 19, and (iii) a sequence or flanking sequence, or a 3′ flank, that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 18 or 20.

According to present embodiments, a DNA molecule, construct, segment, amplicon, fragment or polynucleotide is provided that contains or comprises (i) SEQ ID NO: 9 or a polynucleotide sequence comprising at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 9, (ii) a polynucleotide sequence comprising one or more polynucleotide sequences selected from SEQ ID NOs: 30-49 and SEQ ID NOs: 50-129, and (iii) a polynucleotide sequence comprising one or more polynucleotide sequences selected from SEQ ID NOs: 130-149 and SEQ ID NOs: 150-229.

According to present embodiments, a DNA molecule, construct, segment, amplicon, fragment or polynucleotide is provided that contains or comprises SEQ ID NO: 9 or a polynucleotide sequence comprising at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 9, wherein the DNA molecule, construct, segment, amplicon, fragment or polynucleotide comprises at its 5′ end at least 50 consecutive nucleotides of SEQ ID NO: 17 or 19. According to present embodiments, a DNA molecule, construct, segment, amplicon, fragment or polynucleotide is provided that contains or comprises SEQ ID NO: 9 or a polynucleotide sequence comprising at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 9, wherein the DNA molecule, construct, segment, amplicon, fragment or polynucleotide comprises at its 3′ end at least 50 consecutive nucleotides of SEQ ID NO: 18 or 20. According to present embodiments, a DNA molecule, construct, segment, amplicon, fragment or polynucleotide is provided that contains or comprises an expression cassette that encodes a microRNA (miRNA) that suppresses the expression of the endogenous GA20ox3 and GA20ox5 genes in corn or maize, wherein the DNA molecule, construct, segment, amplicon, fragment or polynucleotide comprises at its 5′ end a polynucleotide sequence selected from SEQ ID NOs: 30-49 and SEQ ID NOs: 50-129. According to present embodiments, a DNA molecule, construct, segment, amplicon, fragment or polynucleotide is provided that contains or comprises SEQ ID NO: 9 or a polynucleotide sequence comprising at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 9, wherein the DNA molecule, construct, segment, amplicon, fragment or polynucleotide comprises at its 3′ end a polynucleotide sequence selected from SEQ ID NOs: 130-149 and SEQ ID NOs: 150-229.

According to present embodiments, a DNA molecule, construct, segment, amplicon, fragment or polynucleotide is provided that contains or comprises SEQ ID NO: 9 or a polynucleotide sequence comprising at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 9, wherein the DNA molecule, construct, segment, amplicon, fragment or polynucleotide comprises at its 5′ end at least 50 consecutive nucleotides of SEQ ID NO: 17 or 19, wherein the DNA molecule, construct, segment, amplicon, fragment or polynucleotide comprises at its 3′ end at least 50 consecutive nucleotides of SEQ ID NO: 18 or 20, wherein the DNA molecule, construct, segment, amplicon, fragment or polynucleotide comprises at its 5′ end a polynucleotide sequence selected from SEQ ID NOs: 30-49 and SEQ ID NOs: 50-129, and/or wherein the DNA molecule, construct, segment, amplicon, fragment or polynucleotide comprises at its 3′ end a polynucleotide sequence selected from SEQ ID NOs: 130-149 and SEQ ID NOS: 150-229.

According to present embodiments, a corn plant, plant part, plant seed, plant tissue, plant cell or commodity product is provided comprising any DNA molecule, construct, segment, fragment, or polynucleotide described herein. A corn plant comprising a construct as described herein may be further characterized as having a short stature or semi-dwarf trait or phenotype and a lodging and/or green snap resistance trait or phenotype, relative to a non-transgenic control plant.

To detect the presence or absence of corn event ZM_BCS216090 or a modified corn event ZM_BCS216090, the target positions and/or the intervening region or sequence of a template DNA molecule may comprise at least one junction sequence and/or at least a portion of the insert of corn event ZM_BCS216090 or a modified corn event ZM_BCS216090. To detect the absence of corn event ZM_BCS216090 or a modified corn event ZM_BCS216090, the target positions and/or the intervening region or sequence of a template DNA molecule may comprise corn genomic DNA that does not include a junction sequence or any portion of the insert of corn event ZM_BCS216090 or a modified corn event ZM_BCS216090. Thus, the presence or absence of an amplicon with a primer pair may be diagnostic of the presence or absence, respectively, of corn event ZM_BCS216090 or a modified corn event ZM_BCS216090 in a DNA molecule or sample, or vice versa. This may also be possible with more than one primer pair. For example, a first primer pair may produce a first amplicon if corn event ZM_BCS216090 or a modified corn event ZM_BCS216090 is present, and a second primer pair may produce a second amplicon if corn event ZM_BCS216090 or a modified corn event ZM_BCS216090 is absent or not present. Alternatively, the size of an amplicon produced in an amplification reaction may also be diagnostic of the presence or absence of corn event ZM_BCS216090 or a modified corn event ZM_BCS216090 in a DNA molecule or sample—e.g., a primer pair may produce a first amplicon of a first size if corn event ZM_BCS216090 or a modified corn event ZM_BCS216090 is present or a second amplicon of a second size if corn event ZM_BCS216090 or a modified corn event ZM_BCS216090 is absent and not present; or a first primer pair may produce a first amplicon of a first size if corn event ZM_BCS216090 or a modified corn event ZM_BCS216090 is present, and a second primer pair may produce a second amplicon of a second size if corn event ZM_BCS216090 or a modified corn event ZM_BCS216090 is absent or not present. According to some of these embodiments, at least two primer pairs may be used wherein at least one of the primer pairs is used as an internal control and is not associated with corn event ZM_BCS216090 or a modified corn event ZM_BCS216090.

According to present embodiments, a primer pair to detect the presence of all or part of corn event ZM_BCS216090 or a modified corn event ZM_BCS216090 in a DNA molecule or sample comprises a first primer and a second primer, wherein the first primer is complementary to a 5′ flanking genomic DNA sequence and the second primer is complementary to a sequence within the transgenic insert; or wherein the first primer is complementary to a 5′ flanking genomic DNA sequence and the second primer is complementary to a 3′ flanking genomic DNA sequence; or wherein the first primer is complementary to a 3′ flanking genomic DNA sequence and the second primer is complementary to a 5′ flanking genomic DNA sequence; or wherein the first primer is complementary to a sequence within the transgenic insert and the second primer is complementary to a 3′ flanking genomic DNA sequence; or wherein the first primer is complementary to a sequence within the transgenic insert and the second primer is complementary to a 5′ flanking genomic DNA sequence; or wherein the first primer is complementary to a 3′ flanking genomic DNA sequence and the second primer is complementary to a sequence within the transgenic insert. Each reference in this paragraph to a primer complementary to a 5′ flanking genomic DNA sequence, a 3′ flanking genomic DNA sequence, or a sequence within the transgenic insert of corn event ZM_BCS216090 or a modified corn event ZM_BCS216090 is also intended to potentially include a primer complementary to the reverse complement or opposing strand of the respective 5′ flanking genomic DNA sequence, 3′ flanking genomic DNA sequence, or sequence within the transgenic insert of corn event ZM_BCS216090 or a modified corn event ZM_BCS216090.

Exemplary DNA molecules useful as primers are provided as SEQ ID NO: 11, SEQ ID NO: 12, SEQ ID NO: 14, and SEQ ID NO: 15. The primer pair SEQ ID NO: 11 and SEQ ID NO: 12 can be useful as a first DNA molecule or primer and a second DNA molecule or primer, wherein each primer has sufficient length of consecutive nucleotides of SEQ ID NO: 10 or a sequence complementary to SEQ ID NO: 10 to function as DNA primers that, when used together in an amplification reaction with template DNA derived from corn event ZM_BCS216090 or a modified corn event ZM_BCS216090, hybridize to opposite strands of the template DNA and produce an amplicon diagnostic for corn event ZM_BCS216090 DNA or DNA from a modified corn event ZM_BCS216090 in a sample. The primer pair SEQ ID NO: 14 and SEQ ID NO: 15 are useful as a first DNA molecule or primer and a second DNA molecule or primer, wherein each primer has sufficient length of consecutive nucleotides of a locus within the corn genome to function as DNA primers that, when used together in a thermal amplification reaction with template DNA derived from corn event ZM_BCS216090 or a modified corn event ZM_BCS216090, to produce an amplicon that serves as an internal control for both the diagnosis of corn event ZM_BCS216090 or a modified corn event ZM_BCS216090, as well as the zygosity of corn event ZM_BCS216090 DNA or DNA from a modified corn event ZM_BCS216090 in a sample.

DNA probes and DNA primers are generally eleven (11) polynucleotides or more in length, and often eighteen (18) polynucleotides or more, twenty-one (21) polynucleotides or more, twenty-four (24) polynucleotides or more, or thirty (30) polynucleotides or more in length. Such probes and primers are selected to be of sufficient length and sequence complementarity to a target sequence to hybridize specifically to the target sequence under high stringency hybridization conditions. Preferably, probes and primers according to the present disclosure have complete sequence complementarity or identity with the target sequence, although probes and primers differing from the target sequence in terms of identity or complementarity but retain the ability to hybridize to the target sequence may be designed by conventional methods.

The nucleic acid probes and primers of the present disclosure hybridize under stringent conditions to a target DNA molecule. Any conventional nucleic acid hybridization or amplification method can be used to detect or identify the presence of a target DNA from a transgenic plant in a sample. Polynucleic acid or DNA molecules, also referred to as nucleic acid or DNA segments or fragments thereof, are capable of specifically hybridizing to other complementary nucleic acid or DNA molecules under certain circumstances.

As used herein, two polynucleic acid molecules are said to be capable of specifically hybridizing to one another if the two molecules are capable of forming an anti-parallel, double-stranded nucleic acid structure. A nucleic acid molecule is said to be the “complement” of another nucleic acid molecule if they exhibit complete complementarity. As used herein, nucleic acid molecules are said to exhibit “complete complementarity” and are “completely complementary” when every nucleotide of one of the molecules is complementary to a nucleotide of the other, in order of their respective sequences. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., 1989, and by Haymes et al., In: Nucleic Acid Hybridization, A Practical Approach, IRL Press, Washington, DC (1985). Departures from complete complementarity are therefore permissible, as long as such departures do not completely preclude the capacity of the molecules to form a double-stranded structure. In order for a nucleic acid molecule to serve as a primer or probe, it need only be sufficiently complementary in sequence to be able to form a stable double-stranded structure under the particular solvent and salt concentrations and other conditions employed.

As used herein, a substantially homologous sequence in relation to a reference nucleic acid sequence is a nucleic acid sequence that will specifically hybridize to the complement of the reference nucleic acid sequence to which it is being compared under high stringency conditions.

Appropriate stringency conditions that promote DNA hybridization, for example, 6.0×sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed. In some embodiments, a polynucleic acid of the present disclosure, such as a primer or probe, will specifically hybridize to one or more of the nucleic acid molecule sequences set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and/or SEQ ID NO: 10, or complements thereof or fragments thereof, under moderately stringent conditions, for example at about 2.0×SSC and about 65° C. In some embodiments, a nucleic acid of the present disclosure, such as a primer or probe, will specifically hybridize to one or more of the nucleic acid molecule sequences set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and/or SEQ ID NO: 10, or complements or fragments thereof, under high stringency conditions. In one aspect of the present invention, a preferred nucleic acid molecule of the present disclosure has or comprises the nucleic acid sequence set forth in one or more of SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8, SEQ ID NO: 9, and/or SEQ ID NO: 10, or complements thereof, or fragments thereof. The hybridization of a nucleic acid molecule, such as a primer or probe, to a target DNA molecule can be detected by any number of methods known to those skilled in the art, these can include, but are not limited to, fluorescent tags, radioactive tags, antibody based tags, and chemiluminescent tags.

Regarding the amplification of a target nucleic acid sequence (e.g., by PCR) using a particular amplification primer pair, “stringent conditions” are conditions that permit the primer pair to hybridize only to the target nucleic acid sequence to which a primer having the corresponding sequence (or its complement) would bind and preferably to produce a unique amplification product, the amplicon, in a DNA thermal amplification reaction.

The term “specific for (a target sequence)” indicates that a probe or primer hybridizes under stringent hybridization conditions only to the target sequence in a sample comprising the target sequence.

As used herein, “amplified DNA” or “amplicon” refers to the nucleic acid or DNA product of a polynucleic acid or DNA amplification reaction or method as further described herein, which is directed to a target polynucleic acid or DNA molecule that is part of a template polynucleic acid or DNA molecule. For example, to determine whether a corn plant, etc., resulting from a sexual cross of two parents contains transgenic plant genomic DNA from a corn plant comprising event ZM_BCS216090 of the present disclosure, DNA may be extracted from a corn plant tissue sample and subjected to an amplification reaction or method using a primer pair that is specific for a target sequence that is uniquely associated or part of event ZM_BCS216090, such as, for example, a first primer derived from a genomic DNA sequence in the region flanking the heterologous inserted DNA of event ZM_BCS216090 that is elongated by polymerase 5′ to 3′ in the direction of the inserted DNA, and a second primer derived from the heterologous inserted DNA molecule that is elongated by the polymerase 5′ to 3′ in the direction of the flanking genomic DNA from which the first primer is derived. The amplicon may range in length from the combined length of the primer pair plus one nucleotide base pair, or plus about fifty nucleotide base pairs, or plus about two hundred-fifty nucleotide base pairs, or plus about four hundred-fifty nucleotide base pairs or more depending on the length of the intervening polynucleic acid, polynucleotide or DNA sequence between the two primer target sequences in the template polynucleic acid, polynucleotide or DNA molecule. Alternatively, a primer pair can be derived from genomic sequence on both sides of the inserted heterologous DNA so as to produce an amplicon that includes the entire insert polynucleotide sequence (e.g., a forward primer isolated from the genomic portion on the 5′ end of SEQ ID NO: 10 and a reverse primer isolated from the genomic portion on the 3′ end of SEQ ID NO: 10 that amplifies a DNA molecule comprising the inserted DNA sequence (SEQ ID NO: 9) identified herein in the event ZM_BCS216090 genome). A member of a primer pair derived from the plant genomic sequence adjacent to the inserted transgenic DNA is located a distance from the inserted DNA sequence, this distance can range from one nucleotide base pair up to about twenty thousand nucleotide base pairs. The use of the term “amplicon” specifically excludes primer dimers that may be formed in a DNA amplification reaction.

For practical purposes, one should design primers which produce amplicons of a limited size range, for example, between 100 to 1000 bases. Smaller (shorter polynucleotide length) sized amplicons in general are more reliably produced in thermal amplification reactions, allow for shorter cycle times, and can be easily separated and visualized on agarose gels or adapted for use in endpoint TaqMan®-like assays. Smaller amplicons can be produced and detected by methods known in the art of DNA amplicon detection. In addition, amplicons produced using the primer pairs can be cloned into vectors, propagated, isolated, and sequenced or can be sequenced directly with methods well established in the art. Any primer pair of forward and reverse primers, which may be identical or complementary to part of SEQ ID NO: 10, such as an appropriate combination of SEQ ID NOs: 11, 12, 14, and 15, that is useful in a DNA amplification method to produce an amplicon diagnostic for event ZM_BCS216090 or a modified corn event ZM_BCS216090 or progeny thereof is an aspect of the disclosure. Any single isolated DNA polynucleotide primer molecule comprising at least 15 contiguous nucleotides of SEQ ID NO: 10, or its complement that is useful in a DNA amplification method to produce an amplicon diagnostic for event ZM_BCS216090 or a modified corn event ZM_BCS216090 or progeny thereof is an aspect of the disclosure. Any single isolated DNA polynucleotide primer molecule comprising at least 15 contiguous nucleotides of SEQ ID NO: 12, or its complement that is useful in a DNA amplification method to produce an amplicon diagnostic for plants comprising event ZM_BCS216090 or a modified corn event ZM_BCS216090 or progeny thereof is an aspect of the disclosure. Any single isolated DNA polynucleotide primer molecule comprising at least 15 contiguous nucleotides of SEQ ID NO: 9, or its complement that is useful in a DNA amplification method to produce an amplicon diagnostic for event ZM_BCS216090 or a modified corn event ZM_BCS216090 or progeny thereof is an aspect of the disclosure.

A diagnostic amplicon produced by the methods described herein may be detected by a plurality of techniques known in the art, such as sequencing, restriction mapping, Northern analysis, Southern analysis, or any other suitable polynucleotide or DNA hybridization, blotting, polymerization and/or amplification based approach or technique. One method is Genetic Bit Analysis (Nikiforov et al., Nucleic Acid Res. 22:4167-4175, 1994) where a DNA oligonucleotide is designed that overlaps both the adjacent flanking genomic DNA sequence and the inserted DNA sequence—i.e., a junction sequence. The oligonucleotide is immobilized in wells of a microtiter plate. Following PCR of the region of interest (using, for example, one primer in the inserted sequence and one in the adjacent flanking genomic sequence), a single-stranded PCR product can be hybridized to the immobilized oligonucleotide and serve as a template for a single base extension reaction using a DNA polymerase and labeled dideoxynucleotide triphosphates (ddNTPs) specific for the expected next base. Readout may be fluorescent or ELISA-based. A signal indicates presence of the transgene/genomic junction sequence due to successful amplification, hybridization, and single base extension.

Another method is the pyrosequencing technique as described by Winge (Innov. Pharma. Tech. 00:18-24, 2000). In this method, an oligonucleotide is designed that overlaps the adjacent genomic DNA and insert DNA junction. The oligonucleotide is hybridized to single-stranded PCR product from the region of interest (one primer in the inserted sequence and one in the flanking genomic sequence) and incubated in the presence of a DNA polymerase, ATP, sulfurylase, luciferase, apyrase, adenosine 5′ phosphosulfate and luciferin. DNTPs are added individually and the incorporation results in a light signal that is measured. A light signal indicates the presence of the transgene/genomic sequence due to successful amplification, hybridization, and single or multi-base extension.

Fluorescence Polarization as described by Chen et al. (Genome Res. 9:492-498, 1999) is a method that can be used to detect the amplicon of the present invention. Using this method an oligonucleotide is designed that overlaps the genomic flanking and inserted DNA junction. The oligonucleotide is hybridized to single-stranded PCR product from the region of interest (one primer in the inserted DNA and one in the flanking genomic DNA sequence) and incubated in the presence of a DNA polymerase and a fluorescent-labeled ddNTP. Single base extension results in incorporation of the ddNTP. Incorporation can be measured as a change in polarization using a fluorometer. A change in polarization indicates the presence of the transgene/genomic sequence due to successful amplification, hybridization, and single base extension.

Real-time polymerase chain reaction (PCR) is the ability to monitor the progress of the PCR as it occurs (i.e., in real time). Data is collected throughout the PCR process, rather than at the end of the PCR. In real-time PCR, reactions are characterized by the point in time during cycling when amplification of a target is first detected rather than the amount of target accumulated after a fixed number of cycles. In a real-time PCR assay, a positive reaction is detected by accumulation of a fluorescent signal. The higher the starting copy number of the nucleic acid target, the sooner a significant increase in fluorescence is observed. The cycle threshold (Ct value) is defined as the number of cycles required for the fluorescent signal to cross the threshold (i.e., exceeds background level). Ct levels are inversely proportional to the amount of target nucleic acid in the sample (i.e., the lower the Ct value, the greater the amount of target nucleic acid in the sample).

Taqman® (PE Applied Biosystems, Foster City, CA) is described as a method of detecting and quantifying the presence of a DNA sequence using real-time PCR and is fully understood in the instructions provided by the manufacturer. Briefly, a FRET oligonucleotide probe is designed that overlaps the genomic flanking and insert DNA junction. The FRET probe and PCR primers (one primer in the insert DNA sequence and one in the flanking genomic sequence) are cycled in the presence of a thermalstable polymerase and dNTPs. Hybridization of the FRET probe results in cleavage and release of the fluorescent moiety away from the quenching moiety on the FRET probe. A fluorescent signal indicates the presence of the transgene/genomic sequence due to successful amplification and hybridization.

Molecular beacons have been described for use in sequence detection as described in Tyangi et al. (Nature Biotech. 14:303-308, 1996). Briefly, a FRET oligonucleotide probe is designed that overlaps the flanking genomic and insert DNA junction. The unique structure of the FRET probe results in it containing secondary structure that keeps the fluorescent and quenching moieties in close proximity. The FRET probe and PCR primers (one primer in the insert DNA sequence and one in the flanking genomic sequence) are cycled in the presence of a thermalstable polymerase and dNTPs. Following successful PCR amplification, hybridization of the FRET probe to the target sequence results in the removal of the probe secondary structure and spatial separation of the fluorescent and quenching moieties. A fluorescent signal results. A fluorescent signal indicates the presence of the flanking/transgene insert sequence due to successful amplification and hybridization.

Other detection methods known in the art may be used. For example, microfluidics (see, e.g., U.S. Patent Publication No. 2006/068398; U.S. Pat. No. 6,544,734) provide methods and devices that can be used to separate and amplify DNA samples or molecules. Optical dyes can be used to detect and measure specific DNA molecules (see, e.g., WO/05017181). Nanotube devices (see, e.g., WO/06024023) that comprise an electronic sensor for the detection of DNA molecules or nanobeads that bind specific DNA molecules can then be detected.

DNA detection kits that are based on DNA amplification methods contain DNA primer molecules that hybridize specifically to a target DNA and amplify a diagnostic amplicon under the appropriate reaction conditions. The kit may provide an agarose gel based detection method or any number of methods of detecting the diagnostic amplicon that are known in the art. DNA detection kits can be developed using the compositions disclosed herein and are useful for identification of corn event ZM_BCS216090 DNA or DNA from a modified corn event ZM_BCS216090 in a sample and can be applied to methods for breeding corn plants containing event ZM_BCS216090 DNA or DNA from a modified corn event ZM_BCS216090. A kit that contains DNA primers that are homologous or complementary to any portion of the corn genomic region as set forth in SEQ ID NO: 10 and to any portion of the inserted transgenic DNA as set forth in SEQ ID NO: 9 is an object of the invention. The DNA molecules can be used in DNA amplification methods (PCR) or as probes in polynucleic acid hybridization methods, i.e., southern analysis, northern analysis, etc. Kits of the invention may optionally also comprise reagents or instructions for performing the detection or diagnostic reactions described herein.

Probes and primers as provided herein may have complete sequence identity with the target sequence, although primers and probes differing from the target sequence that retain the ability to hybridize preferentially to target sequences may be designed by conventional methods. In order for a nucleic acid molecule to serve as a primer or probe it need only be sufficiently complementary in sequence to be able to form a stable double-stranded structure under the particular solvent and salt concentrations employed. Any conventional nucleic acid hybridization or amplification method can be used to identify the presence of transgenic DNA from corn event ZM_BCS216090 or a modified corn event ZM_BCS216090 in a sample.

Any number of methods well known to those skilled in the art can be used to isolate and manipulate a DNA molecule, or fragment thereof, disclosed herein, including DNA isolation or thermal amplification or PCR methods. Such DNA molecule or fragment may be inserted or placed into any suitable vector or plasmid or combined with other elements, sequences or fragments using molecular or recombinant techniques.

The DNA molecules and corresponding nucleotide sequences provided herein are therefore useful for, among other things, identifying corn event ZM_BCS216090 or a modified corn event ZM_BCS216090, detecting the presence of DNA derived from the transgenic corn event ZM_BCS216090 or a modified corn event ZM_BCS216090 in a sample, and monitoring samples for the presence and/or absence of corn event ZM_BCS216090 or a modified corn event ZM_BCS216090 or plant parts derived from corn plants comprising event ZM_BCS216090 or a modified corn event ZM_BCS216090.

Reference herein to “corn” generally is intended to include corn plants, corn plant cells, corn plant tissues, corn seeds, corn plant parts, corn progeny plants, and/or corn commodity products, depending on the context of its use herein, unless otherwise provided. The present disclosure provides corn plants, corn plant cells, corn plant tissues, corn seeds, corn plant parts (such as pollen, ovule, silk, spike, anther, cob, root tissue, stalk tissue, leaf tissue), corn progeny plants, and corn commodity products. These corn plants, corn plant cells, corn plant tissues, corn seeds, corn plant parts, corn progeny plants, and corn commodity products contain a detectable amount of a polynucleotide or DNA molecule or sequence comprising at least one junction sequence and/or heterologous insert sequence of corn event ZM_BCS216090 or a modified corn event ZM_BCS216090, such as a polynucleotide or DNA molecule or sequence having or comprising at least one of the sequences provided as SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

The invention provides corn plants, corn plant cells, corn plant tissues, corn seeds, corn plant parts (such as pollen, ovule, silk, spike, anther, cob, root tissue, stalk tissue, leaf tissue), corn progeny plants, or corn commodity products that either contain or comprise ZM_BCS216090 DNA or DNA from a modified corn event ZM_BCS216090 or are derived from a transgenic corn plant, corn plant cell, corn plant tissue, corn seed, corn plant part, corn progeny plant, or corn commodity product containing or comprising event ZM_BCS216090 DNA or DNA from a modified corn event ZM_BCS216090. A representative sample of corn seed containing event ZM_BCS216090 DNA has been deposited according to the Budapest Treaty with the American Type Culture Collection (ATCC®). The ATCC repository has assigned the Patent Deposit Designation PTA-127050 to the seed containing event ZM_BCS216090 DNA.

The present disclosure further provides a microorganism, such as a bacterial or fungal cell, comprising a DNA molecule having at least one sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10, which may be present in its genome. A microorganism is intended to include any microscopic cell or organism, whether prokaryote or eukaryote or otherwise, that contains DNA within a genome or chromosome or an extra-chromosomal DNA structure, such as a plasmid or vector, in such microscopic cell. Microscopic cells or organisms include bacteria (prokaryotes) and cells corresponding to higher life forms (eukaryotes) which are beneath the visual range of the average human. An example of such a microorganism is a transgenic plant cell. Microorganisms, such as a plant cell of the present disclosure, are useful in many industrial applications, including but not limited to: (i) use as research tool for scientific inquiry or industrial research; (ii) use in culture for producing endogenous or recombinant carbohydrate, lipid, nucleic acid, or protein products or small molecules that may be used for subsequent scientific research or as industrial products; and (iii) use with modern plant tissue culture techniques to produce transgenic plants, plant parts, plant organs or plant tissue cultures that may then be used for agricultural research or production. The production and use of microorganisms, such as transgenic plant cells, utilizes modern microbiological techniques and human intervention to produce a man-made, unique microorganism. In this process, recombinant DNA is inserted into a plant cell's genome to create a transgenic plant cell that is separate and unique from naturally occurring plant cells. This transgenic plant cell can then be cultured much like bacteria and yeast cells using modern microbiology techniques and may exist in an undifferentiated, unicellular state. The transgenic plant cell's new genetic composition and phenotype is a technical effect created by the integration or insertion of the heterologous DNA into the genome of the cell. Another aspect of the present disclosure is a method of using a microorganism provided herein. Methods of using microorganisms of the present disclosure, such as transgenic plant cells, include (i) methods of producing transgenic cells by integrating recombinant DNA into the genome of the cell and then using this cell to derive additional cells possessing the same heterologous DNA; (ii) methods of culturing cells that contain recombinant DNA using modern microbiology techniques; (iii) methods of producing and purifying endogenous or recombinant carbohydrate, lipid, nucleic acid, or protein products from cultured cells; and (iv) methods of using modern plant tissue culture techniques with transgenic plant cells to produce transgenic plants or transgenic plant tissue cultures.

Corn plants of the present disclosure may pass along the event ZM_BCS216090 DNA or DNA from a modified corn event ZM_BCS216090, including the transgene inserted as part of corn event ZM_BCS216090 or a modified corn event ZM_BCS216090, to progeny or offspring. Such progeny may include any corn plant, plant cell, seed, gamete and/or regenerable plant part containing or comprising the event ZM_BCS216090 DNA or DNA from a modified corn event ZM_BCS216090 inherited or derived from an ancestor or parental corn plant(s), at least one of which comprises a DNA molecule having or comprising at least one sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10. Corn plants, progeny, and seeds may be homozygous or heterozygous for the event ZM_BCS216090 or a modified corn event ZM_BCS216090 and the transgene of event ZM_BCS216090 or a modified corn event ZM_BCS216090. Progeny may be grown from seeds produced by a corn plant comprising or containing event ZM_BCS216090 or a modified corn event ZM_BCS216090 and/or from seeds produced by a plant fertilized with pollen from a corn plant comprising or containing event ZM_BCS216090 or a modified corn event ZM_BCS216090 (i.e., fertilized with pollen comprising or containing event ZM_BCS216090 or a modified corn event ZM_BCS216090).

Methods for producing corn plants and seeds containing or comprising maize event ZM_BCS216090 or a modified corn event ZM_BCS216090 are provided. Corn plants may be bred using any method known in the art, for example, descriptions of breeding methods that are commonly used can be found in WR Fehr, in Breeding Methods for Cultivar Development, Wilcox J. ed., American Society of Agronomy, Madison WI (1987). Corn plants or progeny plants containing or comprising maize event ZM_BCS216090 or a modified corn event ZM_BCS216090 may be self-pollinated (also known as “selfing”) to generate a true breeding line of corn plants, i.e., corn plants homozygous for the transgene and event ZM_BCS216090 or a modified corn event ZM_BCS216090. Selfing can result in progeny known as an “inbred” that can be used to produce corn inbred lines that are genetically uniform.

Alternatively, corn plants or progeny plants containing or comprising maize event ZM_BCS216090 or a modified corn event ZM_BCS216090 may be out-crossed or cross-pollinated (also known as “crossing”), e.g., bred with another plant having a different germplasm or genotype, to produce a varietal or hybrid seed or plant that may be homozygous or heterozygous for the transgene and event ZM_BCS216090 or a modified corn event ZM_BCS216090 depending on whether the other parental plant also comprises or contains the transgene and event ZM_BCS216090 or a modified corn event ZM_BCS216090. The other parental plant may be transgenic or non-transgenic for the same and/or different trait, transgene, or event. A varietal or hybrid seed or plant of the invention may thus be derived by sexually crossing a first parent that lacks the specific and unique corn event ZM_BCS216090 or a modified corn event ZM_BCS216090 with a second parent comprising corn event ZM_BCS216090 or a modified corn event ZM_BCS216090, resulting in a hybrid plant or progeny plant containing or comprising the specific and unique corn event ZM_BCS216090 or a modified corn event ZM_BCS216090. Each parent can be a hybrid or an inbred/varietal plant, so long as a parent or progeny plant or seed of the cross has or comprises at least one copy of the corn event ZM_BCS216090 or a modified corn event ZM_BCS216090 and/or a DNA molecule having or comprising at least one sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

Sexually crossing one plant with another plant, i.e., cross-pollinating, may be accomplished or facilitated by human intervention, for example: by human hands or other mechanical means under human, computer or automated control collecting the pollen of one plant and contacting this pollen with the style or stigma of a second plant; by human hands and/or human actions or other mechanical means under human, computer or automated control removing, destroying, devitalizing or covering the stamen or anthers of a plant (e.g., by manual intervention or by application of a chemical gametocide) so that natural self-pollination is prevented and cross-pollination would have to take place in order for fertilization to occur; by human placement of pollinating insects in a position for “directed pollination” (e.g., by placing beehives in orchards or fields or by caging plants with pollinating insects); by human opening or removing of parts of the flower to allow for placement or contact of foreign pollen on the style or stigma; by selective placement of plants (e.g., intentionally planting plants in pollinating proximity); and/or by application of chemicals to precipitate flowering or to foster receptivity (of the stigma for pollen).

Two different transgenic plants may thus be crossed to produce hybrid offspring plants, plant parts and/or seeds that contain two independently segregating transgenes or events wherein at least one of those transgenes or events comprises or is contained within corn event ZM_BCS216090 or a modified corn event ZM_BCS216090. For example, transgenic plants comprising corn event ZM_BCS216090 or a modified corn event ZM_BCS216090 can be crossed with other transgenic corn plants to produce a plant having the characteristics of both transgenic parents. Back-crossing to a parental plant and out-crossing with a non-transgenic plant are also contemplated, as is vegetative propagation. Descriptions of other breeding methods that are commonly used for different traits and crops are known in the art and can be found in one of several references, e.g., Fehr, in Breeding Methods for Cultivar Development, Wilcox J. ed., American Society of Agronomy, Madison WI (1987).

According to some embodiments, a reduced plant height trait or phenotype may be used to select one or more progeny corn plants, plant parts or seeds that contain corn or maize event ZM_BCS216090 or a modified corn event ZM_BCS216090. Alternatively, progeny plants, plant parts or seeds may be analyzed using diagnostic methods as described herein to select for plants, plant parts or seeds containing or comprising corn or maize event ZM_BCS216090 or a modified corn event ZM_BCS216090.

Corn plants and progeny plants comprising corn event ZM_BCS216090 or a modified corn event ZM_BCS216090 may have a short stature or semi-dwarf trait or phenotype and a lodging and/or green snap resistance trait or phenotype as described, for example, in PCT Application No. PCT/US2017/047405 (PCT Application Publication No. WO 2018/035354), the entire contents and disclosure of which are incorporated herein by reference. Corn plants, progeny, seeds, tissues, cells and plant parts comprising corn event ZM_BCS216090 or a modified corn event ZM_BCS216090 as provided herein may also contain one or more additional corn trait(s) or transgenic event(s), such as by crossing a corn plant containing corn event ZM_BCS216090 or a modified corn event ZM_BCS216090 with another corn plant containing the additional trait(s) or transgenic event(s). Such additional trait(s) or transgenic event(s) may include, but are not limited to, increased insect resistance, herbicide tolerance, increased water use efficiency, increased yield performance, increased drought resistance, increased seed quality, improved nutritional quality, hybrid seed production, or disease or fungal resistance. A corn trait may include any transgenic traits or mutant or edited traits or alleles. Mutant traits or alleles of a gene may be created by any mutagenesis technique known in the art, whereas edited traits may be generated by any genome editing technique or method known in the art. Many corn transgenic event(s) are known to those of skill in the art. For example, a list of such traits is provided by the United States Department of Agriculture's (USDA) Animal and Plant Health Inspection Service (APHIS) and can be found on their website: www.aphis.usda.gov. Two or more allele(s) and/or transgenic event(s) comprising or including at least one copy of corn event ZM_BCS216090 or a modified corn event ZM_BCS216090 may thus be combined in a progeny seed or plant by crossing two parent plants each comprising one or more allele(s) and/or transgenic event(s), collecting the progeny seed, and selecting for progeny seed or plants that contain the two or more allele(s) and/or transgenic event(s). These steps may be repeated until the desired combination of transgenic event(s) and/or allele(s) in a progeny plant is achieved. For the present application, the progeny plant will generally comprise corn event ZM_BCS216090 or a modified corn event ZM_BCS216090. Back-crossing to a parental plant and out-crossing with a non-transgenic plant are also contemplated and is vegetative propagation.

A plant part is provided that comprises event ZM_BCS216090 or a modified corn event ZM_BCS216090 and/or is derived from corn plants comprising event ZM_BCS216090 or a modified corn event ZM_BCS216090. As used herein, a “plant part” refers to any part of a plant which may comprise event ZM_BCS216090 or a modified corn event ZM_BCS216090 and/or material derived from a corn plant comprising event ZM_BCS216090 or a modified corn event ZM_BCS216090. Plant parts include, but are not limited to, plant tissue, pollen, ovule, silk, spike, anther, cob, root tissue, stalk tissue, and leaf tissue. Plant parts may be viable, nonviable, regenerable, and/or non-regenerable.

A commodity product is provided that is derived from one or more corn plants, plant parts, seeds and/or plant tissues comprising event ZM_BCS216090 or a modified corn event ZM_BCS216090 and that contains a detectable amount of a nucleic acid or DNA molecule, segment or sequence specific for event ZM_BCS216090 or a modified corn event ZM_BCS216090. As used herein, a “commodity product” refers to any composition or product comprising material derived from one or more corn plants, whole or processed corn seed, one or more plant cells, and/or one or more plant parts containing or comprising the corn event ZM_BCS216090 DNA or DNA from a modified corn event ZM_BCS216090. Nonviable commodity products include, but are not limited to, nonviable seeds, whole or processed seeds, seed parts, and plant parts; animal feed comprising corn, corn oil, corn meal, corn flour, corn flakes, corn bran, pasta made with corn, corn biomass, and fuel products produced using corn and corn parts. Viable commodity products include, but are not limited to, seeds, plants, and plant cells. The corn plants comprising event ZM_BCS216090 or a modified corn event ZM_BCS216090 can thus be used to manufacture any commodity product typically acquired from corn. Any such commodity product that is derived from corn plants comprising event ZM_BCS216090 or a modified corn event ZM_BCS216090 may contain at least a detectable amount of the specific and unique DNA corresponding to corn event ZM_BCS216090 or a modified corn event ZM_BCS216090, and specifically may contain a detectable amount of a polynucleotide or DNA molecule having or comprising at least one sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10. Detection of one or more of these polynucleotide or DNA sequences in a sample may be used to determine or diagnose that the sample is taken from a corn plant, corn plant part, corn plant tissue, corn plant cell, and/or corn plant product, such as a corn commodity product, comprising event ZM_BCS216090 or a modified corn event ZM_BCS216090, or to determine the content or source of a corn plant, corn plant part, corn plant tissue, corn plant cell, and/or corn plant product, such as a corn commodity product. Any standard method of detection for nucleotide molecules may be used, including methods of detection disclosed herein. A commodity product is within the scope of the present disclosure if there is any detectable amount of a DNA molecule having at least one sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10 contained or comprised in the commodity product.

The corn plants, corn plant cells, corn seeds, corn plant parts (such as pollen, ovule, silk, spike, anther, cob, root tissue, stalk tissue, leaf tissue), corn progeny plants, and commodity products of the present disclosure are therefore, useful for, among other things, growing plants for the purpose of producing seed and/or plant parts comprising corn event ZM_BCS216090 or a modified corn event ZM_BCS216090 for agricultural purposes, producing progeny comprising corn event ZM_BCS216090 or a modified corn event ZM_BCS216090 for plant breeding and research purposes, use with microbiological techniques for industrial and research applications, and sale to consumers.

Methods for producing corn plant(s) comprising the DNA sequences specific and unique to event ZM_BCS216090 or a modified corn event ZM_BCS216090 of the present disclosure are provided. A progeny corn plant comprising the event ZM_BCS216090 or a modified corn event ZM_BCS216090 may be produced, for example, by selfing a parent plant or line comprising the event ZM_BCS216090 or a modified corn event ZM_BCS216090, wherein such parent plant or line is homozygous or hemizygous for the event ZM_BCS216090 or a modified corn event ZM_BCS216090, or by crossing a first parent plant or line comprising the event ZM_BCS216090 or a modified corn event ZM_BCS216090, wherein such parent plant or line is homozygous or hemizygous for the event ZM_BCS216090 or a modified corn event ZM_BCS216090, with a second parent plant or line having a different genotype or germplasm than the first parent line, wherein the second parent plant or line may or may not contain or comprise the event ZM_BCS216090 or a modified corn event ZM_BCS216090. As explained further herein, corn event ZM_BCS216090 or a modified corn event ZM_BCS216090 may contain an expression cassette or transgene encoding a miRNA that targets GA20 oxidase genes for suppression leading to lower active GA levels in the plant and a reduced plant height (i.e., a semi-dwarf phenotype or trait). According to some embodiments, the transgenic corn plant(s) comprising the event ZM_BCS216090 or a modified corn event ZM_BCS216090 of the present disclosure is/are semi-dwarf and/or have a shorter plant height relative to a non-transgenic control plant. According to some embodiments, the transgenic corn plant(s) comprising the event ZM_BCS216090 or a modified corn event ZM_BCS216090 of the present disclosure is/are lodging resistant (resistant to root and/or stalk lodging) and/or resistant to green snap, relative to a non-transgenic control plant. Transgenic plants used in these methods may be homozygous or heterozygous for the transgene. Progeny plants produced by these methods may be varietal or hybrid plants; may be grown from seeds produced by plants containing corn event ZM_BCS216090 or a modified corn event ZM_BCS216090 and/or from seeds produced by a plant fertilized with pollen from a plant containing corn event ZM_BCS216090 or a modified corn event ZM_BCS216090; and may be homozygous or heterozygous for the transgene and/or event ZM_BCS216090 or a modified corn event ZM_BCS216090. Progeny plants may be subsequently self-pollinated to generate a true breeding line of plants, i.e., plants homozygous for the transgene, or alternatively may be out-crossed, e.g., bred with another unrelated plant, to produce a varietal or a hybrid seed or plant.

Methods of detecting the presence of DNA derived from a corn cell, corn tissue, corn seed, corn plant part, or corn plant comprising corn event ZM_BCS216090 or a modified corn event ZM_BCS216090 in a sample are provided. One method comprises (i) extracting a DNA sample from at least one corn cell, corn tissue, corn seed, or corn plant; (ii) contacting the DNA sample with at least one primer that is capable of producing DNA sequence specific to event ZM_BCS216090 DNA or a modified corn event ZM_BCS216090 under conditions appropriate for DNA sequencing; (iii) performing a DNA sequencing reaction; and then (iv) confirming that the nucleotide sequence comprises a nucleotide sequence specific for event ZM_BCS216090 or a modified corn event ZM_BCS216090, of the construct comprised therein, such as one selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

Another method comprises (i) extracting a DNA sample from at least one corn cell, corn tissue, corn seed, or corn plant; (ii) contacting the DNA sample with a primer pair that is capable of producing an amplicon from event ZM_BCS216090 DNA or DNA from a modified corn event ZM_BCS216090 under conditions appropriate for DNA amplification; (iii) performing a DNA amplification reaction; and then (iv) detecting the amplicon molecule and/or confirming that the nucleotide sequence of the amplicon comprises a nucleotide sequence specific for event ZM_BCS216090 or a modified corn event ZM_BCS216090, such as one selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10. The amplicon should be one that is specific for event ZM_BCS216090 or a modified corn event ZM_BCS216090, such as an amplicon that comprises SEQ ID NO: 1, or SEQ ID NO: 2, or SEQ ID NO: 3, or SEQ ID NO: 4, or SEQ ID NO: 5, or SEQ ID NO: 6, or SEQ ID NO: 7, or SEQ ID NO: 8, or SEQ ID NO: 9, or SEQ ID NO: 10. The detection of a nucleotide sequence specific for event ZM_BCS216090 or a modified corn event ZM_BCS216090 in the amplicon is determinative and/or diagnostic for the presence of the corn event ZM_BCS216090 specific DNA or DNA specific for a modified corn event ZM_BCS216090 in the sample. An example of a primer pair that is capable of producing an amplicon from event ZM_BCS216090 DNA under conditions appropriate for DNA amplification is provided as SEQ ID NO: 11 and SEQ ID NO: 12. Other primer pairs may be readily designed by one of skill in the art to produce an amplicon comprising SEQ ID NO: 1, or SEQ ID NO: 2, or SEQ ID NO: 3, or SEQ ID NO: 4, or SEQ ID NO: 5, or SEQ ID NO: 6, or SEQ ID NO: 7, or SEQ ID NO: 8, or SEQ ID NO: 9, or SEQ ID NO: 10, wherein such a primer pair comprises at least one primer within the genomic region flanking the insert and a second primer within the insert, provided that any primer pair could be designed and used that produces an amplicon comprising a junction sequence and/or all or part of the insert or transgene sequence. Detection of an amplicon could be based on any suitable method, such as sequencing, determining fragment size or migration of the amplicon in a matrix or gel, or a hybridization based method.

Another method of detecting the presence of DNA derived from a corn cell, corn tissue, corn seed, or corn plant comprising corn event ZM_BCS216090 or a modified corn event ZM_BCS216090 in a sample consists of (i) extracting a DNA sample from at least one corn cell, corn tissue, corn seed, or corn plant; (ii) contacting the DNA sample with a DNA probe specific for event ZM_BCS216090 DNA or a modified corn event ZM_BCS216090; (iii) allowing the probe and the DNA sample to hybridize under stringent hybridization conditions; and then (iv) detecting hybridization between the probe and the target DNA sample. An example of the sequence of a DNA probe that is specific for event ZM_BCS216090 is provided as SEQ ID NO: 13. Other probes may be readily designed by one of skill in the art and would comprise a junction sequence, at least one fragment of genomic DNA flanking the insert and at least one fragment of insert DNA, and/or one or more sequences provided in SEQ ID NO: 1, SEQ ID NO: 2, SEQ ID NO: 3, SEQ ID NO: 4, SEQ ID NO: 5, SEQ ID NO: 6, and SEQ ID NO: 10. Detection of probe hybridization to the DNA sample is diagnostic for the presence of corn event ZM_BCS216090 specific DNA or DNA specific for a modified corn event ZM_BCS216090 in the sample. Absence of hybridization is alternatively diagnostic of the absence of corn event ZM_BCS216090 specific DNA or DNA specific for a modified corn event ZM_BCS216090 in the sample.

DNA detection kits are provided that are useful for the identification of corn event ZM_BCS216090 DNA or DNA from a modified corn event ZM_BCS216090 in a sample and can also be applied to methods for breeding corn plants containing the appropriate event DNA. Such kits contain DNA primers and/or probe(s) which are specific to corn event ZM_BCS216090 or a modified corn event ZM_BCS216090. Such DNA primers and/or probe(s) may comprise one or more of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10, or a fragment thereof. One example of such a kit comprises at least one DNA molecule of sufficient length of continuous nucleotides of SEQ ID NO: 10 to function as a DNA probe useful for detecting the presence and/or absence of DNA derived from transgenic corn plants comprising event ZM_BCS216090 or a modified corn event ZM_BCS216090 in a sample. The DNA derived from transgenic corn plants comprising event ZM_BCS216090 would comprise a DNA molecule having at least one sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10. Likewise, the primers may comprise a primer pair including a first primer and a second primer, wherein at least one of the primers hybridizes to a flanking sequence and the other primer hybridizes to either an insert sequence of event ZM_BCS216090 or a modified corn event ZM_BCS216090 in the plant genome or the flanking sequence on the opposite side of the insert. The first and second primers hybridize to opposing strands of the corn plant genomic DNA at different spaced apart positions such that an amplification reaction involving the two primers produces an amplicon comprising the primer sequences and the intervening sequence between the two primers. A probe may be chosen to correspond or hybridize to the amplicon produced with a primer pair or set of primers and may comprise all or part of the primer sequence(s) and/or the intervening sequence of the amplicon between the two primers. Suitable probes may be readily designed by one of skill in the art and should comprise at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, or at least 40 contiguous nucleotides of SEQ ID NO: 10 and be sufficiently unique to corn event ZM_BCS216090 DNA or DNA from a modified corn event ZM_BCS216090 in order to identify DNA derived from the event.

Another type of kit comprises a primer pair useful for producing an amplicon useful for detecting the presence and/or absence of DNA derived from transgenic corn event ZM_BCS216090 or a modified corn event ZM_BCS216090 in a sample. Such a kit would employ a method comprising contacting a target DNA sample with a primer pair as described herein, then performing a nucleic acid amplification reaction sufficient to produce an amplicon comprising a DNA molecule having at least one sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10 and then detecting the presence and/or absence of the amplicon. Such a method may also include sequencing the amplicon or a fragment thereof, which would be determinative of, i.e., diagnostic for, the presence of the corn event ZM_BCS216090 specific DNA of DNA specific for a modified corn event ZM_BCS216090 in the target DNA sample. Other primer pairs may be readily designed by one of skill in the art and should comprise at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides of sequences provided in, but not limited to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10, and be sufficiently unique to corn event ZM_BCS216090 DNA in order to identify DNA derived from the event.

The kits and detection methods of the invention are useful for, among other things, identifying corn event ZM_BCS216090 or a modified corn event ZM_BCS216090, selecting plant varieties or hybrids comprising corn event ZM_BCS216090 or a modified corn event ZM_BCS216090, detecting the presence of DNA derived from the transgenic corn plant comprising event ZM_BCS216090 or a modified corn event ZM_BCS216090 in a sample, and monitoring samples for the presence and/or absence of corn plants comprising event ZM_BCS216090 or a modified corn event ZM_BCS216090, or plant parts derived from corn plants comprising event ZM_BCS216090 or a modified corn event ZM_BCS216090.

The sequences of the heterologous DNA insert, junction sequences, or flanking sequence from corn event ZM_BCS216090 or a modified corn event ZM_BCS216090 can be determined or verified by amplifying such sequences from the event using primers derived from the sequences provided herein followed by standard DNA sequencing of the amplicon or of the cloned DNA.

Methods of detecting the zygosity of the event and transgene with genomic DNA derived from at least one corn cell, corn tissue, corn seed, corn plant part or corn plant comprising corn event ZM_BCS216090 or a modified corn event ZM_BCS216090 in a sample are provided. One method consists of (i) extracting a DNA sample from at least one corn cell, corn tissue, corn seed, corn plant part, or corn plant; (ii) contacting the DNA sample with a first primer pair that is capable of producing a first amplicon diagnostic for event ZM_BCS216090 or a modified corn event ZM_BCS216090; (iii) contacting the DNA sample with a second primer pair that is capable of producing a second amplicon diagnostic for native corn genomic DNA not comprising event ZM_BCS216090 or a modified corn event ZM_BCS216090; (iv) performing a DNA amplification reaction; and then (v) detecting the amplicons, wherein the presence of only the first amplicon is diagnostic of a homozygous event ZM_BCS216090 DNA or DNA from a modified corn event ZM_BCS216090 in the sample, the presence of both the first amplicon and the second amplicon is diagnostic of a corn plant heterozygous for event ZM_BCS216090 or a modified corn event ZM_BCS216090, and the presence of only the second amplicon is diagnostic for the absence of event ZM_BCS216090 DNA or DNA from a modified corn event ZM_BCS216090 in the sample. An exemplary set of primers pairs are presented as SEQ ID NO: 11 and SEQ ID NO: 12 which produce an amplicon diagnostic for event ZM_BCS216090 or a modified corn event ZM_BCS216090; and SEQ ID NO: 14 and SEQ ID NO: 15 which produces an amplicon diagnostic for non-inserted wild-type corn genomic DNA not comprising event ZM_BCS216090 or a modified corn event ZM_BCS216090. A set of probes can also be incorporated into such an amplification method to be used in a real-time PCR format using the primer pair sets described above. An exemplary set of probes are presented as SEQ ID NO: 13 (diagnostic for the amplicon for the event ZM_BCS216090 or a modified corn event ZM_BCS216090) and SEQ ID NO: 16 (diagnostic for the amplicon for wild-type corn genomic DNA not comprising event ZM_BCS216090 or a modified corn event ZM_BCS216090).

Another method for determining zygosity consists of (i) extracting a DNA sample from at least one corn cell, corn tissue, corn seed, corn plant part, or corn plant; (ii) contacting the DNA sample with a probe set which contains at least a first probe that specifically hybridizes to event ZM_BCS216090 DNA or DNA from a modified corn event ZM_BCS216090 and at least a second probe that specifically hybridizes to corn genomic DNA that was disrupted by insertion of the heterologous DNA of event ZM_BCS216090 or a modified corn event ZM_BCS216090 and does not hybridize to event ZM_BCS216090 DNA or DNA from a modified corn event ZM_BCS216090; (iii) hybridizing the probe set with the sample under stringent hybridization conditions, wherein detecting hybridization of only the first probe under the hybridization conditions is diagnostic for a homozygous corn cell, corn tissue, corn seed, corn plant part, or corn plant for event ZM_BCS216090 DNA or DNA from a modified corn event ZM_BCS216090 in the sample; wherein detecting hybridization of both the first probe and the second probe under the hybridization conditions is diagnostic for a heterozygous corn cell, corn tissue, corn seed, corn plant part, or corn plant for event ZM_BCS216090 or a modified corn event ZM_BCS216090 in a DNA sample; and detecting hybridization of only the second probe under the hybridization conditions is diagnostic for the absence of event ZM_BCS216090 DNA or DNA from a modified corn event ZM_BCS216090 in the sample.

Yet another method for determining zygosity consists of (i) extracting a DNA sample from at least one corn cell, corn tissue, corn seed, corn plant part, or corn plant; (ii) contacting the DNA sample with a first primer pair that is capable of producing a first amplicon diagnostic for event ZM_BCS216090 or a modified corn event ZM_BCS216090; (iii) contacting the DNA sample with a second primer pair that is capable of producing a second amplicon of an internal standard known to be single-copy and homozygous in the corn plant; (iv) contacting the DNA sample with a probe set which contains at least a first probe that specifically hybridizes to the first amplicon, and at least a second probe that specifically hybridizes to the second amplicon; (v) performing a DNA amplification reaction using real-time PCR and determining the cycle thresholds (Ct values) of the first and second amplicons; (vi) calculating the difference (ΔCt) between the Ct value of the first amplicon and the second amplicon; and (vii) determining zygosity, wherein a ΔCt of about zero (0) indicates homozygosity of the event or inserted T-DNA, and a ΔCt of about one (1) indicates heterozygosity of the event or inserted T-DNA. Heterozygous and homozygous events are differentiated by a ΔCt value unit of approximately one (1). Given the normal variability observed in real-time PCR due to multiple factors such as amplification efficiency and ideal annealing temperatures, the range of “about one (1)” is defined as a ΔCt of 0.75 to 1.25, and the range of “about zero (0)” is defined as a ΔCt of −0.25 to 0.25 (or of 0.0 to 0.25 if the ΔCt is measured as an absolute value). Primer pairs and probes for the above method for determining zygosity can amplify and detect amplicons from the transgene or event DNA and the internal DNA standard.

According to embodiments of the present disclosure, a transgenic corn plant or plant part, one or more transgenic corn plants or plant parts or a plurality transgenic corn plants or plant parts as provided herein, or an agricultural field or soil in which a transgenic corn plant or plant part, one or more transgenic corn plants or plant parts or a plurality of transgenic corn plants or plant parts as provided herein are planted or grown, can be treated with an agricultural composition comprising one or more active ingredients or other agents, such as, for example and without limitation, an herbicide or one or more herbicides, a fungicide or one or more fungicides, an insecticide or one or more insecticides, a plant growth regulator or plant stimulant or one or more plant growth regulators and/or plant stimulants, and/or a safener or one or more safeners. Provided below are lists of possible or representative compounds for each of these types of actives or agents, and an agricultural composition may comprise one or any combination or multiplicity of these actives, agents, or compounds. Such an agricultural composition may be applied, for example, as a foliar, soil or in-furrow treatment, as a pre-emergent, pre-sowing and/or post-emergent treatment, and/or in some cases, may be applied to a transgenic plant part or seed provided herein.

An agricultural composition may be formulated according to its intended use and application. The appropriate formulation of the agricultural composition may be chosen to have different physicochemical parameters, components, and stabilities of the respective compound(s). Possible types of formulations for an agricultural composition can include, for example: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), dispersions based on oil or water, oil-miscible solutions, capsule suspensions (CS), dusting products (DP), dressings, granules for scattering and soil application, granules (GR) in the form of microgranules, spray granules, absorption and adsorption granules, water-dispersible granules (WG), water-soluble granules (SG), ULV formulations, microcapsules and waxes. If appropriate, some agricultural compositions of a pesticidal compound or one or more pesticidal compounds might be formulated and used as a seed coating applied to a plant part or seed as provided herein.

Plants, progeny, plant parts, plant seeds, plant tissues, and plant cells may contain or comprise one or more additional desirable trait(s). Such desirable traits may be transgenic traits, native traits, or traits produced by other methods, such as genome editing, base editing, prime editing, or other conventional mutagenesis methods. Such desirable trait(s) may provide an agronomic, agricultural or commodity benefit to a plant, plant part, plant seed or plant product. Desirable traits may be combined with corn event ZM_BCS216090 or a modified corn event ZM_BCS216090 by, for example, crossing a corn plant comprising corn event ZM_BCS216090 or a modified corn event ZM_BCS216090 with another corn plant containing the additional trait(s). Alternatively, a trait may be created by mutagenesis, editing or site-directed integration of or into a plant, plant part or plant cell comprising corn event ZM_BCS216090 or a modified corn event ZM_BCS216090. Such traits may include, but are not limited to, increased insect resistance, increased water use efficiency, increased nitrogen use efficiency, increased yield performance, increased drought resistance, increased disease resistance, increased seed quality, improved nutritional quality, hybrid seed production, and/or increase herbicide tolerance, in which the trait is measured with respect to a corn plant lacking such transgenic trait. For example, the ZM_BCS216090 event or a modified corn event ZM_BCS216090 could be stacked by breeding or introgression with another event(s), or a combination of events, known in the art including, but not limited to:
MON00603 (also known as NK603 or MON603; Roundup Ready™ 2 Maize for herbicide tolerance; deposited as ATCC PTA-2478 and described in US Patent Application Publication No. 2007/292854 and U.S. Pat. No. 6,825,400, the entire contents and disclosure of each of which are incorporated herein by reference).
MON89034 (YieldGard™ VT Pro™ for insect resistance; deposited as ATCC PTA-7455 and described in PCT Publication No. WO2007/140256 and US Patent Application Publication No. US2008/260932, the entire contents and disclosure of each of which are incorporated herein by reference).
MON88017 (YieldGard™ VT™ Rootworm™ RR2 for herbicide tolerance and insect resistance; deposited as PTA-5582 and described in US Patent Application Publication No. 2008/028482 and PCT Publication No. WO2005/059103, the entire contents and disclosure of each of which are incorporated herein by reference),
MON87427 (Roundup Ready™ Maize for herbicide tolerance, deposited as ATCC PTA-7899, described in U.S. Pat. No. 8,618,358 and PCT Publication No. WO2011/062904, the entire contents and disclosure of each of which are incorporated herein by reference),
MON87411 (for insect resistance; deposited as ATCC No. PTA-12669 and described in U.S. Pat. No. 10,316,330 and PCT Publication No. WO2013/169923, the entire contents and disclosure of each of which are incorporated herein by reference),
MON87429 (for herbicide tolerance; deposited as ATCC PTA-124635 and described in U.S. Pat. No. 10,920,239 and PCT Publication No. WO2019/152316, the entire contents and disclosure of each of which are incorporated herein by reference),
MON87460 (Genuity® DroughtGard™ for abiotic stress tolerance; deposited as ATCC No. PTA-8910 and described in PCT Publication No. WO2009/111263 and US Patent Application Publication No. 2011/0138504, the entire contents and disclosure of each of which are incorporated herein by reference),
MON87419 (for herbicide tolerance; deposited as ATCC PTA-120860 and described in U.S. Pat. No. 11,098,321 and PCT Publication WO2015/142571, the entire contents and disclosure of each of which are incorporated herein by reference),
MON95275 (for insect resistance, deposited as ATCC PTA-126049 and described in US Patent Application Publication No. US2021/332380 and PCT Publication No. WO2021/216571, the entire contents and disclosure of each of which are incorporated herein by reference).
MON95379 (for insect resistance; deposited as ATCC PTA-125027 and described in PCT Publication WO2020/028172) and US Patent Application Publication No. US2020/032289, the entire contents and disclosure of each of which are incorporated herein by reference.
MON00810 (for insect resistance; also known as MON810; described in US Patent Application Publication No. 2002/102582, the entire contents and disclosure of which are incorporated herein by reference),
MON00021 (also known as GA21; Roundup Ready™ Maize, AgrisureTMGT for herbicide tolerance, deposited as ATCC 209033 and described in US Patent Application Publication No. 2005/086719 and PCT Publication No. WO1998/044140, the entire contents and disclosure of each of which are incorporated herein by reference),
MON832 (Roundup Ready™ Maize for herbicide tolerance),
MON863 (YieldGard™ Rootworm RW, MaxGard™ for insect resistance; deposited as ATCC PTA-2605 and described in PCT Publication No. WO2004/011601 and US Patent Application Publication No. 2006/095986, the entire contents and disclosure of each of which are incorporated herein by reference),
AGV-PY203-4 (GralNzyme Phytase for modified product quality),
ACS-ZM004-3 (Starlink™ Maize for herbicide tolerance and insect resistance),
ACS-ZM001-9 (InVigor™ Maize for pollination control system),
ACS-ZM005-4 (InVigor™ Maize for pollination control system),
ACS-ZM002-1 (Liberty Link™ Maize for herbicide tolerance),
ACS-ZM003-2 (Liberty Link™ Maize for herbicide tolerance),
DAS-40278-9 (Enlist™ Maize for herbicide tolerance, deposited as ATCC No. PTA-10244 and described in U.S. Pat. No. 11,098,322 and PCT Publication No. WO2011/022469),
DAS-01507-1 (also known as TC1507; Herculex™ I, Herculex™ CB for herbicide tolerance and insect resistance; described in US Patent Application Publication No. 2005039226 and PCT Publication No WO2004/099447),
DAS-59122-7 (Herculex™ RW for herbicide tolerance and insect resistance; described in US Patent Application Publication No. 2006/070139),
DKB-89614-9 (Bt Xtra™ Maize for herbicide tolerance and insect resistance),
DP-32138-1 (32138 SPT maintainer for pollination control system; deposited as ATCC No. PTA-9158 and described in US Patent Application Publication No. 2009/0210970 and PCT Publication No. WO2009/103049),
DP-098140-6 (Optimum™ GAT™ for herbicide tolerance; deposited as ATCC No. PTA-8296 and described in US Patent Application Publication No. 2009/137395 and PCT Publication No. WO2008/112019),
MIR162 (Agrisure™ Viptera for insect resistance; deposited as ATCC No. PTA-6188 and described in US Patent Application Publication No. 2009/300784 and PCT Publication No. WO2007/142840),
MIR604 (Agrisure™ RW for insect resistance, described in US Patent Application Publication No. 2008/167456 and PCT Publication No. WO2005/103301),
REN-00038-3 (also known as LY038; Mavera™ Maize for modified product quality; deposited as ATCC No. PTA-5623 and described in PCT Publication No. WO2005/061720 and U.S. Pat. No. 7,615,621, the entire contents and disclosure of each of which are incorporated herein by reference),
SYN-E3272-5 (Enogen™ for modified product quality, described in U.S. Pat. No. 7,635,799 and PCT Application Publication No. WO2006/098952),
SYN-05307-1 (Agrisure® Duracade™ for insect resistance; deposited as ATCC No. PTA-9561 and described in PCT Publication No. WO2010/077816 and U.S. Pat. No. 10,100,371),
Bt10 (for herbicide tolerance and insect resistance),
SYN-BT011-1 (Agrisure™ CB/LL for herbicide tolerance and insect resistance),
SYN-EV176-9 (NaturGard KnockOut™, Maximizer™ for herbicide tolerance and insect resistance),
MON89034×DAS-01507-1×MON603×MIR162×DAS-40278-9 (Power Core™×MIR162×Enlist™ for herbicide tolerance and insect resistance),
DAS-01507-1×DAS-59122-7 (Herculex XTRA™ for herbicide tolerance and insect resistance),
DAS-01507-1×DAS-59122-7×MON603 (Herculex XTRA™ RR for herbicide tolerance and insect resistance),
DAS-01507-1×MON603 (Herculex™ I RR for herbicide tolerance and insect resistance),
DAS-59122-7×MON603 (Herculex™ RW Roundup Ready™ 2 for herbicide tolerance and insect resistance),
DAS-01507-1×DAS-59122-7×MON00810×MIR604×MON603 (Optimum™ Intrasect Xtreme for herbicide tolerance and insect resistance),
DAS-01507-1×DAS-59122-7×MON810×MON603 (Optimum™ Intrasect XTRA for herbicide tolerance and insect resistance),
DAS-01507-1×MIR604×MON603 (Optimum™ TRIsect for herbicide tolerance and insect resistance),
DAS-01507-1×MON810×MON603 (Optimum™ Intrasect for herbicide tolerance and insect resistance).
MON00021×MON810 (Roundup Ready™ YieldGard™ Maize for herbicide tolerance and insect resistance).
MON810×MON88017 (YieldGard™ VT Triple for herbicide tolerance and insect resistance).
MON863×MON810 (YieldGard™ Plus for insect resistance).
MON603×MON810×MON863 (YieldGard™ Plus with RR for herbicide tolerance and insect resistance).
MON863×MON603 (YieldGard™ RW+RR for herbicide tolerance and insect resistance),
MON87427×MON89034×DAS-01507-1×MON87411×DAS-59122-7×DAS-40278-9 (SmartStax™ Pro×Enlist™ for herbicide tolerance and insect resistance),
MON89034×MON88017 (Genuity® VT Triple Pro™ for herbicide tolerance and insect resistance),
MON89034×MON603 (Genuity® VT Double Pro™ for herbicide tolerance and insect resistance),
MON89034×DAS-01507-1×MON88017×DAS-59122-7 (Genuity® SmartStax™ for herbicide tolerance and insect resistance),
MON89034×DAS-01507-1×MON603 (Power Core™ for herbicide tolerance and insect resistance).
MON603×MON810 (YieldGard™ CB+RR for herbicide tolerance and insect resistance).
MON603×ACS-ZM003-2 (Roundup Ready™ Liberty Link™ Maize for herbicide tolerance),
ACS-ZM003-2×MON810 (Liberty Link™ Yieldgard™ Maize for herbicide tolerance and insect resistance).
REN-00038-3×MON810 (Mavera™ YieldGard™ Maize for insect resistance and modified product quality),
SYN-05307-1×MIR604×SYN-BT011-1×DAS-01507-1×MON00021 (Agrisure® Duracade™ 5122 for herbicide tolerance and insect resistance),
SYN-05307-1×MIR604×SYN-BT011-1×DAS-01507-1×MON00021×MIR162 (Agrisure® Duracade™ 5222 for herbicide tolerance and insect resistance),
SYN-BT011-1×DAS-59122-7×MIR604×DAS-01507-1×MON00021 (Agrisure® 3122 for herbicide tolerance and insect resistance),
SYN-BT011-1×MON00021 (Agrisure™ GT/CB/LL for herbicide tolerance and insect resistance),
SYN-BT011-1×MIR162 (Agrisure® Viptera™ 2100 for herbicide tolerance and insect resistance),
SYN-BT011-1×MIR162×MON00021 (Agrisure® Viptera™ 3110 for herbicide tolerance and insect resistance),
SYN-BT011-1×MIR162×MIR604 (Agrisure® Viptera™ 3100 for herbicide tolerance and insect resistance),
SYN-BT011-1×MIR162×DAS-01507-1×MON00021 (Agrisure® Viptera™ 3220 for herbicide tolerance and insect resistance),
SYN-BT011-1×MIR604 (Agrisure™ CB/LL/RW for herbicide tolerance and insect resistance),
SYN-BT011-1×MIR604×MON00021 (Agrisure™ 3000GT for herbicide tolerance and insect resistance), and/or
MIR604×MON00021 (Agrisure™ GT/RW for herbicide tolerance and insect resistance).

### Deposit Information

A deposit of a representative sample of corn seed containing event ZM_BCS216090 was made on Jul. 14, 2021, according to the Budapest Treaty with the American Type Culture Collection (ATCC) having an address at 10801 University Boulevard, Manassas, Virginia 20110, USA, and assigned ATCC Accession No. PTA-127050.

### EXAMPLES

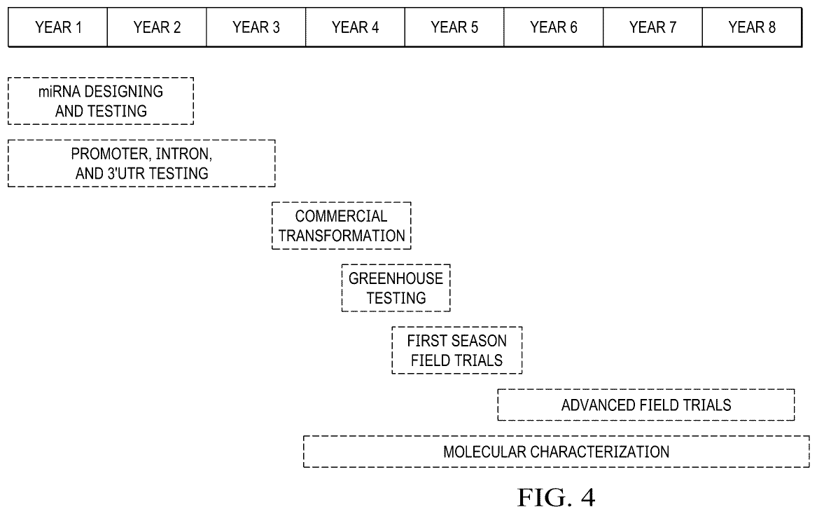

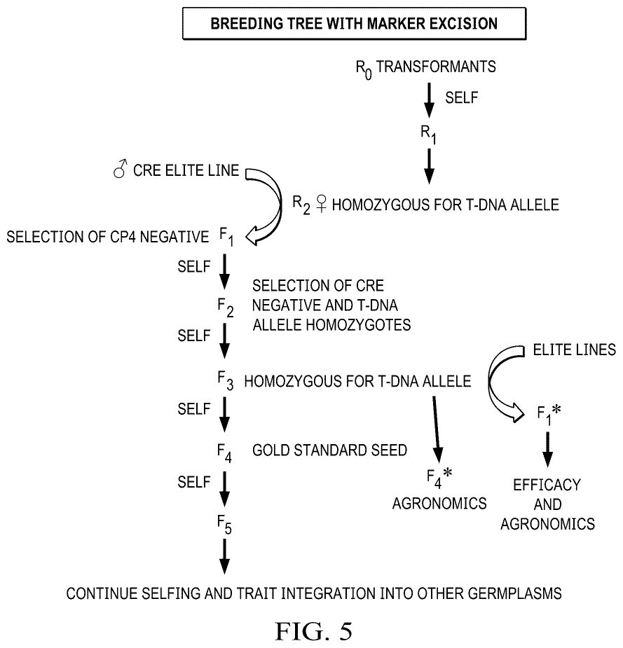

The following examples are included to more fully describe the invention. Summarized are the production and testing of over fifteen thousand (15,000) unique events for miRNA transgenic construct pM578, and the analysis of hundreds of thousands of individual plants over seven years through the rigorous molecular, agronomic, and field testing required for the creation and ultimate selection of corn lead event ZM_BCS216090 (see, e.g., FIG. 4 and FIG. 5).

While the Examples demonstrate certain embodiments of the present invention, it should be appreciated that all specific embodiments and examples provided herein are illustrative and not exhaustive. Those of skill in the art should, in light of the present disclosure, appreciate that changes and modifications can be made to the embodiments, examples and details that are disclosed herein without departing from the spirit and scope of the present invention.

### Example 1

### Expression Cassette Testing, Construct Design, Plant Testing, and Construct Selection.

Transgene expression in plants is influenced by numerous different factors. The right combination of a gene of interest (GOI) and different expression elements driving expression in plants must be found, while not resulting in off-phenotypes that have deleterious traits. Further, beyond the expression elements themselves and their combination and orientation in a cassette, the expression of transgenes in plants can be influenced by chromosomal insertion position, perhaps due to chromatin structure (e.g., heterochromatin) or the proximity of transcriptional regulation elements (e.g., enhancers, etc.) close to the integration site (Kurt Weising et al., Annu. Rev. Genet. 22:421-77, 1988). For example, there can be variation in the levels of expression of an introduced gene from the same construct among different events with different chromosomal insertion positions in the plant genome. Different chromosomal insertion positions may also produce differences in spatial or temporal patterns of expression that may not correspond to the patterns expected from transcriptional regulatory elements present in the introduced gene construct.

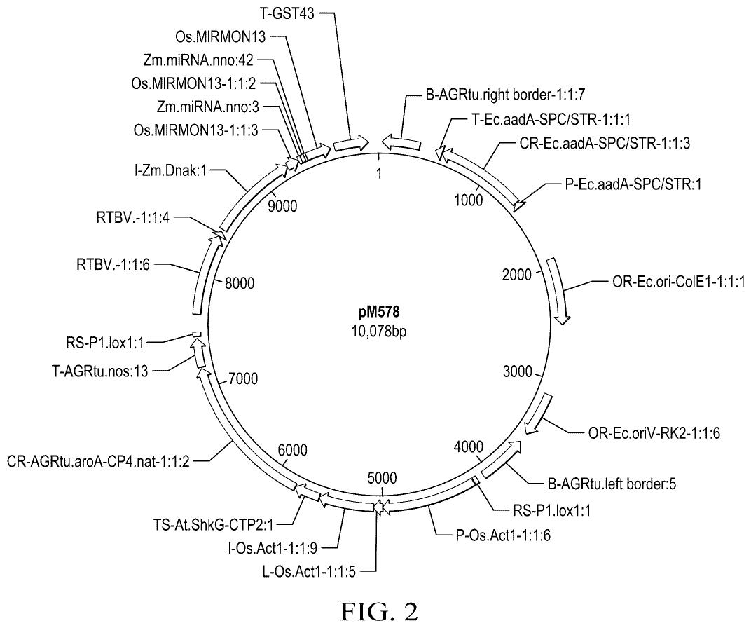

For these reasons, it is often necessary to create and screen multiple constructs and a large number of transformation events for a given construct in order to identify the construct, and then the optimal event (the lead event), which demonstrates optimal traits and suitable expression of the introduced gene of interest along with an absence of molecular and genomic insertion concerns (e.g., selecting clean insertions without transgene truncations or extraneous vector sequence and avoiding insertion positions that are near existing genes, loci or transgenes of interest), while also not producing agronomic or phenotypic off-types. Prior to such studies, it may not be possible to determine whether a particular beneficial event phenotype can be obtained or which event will provide these optimal traits and characteristics. In an initial proof of concept and developmental stage over several years and multiple growing seasons, a number of different constructs with different expression elements were transformed into corn plants. The transformed plants were tested and selected progressively for subsequent testing, based on a desirable range of reduced plant height and ear height, and absence of phenotypic and agronomic off-types. The resulting leading construct, designated pM578 (see FIG. 2), was chosen for extensive further transformation event testing.

Agrobacterium tumefaciens (AB32 strain) cells carrying construct pM578, which contains the CP4 EPSPS gene as a selectable marker and a transgenic miRNA gene (the transgene), were used in plant transformation of an elite corn line designated “EL0” to generate short stature corn plants comprising the transgene. A total of 1,533 transgenic plants were obtained from Agrobacterium-mediated transformation of wild-type embryo explants using the Agrobacterium tumefaciens AB32 strain cells carrying the pM578 vector or plasmid construct.

The transgenic plants were subjected to a series of molecular screenings and agronomic field trials to identify backbone-free events carrying a single copy of the intact T-DNA. A superior event was then selected as a lead candidate based on the molecular data and agronomic phenotypes.

For molecular quality control (MQC) screening, three TaqMan quantitative PCR assays were utilized for genotyping, to detect the presence and copy number of the gene of interest (GOI), selection marker, and backbone. Events with one copy of GOI, one copy of marker but no backbone passed MQC screenings. Events with more than one copy of the GOI or marker or that were backbone positive did not pass the MQC screenings. If events had inconclusive results from any one of the assays, they were classified as uncallable and excluded from further evaluations.

From the 1,533 transgenic plants, 225 events (15%) that were backbone-free and carrying only a single copy of the insertion in plants were identified through Ro MQC screenings. FIG. 5 shows an illustration of the full breeding process used to generate and select the marker-free ZM_BCS216090 corn event. Ro MQC screening results indicated that the precursor event for corn event ZM_BCS216090 (prior to Cre marker excision-see below), later selected as the lead event, had one copy of the CP4 EPSPS marker gene and the transgene, but no backbone of the transformation vector.

Table 2 illustrates that of these one-copy and backbone-free events, 80 events (36%) and 15 events (7%) were discarded for off-type phenotypes or plant health, respectively, while 130 events (58%) passed MQC screening and RO plants with these events were grown to maturity. The numbers of events advanced to each successive generation are pooled across multiple rounds of transformations.

TABLE 2
Breakdown of corn R₀ transgenic plants.

| Description | Number | Percentage |
|---|---|---|
| Total events generated | 1,533 | 100% |
| Did not pass MQC screenings | 1,293 | 84% |
| Uncallable | 15 | 1% |
| Passed MQC screenings | 225 | 15% |
| Discarded-off phenotypes | 80 | 35% |
| Discarded-poor plant health | 15 | 7% |
| Passed MQC R₀ Screening | 130 | 58% |

The transgenic plants were subjected to additional molecular screenings, such as whole genome sequencing (E-Southern), to determine the sequence and genomic context for the R₀ events that were grown to maturity, and 84 of those events were selected and selfed to produce R₁ seed based on these additional genomic considerations. Based on sequence information mapped against the genomes of B73 and in-house elite lines, the genomic location of the in planta T-DNA insertion was identified on chromosome 1 with a deletion of 41 base pairs in the genome at the site of insertion.

R₁ MQC screening was performed to confirm that individual events in the R₀ plants were present in a single copy and segregated in an expected 1:2:1 ratio (homozygotes:hemizygotes:wild-type nulls). For example, as shown in Table 3, R₁ MQC screening results for the precursor event to corn event ZM_BCS216090 (prior to Cre marker excision) showed the 1:2:1 segregation for both the CP4-EPSPS and the miRNA encoding transgene. Homozygous R₁ plants for selected events were selfed to produce R₂ plants, and R₂ MQC screening was performed to confirm that all R₂ offspring derived from the R₁ homozygous plants were also homozygous for the respective event. For example, as shown in Table 4, R₂ MQC screening results for the precursor event to corn event ZM_BCS216090 (prior to Cre marker excision) revealed that all R₂ plants from the were homozygous for both the CP4-EPSPS and the miRNA encoding transgene, confirming again that the T-DNA insertion was a single copy in the lead event. From the 84 selected events at the R₀ stage that were selfed to produce R₁ seed, 66 events were selected and selfed in R₁ plants to produce homozygous R₂ seeds. Selection and attrition in the numbers of events advanced to each subsequent generation was due to multiple factors including, for example, seed availability and limited capacity.

TABLE 3
R₁ MQC screening results for
precursor of corn event ZM_BCS216090.

| | Corn Event ZM_BCS216090 | |
|---|---|---|
| Zygosity | (count and %) | |
| Homozygotes | 5 | 23% |
| Hemizygotes | 12 | 55% |
| Wild-type (nulls) | 5 | 23% |

Note:
The calls for both the CP4 EPSPS and the miRNA transgene gene were the same for each plant.

TABLE 4
R₂ MQC screening results for precursor of corn event ZM_BCS216090.

| | Corn Event ZM_BCS216090 | |
|---|---|---|
| Zygosity | (count and %) | |
| Homozygotes | 73 | 100% |
| Hemizygotes | 0 | 0% |
| Wild-type (nulls) | 0 | 0% |

Note:
Three batches of genotyping data were combined. The calls for both the CP4 EPSPS and the miRNA transgene were the same for each plant.

### Example 2

### Cre-Excision of the Glyphosate Selection Cassette in Corn Event ZM_BCS216090.

This example describes the removal of the glyphosate selection cassette from corn event ZM_BCS216090 through in planta Cre-excision. The glyphosate selection cassette was used to select transformed events. By removal of the selection cassette, a “marker-free” event was created wherein only the transgenic miRNA expression cassette remained in the final event.

FIG. 5 illustrates the breeding process used to generate the marker-free ZM_BCS216090 corn event. Construct pM578 comprises the miRNA expression cassette and a cassette used for the selection of transformed plant cells using glyphosate selection. The selection cassette was flanked on both sides with LoxP Cre-recombinase recognition sites. As described above, elite corn line embryo explants were transformed using an Agrobacterium-mediated transformation process with construct pM578. After transformation, the R₀ transformants were self-pollinated for two (2) generations, during which time many events were removed based on various assays and normal attrition as mentioned above, such as seed return, plant health, and molecular characterization. At this stage, plants in the R₂ generation comprising a transgenic event were bred with plants expressing Cre-recombinase as shown in FIG. 5. Specifically, de-tasseled (female) R₂ generation plants homozygous for the miRNA expression cassette were cross-pollinated with transgenic corn (male) plants homozygous for a transgene cassette encoding a Cre-recombinase enzyme. The Cre-recombinase expressing male donor pollen germinates after landing on the silk tissue of the female plant comprising the miRNA expression cassette, setting free the two sperms of the Cre-recombinase expressing male donor. The nucleus of one sperm fuses with an egg nucleus of the female plant forming a zygote, and the other sperm nucleus fuses with one of the two polar nuclei which in turn fuses with the other polar nucleus, thereby establishing the primary endosperm nucleus. Thus, by using the Cre-recombinase expressing plant as the male pollen donor, both the embryo and endosperm of the resulting cross will express Cre-recombinase as the cells divide and develop and become a corn kernel (i.e., seed). The Cre-recombinase binds to inverted repeats in the LoxP site and catalyzes a crossover in an eight-base pair spacer region of the two LoxP sites that flank the expression cassette, resulting in the excision of the marker cassette with one LoxP site remaining in the integrated T-DNA due to recombination.

As shown in FIG. 5, R₂ plants for 43 events were crossed to plants expressing the Cre recombinase (Cre Elite Line). F₁ progeny resulting from this cross were selected for the absence of the CP4 EPSPS selection cassette, and F₁ progeny for 38 marker-free events were allowed to self-pollinate to produce F₂ progeny plants. This self-pollination of F₁ progeny is identified as the F₁ Self in the timeline presented in FIG. 5. Through this process, the two remaining expression cassettes—the Cre-recombinase expression cassette and the miRNA encoding transgene—segregated in the resulting F₂ population, resulting in progeny homozygous or heterozygous for one or both expression cassettes or transgenes.

F₂ progeny were selected that demonstrated the absence of the Cre-recombinase expression cassette and homozygosity for the transgenic miRNA expression cassette without the marker cassette. These selected F₂ progeny for 23 events were self-pollinated (identified as the F₂ Self in the timeline presented in FIG. 5), giving rise to an F₃ generation homozygous for the miRNA expression cassette. In addition to the absence of the Cre-recombinase cassette and homozygosity of the transgene, the events for the F₂ Self were also selected based on additional genomic, trait integration and breeding considerations.

A further self-pollination of F₃ progeny for 22 events (identified as the F₃ Self in the timeline presented in FIG. 5) was performed to produce F₄ progeny and seed which were assayed for purity and were designated as “Gold Standard Seed” (or “GSS”). Gold Standard Seed is seed that has been assayed for purity to assure the absence of transgenic events other than corn event ZM_BCS216090. F₄ was the first generation of Gold Standard Seed. After creation of the of the GSS, F₄ progeny plants for only 10 events were selected and further self-pollinated to produce additional generations of progeny plants and seed homozygous for the miRNA expression cassette or transgene, or alternatively progeny plants homozygous for the miRNA expression cassette or transgene from the F₄ or subsequent generations may be crossed to other plants having a different genotype or germplasm, such as to allow for trait integration of an event described herein, such as corn event ZM_BCS216090, in other germplasms or genetic backgrounds and/or with other traits.

Excision of the glyphosate selection marker cassette did not affect the transgene expression. Removing the glyphosate selection cassette from corn event ZM_BCS216090 through Cre-excision provided a transgenic corn event which produced short stature corn plants without the CP4 EPSPS cassette in the final event. This “marker-free” event assures flexibility when building corn breeding stacks with other corn transgenic events to provide a multiplicity of products incorporating corn event ZM_BCS216090 and allowing multiple options for providing additional traits in final commercial breeding stacks.

### Example 3

### Corn Event ZM_BCS216090 Event-Specific Endpoint TaqMan® Assays.

The following example describes methods useful in identifying the presence of corn event ZM_BCS216090 in a corn sample.

Detection of corn event ZM_BCS216090 in a sample can be done using DNA-or RNA-based detection techniques. Exemplary detection methods and materials are provided herein. Detection may determine the presence or absence of the event in a sample. Detection may also indicate the number of genomic copies of corn event ZM_BCS216090 (that is, hemizygous, homozygous, or heterozygous) in a sample of genomic DNA.

An event-specific endpoint Applied Biosystems™ TaqMan thermal amplification method (Thermo Fisher Scientific) was developed to identify corn event ZM_BCS216090 in a sample. The DNA primers and probe used in the endpoint assay for this example are shown in Table 5, although it is appreciated that other primers and probes may also be used.

TABLE 5
ZM_BCS216090 event-specific and internal control primers and probes.

| SEQ ID NO. | Name | Type |
|---|---|---|
| 11 | Primer SQ51606 | Event-specific |
| 12 | Primer SQ51629 | Event-specific |
| 13 | 6FAM ™ probe PB50583 | Event-specific |
| 14 | Primer SQ20222 | Internal control |
| 15 | Primer SQ20221 | Internal control |
| 16 | VIC ® Probe PB50298 | Internal control |

6-FAM is a fluorescent dye product of Applied Biosystems (Foster City, CA) and is attached to the DNA probe. For TaqMan MGB probes, the 5′ exonuclease activity of Taq DNA polymerase cleaves the probe from the 5′-end, between the fluorophore and quencher. When hybridized to the target DNA strand, quencher and fluorophore are separated enough to produce a fluorescent signal, thus releasing fluorescence. The pair of primers when used with these reaction methods and the probe produce a DNA amplicon that is diagnostic for corn event ZM_BCS216090. The controls for this analysis should include a positive control containing corn event ZM_BCS216090, a negative control from non-transgenic plant, and a negative control that contains no template DNA. Additionally, a control for the PCR reaction should optimally include internal control primers and an internal control probe, specific to a single copy gene in the corn genome. These assays are optimized for use with the Applied Biosystems GeneAmp® PCR System 9700 (Thermo Fisher Scientific) run at maximum speed, but other equipment may be used.

Event-specific PCR assays for line identification (Line ID) were developed and validated. Based on integrated T-DNA and flanking genomic sequences, TaqMan PCR assays were designed. Simplified validation (sValid) tests were conducted to identify good assays by running endpoint PCR on replicates of 8 positive, 6 negative, and 2 water samples. Scores were calculated based on the difference of FAM readings, using the formula defined as score=(average of positive samples−average of negative samples). Assay performance was ranked based on the score in a five-star system, an assay with three or more stars being considered acceptable. A purity test was performed to ensure that leaf tissues containing target sequences show the positive for corresponding assays. A proficiency test (PT) was conducted to ensure that the procedure can be replicated to obtain the expected results with the assay.

Event-specific endpoint TaqMan PCR assays (Line IDs) are used for GSS nurseries and other quality screening programs to verify the presence of traits of interest. Based on plant flanking sequences and the in planta T-DNA sequences identified by R₀ rapid unlinked sequence hybridization (RUSH), two line IDs, which incorporate a transgene oligonucleotide, a corresponding second oligonucleotide appropriately positioned in either the 5′ or 3′ flanking sequence and a fluorescently labeled probe, for the lead event were designed and validated.

The line ID assay that has been selected and used in subsequent molecular characterizations for corn event ZM_BCS216090 is an event-specific qualitative endpoint TaqMan PCR assay. Setup and sequence information for the corn event ZM_BCS216090 line ID assay are listed in Table 6, and cycling conditions are presented in Table 7.

TABLE 6
ZM_BCS216090 event-specific endpoint TaqMan PCR reaction components.

| | | Stock | | Final | |
|---|---|---|---|---|---|
| | | Concentration | Volume | Concentration | |
| Step | Reagent | (μ) | (μl) | (μM) | Comments |
| | Reaction Volume | | 5 | | |
| 1 | 18 megohm water | | 0.00 | | Adjust for final |
| | | | | | volume |
| 2 | 2 × Master Mix | | 2.28 | | 1 × final |
| | | | | | concentration of |
| | | | | | buffer |
| 3 | Event Specific Primer | 100 | 0.05 | 0.9 | |
| | SQ51606 | | | | |
| 4 | Event Specific Primer | 100 | 0.05 | 0.9 | |
| | SQ51629 | | | | |
| 5 | Event Specific 6FAM ™ probe | 100 | 0.01 | 0.2 | Probe is light |
| | PB50583 | | | | sensitive |
| 6 | Internal Control Primer | 100 | 0.05 | 0.9 | |
| | SQ20222 | | | | |
| 7 | Internal Control Primer | 100 | 0.05 | 0.9 | |
| | SQ20221 | | | | |
| 8 | Internal Control VIC ® Probe | 100 | 0.01 | 0.2 | Probe is light |
| | PB50298 | | | | sensitive |
| 9 | Extracted DNA (template): | | 2.5 | | Separate reactions are |
| | Leaf Samples to be analyzed | | | | made for each |
| | Negative control (non- | | | | template. |
| | transgenic DNA) | | | | |
| | Negative water control (No | | | | |
| | template control) | | | | |
| | Positive Qualitative | | | | |
| | control(s) ZM_BCS216090 | | | | |
| | DNA | | | | |

TABLE 7
Endpoint TaqMan thermocycler conditions.

| Step No. | Cycle No. | Settings |
|---|---|---|
| 1 | 1 | 95° C., 20 seconds |
| 2 | 35 | 95° C., 3 seconds |
| | | 60° C., 20 seconds |
| 3 | 1 | 10° C. |

A zygosity assay is described here to determine whether a plant comprising the lead event is heterozygous or homozygous for the event or the wild-type sequence. An amplification reaction assay can be designed using the sequence information provided herein. For example, such a PCR assay would include design of at least three primers: primer-1, primer-2, and primer-3, where primer-1 is specific to the genomic DNA on the 3′ flank of the lead event; primer-2 is specific to the lead event transgenic insert; and primer-3 is specific to the wild-type sequence. When used as a primer pair in an amplification reaction, primer-1 with primer-2 will produce a PCR amplicon specific for the lead event. When used as a primer pair in an amplification reaction, primer-1 with primer-3 will produce a PCR amplicon specific for wild-type sequence. In a PCR reaction performed on corn genomic DNA, the respective PCR amplicons generated from primer-1+primer-2 and that generated from primer-1+primer-3 will differ in sequence and size of the amplicon. When the three primers are included in a PCR reaction with DNA extracted from a plant homozygous for the lead event, only the primer-1+primer-2 amplicon (specific for the lead event insertion) will be generated. When the three primers are included in a PCR reaction with DNA extracted from a plant heterozygous for the lead event, both the primer-1+primer-2 amplicon (specific for the lead event insertion) and the primer-1+primer-3 amplicon (specific for wild-type sequence or absence of the lead event insertion) will be generated. When the three primers are mixed together in a PCR reaction with DNA extracted from a plant that is null for the lead event (wild-type), only the primer-1+primer-3 amplicon (specific for wild-type sequence) will be generated. The amplicons produced using the PCR reaction may be identified or distinguished using any method known in the art.

Another zygosity assay for the lead event is a TaqMan thermal amplification reaction. For this type of assay, in addition to primers as described above, the assay would include two fluorescently labeled probes. Probe-1 would be specific for the lead event, and probe-2 would be specific for a corn plant that is null for the lead event (wild-type), and where the two probes contain different fluorescent labels, for example the 6-FAM-label or VIC-label. When used in a TaqMan reaction, primer-1+primer-2+probe-1 will produce a first fluorescent signal specific for the lead event and primer-1+primer-3+probe-2 will produce a second fluorescent signal specific for a wild-type plant. When the three primers and two probes are included in a TaqMan reaction with DNA extracted from a plant homozygous for the lead event, only the first fluorescent signal (specific to primer-1+primer-2+probe-1) will be generated. When the three primers and two probes are included in a TaqMan reaction with DNA extracted from a plant heterozygous for the lead event, both the first fluorescent signal (specific to primer-1+primer-2+probe-1) and the second fluorescent signal (specific to primer-1+primer-3+probe-2) will be generated. When the three primers are mixed together in a TaqMan reaction with DNA extracted from a plant which is null for the lead event (wild-type), only the second fluorescent signal (specific to primer-1+primer-3+probe-2) will be generated.

Another method to detect the presence of the lead event in a plant sample would be Southern analysis as generally understood in the art. One of skill in art, based on the present disclosure and description of the lead event, would understand how to design Southern hybridization probe(s) specific for the lead event and a second southern hybridization probe specific for a plant which is null for the lead event (wild-type). With Southern analysis, a signal detected only from the first Southern hybridization probe will be indicative of a plant homozygous for the lead event; a signal detected from both the first Southern hybridization probe and the second Southern hybridization probe will be indicative of a plant heterozygous for the lead event; and a signal detected only from the second Southern hybridization probe will be indicative that the DNA was extracted from a plant that is null for the lead event (wild-type).

### Example 4

### Yield and Agronomic Traits of Corn Event ZM_BCS216090 Based on Field Trials in Multiple Growing Seasons.

This example demonstrates that transgenic corn event ZM_BCS216090 minimizes crop yield losses for farmers though improved standability and increased lodging resistance due to its short stature or semi-dwarf phenotype. In addition, the reduced stature of corn plants comprising event ZM_BCS216090 enables season-long field access which leads to improved precision of crop input and sustainability. Inbreds for these field studies (designated as the F₄* generation in FIG. 5) were made by selfing F₃ plants having the same 10 events that were advanced as GSS in F₄ and subsequent generations, and hybrids for these field studies were made by crossing F₃ plants having the same 10 events with four other elite lines (EL1-4) that lack the transgene to produce F₁* progeny plants (the “*” is intended to distinguish the F₁ and F₄ generations for marker excision and generating gold standard seed; F₃ plants comprising each of the events and isogenic control plants lacking any transgenic event were each designated as elite line 0 or EL0 in the hybrid crosses).

In consecutive years, transgenic plants comprising each of the 10 events selected for GSS, including corn event ZM_BCS216090 and 9 other events (designated Events 2-9), were tested in the field across 34 locations in agronomic yield trials across U.S. Midwestern states. Compared to untransformed control plants, hybrid plants comprising corn event ZM_BCS216090 demonstrated consistent yields, reduced plant height and increased lodging resistance, and inbred plants comprising corn event ZM_BCS216090 showed reduced plant height and consistent flowering.

Measurements of yield were calculated by adjusting for moisture and expressed as bushels per acre (bu/acre). Plant height is measured as the distance in inches (in) from the soil line to the collar of the flag leaf. Ear height is measured as the distance in inches between the soil line and the primary ear node. Measurements of lodging is expressed as the percentage of plants with a plot leaning 30 degrees or more from perpendicular. Fifty percent (50%) pollen shed and fifty percent (50%) silking were expressed as days after planting (DAP).

Table 8 shows the yield and agronomic characteristics measured for corn event ZM_BCS216090 hybrids. Table 9 shows the agronomic characteristics measured for corn event ZM_BCS216090 inbreds produced by selfing the transformed EL0 line. Inbred lines containing event ZM_BCS216090 or each of Events 2-9 were compared to the non-transformed control of the self-crossed EL0 line, and hybrid lines containing event ZM_BCS216090 or each of Events 2-9 and crossed to each of EL1-EL4 were compared to the non-transformed control hybrids of the same cross. The mean measurements and standard error (SE) are reported in the tables.

TABLE 8
Yield and agronomics for event ZM_BCS216090 hybrids relative
to non-transgenic controls in year one of field testing.

| | Plant Height | | Ear Height | | Yield | | Root | |
|---|---|---|---|---|---|---|---|---|
| | (in) | | (in) | | (bu/acre) | | Lodging % | |
| | Mean | SE | Mean | SE | Mean | SE | Mean | SE |
| ZM_BCS216090 | 63.51 | 1.25 | 24.85 | 0.77 | 240.46 | 6.13 | 4.20 | 8.17 |
| EL0 × EL1 | | | | | | | | |
| ZM_BCS216090 | 62.84 | 1.25 | 26.74 | 0.77 | 234.10 | 6.16 | 10.47 | 8.20 |
| EL0 × EL2 | | | | | | | | |
| ZM_BCS216090 | 61.64 | 1.25 | 25.08 | 0.77 | 240.64 | 6.15 | 12.18 | 8.17 |
| EL0 × EL3 | | | | | | | | |
| ZM_BCS216090 | 62.29 | 1.25 | 24.81 | 0.77 | 244.86 | 6.12 | 12.64 | 8.17 |
| EL0 × EL4 | | | | | | | | |
| Event 2 EL0 × EL1 | 62.81 | 1.25 | 24.67 | 0.77 | 234.65 | 6.14 | 2.88 | 8.20 |
| Event 2 EL0 × EL2 | 61.78 | 1.25 | 26.22 | 0.77 | 228.82 | 6.14 | 9.82 | 8.17 |
| Event 2 EL0 × EL3 | 60.76 | 1.25 | 25.41 | 0.77 | 236.36 | 6.13 | 10.00 | 8.17 |
| Event 2 EL0 × EL4 | 61.47 | 1.25 | 24.80 | 0.77 | 235.27 | 6.14 | 12.30 | 8.20 |
| Event 3 EL0 × EL1 | 62.52 | 1.25 | 24.22 | 0.77 | 234.98 | 6.14 | 0.98 | 8.20 |
| Event 3 EL0 × EL2 | 61.32 | 1.25 | 26.40 | 0.77 | 231.41 | 6.14 | 7.04 | 8.17 |
| Event 3 EL0 × EL3 | 59.93 | 1.25 | 24.43 | 0.77 | 236.94 | 6.15 | 9.62 | 8.25 |
| Event 3 EL0 × EL4 | 60.91 | 1.25 | 24.73 | 0.77 | 239.70 | 6.13 | 13.29 | 8.20 |
| Event 4 EL0 × EL1 | 61.90 | 1.25 | 23.87 | 0.77 | 232.53 | 6.12 | 1.54 | 8.20 |
| Event 4 EL0 × EL2 | 60.58 | 1.25 | 25.66 | 0.77 | 228.24 | 6.14 | 5.50 | 8.17 |
| Event 4 EL0 × EL3 | 58.56 | 1.25 | 24.17 | 0.77 | 232.67 | 6.14 | 7.96 | 8.17 |
| Event 4 EL0 × EL4 | 60.63 | 1.25 | 24.18 | 0.77 | 235.62 | 6.14 | 10.80 | 8.20 |
| Event 5 EL0 × EL1 | 62.85 | 1.25 | 24.71 | 0.77 | 236.29 | 6.11 | 2.84 | 8.17 |
| Event 5 EL0 × EL2 | 60.90 | 1.25 | 25.93 | 0.77 | 227.79 | 6.13 | 7.07 | 8.17 |
| Event 5 EL0 × EL3 | 59.75 | 1.25 | 24.65 | 0.77 | 238.91 | 6.14 | 10.81 | 8.17 |
| Event 5 EL0 × EL4 | 61.11 | 1.25 | 24.72 | 0.77 | 237.32 | 6.12 | 12.67 | 8.20 |
| Event 6 EL0 × EL1 | 62.60 | 1.25 | 24.74 | 0.77 | 235.61 | 6.14 | 3.14 | 8.17 |
| Event 6 EL0 × EL2 | 61.80 | 1.25 | 26.20 | 0.77 | 225.95 | 6.13 | 8.07 | 8.17 |
| Event 6 EL0 × EL3 | 59.80 | 1.25 | 24.73 | 0.77 | 236.20 | 6.13 | 7.44 | 8.17 |
| Event 6 EL0 × EL4 | 60.83 | 1.25 | 24.83 | 0.77 | 238.59 | 6.14 | 10.01 | 8.20 |
| Event 7 EL0 × EL1 | 63.00 | 1.25 | 24.17 | 0.77 | 234.18 | 6.14 | 2.31 | 8.17 |
| Event 7 EL0 × EL2 | 61.51 | 1.25 | 25.99 | 0.77 | 225.86 | 6.14 | 9.60 | 8.17 |
| Event 7 EL0 × EL3 | 60.38 | 1.25 | 24.70 | 0.77 | 238.15 | 6.13 | 12.23 | 8.17 |
| Event 7 EL0 × EL4 | 61.41 | 1.25 | 25.27 | 0.77 | 237.34 | 6.13 | 12.99 | 8.20 |
| Event 8 EL0 × EL1 | 62.43 | 1.25 | 24.59 | 0.77 | 239.77 | 6.14 | 2.98 | 8.20 |
| Event 8 EL0 × EL2 | 61.97 | 1.25 | 26.29 | 0.77 | 231.32 | 6.13 | 6.33 | 8.17 |
| Event 8 EL0 × EL3 | 59.97 | 1.25 | 24.52 | 0.77 | 237.51 | 6.14 | 9.86 | 8.20 |
| Event 8 EL0 × EL4 | 61.42 | 1.24 | 24.71 | 0.77 | 240.74 | 6.13 | 13.82 | 8.17 |
| Event 9 EL0 × EL1 | 62.05 | 1.25 | 23.69 | 0.77 | 231.46 | 6.14 | 4.03 | 8.17 |
| Event 9 EL0 × EL2 | 61.43 | 1.25 | 26.29 | 0.77 | 229.36 | 6.15 | 8.47 | 8.20 |
| Event 9 EL0 × EL3 | 60.50 | 1.25 | 24.71 | 0.77 | 237.99 | 6.12 | 7.92 | 8.20 |
| Event 9 EL0 × EL4 | 60.81 | 1.25 | 24.72 | 0.77 | 237.72 | 6.15 | 10.16 | 8.20 |
| Control EL0 × EL1 | 95.48 | 1.24 | 44.26 | 0.74 | 230.62 | 5.96 | 9.50 | 8.01 |
| Control EL0 × EL2 | 96.78 | 1.24 | 46.78 | 0.74 | 227.92 | 5.97 | 11.76 | 8.00 |
| Control EL0 × EL3 | 92.84 | 1.24 | 43.69 | 0.74 | 231.94 | 5.97 | 18.15 | 8.00 |
| Control EL0 × EL4 | 98.59 | 1.24 | 46.66 | 0.74 | 234.87 | 5.96 | 26.48 | 8.01 |

TABLE 9
Agronomics for event ZM_BCS216090 inbreds
relative to non-transgenic controls in year one.

| | Plant Height | | Ear Height | | 50% Pollen Shed | | 50% Silking | |
|---|---|---|---|---|---|---|---|---|
| | (in) | | (in) | | (DAP) | | (DAP) | |
| | Mean | SE | Mean | SE | Mean | SE | Mean | SE |
| ZM_BCS216090 | 61.17 | 3.07 | 19.92 | 0.93 | 64.10 | 3.70 | 64.60 | 3.45 |
| Event 2 | 59.33 | 3.07 | 20.08 | 0.93 | 64.50 | 3.70 | 64.70 | 3.45 |
| Event 3 | 57.76 | 3.09 | 18.33 | 0.93 | 63.20 | 3.70 | 63.60 | 3.45 |
| Event 4 | 57.50 | 3.07 | 18.50 | 0.93 | 63.60 | 3.70 | 64.00 | 3.45 |
| Event 5 | 58.91 | 3.10 | 19.83 | 1.00 | 63.70 | 3.70 | 64.40 | 3.45 |
| Event 6 | 59.42 | 3.07 | 19.17 | 0.93 | 63.80 | 3.70 | 64.70 | 3.45 |
| Event 7 | 59.72 | 3.09 | 18.56 | 0.97 | 63.80 | 3.70 | 64.70 | 3.45 |
| Event 8 | 58.42 | 3.07 | 18.75 | 0.93 | 64.00 | 3.70 | 64.30 | 3.45 |
| Event 9 | 57.33 | 3.07 | 17.83 | 0.93 | 63.20 | 3.70 | 63.50 | 3.45 |
| Control | 83.32 | 2.97 | 32.88 | 0.70 | 63.77 | 3.69 | 64.77 | 3.44 |

As can be seen in Table 8, the measures of yield were relatively the same or better for corn event ZM_BCS216090 hybrids relative to the respective controls, and plant height, ear height, and lodging were reduced relative to the respective hybrid controls. Increases in yield in hybrids comprising corn event ZM_BCS216090 in comparison to the respective controls may be due to the reduced root lodging. Additionally, Table 9 demonstrates that timing of 50% pollen shed and 50% silking is relatively similar for inbred plants comprising corn event ZM_BCS216090 when compared to the inbred control plants, and plant height and ear height are reduced relative to the inbred control.

During the subsequent year of testing, field trials were conducted using hybrids comprising corn event ZM_BCS216090 and the other 9 events (Events 2-9) in comparison to non-transformed hybrid controls. Table 10 shows that the yield of corn event ZM_BCS216090 hybrids was generally similar to or better than the yields observed in control plants, while plant height, ear height, and root lodging were decreased in plants comprising event ZM_BCS216090 when compared to the respective controls. A decrease in root lodging may at least partly explain any observed yield improvement in plants comprising event ZM_BCS216090 compared to the control plants.

TABLE 10
Yield and agronomics for event ZM_BCS216090 hybrids relative
to non-transgenic controls (year two growing season).

| | Plant Height | | Ear Height | | Yield | | Root | |
|---|---|---|---|---|---|---|---|---|
| | (in) | | (in) | | (bu/acre) | | Lodging % | |
| | Mean | SE | Mean | SE | Mean | SE | Mean | SE |
| ZM_BCS216090 × | 66.69 | 1.40 | 26.27 | 0.75 | 219.84 | 6.10 | 13.11 | 13.38 |
| EL1 | | | | | | | | |
| ZM_BCS216090 × | 67.06 | 1.40 | 27.80 | 0.75 | 218.60 | 6.12 | 17.25 | 13.41 |
| EL2 | | | | | | | | |
| ZM_BCS216090 × | 64.72 | 1.40 | 25.42 | 0.75 | 218.51 | 6.13 | 21.00 | 13.38 |
| EL3 | | | | | | | | |
| ZM_BCS216090 × | 68.21 | 1.40 | 25.94 | 0.75 | 227.96 | 6.14 | 10.16 | 13.38 |
| EL4 | | | | | | | | |
| Control EL0 × EL1 | 99.34 | 1.40 | 45.51 | 0.75 | 215.76 | 6.15 | 31.68 | 12.85 |
| Control EL0 × EL2 | 100.92 | 1.40 | 48.66 | 0.75 | 215.35 | 6.18 | 42.32 | 12.87 |
| Control EL0 × EL3 | 95.98 | 1.40 | 44.95 | 0.75 | 212.89 | 6.19 | 45.53 | 12.85 |
| Control EL0 × EL4 | 103.11 | 1.40 | 48.18 | 0.75 | 215.97 | 6.15 | 40.46 | 12.85 |

To summarize the observations from two consecutive years of field trials, plants comprising corn event ZM_BCS216090 demonstrate similar or better hybrid yield, similar inbred flowering, and reduced plant height and lodging when compared to non-transformed controls.

### Example 5

Modification of Corn Event ZM_BCS216090 with Genome Editing Techniques Using a Single Guide RNA

This example describes how all or part of the transgenic insertion present in corn event ZM_BCS216090, and/or a flanking genomic DNA segment(s), may be altered, excised or modified, such as by making one or more insertions, deletions, substitutions, inversions, transpositions, mutations or other genetic modifications, at or near a target, hybridization or recognition site(s) or sequence(s), which may be referred to jointly as a target site(s), using a targeted genome editing technique. According to present embodiments, a targeted genome editing technique may comprise use of a site-specific nuclease, such as a meganuclease, a zinc-finger nuclease (ZFN), an RNA-guided endonuclease (e.g., the CRISPR/Cas9 system), a TALE-endonuclease (TALEN), a recombinase, or a transposase. For example, genetic modifications can be made by targeted genome editing techniques and methods using a Clustered Regularly Interspersed Short Palindromic Repeats (CRISPR) editing system with one or more guide RNAs. Target sites useful for alteration, excision or modification of all or part of the event ZM_BCS216090 transgenic insertion, the expression cassette within SEQ ID NO:9 or SEQ ID NO: 10, and/or a flanking sequence(s) at or near event ZM_BCS216090 can be used with a guide RNA(s) specific for such target site(s) to create a genetic modification (e.g., an excision, mutation or edit) at, near or between such target site(s).

According to some embodiments, a polynucleotide segment or sequence spanning all or part of the event ZM_BCS216090 transgenic insertion, the event ZM_BCS216090 junction sequence(s), the miRNA expression cassette within SEQ ID NO:9 or SEQ ID NO:10, and/or a flanking sequence(s) at or near event ZM_BCS216090 can be excised or deleted using a guide RNA with two target sites having the same target sequence that are present on both sides (i.e., on the 5′ and 3′ sides) of the polynucleotide segment or sequence to be excised or deleted. Such target sites may each be present in the flanking genomic DNA or introduced by the transgenic insertion of event ZM_BCS216090, either at the 5′ or 3′ junction or within the transgenic insertion itself. In one embodiment, a CRISPR nuclease system cleaves at two identical guide RNA target sites thereby permitting the excision or deletion of the intervening sequence. According to some embodiments, one of the target sites, a first target site, may be present in a 5′ or 3′ flanking sequence, or a 5′ flank or a 3′ flank, and the other target site, a second target site, may be present within the transgenic insertion or at the 5′ or 3′ junction. Alternatively, one of the target sites, a first target site, may be present in a 5′ flanking sequence or a 5′ flank, and the other target site, a second target site, may be present in a 3′ flanking sequence or a 3′ flank. As another alternative, one of the target sites, a first target site, may be present within the transgenic insertion or at the 5′ junction, and the other target site, a second target site, may be present within the transgenic insertion or at the 3′ junction.

While a target site that happens to be present at two desired or approximate locations within the transgenic insertion, junction sequence(s), and/or flanking sequence(s) may be used to make a desired excision or deletion of the intervening polynucleotide sequence by a targeted genome editing technique, two target sites may not be present at the desired locations to make a desired excision or deletion. In these cases, either (i) two different guide RNAs and target sites could be used, or (ii) a second guide RNA target site could be introduced or engineered at a desired location within or near event ZM_BCS216090 that has the same target sequence fort a given guide RNA as another (or first) target site already present in the transgenic insert, junction or flanking sequence, or (iii) two guide RNA target sites, a first and a second target sequence, could be introduced or engineered at the desired locations within or near event ZM_BCS216090 that have the same or different target sequences for the same or different gRNA(s). In these cases, an introduced or engineered target site may be introduced at or near an existing target site for a site-specific nuclease within or near event ZM_BCS216090 by a targeted genome editing technique with the site-specific nuclease, which may further involve a donor DNA template based repair, to direct the insertion of the engineered target site. An engineered target site introduced or engineered within or near event ZM_BCS216090 may be referred to as a “cognate target site” having the same target sequence as another target site within or near event ZM_BCS216090, which may be referred to as an “originator target site.”

Sequences corresponding to the 5′ and 3′ flanking genomic sequences of event ZM_BCS216090 (presented as SEQ ID NOs: 17, 18, 19 and 20), the 5′ and 3′ junction regions (presented as SEQ ID NOs: 1-6) and the transgenic insertion (presented as SEQ ID NO: 9) were scanned for potential originator guide RNA recognition or target sites (OgRRS). As used herein, the term “originator guide RNA recognition site” or “OgRRS” refers to an endogenous DNA polynucleotide sequence of a target site in a flanking genomic sequence, a junction sequence or the transgenic insertion, which in the case of a CRISPR/Cas enzyme requires a protospacer adjacent motif (PAM) site or sequence adjacent and operably linked with required positioning relative to the guide RNA hybridization or target site. Such target site may also be referred to as a protospacer sequence. In some embodiments, an OgRRS can be located in the flanking 5′ or 3′ genomic sequence (i.e., in non-transgenic DNA of a junction polynucleotide). In some embodiments, an OgRRS can be located in the 5′ or 3′ junction region (i.e., in both transgenic DNA and non-transgenic DNA of a junction polynucleotide or spanning transgenic and non-transgenic DNA of a junction polynucleotide). In some embodiments, an OgRRS can be located in the transgenic insert. The OgRRS can be determined based upon the specific CRISPR editing system chosen. For example, Cas9 recognizes a G-rich protospacer-adjacent motif (PAM) that is 3′ to its guide RNA hybridization or target site, whereas Cas12a/Cpf1 systems recognize a T-rich protospacer-adjacent motif (PAM) that is 5′ to its guide RNA hybridization or target site.

To engineer a second target site identical to an OgRRS sequence, the OgRRS sequence can then be used to make a cognate guide RNA recognition or target site (CgRRS) which is inserted or created at a desired location within or near event ZM_BCS216090 using a site-specific nuclease or CRISPR editing system. As used herein, the term “cognate guide RNA recognition site” or “CgRRS” refers to a DNA polynucleotide comprising a cognate target site that is inserted or engineered at a desired location within or near event ZM_BCS216090, and which in the case of a cognate target site for a CRISPR/Cas enzyme is adjacent to, and operably linked with, a protospacer adjacent motif (PAM) site or sequence with required positioning relative to the inserted or engineered cognate target site. Such a CgRRS is absent from the desired location within or near event ZM_BCS216090 prior to introducing or engineering the CgRRS as described herein. After the CgRRS is introduce or engineered at the desired location within or near event ZM_BCS216090, the CgRRS and its corresponding OgRRS can hybridize to a single gRNA and direct the excision or deletion of the intervening polynucleotide sequence.

According to present embodiments, a CgRRS can be located in the flanking 5′ or 3′ genomic sequence or 5′ or 3′ flank, in the 5′ or 3′ junction region (i.e., in both transgenic DNA and non-transgenic DNA of a junction polynucleotide or spanning transgenic and non-transgenic DNA of a junction polynucleotide), or in the transgenic insert. A CgRRS comprises the same gRNA target site and sequence as the corresponding OgRRS. The CgRRS may be inserted or introduced at a location within the transgenic insertion locus of event ZM_BCS216090 that is on the opposite side of the transgenic insertion or miRNA expression cassette or regulatory element of the transgene, relative to the OgRRS, in a manner that will permit the excision or deletion of a fragment of DNA encompassing either the entire transgenic insertion of event ZM_BCS216090 or the miRNA expression cassette, or a fragment within the transgene insert of event ZM_BCS216090 but encompassing a genetic or regulatory element within the transgene cassette, using a single gRNA. For example, if the OgRRS is located within the 3′ flanking genomic sequence or the 3′ junction region, then the CgRRS can be inserted within the 5′ flanking genomic sequence, or the 5′ junction region, or within the transgene insert such as between genetic or regulatory elements within the expression cassette. Insertion of the CgRRS on the opposite side of the transgenic insertion or within the transgenic insert, relative to the OgRRS allows for excision or deletion of the transgenic insertion or specific expression element(s) or expression cassette to be excised or deleted using a single gRNA. An OgRRS located in the expression cassette of event ZM_BCS216090 can be used to design a CgRRS that can be inserted in either the 5′ or 3′-flanking genomic sequence to permit excision or deletion of one or more expression elements or the expression cassette using a single gRNA.

Table 11 shows OgRRS sequences located within the 5′ and 3′-flanking genomic sequences, spanning the 5′ and 3′ junction sequences, and in the transgenic insertion of event ZM_BCS216090 that can be used in a CRISPR editing system employing Cas12a, a Type V CRISPR-associated protein. The analysis was performed for four Cas12a endonucleases. FnCas12a (SEQ ID NO: 25) refers to Francisella novicida U112 Cas12a (also known as FnCpf1) and requires the PAM sequence of 5′-TTN, where N is A, C, G or T (Zetsche et al., 2015). LbCas12a (SEQ ID NO: 22) refers to the Cas12a from Lachnospiraceae bacterium ND2006 (also known as LbCpf1) and requires the PAM sequence of 5′-TTTV, where V is A, C, or G. LbCas12a-TYC and LbCas12a-TAT refer to engineered variants of Lachnospiraceae bacterium ND2006 Cas12a (Gao et al., 2017). The LbCas12a-TYC variant (SEQ ID NO: 23) contains the G532R/K595R mutations and recognizes 5′-TYCV PAM; whereas the LbCas12a-TAT variant (SEQ ID NO: 24) contains the G532R/K538V/Y542R mutations and recognizes 5′-TATV PAM, where Y is C or T, and V is A, C or G. The PAM sequence, the coordinates of the gRNA hybridization or target site (also known as OgRRS) relative to SEQ ID NO: 10, and the corresponding Cas12a endonuclease are shown under the headings of “PAM”, “Cas12a Nuclease”, and “Start . . . End of gRNA Hybridization o Targeting Site in SED ID NO: 10”, respectively. “Strand of SEQ ID NO: 10” indicates whether the identified gRNA hybridization site along with its PAM sequence is on the forward strand (+) or the complementary strand (−). “Target Site” refers to the location of the gRNA hybridization site in the corn event CSM63715 locus.

TABLE 11
OgRRS sequences within or near event ZM_BCS216090

| Name of | | | Start..End of | | |
|---|---|---|---|---|---|
| gRNA | | | gRNA | Strand | |
| Hybridization | | | Hybridization or | of | |
| Site | | Cas12a | Target Site | SEQ ID | |
| (ogRRS) | PAM | Nuclease | in SEQ ID NO: 10 | NO: 10 | Target Site |
| 5F-1 | GTTA | FnCas12a | 252..274 | + | 5′ Flanking genomic |
| | | | | | DNA |
| 5F-2 | TATC | LbCas12a- | 261..283 | − | 5′ Flanking genomic |
| | | TAT | | | DNA |
| 5F-3 | TATG | LbCas12a- | 268..290 | + | 5′ Flanking genomic |
| | | TAT | | | DNA |
| 5F-4 | TTTG | FnCas12a | 273..295 | − | 5′ Flanking genomic |
| | | | | | DNA |
| 5F-5 | TTTC | LbCas12a | 274..296 | + | 5′ Flanking genomic |
| | | | | | DNA |
| 5F-6 | TTCC | LbCas12a- | 275..297 | + | 5′ Flanking genomic |
| | | TYC | | | DNA |
| 5F-7 | TCCA | LbCas12a- | 276..298 | + | 5′ Flanking genomic |
| | | TYC | | | DNA |
| 5F-8 | CTTA | FnCas12a | 280..302 | − | 5′ Flanking genomic |
| | | | | | DNA |
| 5F-9 | TATA | LbCas12a- | 288..310 | − | 5′ Flanking genomic |
| | | TAT | | | DNA |
| 5F-10 | ATTA | FnCas12a | 290..312 | − | 5′ Flanking genomic |
| | | | | | DNA |
| 5F-11 | TTCA | LbCas12a- | 299..321 | − | 5′ Flanking genomic |
| | | TYC | | | DNA |
| 5F-12 | TTTC | LbCas12a | 300..322 | − | 5′ Flanking genomic |
| | | | | | DNA |
| 5F-13 | ATTA | FnCas12a | 314..336 | + | 5′ Flanking genomic |
| | | | | | DNA |
| 5F-14 | TATC | LbCas12a- | 326..348 | − | 5′ Flanking genomic |
| | | TAT | | | DNA |
| 5F-15 | TATA | LbCas12a- | 328..350 | − | 5′ Flanking genomic |
| | | TAT | | | DNA |
| 5F-16 | TATA | LbCas12a- | 333..355 | + | 5′ Flanking genomic |
| | | TAT | | | DNA |
| 5F-17 | ATTC | FnCas12a | 336..358 | + | 5′ Flanking genomic |
| | | | | | DNA |
| 5F-18 | TTCC | LbCas12a- | 337..359 | + | 5′ Flanking genomic |
| | | TYC | | | DNA |
| 5F-19 | TCCA | LbCas12a- | 338..360 | + | 5′ Flanking genomic |
| | | TYC | | | DNA |
| 5F-20 | TATG | LbCas12a- | 340..362 | − | 5′ Flanking genomic |
| | | TAT | | | DNA |
| 5F-21 | CTTG | FnCas12a | 350..372 | + | 5′ Flanking genomic |
| | | | | | DNA |
| 5F-22 | CTTG | FnCas12a | 363..385 | + | 5′ Flanking genomic |
| | | | | | DNA |
| 5F-23 | TTTG | LbCas12a | 368..390 | + | 5′ Flanking genomic |
| | | | | | DNA |
| 5F-24 | TTTG | LbCas12a | 369..391 | − | 5′ Flanking genomic |
| | | | | | DNA |
| 5F-25 | ATTA | FnCas12a | 376..398 | − | 5′ Flanking genomic |
| | | | | | DNA |
| 5F-26 | ATTA | FnCas12a | 383..405 | + | 5′ Flanking genomic |
| | | | | | DNA |
| 5F-27 | TATG | LbCas12a- | 385..407 | + | 5′ Flanking genomic |
| | | TAT | | | DNA |
| 5F-28 | ATTG | FnCas12a | 386..408 | − | 5′ Flanking genomic |
| | | | | | DNA |
| 5F-29 | ATTA | FnCas12a | 391..413 | − | 5′ Flanking genomic |
| | | | | | DNA |
| 5F-30 | TTTA | LbCas12a | 394..416 | + | 5′ Flanking genomic |
| | | | | | DNA |
| 5F-31 | ATTC | FnCas12a | 398..420 | + | 5′ Flanking genomic |
| | | | | | DNA |
| 5F-32 | TTTA | LbCas12a | 401..423 | − | 5′ Flanking genomic |
| | | | | | DNA |
| 5F-33 | CTTG | FnCas12a | 401..423 | + | 5′ Flanking genomic |
| | | | | | DNA |
| 5F-34 | GTTA | FnCas12a | 411..433 | + | 5′ Flanking genomic |
| | | | | | DNA |
| 5F-35 | TATG | LbCas12a- | 414..436 | − | 5′ Flanking genomic |
| | | TAT | | | DNA |
| 5F-36 | TTTA | LbCas12a | 415..437 | + | 5′ Flanking genomic |
| | | | | | DNA |
| 5F-37 | TTTA | LbCas12a | 416..438 | − | 5′ Flanking genomic |
| | | | | | DNA |
| 5F-38 | CTTG | FnCas12a | 422..444 | − | 5′ Flanking genomic |
| | | | | | DNA |
| 5F-39 | TATG | LbCas12a- | 429..451 | − | 5′ Flanking genomic |
| | | TAT | | | DNA |
| 5F-40 | TATC | LbCas12a- | 433..455 | − | 5′ Flanking genomic |
| | | TAT | | | DNA |
| 5F-41 | ATTA | FnCas12a | 439..461 | − | 5′ Flanking genomic |
| | | | | | DNA |
| 5F-42 | TATA | LbCas12a- | 448..470 | + | 5′ Flanking genomic |
| | | TAT | | | DNA |
| 5F-43 | TATG | LbCas12a- | 456..478 | + | 5′ Flanking genomic |
| | | TAT | | | DNA |
| 5F-44 | ATTA | FnCas12a | 472..494 | + | 5′ Flanking genomic |
| | | | | | DNA |
| 5F-45 | ATTA | FnCas12a | 478..500 | − | 5′ Flanking genomic |
| | | | | | DNA |
| 5F-46 | TATC | LbCas12a- | 487..509 | − | 5′ Flanking genomic |
| | | TAT | | | DNA |
| 5F-47 | TATC | LbCas12a- | 504..526 | − | 5′ Flanking genomic |
| | | TAT | | | DNA |
| 5F-48 | TTTA | LbCas12a | 572..594 | + | 5′ Flanking genomic |
| | | | | | DNA |
| 5F-49 | TATA | LbCas12a- | 586..608 | + | 5′ Flanking genomic |
| | | TAT | | | DNA |
| 5F-50 | ATTC | FnCas12a | 596..618 | + | 5′ Flanking genomic |
| | | | | | DNA |
| 5F-51 | TTCA | LbCas12a- | 597..619 | + | 5′ Flanking genomic |
| | | TYC | | | DNA |
| 5F-52 | TTTC | LbCas12a | 602..624 | + | 5′ Flanking genomic |
| | | | | | DNA |
| 5F-53 | TTCC | LbCas12a- | 603..625 | + | 5′ Flanking genomic |
| | | TYC | | | DNA |
| 5F-54 | TCCA | LbCas12a- | 604..626 | + | 5′ Flanking genomic |
| | | TYC | | | DNA |
| 5F-55 | CTTA | FnCas12a | 610..632 | + | 5′ Flanking genomic |
| | | | | | DNA |
| 5F-56 | TTTA | LbCas12a | 614..636 | + | 5′ Flanking genomic |
| | | | | | DNA |
| 5F-57 | TTTG | LbCas12a | 620..642 | − | 5′ Flanking genomic |
| | | | | | DNA |
| 5F-58 | ATTA | FnCas12a | 624..646 | − | 5′ Flanking genomic |
| | | | | | DNA |
| 5F-59 | TCCA | LbCas12a- | 631..653 | − | 5′ Flanking genomic |
| | | TYC | | | DNA |
| 5F-60 | TCCG | LbCas12a- | 638..660 | − | 5′ Flanking genomic |
| | | TYC | | | DNA |
| 5F-61 | TTTG | LbCas12a | 644..666 | − | 5′ Flanking genomic |
| | | | | | DNA |
| 5F-62 | TTTA | LbCas12a | 663..685 | + | 5′ Flanking genomic |
| | | | | | DNA |
| 5F-63 | GTTC | FnCas12a | 669..691 | + | 5′ Flanking genomic |
| | | | | | DNA |
| 5F-64 | TTCA | LbCas12a- | 670..692 | + | 5′ Flanking genomic |
| | | TYC | | | DNA |
| 5F-65 | TTTG | LbCas12a | 674..696 | − | 5′ Flanking genomic |
| | | | | | DNA |
| 5F-66 | TCCC | LbCas12a- | 682..704 | − | 5′ Flanking genomic |
| | | TYC | | | DNA |
| 5F-67 | TTTG | LbCas12a | 707..729 | + | 5′ Flanking genomic |
| | | | | | DNA |
| 5F-68 | TTTA | LbCas12a | 733..755 | − | 5′ Flanking genomic |
| | | | | | DNA |
| 5F-69 | CTTG | FnCas12a | 768..790 | + | 5′ Flanking genomic |
| | | | | | DNA |
| 5F-70 | TATC | LbCas12a- | 782..804 | + | 5′ Flanking genomic |
| | | TAT | | | DNA |
| 5F-71 | ATTG | FnCas12a | 784..806 | − | 5′ Flanking genomic |
| | | | | | DNA |
| 5F-72 | TCCA | LbCas12a- | 796..818 | − | 5′ Flanking genomic |
| | | TYC | | | DNA |
| 5F-73 | TCCA | LbCas12a- | 803..825 | − | 5′ Flanking genomic |
| | | TYC | | | DNA |
| 5F-74 | TTTA | LbCas12a | 821..843 | − | 5′ Flanking genomic |
| | | | | | DNA |
| 5F-75 | ATTA | FnCas12a | 824..846 | + | 5′ Flanking genomic |
| | | | | | DNA |
| 5F-76 | ATTA | FnCas12a | 829..851 | + | 5′ Flanking genomic |
| | | | | | DNA |
| 5F-77 | TATC | LbCas12a- | 831..853 | + | 5′ Flanking genomic |
| | | TAT | | | DNA |
| 5F-78 | GTTA | FnCas12a | 835..857 | + | 5′ Flanking genomic |
| | | | | | DNA |
| 5F-79 | TTCA | LbCas12a- | 837..859 | − | 5′ Flanking genomic |
| | | TYC | | | DNA |
| 5F-80 | ATTC | FnCas12a | 838..860 | − | 5′ Flanking genomic |
| | | | | | DNA |
| 5F-81 | TATG | LbCas12a- | 840..862 | + | 5′ Flanking genomic |
| | | TAT | | | DNA |
| 5F-82 | TATC | LbCas12a- | 843..865 | − | 5′ Flanking genomic |
| | | TAT | | | DNA |
| 5F-83 | ATTG | FnCas12a | 849..871 | − | 5′ Flanking genomic |
| | | | | | DNA |
| 5F-84 | ATTC | FnCas12a | 851..873 | + | 5′ Flanking genomic |
| | | | | | DNA |
| 5F-85 | TTCA | LbCas12a- | 852..874 | + | 5′ Flanking genomic |
| | | TYC | | | DNA |
| 5F-86 | TTCC | LbCas12a- | 854..876 | − | 5′ Flanking genomic |
| | | TYC | | | DNA |
| 5F-87 | GTTC | FnCas12a | 855..877 | − | 5′ Flanking genomic |
| | | | | | DNA |
| 5F-88 | TATC | LbCas12a- | 864..886 | − | 5′ Flanking genomic |
| | | TAT | | | DNA |
| 5F-89 | TATC | LbCas12a- | 869..891 | − | 5′ Flanking genomic |
| | | TAT | | | DNA |
| 5F-90 | TATG | LbCas12a- | 876..898 | + | 5′ Flanking genomic |
| | | TAT | | | DNA |
| 5F-91 | TTTA | LbCas12a | 892..914 | + | 5′ Flanking genomic |
| | | | | | DNA |
| 5F-92 | TATG | LbCas12a- | 894..916 | + | 5′ Flanking genomic |
| | | TAT | | | DNA |
| 5F-93 | GTTG | FnCas12a | 897..919 | + | 5′ Flanking genomic |
| | | | | | DNA |
| 5F-94 | TTTG | LbCas12a | 898..920 | − | 5′ Flanking genomic |
| | | | | | DNA |
| 5F-95 | TCCA | LbCas12a- | 902..924 | − | 5′ Flanking genomic |
| | | TYC | | | DNA |
| 5F-96 | GTTC | FnCas12a | 915..937 | + | 5′ Flanking genomic |
| | | | | | DNA |
| 5F-97 | TTCA | LbCas12a- | 916..938 | + | 5′ Flanking genomic |
| | | TYC | | | DNA |
| 5F-98 | TATC | LbCas12a- | 927..949 | + | 5′ Flanking genomic |
| | | TAT | | | DNA |
| 5F-99 | GTTG | FnCas12a | 928..950 | − | 5′ Flanking genomic |
| | | | | | DNA |
| 5F-100 | ATTC | FnCas12a | 944..966 | + | 5′ Flanking genomic |
| | | | | | DNA |
| 5F-101 | TTCC | LbCas12a- | 945..967 | + | 5′ Flanking genomic |
| | | TYC | | | DNA |
| 5F-102 | CTTA | FnCas12a | 948..970 | + | 5′ Flanking genomic |
| | | | | | DNA |
| 5F-103 | TTCG | LbCas12a- | 951..973 | − | 5′ Flanking genomic |
| | | TYC | | | DNA |
| 5F-104 | GTTC | FnCas12a | 952..974 | − | 5′ Flanking genomic |
| | | | | | DNA |
| 5F-105 | TCCC | LbCas12a- | 953..975 | + | 5′ Flanking genomic |
| | | TYC | | | DNA |
| 5F-106 | TATC | LbCas12a- | 957..979 | − | 5′ Flanking genomic |
| | | TAT | | | DNA |
| 5F-107 | GTTA | FnCas12a | 959..981 | − | 5′ Flanking genomic |
| | | | | | DNA |
| 5F-108 | ATTA | FnCas12a | 970..992 | − | 5′ Flanking genomic |
| | | | | | DNA |
| 5F-109 | CTTC | FnCas12a | 971..993 | + | 5′ Flanking genomic |
| | | | | | DNA |
| 5F-110 | TCCG | LbCas12a- | 978..1000 | + | 5′ Flanking genomic |
| | | TYC | | | DNA |
| 5J-1 | TCCA | LbCas 12a- | 989..1011 | − | 5′ Junction |
| | | TYC | | | |
| 5J-2 | TTCG | LbCas12a- | 995..1017 | − | 5′ Junction |
| | | TYC | | | |
| 5J-3 | CTTC | FnCas12a | 996..1018 | − | 5′ Junction |
| 5J-4 | TCCG | LbCas12a- | 998..1020 | + | 5′ Junction |
| | | TYC | | | |
| TI-1 | TTTG | LbCas12a | 1020..1042 | − | Transgenic insert |
| TI-2 | CTTA | FnCas12a | 1035..1057 | + | Transgenic insert |
| TI-3 | CTTC | FnCas12a | 1048..1070 | + | Transgenic insert |
| TI-4 | TTCA | LbCas12a- | 1049..1071 | + | Transgenic insert |
| | | TYC | | | |
| TI-5 | TTTG | LbCas12a | 1056..1078 | + | Transgenic insert |
| TI-6 | GTTA | FnCas12a | 1062..1084 | − | Transgenic insert |
| TI-7 | CTTA | FnCas12a | 1066..1088 | − | Transgenic insert |
| TI-8 | TCCC | LbCas12a- | 1081..1103 | − | Transgenic insert |
| | | TYC | | | |
| TI-9 | TATO | LbCas12a- | 1089..1111 | − | Transgenic insert |
| | | TAT | | | |
| TI-10 | TTCG | LbCas12a- | 1094..1116 | − | Transgenic insert |
| | | TYC | | | |
| TI-11 | ATTC | FnCas12a | 1095..1117 | | Transgenic insert |
| TI-12 | TATC | LbCas12a- | 1096..1118 | + | Transgenic insert |
| | | TAT | | | |
| TI-13 | CTTG | FnCas12a | 1100..1122 | − | Transgenic insert |
| TI-14 | ATTC | FnCas12a | 1102..1124 | + | Transgenic insert |
| TI-15 | TTCA | LbCas12a- | 1103..1125 | + | Transgenic insert |
| | | TYC | | | |
| TI-16 | TATA | LbCas12a- | 1106..1128 | − | Transgenic insert |
| | | TAT | | | |
| TI-17 | GTTA | FnCas12a | 1108..1130 | − | Transgenic insert |
| TI-18 | CTTA | FnCas12a | 1109..1131 | + | Transgenic insert |
| TI-19 | TATA | LbCas12a- | 1111..1133 | + | Transgenic insert |
| | | TAT | | | |
| TI-20 | CTTC | FnCas12a | 1116..1138 | + | Transgenic insert |
| TI-21 | TATA | LbCas12a- | 1116..1138 | − | Transgenic insert |
| | | TAT | | | |
| TI-22 | TTCG | LbCas12a- | 1117..1139 | + | Transgenic insert |
| | | TYC | | | |
| TI-23 | ATTA | FnCas12a | 1118..1140 | − | Transgenic insert |
| TI-24 | TATA | LbCas12a- | 1121..1143 | + | Transgenic insert |
| | | TAT | | | |
| TI-25 | TATG | LbCas12a- | 1128..1150 | + | Transgenic insert |
| | | TAT | | | |
| TI-26 | TATA | LbCas 12a- | 1128..1150 | − | Transgenic insert |
| | | TAT | | | |
| TI-27 | TTCG | LbCas12a- | 1132..1154 | − | Transgenic insert |
| | | TYC | | | |
| TI-28 | CTTC | FnCas12a | 1133..1155 | − | Transgenic insert |
| TI-29 | TATA | LbCas12a- | 1133..1155 | + | Transgenic insert |
| | | TAT | | | |
| TI-30 | GTTA | FnCas12a | 1141..1163 | + | Transgenic insert |
| TI-31 | TATG | LbCas12a- | 1143..1165 | + | Transgenic insert |
| | | TAT | | | |
| TI-32 | GTTA | FnCas12a | 1147..1169 | − | Transgenic insert |
| TI-33 | TATA | LbCas12a- | 1151..1173 | − | Transgenic insert |
| | | TAT | | | |
| TI-34 | GTTA | FnCas12a | 1153..1175 | − | Transgenic insert |
| TI-35 | TATA | LbCas12a- | 1156..1178 | + | Transgenic insert |
| | | TAT | | | |
| TI-36 | CTTA | FnCas12a | 1162..1184 | − | Transgenic insert |
| TI-37 | CTTA | FnCas12a | 1178..1200 | + | Transgenic insert |
| TI-38 | TATG | LbCas12a- | 1200..1222 | − | Transgenic insert |
| | | TAT | | | |
| TI-39 | ATTA | FnCas12a | 1202..1224 | − | Transgenic insert |
| TI-40 | TATG | LbCas12a- | 1209..1231 | − | Transgenic insert |
| | | TAT | | | |
| TI-41 | ATTA | FnCas12a | 1211..1233 | − | Transgenic insert |
| TI-42 | TTTG | LbCas12a | 1225..1247 | + | Transgenic insert |
| TI-43 | ATTG | FnCas12a | 1225..1247 | − | Transgenic insert |
| TI-44 | GTTC | FnCas12a | 1235..1257 | + | Transgenic insert |
| TI-45 | CTTC | FnCas12a | 1238..1260 | + | Transgenic insert |
| TI-46 | CTTG | FnCas12a | 1241..1263 | + | Transgenic insert |
| TI-47 | CTTG | FnCas12a | 1246..1268 | − | Transgenic insert |
| TI-48 | TTTG | LbCas12a | 1253..1275 | − | Transgenic insert |
| TI-49 | TCCG | LbCas12a- | 1259..1281 | − | Transgenic insert |
| | | TYC | | | |
| TI-50 | GTTG | FnCas12a | 1294..1316 | + | Transgenic insert |
| TI-51 | TTTG | LbCas12a | 1301..1323 | − | Transgenic insert |
| TI-52 | TCCC | LbCas12a- | 1306..1328 | − | Transgenic insert |
| | | TYC | | | |
| TI-53 | ATTA | FnCas12a | 1314..1336 | − | Transgenic insert |
| TI-54 | ATTA | FnCas12a | 1321..1343 | − | Transgenic insert |
| TI-55 | TTTA | LbCas12a | 1324..1346 | + | Transgenic insert |
| TI-56 | TTCA | LbCas12a- | 1330..1352 | − | Transgenic insert |
| | | TYC | | | |
| TI-57 | CTTC | FnCas12a | 1331..1353 | − | Transgenic insert |
| TI-58 | CTTG | FnCas12a | 1333..1355 | + | Transgenic insert |
| TI-59 | TATC | LbCas12a- | 1338..1360 | − | Transgenic insert |
| | | TAT | | | |
| TI-60 | GTTG | FnCas12a | 1343..1365 | − | Transgenic insert |
| TI-61 | TATC | LbCas12a- | 1345..1367 | + | Transgenic insert |
| | | TAT | | | |
| TI-62 | CTTG | FnCas12a | 1353..1375 | − | Transgenic insert |
| TI-63 | TCCA | LbCas12a- | 1356..1378 | + | Transgenic insert |
| | | TYC | | | |
| TI-64 | TTTA | LbCas12a | 1363..1385 | + | Transgenic insert |
| TI-65 | TCCA | LbCas12a- | 1363..1385 | − | Transgenic insert |
| | | TYC | | | |
| TI-66 | ATTG | FnCas12a | 1366..1388 | + | Transgenic insert |
| TI-67 | TTTA | LbCas12a | 1376..1398 | + | Transgenic insert |
| TI-68 | TATG | LbCas12a- | 1378..1400 | + | Transgenic insert |
| | | TAT | | | |
| TI-69 | TTTA | LbCas12a | 1390..1412 | + | Transgenic insert |
| TI-70 | TTTA | LbCas12a | 1390..1412 | − | Transgenic insert |
| TI-71 | ATTA | FnCas12a | 1393..1415 | + | Transgenic insert |
| TI-72 | TTTG | LbCas12a | 1394..1416 | − | Transgenic insert |
| TI-73 | TTTA | LbCas12a | 1398..1420 | − | Transgenic insert |
| TI-74 | GTTG | FnCas12a | 1407..1429 | − | Transgenic insert |
| TI-75 | CTTC | FnCas12a | 1407..1429 | + | Transgenic insert |
| TI-76 | TTCA | LbCas12a- | 1408..1430 | + | Transgenic insert |
| | | TYC | | | |
| TI-77 | CTTG | FnCas12a | 1411..1433 | − | Transgenic insert |
| TI-78 | GTTC | FnCas12a | 1420..1442 | + | Transgenic insert |
| TI-79 | TTCA | LbCas12a- | 1420..1442 | − | Transgenic insert |
| | | TYC | | | |
| TI-80 | CTTC | FnCas12a | 1421..1443 | − | Transgenic insert |
| TI-81 | TTCC | LbCas12a- | 1421..1443 | + | Transgenic insert |
| | | TYC | | | |
| TI-82 | ATTG | FnCas12a | 1434..1456 | − | Transgenic insert |
| TI-83 | TATG | LbCas12a- | 1438..1460 | − | Transgenic insert |
| | | TAT | | | |
| TI-84 | TCCG | LbCas12a- | 1443..1465 | − | Transgenic insert |
| | | TYC | | | |
| TI-85 | TTCC | LbCas12a- | 1444..1466 | − | Transgenic insert |
| | | TYC | | | |
| TI-86 | TTTC | LbCas12a | 1445..1467 | − | Transgenic insert |
| TI-87 | CTTC | FnCas12a | 1456..1478 | + | Transgenic insert |
| TI-88 | TTCA | LbCas12a- | 1457..1479 | + | Transgenic insert |
| | | TYC | | | |
| TI-89 | TATA | LbCas12a- | 1465..1487 | + | Transgenic insert |
| | | TAT | | | |
| TI-90 | GTTC | FnCas12a | 1470..1492 | + | Transgenic insert |
| TI-91 | TTCC | LbCas12a- | 1471..1493 | + | Transgenic insert |
| | | TYC | | | |
| TI-92 | TCCA | LbCas12a- | 1471..1493 | − | Transgenic insert |
| | | TYC | | | |
| TI-93 | TCCC | LbCas12a- | 1472..1494 | + | Transgenic insert |
| | | TYC | | | |
| TI-94 | TTTG | LbCas12a | 1476..1498 | − | Transgenic insert |
| TI-95 | ATTA | FnCas12a | 1488..1510 | − | Transgenic insert |
| TI-96 | CTTA | FnCas12a | 1491..1513 | + | Transgenic insert |
| TI-97 | GTTC | FnCas12a | 1493..1515 | − | Transgenic insert |
| TI-98 | ATTC | FnCas12a | 1507..1529 | + | Transgenic insert |
| TI-99 | TTCC | LbCas12a- | 1508..1530 | + | Transgenic insert |
| | | TYC | | | |
| TI-100 | TCCA | LbCas12a- | 1511..1533 | − | Transgenic insert |
| | | TYC | | | |
| TI-101 | TTCC | LbCas12a- | 1512..1534 | − | Transgenic insert |
| | | TYC | | | |
| TI-102 | GTTC | FnCas12a | 1513..1535 | − | Transgenic insert |
| TI-103 | ATTG | FnCas12a | 1519..1541 | − | Transgenic insert |
| TI-104 | CTTC | FnCas12a | 1521..1543 | + | Transgenic insert |
| TI-105 | TTCA | LbCas12a- | 1522..1544 | − | Transgenic insert |
| | | TYC | | | |
| TI-106 | TTCA | LbCas12a- | 1522..1544 | + | Transgenic insert |
| | | TYC | | | |
| TI-107 | TTTC | LbCas12a | 1523..1545 | − | Transgenic insert |
| TI-108 | TTCA | LbCas12a- | 1527..1549 | − | Transgenic insert |
| | | TYC | | | |
| TI-109 | TTTC | LbCas12a | 1528..1550 | − | Transgenic insert |
| TI-110 | TTTC | LbCas12a | 1535..1557 | − | Transgenic insert |
| TI-111 | GTTG | FnCas12a | 1552..1574 | − | Transgenic insert |
| TI-112 | TATA | LbCas12a- | 1557..1579 | − | Transgenic insert |
| | | TAT | | | |
| TI-113 | TATA | LbCas12a- | 1559..1581 | − | Transgenic insert |
| | | TAT | | | |
| TI-114 | CTTA | FnCas12a | 1560..1582 | + | Transgenic insert |
| TI-115 | TATA | LbCas12a- | 1562..1584 | + | Transgenic insert |
| | | TAT | | | |
| TI-116 | TATA | LbCas12a- | 1564..1586 | + | Transgenic insert |
| | | TAT | | | |
| TI-117 | TATC | LbCas12a- | 1566..1588 | + | Transgenic insert |
| | | TAT | | | |
| TI-118 | CTTG | FnCas12a | 1567..1589 | − | Transgenic insert |
| TI-119 | CTTC | FnCas12a | 1569..1591 | + | Transgenic insert |
| TI-120 | ATTC | FnCas12a | 1570..1592 | − | Transgenic insert |
| TI-121 | TTCA | LbCas12a- | 1570..1592 | + | Transgenic insert |
| | | TYC | | | |
| TI-122 | ATTA | FnCas12a | 1577..1599 | + | Transgenic insert |
| TI-123 | TATC | LbCas12a- | 1579..1601 | + | Transgenic insert |
| | | TAT | | | |
| TI-124 | TTTC | LbCas12a | 1587..1609 | + | Transgenic insert |
| TI-125 | TATG | LbCas12a- | 1591..1613 | + | Transgenic insert |
| | | TAT | | | |
| TI-126 | ATTC | FnCas12a | 1598..1620 | + | Transgenic insert |
| TI-127 | TTCA | LbCas12a- | 1599..1621 | + | Transgenic insert |
| | | TYC | | | |
| TI-128 | CTTA | FnCas12a | 1602..1624 | − | Transgenic insert |
| TI-129 | ATTA | FnCas12a | 1605..1627 | + | Transgenic insert |
| TI-130 | TATG | LbCas12a- | 1608..1630 | − | Transgenic insert |
| | | TAT | | | |
| TI-131 | TTCG | LbCas12a- | 1612..1634 | − | Transgenic insert |
| | | TYC | | | |
| TI-132 | GTTC | FnCas12a | 1613..1635 | − | Transgenic insert |
| TI-133 | TATA | LbCas12a- | 1618..1640 | − | Transgenic insert |
| | | TAT | | | |
| TI-134 | GTTA | FnCas12a | 1620..1642 | − | Transgenic insert |
| TI-135 | TATA | LbCas12a- | 1623..1645 | + | Transgenic insert |
| | | TAT | | | |
| TI-136 | TTCA | LbCas12a- | 1627..1649 | − | Transgenic insert |
| | | TYC | | | |
| TI-137 | CTTC | FnCas12a | 1628..1650 | − | Transgenic insert |
| TI-138 | ATTC | FnCas12a | 1631..1653 | − | Transgenic insert |
| TI-139 | TTTG | LbCas12a | 1639..1661 | + | Transgenic insert |
| TI-140 | TTTG | LbCas12a | 1645..1667 | + | Transgenic insert |
| TI-141 | ATTA | FnCas12a | 1649..1671 | + | Transgenic insert |
| TI-142 | TCCC | LbCas12a- | 1656..1678 | − | Transgenic insert |
| | | TYC | | | |
| TI-143 | TTCC | LbCas12a- | 1657..1679 | − | Transgenic insert |
| | | TYC | | | |
| TI-144 | TTTC | LbCas12a | 1658..1680 | − | Transgenic insert |
| TI-145 | GTTG | FnCas12a | 1658..1680 | + | Transgenic insert |
| TI-146 | GTTG | FnCas12a | 1672..1694 | + | Transgenic insert |
| TI-147 | TTCA | LbCas12a- | 1672..1694 | − | Transgenic insert |
| | | TYC | | | |
| TI-148 | CTTC | FnCas12a | 1673..1695 | − | Transgenic insert |
| TI-149 | TTCG | LbCas12a- | 1678..1700 | − | Transgenic insert |
| | | TYC | | | |
| TI-150 | CTTC | FnCas12a | 1679..1701 | − | Transgenic insert |
| TI-151 | CTTC | FnCas12a | 1685..1707 | − | Transgenic insert |
| TI-152 | TATA | LbCas12a- | 1692..1714 | − | Transgenic insert |
| | | TAT | | | |
| TI-153 | GTTG | FnCas12a | 1693..1715 | + | Transgenic insert |
| TI-154 | TATA | LbCas12a- | 1697..1719 | + | Transgenic insert |
| | | TAT | | | |
| TI-155 | CTTG | FnCas12a | 1702..1724 | − | Transgenic insert |
| TI-156 | TCCA | LbCas12a- | 1705..1727 | + | Transgenic insert |
| | | TYC | | | |
| TI-157 | TATA | LbCas12a- | 1706..1728 | − | Transgenic insert |
| | | TAT | | | |
| TI-158 | CTTA | FnCas12a | 1711..1733 | − | Transgenic insert |
| TI-159 | TATA | LbCas12a- | 1711..1733 | + | Transgenic insert |
| | | TAT | | | |
| TI-160 | TTTG | LbCas12a | 1720..1742 | − | Transgenic insert |
| TI-161 | ATTA | FnCas12a | 1720..1742 | + | Transgenic insert |
| TI-162 | TATC | LbCas12a- | 1722..1744 | + | Transgenic insert |
| | | TAT | | | |
| TI-163 | TTTA | LbCas12a | 1729..1751 | − | Transgenic insert |
| TI-164 | CTTG | FnCas12a | 1751..1773 | + | Transgenic insert |
| TI-165 | GTTC | FnCas12a | 1754..1776 | − | Transgenic insert |
| TI-166 | TCCA | LbCas12a- | 1762..1784 | − | Transgenic insert |
| | | TYC | | | |
| TI-167 | ATTG | FnCas12a | 1770..1792 | − | Transgenic insert |
| TI-168 | ATTG | FnCas12a | 1788..1810 | − | Transgenic insert |
| TI-169 | CTTC | FnCas12a | 1793..1815 | − | Transgenic insert |
| TI-170 | TTCA | LbCas12a- | 1798..1820 | − | Transgenic insert |
| | | TYC | | | |
| TI-171 | ATTC | FnCas12a | 1799..1821 | − | Transgenic insert |
| TI-172 | TCCA | LbCas12a- | 1802..1824 | − | Transgenic insert |
| | | TYC | | | |
| TI-173 | TCCA | LbCas12a- | 1811..1833 | + | Transgenic insert |
| | | TYC | | | |
| TI-174 | TCCA | LbCas12a- | 1815..1837 | − | Transgenic insert |
| | | TYC | | | |
| TI-175 | TATG | LbCas12a- | 1818..1840 | + | Transgenic insert |
| | | TAT | | | |
| TI-176 | ATTG | FnCas12a | 1819..1841 | − | Transgenic insert |
| TI-177 | TTTA | LbCas12a | 1826..1848 | − | Transgenic insert |
| TI-178 | CTTA | FnCas12a | 1829..1851 | + | Transgenic insert |
| TI-179 | CTTC | FnCas12a | 1830..1852 | − | Transgenic insert |
| TI-180 | TTTG | LbCas12a | 1840..1862 | − | Transgenic insert |
| TI-181 | TTTA | LbCas12a | 1840..1862 | + | Transgenic insert |
| TI-182 | TATC | LbCas12a- | 1842..1864 | + | Transgenic insert |
| | | TAT | | | |
| TI-183 | GTTG | FnCas12a | 1846..1868 | − | Transgenic insert |
| TI-184 | CTTA | FnCas12a | 1852..1874 | − | Transgenic insert |
| TI-185 | TTTA | LbCas12a | 1855..1877 | + | Transgenic insert |
| TI-186 | TTCG | LbCas12a- | 1858..1880 | − | Transgenic insert |
| | | TYC | | | |
| TI-187 | ATTC | FnCas12a | 1859..1881 | − | Transgenic insert |
| TI-188 | ATTG | FnCas12a | 1863..1885 | − | Transgenic insert |
| TI-189 | TTTA | LbCas12a | 1866..1888 | − | Transgenic insert |
| TI-190 | CTTC | FnCas12a | 1870..1892 | − | Transgenic insert |
| TI-191 | CTTC | FnCas12a | 1879..1901 | − | Transgenic insert |
| TI-192 | TATC | LbCas12a- | 1882..1904 | − | Transgenic insert |
| | | TAT | | | |
| TI-193 | TATA | LbCas12a- | 1884..1906 | − | Transgenic insert |
| | | TAT | | | |
| TI-194 | TTTA | LbCas12a | 1886..1908 | − | Transgenic insert |
| TI-195 | TATA | LbCas12a- | 1889..1911 | + | Transgenic insert |
| | | TAT | | | |
| TI-196 | TCCC | LbCas12a- | 1892..1914 | − | Transgenic insert |
| | | TYC | | | |
| TI-197 | TTCC | LbCas12a- | 1893..1915 | − | Transgenic insert |
| | | TYC | | | |
| TI-198 | GTTC | FnCas12a | 1894..1916 | − | Transgenic insert |
| TI-199 | CTTC | FnCas12a | 1905..1927 | + | Transgenic insert |
| TI-200 | TTCA | LbCas12a- | 1906..1928 | + | Transgenic insert |
| | | TYC | | | |
| TI-201 | TTTA | LbCas12a | 1933..1955 | + | Transgenic insert |
| TI-202 | TTTA | LbCas12a | 1938..1960 | + | Transgenic insert |
| TI-203 | ATTA | FnCas12a | 1950..1972 | + | Transgenic insert |
| TI-204 | CTTA | FnCas12a | 1952..1974 | − | Transgenic insert |
| TI-205 | ATTG | FnCas12a | 1953..1975 | + | Transgenic insert |
| TI-206 | TATA | LbCas12a- | 1960..1982 | − | Transgenic insert |
| | | TAT | | | |
| TI-207 | ATTA | FnCas12a | 1962..1984 | − | Transgenic insert |
| TI-208 | TATA | LbCas12a- | 1965..1987 | + | Transgenic insert |
| | | TAT | | | |
| TI-209 | TCCA | LbCas12a- | 1980..2002 | − | Transgenic insert |
| | | TYC | | | |
| TI-210 | TCCA | LbCas12a- | 1990..2012 | + | Transgenic insert |
| | | TYC | | | |
| TI-211 | TATA | LbCas12a- | 1990..2012 | − | Transgenic insert |
| | | TAT | | | |
| TI-212 | TATA | LbCas12a- | 1992..2014 | − | Transgenic insert |
| | | TAT | | | |
| TI-213 | CTTA | FnCas12a | 1994..2016 | − | Transgenic insert |
| TI-214 | TATA | LbCas12a- | 1995..2017 | + | Transgenic insert |
| | | TAT | | | |
| TI-215 | TATA | LbCas12a- | 1997..2019 | + | Transgenic insert |
| | | TAT | | | |
| TI-216 | ATTA | FnCas12a | 2014..2036 | − | Transgenic insert |
| TI-217 | CTTG | FnCas12a | 2029..2051 | − | Transgenic insert |
| TI-218 | CTTG | FnCas12a | 2033..2055 | − | Transgenic insert |
| TI-219 | TTTG | LbCas12a | 2045..2067 | − | Transgenic insert |
| TI-220 | CTTA | FnCas12a | 2053..2075 | − | Transgenic insert |
| TI-221 | CTTC | FnCas12a | 2054..2076 | + | Transgenic insert |
| TI-222 | CTTC | FnCas12a | 2092..2114 | + | Transgenic insert |
| TI-223 | TTCG | LbCas12a- | 2093..2115 | + | Transgenic insert |
| | | TYC | | | |
| TI-224 | TCCG | LbCas12a- | 2109..2131 | + | Transgenic insert |
| | | TYC | | | |
| TI-225 | TTTG | LbCas12a | 2122..2144 | + | Transgenic insert |
| TI-226 | GTTA | FnCas12a | 2125..2147 | + | Transgenic insert |
| TI-227 | TTTC | LbCas12a | 2137..2159 | + | Transgenic insert |
| TI-228 | TTCA | LbCas12a- | 2138..2160 | − | Transgenic insert |
| | | TYC | | | |
| TI-229 | ATTC | FnCas12a | 2139..2161 | − | Transgenic insert |
| TI-230 | CTTG | FnCas12a | 2153..2175 | + | Transgenic insert |
| TI-231 | ATTG | FnCas12a | 2164..2186 | + | Transgenic insert |
| TI-232 | TTTA | LbCas12a | 2178..2200 | + | Transgenic insert |
| TI-233 | TCCA | LbCas12a- | 2179..2201 | − | Transgenic insert |
| | | TYC | | | |
| TI-234 | ATTC | FnCas12a | 2187..2209 | − | Transgenic insert |
| TI-235 | ATTA | FnCas12a | 2194..2216 | + | Transgenic insert |
| TI-236 | TTCA | LbCas12a- | 2198..2220 | − | Transgenic insert |
| | | TYC | | | |
| TI-237 | TTTC | LbCas12a | 2199..2221 | − | Transgenic insert |
| TI-238 | GTTC | FnCas12a | 2212..2234 | + | Transgenic insert |
| TI-239 | ATTA | FnCas12a | 2227..2249 | + | Transgenic insert |
| TI-240 | CTTG | FnCas12a | 2231..2253 | + | Transgenic insert |
| TI-241 | TTTG | LbCas12a | 2241..2263 | + | Transgenic insert |
| TI-242 | TTTG | LbCas12a | 2246..2268 | − | Transgenic insert |
| TI-243 | TATA | LbCas12a- | 2251..2273 | − | Transgenic insert |
| | | TAT | | | |
| TI-244 | TCCC | LbCas12a- | 2255..2277 | − | Transgenic insert |
| | | TYC | | | |
| TI-245 | TATA | LbCas12a- | 2256..2278 | + | Transgenic insert |
| | | TAT | | | |
| TI-246 | TTCG | LbCas12a- | 2269..2291 | − | Transgenic insert |
| | | TYC | | | |
| TI-247 | TTTC | LbCas12a | 2270..2292 | − | Transgenic insert |
| TI-248 | TTCG | LbCas12a- | 2274..2296 | − | Transgenic insert |
| | | TYC | | | |
| TI-249 | CTTC | FnCas12a | 2275..2297 | − | Transgenic insert |
| TI-250 | TATC | LbCas12a- | 2278..2300 | − | Transgenic insert |
| | | TAT | | | |
| TI-251 | GTTC | FnCas12a | 2282..2304 | − | Transgenic insert |
| TI-252 | ATTG | FnCas12a | 2294..2316 | − | Transgenic insert |
| TI-253 | TTTC | LbCas12a | 2302..2324 | − | Transgenic insert |
| TI-254 | ATTA | FnCas12a | 2310..2332 | − | Transgenic insert |
| TI-255 | TTTG | LbCas12a | 2318..2340 | + | Transgenic insert |
| TI-256 | CTTA | FnCas12a | 2325..2347 | + | Transgenic insert |
| TI-257 | TTTA | LbCas12a | 2335..2357 | + | Transgenic insert |
| TI-258 | CTTA | FnCas12a | 2336..2358 | − | Transgenic insert |
| TI-259 | TTTA | LbCas12a | 2339..2361 | + | Transgenic insert |
| TI-260 | GTTG | FnCas12a | 2350..2372 | + | Transgenic insert |
| TI-261 | TATA | LbCas12a- | 2353..2375 | − | Transgenic insert |
| | | TAT | | | |
| TI-262 | GTTA | FnCas12a | 2356..2378 | + | Transgenic insert |
| TI-263 | TATA | LbCas12a- | 2358..2380 | + | Transgenic insert |
| | | TAT | | | |
| TI-264 | TCCA | LbCas 12a- | 2364..2386 | − | Transgenic insert |
| | | TYC | | | |
| TI-265 | CTTG | FnCas12a | 2367..2389 | + | Transgenic insert |
| TI-266 | CTTC | FnCas12a | 2372..2394 | − | Transgenic insert |
| TI-267 | ATTC | FnCas12a | 2372..2394 | + | Transgenic insert |
| TI-268 | TTCA | LbCas12a- | 2373..2395 | + | Transgenic insert |
| | | TYC | | | |
| TI-269 | TTTG | LbCas12a | 2381..2403 | + | Transgenic insert |
| TI-270 | ATTA | FnCas12a | 2384..2406 | − | Transgenic insert |
| TI-271 | GTTA | FnCas12a | 2387..2409 | + | Transgenic insert |
| TI-272 | TTTA | LbCas12a | 2392..2414 | + | Transgenic insert |
| TI-273 | TATG | LbCas12a- | 2402..2424 | − | Transgenic insert |
| | | TAT | | | |
| TI-274 | CTTC | FnCas12a | 2404..2426 | + | Transgenic insert |
| TI-275 | TTCA | LbCas12a- | 2405..2427 | + | Transgenic insert |
| | | TYC | | | |
| TI-276 | ATTG | FnCas12a | 2416..2438 | − | Transgenic insert |
| TI-277 | TATA | LbCas12a- | 2422..2444 | − | Transgenic insert |
| | | TAT | | | |
| TI-278 | TCCC | LbCas12a- | 2426..2448 | − | Transgenic insert |
| | | TYC | | | |
| TI-279 | TATA | LbCas12a- | 2427..2449 | + | Transgenic insert |
| | | TAT | | | |
| TI-280 | TATG | LbCas12a- | 2432..2454 | − | Transgenic insert |
| | | TAT | | | |
| TI-281 | ATTC | FnCas12a | 2434..2456 | + | Transgenic insert |
| TI-282 | TTCA | LbCas12a- | 2435..2457 | + | Transgenic insert |
| | | TYC | | | |
| TI-283 | TATA | LbCas12a- | 2437..2459 | − | Transgenic insert |
| | | TAT | | | |
| TI-284 | ATTA | FnCas12a | 2440..2462 | + | Transgenic insert |
| TI-285 | TATA | LbCas12a- | 2442..2464 | + | Transgenic insert |
| | | TAT | | | |
| TI-286 | TATC | LbCas12a- | 2444..2466 | − | Transgenic insert |
| | | TAT | | | |
| TI-287 | TATA | LbCas12a- | 2449..2471 | − | Transgenic insert |
| | | TAT | | | |
| TI-288 | ATTA | FnCas12a | 2451..2473 | − | Transgenic insert |
| TI-289 | ATTA | FnCas12a | 2454..2476 | − | Transgenic insert |
| TI-290 | TATA | LbCas12a- | 2454..2476 | + | Transgenic insert |
| | | TAT | | | |
| TI-291 | TTTG | LbCas12a | 2462..2484 | + | Transgenic insert |
| TI-292 | GTTC | FnCas12a | 2465..2487 | + | Transgenic insert |
| TI-293 | TTCG | LbCas12a- | 2466..2488 | + | Transgenic insert |
| | | TYC | | | |
| TI-294 | CTTA | FnCas12a | 2479..2501 | + | Transgenic insert |
| TI-295 | ATTA | FnCas12a | 2482..2504 | + | Transgenic insert |
| TI-296 | TTTG | LbCas12a | 2486..2508 | − | Transgenic insert |
| TI-297 | TTTG | LbCas12a | 2486..2508 | + | Transgenic insert |
| TI-298 | TATC | LbCas12a- | 2494..2516 | − | Transgenic insert |
| | | TAT | | | |
| TI-299 | TTTG | LbCas12a | 2516..2538 | + | Transgenic insert |
| TI-300 | TTTG | LbCas12a | 2520..2542 | + | Transgenic insert |
| TI-301 | GTTA | FnCas12a | 2530..2552 | + | Transgenic insert |
| TI-302 | GTTA | FnCas12a | 2535..2557 | − | Transgenic insert |
| TI-303 | ATTG | FnCas12a | 2535..2557 | + | Transgenic insert |
| TI-304 | GTTA | FnCas12a | 2538..2560 | + | Transgenic insert |
| TI-305 | TTCA | LbCas12a- | 2542..2564 | − | Transgenic insert |
| | | TYC | | | |
| TI-306 | CTTC | FnCas12a | 2543..2565 | − | Transgenic insert |
| TI-307 | TTCC | LbCas12a- | 2546..2568 | − | Transgenic insert |
| | | TYC | | | |
| TI-308 | ATTC | FnCas12a | 2547..2569 | − | Transgenic insert |
| TI-309 | TTTA | LbCas12a | 2550..2572 | − | Transgenic insert |
| TI-310 | TATA | LbCas12a- | 2554..2576 | − | Transgenic insert |
| | | TAT | | | |
| TI-311 | TTTA | LbCas12a | 2556..2578 | − | Transgenic insert |
| TI-312 | TATA | LbCas12a- | 2559..2581 | + | Transgenic insert |
| | | TAT | | | |
| TI-313 | TTCG | LbCas12a- | 2563..2585 | − | Transgenic insert |
| | | TYC | | | |
| TI-314 | TTTC | LbCas12a | 2564..2586 | − | Transgenic insert |
| TI-315 | TTTG | LbCas12a | 2574..2596 | + | Transgenic insert |
| TI-316 | TTTA | LbCas12a | 2581..2603 | + | Transgenic insert |
| TI-317 | TATC | LbCas12a- | 2583..2605 | + | Transgenic insert |
| | | TAT | | | |
| TI-318 | TTTA | LbCas12a | 2595..2617 | + | Transgenic insert |
| TI-319 | ATTG | FnCas12a | 2598..2620 | + | Transgenic insert |
| TI-320 | TATG | LbCas12a- | 2601..2623 | − | Transgenic insert |
| | | TAT | | | |
| TI-321 | CTTA | FnCas12a | 2603..2625 | − | Transgenic insert |
| TI-322 | CTTG | FnCas12a | 2608..2630 | − | Transgenic insert |
| TI-323 | CTTG | FnCas12a | 2627..2649 | + | Transgenic insert |
| TI-324 | TTTA | LbCas12a | 2629..2651 | − | Transgenic insert |
| TI-325 | TTTA | LbCas12a | 2632..2654 | + | Transgenic insert |
| TI-326 | ATTG | FnCas12a | 2639..2661 | + | Transgenic insert |
| TI-327 | GTTG | FnCas12a | 2642..2664 | + | Transgenic insert |
| TI-328 | TTCA | LbCas12a- | 2643..2665 | − | Transgenic insert |
| | | TYC | | | |
| TI-329 | CTTC | FnCas12a | 2644..2666 | − | Transgenic insert |
| TI-330 | TTTC | LbCas12a | 2647..2669 | − | Transgenic insert |
| TI-331 | CTTA | FnCas12a | 2651..2673 | − | Transgenic insert |
| TI-332 | TTTG | LbCas12a | 2672..2694 | + | Transgenic insert |
| TI-333 | ATTG | FnCas12a | 2680..2702 | + | Transgenic insert |
| TI-334 | GTTA | FnCas12a | 2695..2717 | − | Transgenic insert |
| TI-335 | TTTG | LbCas12a | 2698..2720 | − | Transgenic insert |
| TI-336 | TTTA | LbCas12a- | 2705..2727 | − | Transgenic insert |
| | | TYC | | | |
| TI-337 | TTTG | LbCas12a | 2730..2752 | + | Transgenic insert |
| TI-338 | GTTG | FnCas12a | 2733..2755 | + | Transgenic insert |
| TI-339 | CTTA | FnCas12a | 2742..2764 | + | Transgenic insert |
| TI-340 | TATC | LbCas12a- | 2757..2779 | + | Transgenic insert |
| | | TAT | | | |
| TI-341 | TATC | LbCas12a- | 2764..2786 | + | Transgenic insert |
| | | TAT | | | |
| TI-342 | GTTG | FnCas12a | 2766..2788 | − | Transgenic insert |
| TI-343 | TATA | LbCas12a- | 2776..2798 | − | Transgenic insert |
| | | TAT | | | |
| TI-344 | TATA | LbCas12a- | 2781..2803 | + | Transgenic insert |
| | | TAT | | | |
| TI-345 | ATTG | FnCas12a | 2784..2806 | + | Transgenic insert |
| TI-346 | CTTC | FnCas12a | 2788..2810 | + | Transgenic insert |
| TI-347 | TATG | LbCas12a- | 2793..2815 | − | Transgenic insert |
| | | TAT | | | |
| TI-348 | TTTA | LbCas12a | 2794..2816 | + | Transgenic insert |
| TI-349 | TATC | LbCas12a- | 2804..2826 | + | Transgenic insert |
| | | TAT | | | |
| TI-350 | CTTG | FnCas12a | 2807..2829 | + | Transgenic insert |
| TI-351 | CTTC | FnCas12a | 2819..2841 | + | Transgenic insert |
| TI-352 | TCCC | LbCas12a- | 2823..2845 | + | Transgenic insert |
| | | TYC | | | |
| TI-353 | GTTG | FnCas12a | 2831..2853 | + | Transgenic insert |
| TI-354 | GTTA | FnCas12a | 2841..2863 | + | Transgenic insert |
| TI-355 | TATG | LbCas12a- | 2844..2866 | − | Transgenic insert |
| | | TAT | | | |
| TI-356 | TTTG | LbCas12a | 2856..2878 | + | Transgenic insert |
| TI-357 | TTTC | LbCas12a | 2864..2886 | + | Transgenic insert |
| TI-358 | TTCA | LbCas12a- | 2865..2887 | + | Transgenic insert |
| | | TYC | | | |
| TI-359 | ATTA | FnCas12a | 2867..2889 | − | Transgenic insert |
| TI-360 | ATTG | FnCas12a | 2868..2890 | + | Transgenic insert |
| TI-361 | TATC | LbCas12a- | 2874..2896 | − | Transgenic insert |
| | | TAT | | | |
| TI-362 | CTTG | FnCas12a | 2879..2901 | − | Transgenic insert |
| TI-363 | TCCC | LbCas12a- | 2912..2934 | + | Transgenic insert |
| | | TYC | | | |
| TI-364 | CTTG | FnCas12a | 2920..2942 | − | Transgenic insert |
| TI-365 | TTTG | LbCas12a | 2945..2967 | + | Transgenic insert |
| TI-366 | GTTA | FnCas12a | 2957..2979 | + | Transgenic insert |
| TI-367 | TTCG | LbCas12a- | 2959..2981 | − | Transgenic insert |
| | | TYC | | | |
| TI-368 | GTTC | FnCas12a | 2960..2982 | − | Transgenic insert |
| TI-369 | TTCA | LbCas12a- | 2967..2989 | − | Transgenic insert |
| | | TYC | | | |
| TI-370 | CTTC | FnCas12a | 2968..2990 | − | Transgenic insert |
| TI-371 | TTCC | LbCas12a- | 2971..2993 | − | Transgenic insert |
| | | TYC | | | |
| TI-372 | CTTC | FnCas12a | 2972..2994 | − | Transgenic insert |
| TI-373 | ATTG | FnCas12a | 2981..3003 | + | Transgenic insert |
| TI-374 | TCCA | LbCas12a- | 2986..3008 | + | Transgenic insert |
| | | TYC | | | |
| TI-375 | GTTG | FnCas12a | 2992..3014 | + | Transgenic insert |
| TI-376 | ATTA | FnCas12a | 3001..3023 | + | Transgenic insert |
| TI-377 | GTTG | FnCas12a | 3005..3027 | + | Transgenic insert |
| TI-378 | TCCA | LbCas12a- | 3014..3036 | − | Transgenic insert |
| | | TYC | | | |
| TI-379 | TTTC | LbCas12a | 3018..3040 | − | Transgenic insert |
| TI-380 | ATTG | FnCas12a | 3030..3052 | + | Transgenic insert |
| TI-381 | TATA | LbCas12a- | 3031..3053 | − | Transgenic insert |
| | | TAT | | | |
| TI-382 | CTTA | FnCas12a | 3034..3056 | + | Transgenic insert |
| TI-383 | TATA | LbCas12a- | 3036..3058 | + | Transgenic insert |
| | | TAT | | | |
| TI-384 | ATTC | FnCas12a | 3039..3061 | + | Transgenic insert |
| TI-385 | ATTG | FnCas12a | 3040..3062 | − | Transgenic insert |
| TI-386 | TTCA | LbCas12a- | 3040..3062 | + | Transgenic insert |
| | | TYC | | | |
| TI-387 | TATA | LbCas12a- | 3043..3065 | − | Transgenic insert |
| | | TAT | | | |
| TI-388 | ATTA | FnCas12a | 3045..3067 | − | Transgenic insert |
| TI-389 | TATA | LbCas12a- | 3048..3070 | + | Transgenic insert |
| | | TAT | | | |
| TI-390 | GTTC | FnCas12a | 3054..3076 | + | Transgenic insert |
| TI-391 | TTCC | LbCas12a- | 3055..3077 | + | Transgenic insert |
| | | TYC | | | |
| TI-392 | TCCA | LbCas12a- | 3059..3081 | + | Transgenic insert |
| | | TYC | | | |
| TI-393 | TCCA | LbCas12a- | 3066..3088 | + | Transgenic insert |
| | | TYC | | | |
| TI-394 | ATTG | FnCas12a | 3068..3090 | − | Transgenic insert |
| TI-395 | ATTA | FnCas12a | 3072..3094 | − | Transgenic insert |
| TI-396 | ATTA | FnCas12a | 3075..3097 | + | Transgenic insert |
| TI-397 | TATA | LbCas12a- | 3075..3097 | − | Transgenic insert |
| | | TAT | | | |
| TI-398 | TATA | LbCas12a- | 3080..3102 | + | Transgenic insert |
| | | TAT | | | |
| TI-399 | TATO | LbCas12a- | 3082..3104 | − | Transgenic insert |
| | | TAT | | | |
| TI-400 | TCCA | LbCas12a- | 3087..3109 | − | Transgenic insert |
| | | TYC | | | |
| TI-401 | TTCC | LbCas12a- | 3088..3110 | − | Transgenic insert |
| | | TYC | | | |
| TI-402 | CTTC | FnCas12a | 3089..3111 | − | Transgenic insert |
| TI-403 | GTTC | FnCas12a | 3092..3114 | − | Transgenic insert |
| TI-404 | GTTA | FnCas12a | 3098..3120 | − | Transgenic insert |
| TI-405 | TATG | LbCas12a- | 3101..3123 | − | Transgenic insert |
| | | TAT | | | |
| TI-406 | TATG | LbCas12a- | 3108..3130 | + | Transgenic insert |
| | | TAT | | | |
| TI-407 | TTTG | LbCas12a | 3115..3137 | + | Transgenic insert |
| TI-408 | TCCA | LbCas12a- | 3136..3158 | − | Transgenic insert |
| | | TYC | | | |
| TI-409 | TTCA | LbCas12a- | 3144..3166 | − | Transgenic insert |
| | | TYC | | | |
| TI-410 | ATTC | FnCas12a | 3145..3167 | − | Transgenic insert |
| TI-411 | ATTG | FnCas12a | 3147..3169 | + | Transgenic insert |
| TI-412 | TATC | LbCas12a- | 3153..3175 | + | Transgenic insert |
| | | TAT | | | |
| TI-413 | GTTC | FnCas12a | 3160..3182 | + | Transgenic insert |
| TI-414 | TTCA | LbCas12a- | 3161..3183 | + | Transgenic insert |
| | | TYC | | | |
| TI-415 | TTTC | LbCas12a | 3169..3191 | + | Transgenic insert |
| TI-416 | TTCA | LbCas12a- | 3170..3192 | + | Transgenic insert |
| | | TYC | | | |
| TI-417 | TTCA | LbCas12a- | 3178..3200 | − | Transgenic insert |
| | | TYC | | | |
| TI-418 | GTTC | FnCas12a | 3179..3201 | − | Transgenic insert |
| TI-419 | TTCA | LbCas12a- | 3185..3207 | − | Transgenic insert |
| | | TYC | | | |
| TI-420 | ATTC | FnCas12a | 3186..3208 | − | Transgenic insert |
| TI-421 | CTTG | FnCas12a | 3198..3220 | + | Transgenic insert |
| TI-422 | GTTA | FnCas12a | 3207..3229 | + | Transgenic insert |
| TI-423 | TTTC | LbCas12a | 3215..3237 | + | Transgenic insert |
| TI-424 | TTCA | LbCas12a- | 3216..3238 | + | Transgenic insert |
| | | TYC | | | |
| TI-425 | TTTA | LbCas12a | 3221..3243 | + | Transgenic insert |
| TI-426 | ATTA | FnCas12a | 3222..3244 | − | Transgenic insert |
| TI-427 | TTTA | LbCas12a | 3230..3252 | + | Transgenic insert |
| TI-428 | TATG | LbCas12a- | 3232..3254 | + | Transgenic insert |
| | | TAT | | | |
| TI-429 | TTCA | LbCas12a- | 3242..3264 | − | Transgenic insert |
| | | TYC | | | |
| TI-430 | TTTC | LbCas12a | 3243..3265 | − | Transgenic insert |
| TI-431 | TCCC | LbCas12a- | 3250..3272 | − | Transgenic insert |
| | | TYC | | | |
| TI-432 | TTCC | LbCas12a- | 3251..3273 | − | Transgenic insert |
| | | TYC | | | |
| TI-433 | TTTC | LbCas12a | 3252..3274 | − | Transgenic insert |
| TI-434 | TTCA | LbCas12a- | 3260..3282 | − | Transgenic insert |
| | | TYC | | | |
| TI-435 | CTTC | FnCas12a | 3261..3283 | − | Transgenic insert |
| TI-436 | ATTC | FnCas12a | 3264..3286 | − | Transgenic insert |
| TI-437 | ATTC | FnCas12a | 3271..3293 | + | Transgenic insert |
| TI-438 | TTTC | LbCas12a | 3277..3299 | + | Transgenic insert |
| TI-439 | TTTC | LbCas12a | 3281..3303 | + | Transgenic insert |
| TI-440 | TTTC | LbCas12a | 3285..3307 | + | Transgenic insert |
| TI-441 | ATTG | FnCas12a | 3287..3309 | − | Transgenic insert |
| TI-442 | TTTC | LbCas12a | 3289..3311 | + | Transgenic insert |
| TI-443 | TTCA | LbCas12a- | 3290..3312 | + | Transgenic insert |
| | | TYC | | | |
| TI-444 | TTTC | LbCas12a | 3297..3319 | + | Transgenic insert |
| TI-445 | TTCG | LbCas12a- | 3298..3320 | + | Transgenic insert |
| | | TYC | | | |
| TI-446 | GTTC | FnCas12a | 3303..3325 | − | Transgenic insert |
| TI-447 | ATTA | FnCas12a | 3319..3341 | − | Transgenic insert |
| TI-448 | GTTA | FnCas12a | 3322..3344 | + | Transgenic insert |
| TI-449 | ATTA | FnCas12a | 3323..3345 | − | Transgenic insert |
| TI-450 | ATTA | FnCas12a | 3326..3348 | + | Transgenic insert |
| TI-451 | TCCA | LbCas12a- | 3331..3353 | + | Transgenic insert |
| | | TYC | | | |
| TI-452 | CTTG | FnCas12a | 3344..3366 | + | Transgenic insert |
| TI-453 | TTTG | LbCas12a | 3348..3370 | + | Transgenic insert |
| TI-454 | CTTG | FnCas12a | 3351..3373 | − | Transgenic insert |
| TI-455 | TTTG | LbCas12a | 3367..3389 | + | Transgenic insert |
| TI-456 | TTTC | LbCas12a | 3383..3405 | − | Transgenic insert |
| TI-457 | TATA | LbCas12a- | 3387..3409 | − | Transgenic insert |
| | | TAT | | | |
| TI-458 | TATA | LbCas12a- | 3389..3411 | − | Transgenic insert |
| | | TAT | | | |
| TI-459 | TTTC | LbCas12a | 3402..3424 | + | Transgenic insert |
| TI-460 | TTTC | LbCas12a | 3406..3428 | + | Transgenic insert |
| TI-461 | TTCA | LbCas12a- | 3407..3429 | + | Transgenic insert |
| | | TYC | | | |
| TI-462 | TTTG | LbCas12a | 3435..3457 | + | Transgenic insert |
| TI-463 | TTTG | LbCas12a | 3439..3461 | + | Transgenic insert |
| TI-464 | TTTC | LbCas12a | 3455..3477 | + | Transgenic insert |
| TI-465 | TCCA | LbCas12a- | 3462..3484 | − | Transgenic insert |
| | | TYC | | | |
| TI-466 | TTTA | LbCas12a | 3487..3509 | − | Transgenic insert |
| TI-467 | TTTG | LbCas12a | 3488..3510 | + | Transgenic insert |
| TI-468 | TCCA | LbCas12a- | 3491..3513 | − | Transgenic insert |
| | | TYC | | | |
| TI-469 | TTCA | LbCas12a- | 3495..3517 | − | Transgenic insert |
| | | TYC | | | |
| TI-470 | TTTC | LbCas12a | 3496..3518 | − | Transgenic insert |
| TI-471 | TTCA | LbCas12a- | 3502..3524 | − | Transgenic insert |
| | | TYC | | | |
| TI-472 | ATTC | FnCas12a | 3503..3525 | − | Transgenic insert |
| TI-473 | TTTA | LbCas12a- | 3506..3528 | − | Transgenic insert |
| | | TYC | | | |
| TI-474 | TTTG | LbCas12a | 3519..3541 | + | Transgenic insert |
| TI-475 | TTTC | LbCas12a | 3523..3545 | + | Transgenic insert |
| TI-476 | CTTG | FnCas12a | 3542..3564 | + | Transgenic insert |
| TI-477 | TTTG | LbCas12a | 3548..3570 | + | Transgenic insert |
| TI-478 | TTTC | LbCas12a | 3581..3603 | + | Transgenic insert |
| TI-479 | TTTG | LbCas12a | 3597..3619 | + | Transgenic insert |
| TI-480 | TTTG | LbCas12a | 3601..3623 | + | Transgenic insert |
| TI-481 | CTTG | FnCas12a | 3605..3627 | + | Transgenic insert |
| TI-482 | TTTC | LbCas12a | 3609..3631 | + | Transgenic insert |
| TI-483 | TATA | LbCas12a- | 3610..3632 | − | Transgenic insert |
| | | TAT | | | |
| TI-484 | TATA | LbCas12a- | 3615..3637 | + | Transgenic insert |
| | | TAT | | | |
| TI-485 | TTCA | LbCas12a- | 3619..3641 | − | Transgenic insert |
| | | TYC | | | |
| TI-486 | CTTC | FnCas12a | 3620..3642 | − | Transgenic insert |
| TI-487 | GTTC | FnCas12a | 3623..3645 | − | Transgenic insert |
| TI-488 | TTTA | LbCas12a | 3638..3660 | − | Transgenic insert |
| TI-489 | TTTA | LbCas12a | 3641..3663 | + | Transgenic insert |
| TI-490 | TCCC | LbCas12a- | 3645..3667 | − | Transgenic insert |
| | | TYC | | | |
| TI-491 | TATC | LbCas12a- | 3647..3669 | − | Transgenic insert |
| | | TAT | | | |
| TI-492 | GTTA | FnCas12a | 3649..3671 | − | Transgenic insert |
| TI-493 | ATTA | FnCas12a | 3657..3679 | − | Transgenic insert |
| TI-494 | TTTG | LbCas12a | 3674..3696 | − | Transgenic insert |
| TI-495 | TTTA | LbCas12a | 3693..3715 | − | Transgenic insert |
| TI-496 | TTTA | LbCas12a | 3696..3718 | + | Transgenic insert |
| TI-497 | GTTG | FnCas12a | 3711..3733 | + | Transgenic insert |
| 3J-1 | TTTG | LbCas12a | 3716..3738 | + | 3′ Junction |
| 3J-2 | GTTG | FnCas12a | 3723..3745 | − | 3′ Junction |
| 3J-3 | TTTG | LbCas12a | 3726..3748 | − | 3′ Junction |
| 3F-1 | GTTA | FnCas12a | 3739..3761 | − | 3′ Flanking genomic |
| | | | | | DNA |
| 3F-2 | TTTG | LbCas12a | 3746..3768 | − | 3′ Flanking genomic |
| | | | | | DNA |
| 3F-3 | ATTA | FnCas12a | 3768..3790 | − | 3′ Flanking genomic |
| | | | | | DNA |
| 3F-4 | ATTG | FnCas12a | 3787..3809 | − | 3′ Flanking genomic |
| | | | | | DNA |
| 3F-5 | TCCA | LbCas12a- | 3790..3812 | − | 3′ Flanking genomic |
| | | TYC | | | DNA |
| 3F-6 | TATC | LbCas12a- | 3792..3814 | − | 3′ Flanking genomic |
| | | TAT | | | DNA |
| 3F-7 | TTTA | LbCas12a | 3794..3816 | − | 3′ Flanking genomic |
| | | | | | DNA |
| 3F-8 | TTTA | LbCas12a | 3824..3846 | − | 3′ Flanking genomic |
| | | | | | DNA |
| 3F-9 | TCCC | LbCas12a- | 3828..3850 | − | 3′ Flanking genomic |
| | | TYC | | | DNA |
| 3F-10 | ATTG | FnCas12a | 3833..3855 | − | 3′ Flanking genomic |
| | | | | | DNA |
| 3F-11 | TTCC | LbCas12a- | 3842..3864 | − | 3′ Flanking genomic |
| | | TYC | | | DNA |
| 3F-12 | CTTC | FnCas12a | 3843..3865 | − | 3′ Flanking genomic |
| | | | | | DNA |
| 3F-13 | GTTC | FnCas12a | 3846..3868 | − | 3′ Flanking genomic |
| | | | | | DNA |
| 3F-14 | TATG | LbCas12a- | 3849..3871 | − | 3′ Flanking genomic |
| | | TAT | | | DNA |
| 3F-15 | TTTA | LbCas12a | 3865..3887 | + | 3′ Flanking genomic |
| | | | | | DNA |
| 3F-16 | GTTG | FnCas12a | 3871..3893 | + | 3′ Flanking genomic |
| | | | | | DNA |
| 3F-17 | TCCC | LbCas12a- | 3879..3901 | − | 3′ Flanking genomic |
| | | TYC | | | DNA |
| 3F-18 | GTTC | FnCas12a | 3894..3916 | + | 3′ Flanking genomic |
| | | | | | DNA |
| 3F-19 | TTCA | LbCas12a- | 3895..3917 | + | 3′ Flanking genomic |
| | | TYC | | | DNA |
| 3F-20 | TCCG | LbCas12a- | 3902..3924 | + | 3′ Flanking genomic |
| | | TYC | | | DNA |
| 3F-21 | TCCG | LbCas12a- | 3917..3939 | + | 3′ Flanking genomic |
| | | TYC | | | DNA |
| 3F-22 | CTTC | FnCas12a | 3923..3945 | + | 3′ Flanking genomic |
| | | | | | DNA |
| 3F-23 | TTCA | LbCas12a- | 3934..3956 | − | 3′ Flanking genomic |
| | | TYC | | | DNA |
| 3F-24 | CTTC | FnCas12a | 3935..3957 | − | 3′ Flanking genomic |
| | | | | | DNA |
| 3F-25 | GTTC | FnCas12a | 3948..3970 | + | 3′ Flanking genomic |
| | | | | | DNA |
| 3F-26 | TCCA | LbCas12a- | 3952..3974 | + | 3′ Flanking genomic |
| | | TYC | | | DNA |
| 3F-27 | TTTG | LbCas12a | 3956..3978 | − | 3′ Flanking genomic |
| | | | | | DNA |
| 3F-28 | CTTG | FnCas12a | 3964..3986 | − | 3′ Flanking genomic |
| | | | | | DNA |
| 3F-29 | TCCC | LbCas12a- | 3967..3989 | − | 3′ Flanking genomic |
| | | TYC | | | DNA |
| 3F-30 | TTCC | LbCas12a- | 3968..3990 | − | 3′ Flanking genomic |
| | | TYC | | | DNA |
| 3F-31 | CTTC | FnCas12a | 3969..3991 | − | 3′ Flanking genomic |
| | | | | | DNA |
| 3F-32 | TTCG | LbCas12a- | 4001..4023 | − | 3′ Flanking genomic |
| | | TYC | | | DNA |
| 3F-33 | GTTC | FnCas12a | 4002..4024 | − | 3′ Flanking genomic |
| | | | | | DNA |
| 3F-34 | CTTG | FnCas12a | 4017..4039 | + | 3′ Flanking genomic |
| | | | | | DNA |
| 3F-35 | ATTC | FnCas12a | 4029..4051 | + | 3′ Flanking genomic |
| | | | | | DNA |
| 3F-36 | TTCA | LbCas12a- | 4030..4052 | + | 3′ Flanking genomic |
| | | TYC | | | DNA |
| 3F-37 | ATTG | FnCas12a | 4052..4074 | − | 3′ Flanking genomic |
| | | | | | DNA |
| 3F-38 | TTTC | LbCas12a | 4053..4075 | + | 3′ Flanking genomic |
| | | | | | DNA |
| 3F-39 | TTCC | LbCas12a- | 4054..4076 | + | 3′ Flanking genomic |
| | | TYC | | | DNA |
| 3F-40 | TCCA | LbCas12a- | 4055..4077 | + | 3′ Flanking genomic |
| | | TYC | | | DNA |
| 3F-41 | GTTC | FnCas12a | 4063..4085 | + | 3′ Flanking genomic |
| | | | | | DNA |
| 3F-42 | TCCG | LbCas12a- | 4071..4093 | + | 3′ Flanking genomic |
| | | TYC | | | DNA |
| 3F-43 | ATTC | FnCas12a | 4082..4104 | + | 3′ Flanking genomic |
| | | | | | DNA |
| 3F-44 | TTCC | LbCas12a- | 4083..4105 | + | 3′ Flanking genomic |
| | | TYC | | | DNA |
| 3F-45 | TCCC | LbCas12a- | 4084..4106 | + | 3′ Flanking genomic |
| | | TYC | | | DNA |
| 3F-46 | TATG | LbCas12a- | 4094..4116 | + | 3′ Flanking genomic |
| | | TAT | | | DNA |
| 3F-47 | GTTA | FnCas12a | 4111..4133 | + | 3′ Flanking genomic |
| | | | | | DNA |
| 3F-48 | TATG | LbCas12a- | 4113..4135 | + | 3′ Flanking genomic |
| | | TAT | | | DNA |
| 3F-49 | GTTG | FnCas12a | 4132..4154 | − | 3′ Flanking genomic |
| | | | | | DNA |
| 3F-50 | TTTA | LbCas12a | 4161..4183 | + | 3′ Flanking genomic |
| | | | | | DNA |
| 3F-51 | ATTC | FnCas12a | 4164..4186 | + | 3′ Flanking genomic |
| | | | | | DNA |
| 3F-52 | TCCC | LbCas12a- | 4164..4186 | − | 3′ Flanking genomic |
| | | TYC | | | DNA |
| 3F-53 | TTCA | LbCas12a- | 4165..4187 | + | 3′ Flanking genomic |
| | | TYC | | | DNA |
| 3F-54 | TCCA | LbCas12a- | 4174..4196 | + | 3′ Flanking genomic |
| | | TYC | | | DNA |
| 3F-55 | GTTC | FnCas12a | 4179..4201 | + | 3′ Flanking genomic |
| | | | | | DNA |
| 3F-56 | TCCC | LbCas12a- | 4183..4205 | − | 3′ Flanking genomic |
| | | TYC | | | DNA |
| 3F-57 | TCCC | LbCas12a- | 4187..4209 | − | 3′ Flanking genomic |
| | | TYC | | | DNA |
| 3F-58 | TATO | LbCas12a- | 4191..4213 | − | 3′ Flanking genomic |
| | | TAT | | | DNA |
| 3F-59 | ATTA | FnCas12a | 4199..4221 | + | 3′ Flanking genomic |
| | | | | | DNA |
| 3F-60 | TCCA | LbCas12a- | 4213..4235 | + | 3′ Flanking genomic |
| | | TYC | | | DNA |
| 3F-61 | CTTG | FnCas12a | 4219..4241 | + | 3′ Flanking genomic |
| | | | | | DNA |
| 3F-62 | GTTG | FnCas12a | 4221..4243 | − | 3′ Flanking genomic |
| | | | | | DNA |
| 3F-63 | GTTG | FnCas12a | 4222..4244 | + | 3′ Flanking genomic |
| | | | | | DNA |
| 3F-64 | CTTG | FnCas12a | 4228..4250 | − | 3′ Flanking genomic |
| | | | | | DNA |
| 3F-65 | TCCA | LbCas12a- | 4232..4254 | − | 3′ Flanking genomic |
| | | TYC | | | DNA |
| 3F-66 | GTTG | FnCas12a | 4241..4263 | + | 3′ Flanking genomic |
| | | | | | DNA |
| 3F-67 | TTTG | LbCas12a | 4249..4271 | + | 3′ Flanking genomic |
| | | | | | DNA |
| 3F-68 | GTTG | FnCas12a | 4252..4274 | + | 3′ Flanking genomic |
| | | | | | DNA |
| 3F-69 | CTTC | FnCas12a | 4266..4288 | − | 3′ Flanking genomic |
| | | | | | DNA |
| 3F-70 | TTCG | LbCas12a- | 4271..4293 | − | 3′ Flanking genomic |
| | | TYC | | | DNA |
| 3F-71 | CTTC | FnCas12a | 4272..4294 | − | 3′ Flanking genomic |
| | | | | | DNA |
| 3F-72 | CTTG | FnCas12a | 4283..4305 | − | 3′ Flanking genomic |
| | | | | | DNA |
| 3F-73 | TCCC | LbCas12a- | 4295..4317 | + | 3′ Flanking genomic |
| | | TYC | | | DNA |
| 3F-74 | GTTG | FnCas12a | 4317..4339 | + | 3′ Flanking genomic |
| | | | | | DNA |
| 3F-75 | TCCC | LbCas12a- | 4331..4353 | − | 3′ Flanking genomic |
| | | TYC | | | DNA |
| 3F-76 | ATTG | FnCas12a | 4341..4363 | − | 3′ Flanking genomic |
| | | | | | DNA |
| 3F-77 | TCCG | LbCas12a- | 4346..4368 | − | 3′ Flanking genomic |
| | | TYC | | | DNA |
| 3F-78 | TCCG | LbCas12a- | 4352..4374 | − | 3′ Flanking genomic |
| | | TYC | | | DNA |
| 3F-79 | TCCG | LbCas12a- | 4355..4377 | + | 3′ Flanking genomic |
| | | TYC | | | DNA |
| 3F-80 | ATTA | FnCas12a | 4360..4382 | + | 3′ Flanking genomic |
| | | | | | DNA |
| 3F-81 | GTTG | FnCas12a | 4371..4393 | + | 3′ Flanking genomic |
| | | | | | DNA |
| 3F-82 | TTCA | LbCas12a- | 4373..4395 | − | 3′ Flanking genomic |
| | | TYC | | | DNA |
| 3F-83 | GTTC | FnCas12a | 4374..4396 | − | 3′ Flanking genomic |
| | | | | | DNA |
| 3F-84 | CTTG | FnCas12a | 4384..4406 | − | 3′ Flanking genomic |
| | | | | | DNA |
| 3F-85 | TTTG | LbCas12a | 4495..4517 | − | 3′ Flanking genomic |
| | | | | | DNA |
| 3F-86 | TTTG | LbCas12a | 4529..4551 | + | 3′ Flanking genomic |
| | | | | | DNA |
| 3F-87 | TTTA | LbCas12a | 4541..4563 | + | 3′ Flanking genomic |
| | | | | | DNA |
| 3F-88 | TATA | LbCas12a- | 4549..4571 | + | 3′ Flanking genomic |
| | | TAT | | | DNA |
| 3F-89 | TTTA | LbCas12a | 4554..4576 | + | 3′ Flanking genomic |
| | | | | | DNA |
| 3F-90 | TATA | LbCas12a- | 4554..4576 | − | 3′ Flanking genomic |
| | | TAT | | | DNA |
| 3F-91 | ATTA | FnCas12a | 4557..4579 | + | 3′ Flanking genomic |
| | | | | | DNA |
| 3F-92 | TATA | LbCas12a- | 4559..4581 | + | 3′ Flanking genomic |
| | | TAT | | | DNA |
| 3F-93 | ATTA | FnCas12a | 4562..4584 | + | 3′ Flanking genomic |
| | | | | | DNA |
| 3F-94 | TATC | LbCas12a- | 4564..4586 | + | 3′ Flanking genomic |
| | | TAT | | | DNA |
| 3F-95 | TTCA | LbCas12a- | 4569..4591 | + | 3′ Flanking genomic |
| | | TYC | | | DNA |
| 3F-96 | CTTA | FnCas12a | 4576..4598 | + | 3′ Flanking genomic |
| | | | | | DNA |
| 3F-97 | TTTG | LbCas12a | 4580..4602 | + | 3′ Flanking genomic |
| | | | | | DNA |
| 3F-98 | TCCA | LbCas12a- | 4582..4604 | − | 3′ Flanking genomic |
| | | TYC | | | DNA |
| 3F-99 | TTCC | LbCas12a- | 4583..4605 | − | 3′ Flanking genomic |
| | | TYC | | | DNA |
| 3F-100 | TTTC | LbCas12a | 4584..4606 | − | 3′ Flanking genomic |
| | | | | | DNA |
| 3F-101 | TTTG | LbCas12a | 4585..4607 | + | 3′ Flanking genomic |
| | | | | | DNA |
| 3F-102 | TTTG | LbCas12a | 4588..4610 | − | 3′ Flanking genomic |
| | | | | | DNA |
| 3F-103 | GTTA | FnCas12a | 4593..4615 | − | 3′ Flanking genomic |
| | | | | | DNA |
| 3F-104 | TATG | LbCas12a- | 4596..4618 | − | 3′ Flanking genomic |
| | | TAT | | | DNA |
| 3F-105 | TATA | LbCas12a- | 4598..4620 | − | 3′ Flanking genomic |
| | | TAT | | | DNA |
| 3F-106 | TATA | LbCas12a- | 4603..4625 | + | 3′ Flanking genomic |
| | | TAT | | | DNA |
| 3F-107 | TTTC | LbCas12a | 4610..4632 | + | 3′ Flanking genomic |
| | | | | | DNA |
| 3F-108 | TTCA | LbCas12a- | 4611..4633 | + | 3′ Flanking genomic |
| | | TYC | | | DNA |
| 3F-109 | TTTG | LbCas12a | 4613..4635 | − | 3′ Flanking genomic |
| | | | | | DNA |
| 3F-110 | ATTA | FnCas12a | 4621..4643 | + | 3′ Flanking genomic |
| | | | | | DNA |
| 3F-111 | ATTG | FnCas12a | 4628..4650 | − | 3′ Flanking genomic |
| | | | | | DNA |
| 3F-112 | TATA | LbCas12a- | 4632..4654 | − | 3′ Flanking genomic |
| | | TAT | | | DNA |
| 3F-113 | TTTA | LbCas12a | 4639..4661 | − | 3′ Flanking genomic |
| | | | | | DNA |
| 3F-114 | ATTA | FnCas12a | 4649..4671 | + | 3′ Flanking genomic |
| | | | | | DNA |
| 3F-115 | TTTC | LbCas12a | 4653..4675 | + | 3′ Flanking genomic |
| | | | | | DNA |
| 3F-116 | ATTA | FnCas12a | 4665..4687 | + | 3′ Flanking genomic |
| | | | | | DNA |
| 3F-117 | TTCA | LbCas12a- | 4666..4688 | − | 3′ Flanking genomic |
| | | TYC | | | DNA |
| 3F-118 | GTTC | FnCas12a | 4667..4689 | − | 3′ Flanking genomic |
| | | | | | DNA |
| 3F-119 | TTTG | LbCas12a | 4674..4696 | − | 3′ Flanking genomic |
| | | | | | DNA |
| 3F-120 | CTTA | FnCas12a | 4681..4703 | − | 3′ Flanking genomic |
| | | | | | DNA |
| 3F-121 | TATG | LbCas12a- | 4693..4715 | − | 3′ Flanking genomic |
| | | TAT | | | DNA |
| 3F-122 | ATTA | FnCas12a | 4695..4717 | − | 3′ Flanking genomic |
| | | | | | DNA |
| 3F-123 | GTTG | FnCas12a | 4701..4723 | − | 3′ Flanking genomic |
| | | | | | DNA |

gRNAs may include a G leader sequence to facilitate transcription start from a pol III promoter positioned 5′ to a gRNA repeat, resulting in GAATTTCTACTAAGTGTAGAT (SEQ ID NO: 26) for LbCas12a, or GTAATTTCTACTGTTGTAGAT (SEQ ID NO: 27) for FnCas12a and an OgRRS sequence (as shown in the fourth column of Table 11). For Agrobacterium-based plant expression vectors, the cassette further comprises a poly-T transcript termination region (TTTTTTT). These gRNAs can be used to target the Cas12a nuclease to cut within both the OgRRS and CgRRS sequences. Illustrative examples of such gRNAs for FnCas 12a expressed in stable plants are shown in Table 12, wherein the gRNA repeat of TAATTTCTACTGTTGTAGAT is underlined, and the poly-T transcript termination sequence of TTTTTTT is shown in italic font.

TABLE 12
Illustrative examples of gRNAs
useful in targeting FnCas12a nuclease

| gRNA | Sequence |
|---|---|
| gRNA_5F-63 | GTAATTTCTACTGTTGTAGAT |
| (SEQ ID NO: 28) | AGTTTGCAAATTCCGGGACCT |
| | GGTTTTTTT |
| gRNA_3F-4 | GTAATTTCTACTGTTGTAGAT |
| (SEQ ID NO: 29) | ATGCACCACCCTAGCATTACT |
| | CTTTTTTTT |

Any of the OgRRS sequences presented in Table 11 can be used alternatively as a site to insert a CgRRS that is designed using a different OgRRS—i.e., an OgRRS can be used as a target site to introduce a cognate target site or CgRRS. For example, a CgRRS derived from an OgRRS in a flanking sequence can be inserted into a flanking sequence on the opposite side of the transgenic insertion of event ZM_BCS216090 to allow for the excision or deletion of the entire transgenic insertion of event ZM_BCS216090. To illustrate this approach, OgRRS 3F-4 is selected as the OgRRS that can be used to design a corresponding CgRRS 3F-4 comprising DNA fragment, and OgRRS 5F-63 is selected as the target site into which the CgRRS 3F-4 comprising DNA fragment is inserted. Using a Cas12a editing system such as with the Fn Cas12a endonuclease, the OgRRS 5F-63 site is targeted using the gRNA, gRNA_5F-63 presented in Table 12 to cut within the OgRRS 5F-63 site. The CgRRS 3F-4 comprising DNA fragment that comprises the OgRRS 3F-4 target site is then inserted within the cut site that was introduced into the OgRRS 5F-63 sequence. After selection of a transgenic event comprising the introduced CgRRS 3F-4 site, the event can be bred into another germplasm. When desired, the transgenic insert of ZM_BCS216090 can be excised or deleted from the plant using a Cas 12a editing system and the gRNA_3F-4 as presented in Table 12.

A CgRRS can be introduced into the transgenic insertion locus through multiple methods using a CRISPR system. For example, a CRISPR system can be utilized for targeting 5′ insertion of a blunt-end double-stranded DNA fragment into a genomic target site of interest such as an OgRRS that is not the OgRRS that has been selected for the design of the CgRRS. The CRISPR-mediated endonuclease activity can introduce a double stand break (DSB) in the selected genomic target site and DNA repair, such as microhomology-driven nonhomologous end-joining DNA repair, results in insertion of the blunt-end double-stranded DNA fragment into the DSB. Blunt-end double-stranded DNA fragments can be designed with 1-10 bp of microhomology, on both the 5′ and 3′ ends of the DNA fragment, that correspond to the 5′ and 3′-flanking sequence at the cut site of the protospacer in the genomic target site.

The CRISPR system or any other site-directed nuclease can be introduced into a plant, plant part or plant cell containing event ZM_BCS216090 by several methods, including but not limited to Agrobacterium-mediated transformation, polyethylene glycol-mediated transformation, biolistic bombardment or delivery, liposome-mediated transfection, viral transduction, one or more delivery particles, microinjection, or electroporation. One or more expression cassettes encoding the gRNA and/or CRISPR associated protein components of a Type I, Type II, Type III, Type IV, Type V, or Type VI CRISPR-Cas system or any other site-directed nuclease can be transformed or transiently introduced into a plant cell. The CRISPR associated protein and guide RNA can be synthesized and assembled in vitro to form a ribonucleoprotein complex (RNP). The site-directed nuclease or ribonucleoprotein along with a DNA fragment encoding the CgRRS can be provided to the plant by several methods, including but not limited to polyethylene glycol-mediated transformation, biolistic bombardment or delivery, liposome-mediated transfection, one or more delivery particles, microinjection, or electroporation. The introduced site-directed nuclease and/or one or more gRNAs, and/or one or more expression cassettes or constructs encoding the gRNA and/or CRISPR associated protein or site-directed nuclease, along with a DNA fragment comprising the CgRRS is provided in sufficient quantity to modify the genome of a plant cell but does not persist after one or more cell divisions or in a subsequent progeny or generation. In such embodiments, no further steps may be needed to remove or segregate the one or more expression cassettes encoding the gRNA and/or CRISPR associated protein from the modified cell. Double-stranded DNA fragments can also be transformed or transiently introduced into a plant cell along with one or more constructs or expression cassettes encoding a site-directed nuclease or gRNA and/or CRISPR associated protein. The introduced double-stranded DNA fragments are provided in sufficient quantity to modify the plant cell without persisting after one or more cell divisions or in a subsequent progeny or generation.

Alternatively, an expression construct comprising one or more expression cassettes for the expression of a site-directed nuclease and/or one or more gRNAs, and an expression construct or cassette encoding a site-directed nuclease or a Type I, Type II, Type III, Type IV, Type V, or Type VI CRISPR associated protein is stably transformed into a plant, plant part or plant cell comprising event ZM_BCS216090 to modify the plant, plant part or plant cell in the targeted region of the transgene insertion locus, to introduce the CgRRS at or near the desired target locus.

### Example 6

Modification of Corn Event ZM_BCS216090 with Genome Editing Techniques Using Two Guide RNAs

This example describes how all or part of the transgenic insertion present in corn event ZM_BCS216090, and/or a flanking genomic DNA segment(s), may be altered, excised or modified, such as by making one or more insertions, deletions, substitutions, inversions, transpositions, mutations or other genetic modifications, at or near a target, hybridization or recognition site(s) or sequence(s), which may be referred to jointly as a target site(s), using a targeted genome editing technique with two or more site-specific nucleases or two or more guide RNAs. According to present embodiments, a targeted genome editing technique may comprise use of a site-specific nuclease, such as a meganuclease, a zinc-finger nuclease (ZFN), an RNA-guided endonuclease (e.g., the CRISPR/Cas9 system), a TALE-endonuclease (TALEN), a recombinase, or a transposase. For example, genetic modifications can be made by targeted genome editing techniques and methods using a Clustered Regularly Interspersed Short Palindromic Repeats (CRISPR) editing system with two or more guide RNAs. Target sites useful for alteration, excision or modification of all or part of the event ZM_BCS216090 transgenic insertion, the expression cassette within SEQ ID NO:9 or SEQ ID NO:10, and/or a flanking sequence(s) at or near event ZM_BCS216090 can be used with a site-specific nuclease or a guide RNA(s), such as two or more guide RNAs, specific for such target site(s) to create a genetic modification (e.g., an excision, mutation or edit) at, near or between such target site(s). According to present embodiments, a targeted genome editing technique may comprise use of a site-specific nuclease, such as a meganuclease, a zinc-finger nuclease (ZFN), an RNA-guided endonuclease (e.g., the CRISPR/Cas9 system), a TALE-endonuclease (TALEN), a recombinase, or a transposase. For example, genetic modifications can be made by targeted genome editing techniques and methods using a CRISPR editing system with guide RNA(s), such as two or more guide RNAs. Two or more target sites useful for alteration, excision or modification of all or part of the event ZM_BCS216090 transgenic insertion, the expression cassette within SEQ ID NO:9 or SEQ ID NO:10, and/or a flanking sequence(s) at or near event ZM_BCS216090 can be used with a site-specific nuclease or a CRISPR system and guide RNA(s), such as two or more guide RNAs, specific for such target site(s) to create a genetic modification (e.g., an excision, mutation or edit) at, near or between such target site(s).

According to some embodiments, a polynucleotide segment or sequence spanning all or part of the event ZM_BCS216090 transgenic insertion, the event ZM_BCS216090 junction sequence(s), the miRNA expression cassette within SEQ ID NO:9 or SEQ ID NO:10, and/or a flanking sequence(s) at or near event ZM_BCS216090 can be excised or deleted using two or more guide RNAs with target sites having the same or different target sequence(s) that are present on both sides (i.e., on the 5′ and 3′ sides) of the polynucleotide segment or sequence to be excised or deleted. Such target sites may each be present in the flanking genomic DNA or introduced by the transgenic insertion of event ZM_BCS216090, either at the 5′ or 3′ junction or within the transgenic insertion itself. In one embodiment, a CRISPR nuclease system cleaves at two different guide RNA target sites thereby permitting the excision or deletion of the intervening sequence. According to some embodiments, one of the target sites, a first target site, may be present in a 5′ or 3′ flanking sequence, or a 5′ flank or a 3′ flank, and the other target site, a second target site, may be present within the transgenic insertion or at the 5′ or 3′ junction. Alternatively, one of the target sites, a first target site, may be present in a 5′ flanking sequence or a 5′ flank, and the other target site, a second target site, may be present in a 3′ flanking sequence or a 3′ flank. As another alternative, one of the target sites, a first target site, may be present within the transgenic insertion or at the 5′ junction, and the other target site, a second target site, may be present within the transgenic insertion or at the 3′ junction. According to some embodiments, two different guide RNAs and target sites and/or two different site-directed nucleases could be used. According to some embodiments, a target site may comprise an originator target site, a cognate target site, a OgRRS or a CgRRS.

Two functional guide RNAs (gRNAs) for a site-specific nuclease(s) or an RNA guided nuclease(s) or CRISPR system can be introduced, created or expressed to excise or delete a polynucleotide segment or sequence spanning all or part of the event ZM_BCS216090 transgenic insertion, the event ZM_BCS216090 junction sequence(s), the miRNA expression cassette within SEQ ID NO:9 or SEQ ID NO:10, and/or a flanking sequence(s) at or near event ZM_BCS216090. For example, a fragment of DNA corresponding to either the entire transgenic insertion or miRNA expression cassette of event ZM_BCS216090, or a fragment within the transgenic insertion of event ZM_BCS216090, such as the expression cassette or genetic or regulatory element within the transgene cassette of event ZM_BCS216090, can be excised or deleted between the two or more target sites. For example, to excise or delete the entire transgenic insertion of event ZM_BCS216090, a first gRNA may target an area in the 5′ flanking genomic sequence, such as 5F-63 (Table 12), and a second gRNA may target a region in the 3′ flanking genomic sequence, such as 3F-4 (Table 12).

Site-specific nuclease(s) and/or two or more guide RNAs, or an expression cassette(s) or construct(s) encoding site-specific nuclease(s) and/or two or more guide RNAs, can be introduced into a plant, plant part or plant cell containing event ZM_BCS216090 by several methods, including but not limited to Agrobacterium-mediated transformation, polyethylene glycol-mediated transformation, biolistic bombardment or delivery, liposome-mediated transfection, viral transduction, one or more delivery particles, microinjection, or electroporation. One or more expression cassettes encoding the gRNAs and/or CRISPR associated protein components or enzymes of a Type I, Type II, Type III, Type IV, Type V, or Type VI CRISPR-Cas system or any other site-directed nuclease(s) can be transformed or transiently introduced into a plant cell. The CRISPR associated protein and guide RNAs can be synthesized and assembled in vitro to form a ribonucleoprotein complex(s) (RNP(s)). The site-directed nuclease or ribonucleoprotein along with a DNA fragment encoding the gRNAs can be provided to the plant by several methods, including but not limited to polyethylene glycol-mediated transformation, biolistic bombardment or delivery, liposome-mediated transfection, one or more delivery particles, microinjection, or electroporation. Two or more expression cassettes and/or constructs may be introduced into a plant cell may comprise a first expression cassette and a second expression cassette. A first expression cassette may comprise a plant expressible promoter operably linked to a polynucleotide encoding a Cas12a or other RNA guided nuclease. A second expression cassette may comprise a plant expressible promoter operably linked to a polynucleotide encoding one or more gRNAs. A third expression cassette may comprise a plant expressible promoter operably linked to a polynucleotide encoding one or more gRNAs, which may be the same or different than the polynucleotide and gRNAs encoded by the first expression cassette. For example, an expression cassette may comprise a Polymerase III or Polymerase II promoters operable in a plant cell operably linked to polynucleotides encoding the two gRNAs, such as a gRNA described herein that is specific for a target sequence, such as those described in Tables 11 or 12. Another expression cassette may encode a selection marker gene, such as aadA for conferring resistance to spectinomycin and/or streptomycin.

1. A recombinant DNA molecule comprising:
(a) a first nucleotide sequence (i) comprising an expression cassette that encodes a microRNA (miRNA) that suppresses the expression of the endogenous GA20ox3 and GA20ox5 genes in corn, or (ii) selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9; and
(b) a second nucleotide sequence that
(i) comprises at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 17 or 19,
(ii) is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 17 or 19,
(iii) is selected from the group consisting of SEQ ID NOs: 30-49 and SEQ ID NOs: 50-129,
(iv) comprises at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 18 or 20,
(v) is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 18 or 20, or
(vi) is selected from the group consisting of SEQ ID NOs: 130-149 and SEQ ID NOS: 150-229.

2. The recombinant DNA molecule of claim 1, further comprising:
(c) a third nucleotide sequence that
(i) comprises at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 18 or 20, or
(ii) is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 18 or 20, or
(iii) is selected from the group consisting of SEQ ID NOs: 130-149 and SEQ ID NOs: 150-229.

3. The recombinant DNA molecule of claim 2,
(i) wherein the third nucleotide sequence is selected from the group consisting of SEQ ID NOs: 130-149 and SEQ ID NOs: 150-229, or
(ii) wherein the recombinant DNA molecule has a deletion of one or more consecutive nucleotides between the second nucleotide sequence and the third nucleotide sequence relative to SEQ ID NO: 10, 18 or 20.

4. (canceled)

5. The recombinant DNA molecule of claim 1,
(i) wherein the second nucleotide sequence is selected from the group consisting of SEQ ID NOs: 30-49 and SEQ ID NOs: 50-129,
(ii) wherein the recombinant DNA molecule has a deletion of one or more consecutive nucleotides between the first nucleotide sequence and the second nucleotide sequence relative to SEQ ID NO: 10, 17 or 19,
(iii) wherein the recombinant DNA molecule further comprises nucleotides 1,000-1,001 or 3,733-3,734 of SEQ ID NO: 10,
(iv) wherein said recombinant DNA molecule is comprised in a corn plant, corn plant part, corn plant cell, corn plant seed, corn progeny plant, or commodity or fuel product made from corn and corn plant parts,
(v) wherein said recombinant DNA molecule comprises an amplicon diagnostic for the presence of DNA comprising a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9, and/or
(vi) wherein the recombinant DNA molecule is derived from a corn plant, corn plant part, corn seed, processed corn seed, corn plant cell or tissue, animal feed comprising corn, corn oil, corn meal, corn flour, corn flakes, corn bran, food made comprising corn, corn biomass, or fuel products made from corn and corn plant parts.

6-13. (canceled)

14. A recombinant DNA molecule comprising a polynucleotide segment of sufficient length to function as a DNA probe that hybridizes specifically under stringent hybridization conditions with a polynucleotide having a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 17, SEQ ID NO: 18, SEQ ID NO: 19, and SEQ ID NO: 20.

15. A pair of DNA molecules that can function as DNA primers when used together in an amplification reaction comprising a first DNA molecule and a second DNA molecule, wherein the first DNA molecule and the second DNA molecule are different,
(a) wherein the first DNA molecule is:
(i) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9, or a complement thereof, or
(ii) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 17 or 19, or a complement thereof; and
(b) wherein the second DNA molecule is:
(i) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9, or a complement thereof, or
(ii) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 17 or 19, or a complement thereof, or
(iii) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 18 or 20, or a complement thereof.

16. The pair of DNA molecules of claim 15,
(i) wherein the first DNA molecule and the second DNA molecule can be used together in an amplification reaction with a sample from a plant, plant part, plant seed, plant cell, food or animal feed, or commodity or fuel product made from a plant or plant part to produce an amplicon diagnostic for the presence of a modified corn event ZM_BCS216090 DNA in said sample, and/or
(ii) wherein the amplicon comprises a nucleotide sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

17. (canceled)

18. The pair of DNA molecules of claim 16- or 17, wherein the modified corn event ZM_BCS216090 DNA is a further modified corn event ZM_BCS216090 DNA.

19. A method of detecting the presence of a DNA segment diagnostic for a modified corn event ZM_BCS216090 DNA in a sample, said method comprising:
a) contacting said sample with the DNA molecule of claim 14;
b) subjecting said sample and said DNA molecule to stringent hybridization conditions; and
c) detecting hybridization of said DNA molecule to said DNA segment in said sample,
wherein said detection is diagnostic for the presence of said modified corn event ZM_BCS216090 DNA in said sample.

20. A method of detecting the presence of a DNA segment diagnostic for a modified corn event ZM_BCS216090 DNA in a sample, said method comprising:
a) contacting said sample with the pair of DNA molecules of claim 15;
b) performing an amplification reaction sufficient to produce a DNA amplicon; and
c) detecting the presence of said DNA amplicon in said reaction,
wherein the presence of said DNA amplicon is diagnostic for the presence of said modified corn event ZM_BCS216090 DNA in said sample.

21. A method of detecting the presence of a DNA segment diagnostic for a modified corn event ZM_BCS216090 DNA in a sample, said method comprising performing a sequencing reaction with the sample, wherein the production in the sequencing reaction of a target nucleotide sequence comprising at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9, or a complement thereof, is diagnostic for the modified corn event ZM_BCS216090 DNA in the sample.

22. The method of claim 19, wherein the modified corn event ZM_BCS216090 is a further modified corn event ZM_BCS216090.

23. A modified corn plant, corn plant part, corn seed, or corn cell comprising a modified corn event ZM_BCS216090 or the recombinant DNA molecule of claim 1.

24. The modified corn plant, corn plant part, corn seed, or corn cell of claim 23 comprising:
a) the recombinant DNA molecule; or
b) a recombinant DNA molecule or DNA segment comprising a nucleotide sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10, or a complement thereof; or
c) a recombinant DNA molecule or DNA segment comprising a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 consecutive nucleotides of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10, SEQ ID NO: 17, SEQ ID NO: 18, SEQ ID NO: 19, or SEQ ID NO: 20, or a complement thereof.

25. The modified corn plant, corn plant part, corn seed, or corn cell of claim 23, wherein the corn plant, corn plant part, corn seed, or corn cell exhibits reduced expression of at least a first endogenous gibberellin 20-oxidase (GA20ox) gene.

26. The modified corn plant, corn plant part, corn seed, or corn cell of claim 25,
(i) wherein the gibberellin 20-oxidase (GA20ox) gene is selected from the group consisting of gibberellin 20-oxidase 3 (GA20ox3) and gibberellin 20-oxidase 5 (GA20ox5),
(ii) wherein the modified corn plant, corn plant part, corn seed, or corn cell has reduced expression of an endogenous gibberellin 20-oxidase 3 (GA20ox3) gene and an endogenous gibberellin 20-oxidase 5 (GA20ox5) gene,
(iii) wherein the corn plant, corn plant part, corn seed, or corn cell is further defined as a progeny plant of any generation of a corn plant comprising a modified corn event ZM_BCS216090, or a corn plant part, corn seed, or corn cell derived therefrom,
(iv) wherein said modified corn plant has a reduced plant height relative to a control corn plant,
(v) wherein said modified corn plant has an increased lodging resistance relative to a control corn plant,
(vi) wherein the recombinant DNA molecule is chromosome 1, or the DNA segment is present in chromosome 1 of the modified corn plant, corn plant part, corn seed, or corn cell,
(vii) wherein the modified corn event ZM_BCS216090 is a further modified corn event ZM_BCS216090, and/or
(viii) wherein the modified corn event ZM_BCS216090 of the modified corn plant, corn plant part, corn seed, or corn cell comprises a genetic modification, mutation or edit, relative to the corn event ZM_BCS216090, introduced via a targeted genome editing technique.

27-33. (canceled)

34. A DNA detection kit comprising:
a) the DNA probe of claim 14; and
b) the pair of DNA molecules of claim 15.

35. A method of producing a progeny corn plant comprising a modified corn event ZM_BCS216090 comprising:
a) sexually crossing a first modified corn plant that comprises a modified corn event ZM_BCS216090 with itself or a second corn plant;
b) collecting one or more seeds produced from said cross;
c) growing said seed to produce one or more progeny plants; and
d) selecting at least a first progeny plant or seed comprising a modified corn event ZM_BCS216090.

36. The method of claim 35, wherein the at least a first progeny plant has a reduced plant height and/or increased lodging resistance relative to a control corn plant.

37. The method of claim 35, wherein the modified corn event ZM_BCS216090 is a further modified corn event ZM_BCS216090.

38. The method of claim 35, further comprising:
e) collecting seed from said at least first progeny plant comprising a modified corn event ZM_BCS216090.

39. A hybrid modified corn plant or seed comprising a modified corn event ZM_BCS216090 produced by the method of claim 35.

40. The hybrid modified corn plan or seed of claim 39, wherein the modified corn event ZM_BCS216090 is a further modified corn event ZM_BCS216090.

41. A nonliving corn plant material comprising a detectable amount of the recombinant DNA molecule of claim 1.

42. A microorganism comprising the recombinant DNA molecule of claim 1.

43. The microorganism of claim 42, wherein the microorganism is a plant cell.

44. A commodity product comprising the recombinant DNA molecule of claim 1.

45. The commodity product of claim 44,
(i) wherein said commodity product is produced from a modified corn plant, corn plant part, corn seed, or corn tissue or cell comprising a modified corn event ZM_BCS216090,
(ii) wherein the modified corn event ZM_BCS216090 is a further modified corn event ZM_BCS216090, and/or
(iii) wherein the commodity product is selected from the group consisting of whole or processed corn seed, animal feed comprising corn, corn oil, corn meal, corn flour, corn flakes, corn bran, corn biomass, and fuel products produced using corn and corn plant parts.

46-47. (canceled)

48. A method of producing a commodity product, said method comprising:
a) obtaining a modified corn plant, corn plant part, or corn seed comprising a modified corn event ZM_BCS216090; and
b) producing a commodity product from the transgenic corn plant, corn plant part, or corn seed.

49. The method of claim 48, wherein the modified corn event ZM_BCS216090 is a further modified corn event ZM_BCS216090.

50. A corn plant, corn plant part, or corn seed comprising a DNA molecule or segment functional as a template when tested in a DNA amplification method to produce an amplicon diagnostic for the presence of a modified corn event ZM_BCS216090 DNA.

51. A method of determining the zygosity of a corn plant, corn plant part, or corn seed comprising a modified corn event ZM_BCS216090 comprising:
I:
a) contacting a sample comprising DNA from the corn plant, corn plant part, or corn seed with the pair of DNA molecules of claim 15;
b) performing a nucleic acid amplification reaction with the sample and the pair of DNA molecules; and
c) detecting in the nucleic acid amplification reaction a first amplicon diagnostic for a modified corn event ZM_BCS216090 and a second amplicon diagnostic for native corn genomic DNA not comprising the modified corn event ZM_BCS216090, wherein the presence of only the first amplicon is diagnostic of a corn plant, corn plant part, or corn seed homozygous for the modified corn event ZM_BCS216090, and the presence of both the first amplicon and the second amplicon is diagnostic of a corn plant, corn plant part, or corn seed heterozygous for the modified corn event ZM_BCS216090;
II:
a) contacting a sample comprising DNA from the corn plant, corn plant part, or corn seed with a first primer pair that can produce a first amplicon of all or part of the modified corn event ZM_BCS216090 and a second primer pair that can produce a second amplicon of a standard genomic sequence known to be single copy and homozygous in the corn plant, corn plant part, or corn seed;
b) contacting the sample with a first probe that specifically hybridizes to the first amplicon and/or all or part of the modified corn event ZM_BCS216090, and a second probe that specifically hybridizes to the standard genomic sequence;
c) performing a DNA amplification reaction using real-time PCR with the sample and determining the cycle thresholds (Ct values) of the first amplicon and the second amplicon;
d) calculating the difference (ΔCt) between the Ct values of the second amplicon and the first amplicon; and
e) determining the zygosity of the modified corn event ZM_BCS216090, wherein a ΔCt of about zero (0) indicates homozygosity of the modified corn event ZM_BCS216090 and a ΔCt of about one (1) indicates heterozygosity of the modified corn event ZM_BCS216090;
III:
a) contacting a sample comprising DNA from the corn plant, corn plant part, or corn seed with a primer pair capable of producing a first amplicon diagnostic for the modified corn event ZM_BCS216090 and a second amplicon diagnostic for native corn genomic DNA not comprising the modified corn event ZM_BCS216090;
b) performing a nucleic acid amplification reaction with the sample and the set of primer pairs; and
c) detecting the first amplicon and the second amplicon, wherein the presence of only the first amplicon is diagnostic of a corn plant, corn plant part, or corn seed homozygous for the modified corn event ZM_BCS216090, the presence of only the second amplicon is diagnostic of a corn plant, corn plant part, or corn seed homozygous for native corn genomic DNA not comprising the modified corn event ZM_BCS216090, and the presence of both the first amplicon and the second amplicon is diagnostic of a corn plant, corn plant part, or corn seed heterozygous for the modified corn event ZM_BCS216090; or
IV:
a) contacting a sample comprising DNA from the corn plant, corn plant part, or corn seed with a probe set which contains at least a first probe that specifically hybridizes to the modified corn event ZM_BCS216090 and at least a second probe that specifically hybridizes to corn genomic DNA that was disrupted by insertion of the heterologous DNA of corn event ZM_BCS216090 and is disrupted by the modified corn event ZM_BCS216090 DNA, wherein the second probe does not hybridize to the modified corn event ZM_BCS216090 DNA; and
b) hybridizing the probe set with the sample under stringent hybridization conditions, wherein detecting hybridization of only the first probe under the hybridization conditions is diagnostic for a corn plant, corn plant part, or corn seed homozygous for the modified corn event ZM_BCS216090, and wherein detecting hybridization of both the first probe and the second probe under the hybridization conditions is diagnostic for a corn plant, corn plant part, or corn seed heterozygous for the modified corn event ZM_BCS216090.

52. (canceled)

53. The method of claim 51, wherein the first and second primer pairs of II comprise SEQ ID NO: 11 combined with SEQ ID NO: 12, and SEQ ID NO: 14 combined with SEQ ID NO: 15.

54-55. (canceled)

56. The method of claim 51,
(i) wherein the probe set of IV comprises SEQ ID NO: 13 and SEQ ID NO: 16, and/or
(ii) wherein the modified corn event ZM_BCS216090 is a further modified corn event ZM_BCS216090.

57. (canceled)

58. A population of transgenic corn plants, wherein each transgenic corn plant comprises a modified corn event ZM_BCS216090.

59. The population of corn plants of claim 58, wherein said population of corn plants has a reduced plant height on average relative to a population of control corn plants lacking the modified corn event ZM_BCS216090.

60. The population of transgenic corn plants of claim 58, wherein the modified corn event ZM_BCS216090 is a further modified corn event ZM_BCS216090.

61. The population of corn plants of claim 58, wherein said population of corn plants has an increased lodging resistance on average relative to a population of control corn plants lacking the modified corn event ZM_BCS216090.

62. A method of modifying a corn plant, the method comprising:
(a) introducing a site-specific nuclease or a recombinant DNA construct comprising an expression cassette encoding a site-specific nuclease into at least one cell of an explant of a corn plant comprising corn event ZM_BCS216090, or a plant part thereof, to produce a modified corn event ZM_BCS216090 via a targeted genome editing technique; and
(b) developing or regenerating a modified corn plant from the explant, wherein the modified corn plant comprises the modified corn event ZM_BCS216090.

63. The method of claim 62,
(i)_wherein the modified corn event ZM_BCS216090 is a further modified corn event ZM_BCS216090,
(ii) wherein the site-specific nuclease is a zinc-finger nuclease (ZFN), a meganuclease, an RNA-guided endonuclease, a TALE-endonuclease (TALEN), a recombinase, or a transposase,
(iii) wherein the site-specific nuclease is an RNA-guided endonuclease or a CRISPR/Cas nuclease,
(iv) wherein the introducing step (a) comprises introducing the recombinant DNA construct into the at least one cell of the explant, and wherein the recombinant DNA construct further comprises an expression cassette encoding a first guide RNA (gRNA),
(v) wherein the recombinant DNA construct further comprises an expression cassette encoding a second guide RNA (gRNA),
(vi) wherein the introducing step (a) further comprises introducing a first guide RNA (gRNA) or a second recombinant DNA construct comprising an expression cassette encoding a first guide RNA (gRNA) into the at least one cell of the explant,
(vii) wherein the introducing step (a) comprises introducing at least two guide RNAs (gRNAs) comprising the first gRNA and a second gRNA into the at least one cell of the explant,
(viii) wherein the introducing step (a) further comprises introducing a second recombinant DNA construct that comprises an expression cassette encoding a second guide RNA (gRNA),
(ix) wherein the introducing step (a) comprises introducing a first gRNA and second recombinant DNA construct comprising an expression cassette encoding a second guide RNA (gRNA) into the at least one cell of the explant,
(x) wherein the site-specific nuclease has a first target site in the genome of the corn plant at or near corn event ZM_BCS216090,
(xi) wherein the site-specific nuclease has a second target site in the genome of the corn plant at or near corn event ZM_BCS216090,
(xii) wherein the introducing step (a) comprises introducing a second site-specific nuclease or a recombinant DNA construct comprising an expression cassette encoding a second site-specific nuclease into at least one cell of the explant, and wherein the second site-specific nuclease has a second target site in the genome of the corn plant at or near corn event ZM_BCS216090, and/or
(xiii) wherein the first gRNA has a first target site in a flanking DNA sequence, 5′ flank, 3′ flank, junction sequence, or insertion sequence of corn event ZM_BCS216090, or a complement thereof.

64-74. (canceled)

75. The method of claim 63, wherein the first gRNA has a first target site in a flanking DNA sequence, 5′ flank, 3′ flank, junction sequence, or insertion sequence of corn event ZM_BCS216090, or a complement thereof.

76. The method of claim 63, wherein the first gRNA has a first target site comprising a target sequence that is:
(i) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 17 or 19, or a complement thereof; or
(ii) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 18 or 20, or a complement thereof; or
(iii) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9, or a complement thereof.

77. The method of claim 76, wherein the first gRNA has a second target site comprising a target sequence that is:
(i) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 17 or 19, or a complement thereof; or
(ii) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 18 or 20, or a complement thereof; or
(iii) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9, or a complement thereof.

78. The method of claim 63,
(i) wherein the first gRNA has a first target site comprising a target sequence that is:
(1) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 17 or 19, or a complement thereof; or
(2) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 18 or 20, or a complement thereof; or
(3) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9, or a complement thereof; and
(ii) wherein the second gRNA has a second target site comprising a target sequence that is:
(1) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 17 or 19, or a complement thereof; or
(2) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: 18 or 20, or a complement thereof; or
(3) at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, or at least 50 consecutive nucleotides of SEQ ID NO: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9, or a complement thereof.

79. The method of claim 78, wherein the modified corn event ZM_BCS216090 comprises a deletion or excision of intervening genomic DNA between the first target site and the second target site, relative to the corn event ZM_BCS216090.

80. The method of claim 63, further comprising:
(c) selecting the modified corn plant comprising the modified corn event ZM_BCS216090, and
(d) sexually crossing the modified corn plant with itself or a second corn plant to produce one or more modified progeny corn plants.

81. A method of introducing a target site into a corn plant, the method comprising:
I:
(a) introducing a cognate target site into the corn event ZM_BCS216090 locus of at least one cell of a corn plant or corn plant part comprising the corn event ZM_BCS216090 or an explant thereof via a targeted genome editing technique, wherein the cognate target site is identical or similar to an originator target site for a site-specific nuclease present in the corn event ZM_BCS216090 locus, and
(b) developing or regenerating a modified corn plant comprising a modified corn event ZM_BCS216090 comprising the cognate target site; or
II:
(a) introducing a cognate target site into the corn event ZM_BCS216090 locus of at least one cell of a corn plant or corn plant part comprising the corn event ZM_BCS216090 or an explant thereof via a targeted genome editing technique to produce a modified corn event ZM_BCS216090 comprising the cognate target site, wherein the cognate target site is identical or similar to an originator target site for a site-specific nuclease present in the corn event ZM_BCS216090 locus, and
(b) introducing a site-specific nuclease or a recombinant DNA construct comprising an expression cassette encoding a site-specific nuclease into at least one cell of an explant of a modified corn plant comprising the modified corn event ZM_BCS216090, or a plant part thereof, to produce a further modified corn event ZM_BCS216090 via a targeted genome editing technique, wherein the target site of the site-specific nuclease includes the cognate target site and the originator target site; and
(c) developing or regenerating a second modified corn plant comprising the further modified corn event ZM_BCS216090.

82. The method of claim 81, further comprising:
(c) introducing a site-specific nuclease or a recombinant DNA construct comprising an expression cassette encoding a site-specific nuclease into at least one cell of an explant of a corn plant comprising the modified corn event ZM_BCS216090 or a plant part thereof, to produce a further modified corn event ZM_BCS216090 via a targeted genome editing technique, wherein the target site of the site-specific nuclease includes the cognate target site and the originator target site; and
(d) developing or regenerating a second modified corn plant comprising the further modified corn event ZM_BCS216090.

83. (canceled)

84. The method of claim 82, wherein the further modified corn event ZM_BCS216090 of the second modified corn plant comprises a deletion or excision of intervening genomic DNA between the originator target site and the cognate target site, relative to the corn event ZM_BCS216090 or modified corn event ZM_BCS216090.

85. The method of any one of claims 82-84, further comprising: selecting the second modified corn plant or a progeny plant of the second modified corn plant comprising the further modified corn event ZM_BCS216090, and sexually crossing the second modified corn plant or the progeny plant with itself or another corn plant to produce one or more modified progeny corn plants comprising the further modified corn event ZM_BCS216090.

86. The method of claim 81, wherein the modified corn event ZM_BCS216090 is a further modified corn event ZM_BCS216090.

87. A method of modifying an explant of a corn plant or plant part, the method comprising: introducing a site-specific nuclease or a recombinant DNA construct comprising an expression cassette encoding a site-specific nuclease into at least one cell of an explant of a corn plant or plant part comprising corn event ZM_BCS216090 to produce a modified corn event ZM_BCS216090 into the at least one cell of the explant.

88. The method of claim 87, wherein the modified corn event ZM_BCS216090 is a further modified corn event ZM_BCS216090.
